(19) United States
(12) Patent Application Publication
(10) Pub. No.: US 2024/0083839 A1
(43) Pub. Date: Mar. 14, 2024

(54) COMPOSITIONS AND METHODS FOR TREATING NEURODEGENERATIVE DISEASES AND DISORDERS

(71) Applicant: The Regents of the University of California, Oakland, CA (US)

(72) Inventors: Varghese John, Los Angeles, CA (US); Jesus Campagna, Playa Del Rey, CA (US); Michael E. Jung, Los Angeles, CA (US); Barbara Jagodzinska, Redwood City, CA (US)

(21) Appl. No.: 18/030,686

(22) PCT Filed: Oct. 6, 2021
(86) PCT No.: PCT/US21/53696

§ 371 Date: Apr. 6, 2023

Related U.S. Application Data
(60) Provisional application No. 63/088,732, filed on Oct. 7, 2020.

(51) Int. Cl. C07C 229/08; C07D 223/16; C07D 233/32; C07C 237/06; C07C 237/04; C07D 209/48; C07D 213/40; C07D 223/12; C07D 211/60; C07D 207/16; C07D 207/12; C07C 237/20; C07D 213/30; C07D 211/50; A61P 25/28

(52) U.S. Cl. CPC C07C 229/08; C07D 223/16; C07D 233/32; C07C 237/06; C07C 237/04; C07D 209/48; C07D 213/40; C07D 223/12; C07D 211/60; C07D 207/16; C07D 207/12; C07C 237/20; C07D 213/30; C07D 211/50; A61P 25/28; C07C 2602/10; C07C 2601/08; C07B 2200/07; C07C 2601/04; C07C 2601/14; C07C 2602/08

(57) ABSTRACT

The present disclosure relates to compounds that are capable of inhibiting PRMT8 and/or upregulating SirT1. The disclosure further relates to methods of treating neurodegenerative diseases and disorders (e.g., Alzheimer's disease).

### RELATED APPLICATIONS

This application claims the benefit of priority to U.S. Provisional Patent Application No. 63/088,732, filed on Oct. 7, 2020, the contents of which are fully incorporated by reference herein.

### STATEMENT OF GOVERNMENT SUPPORT

This invention was made with government support under Grant Number AG05138, awarded by the National Institutes of Health. The government has certain rights in the invention.

### BACKGROUND

Alzheimer's disease (AD), which currently affects ˜6 million Americans—a number that is predicted to increase to 14 million by 2050—is a progressive neurodegenerative disorder characterized by the presence of senile plaques composed mainly of amyloid μ-protein (Aβ), and the development of neurofibrillary tangles resulting from the hyper-phosphorylation of microtubule-stabilizing protein tau in brain tissue. In AD, impairment of cholinergic transmission starting in the nucleus basalis of Maynert contributes to cognitive decline, and thus has been a target for therapeutic intervention. The currently available FDA-approved treatments for AD are acetylcholinesterase inhibitors or antagonist of the NMDA receptor. The clinical benefits of these drugs are modest, temporary and do not specifically target the cellular mechanisms of AD including generation of neurotoxic Aβ, p-tau or apolipoprotein ε4 (ApoE4)-related changes that precipitate onset of the disease. Thus, there is an unmet ongoing need for new treatments of AD.

### SUMMARY OF THE INVENTION

In one aspect, the present disclosure provides compounds represented formula Ia, Ib, Ic, Id, or Ie or a pharmaceutically acceptable salt thereof:

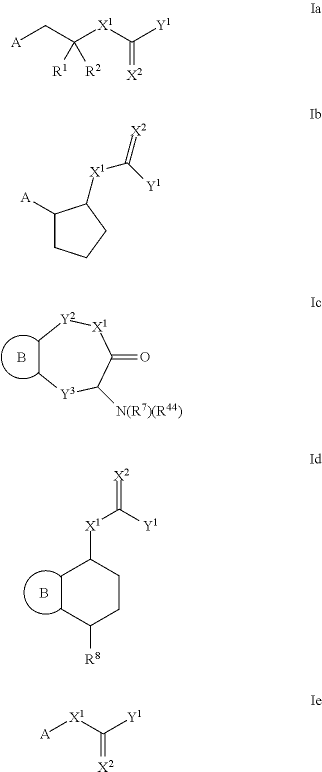

wherein
A and B are each independently, cycloalkyl, aryl, heteroaryl, or heterocyclyl;
X¹ is O, NR⁴¹, S, or C(R³)(R⁴);
X² is O, NR⁴², or S;
Y¹ is alkyl, aminoalkyl, amino, aminoaralkyl, or carbamate; or Y¹ combines with X¹ to form a heterocyclyl;
Y² is NR⁴³ or C(R⁵)(R⁶);
Y³ is a bond or C(R¹³)(R¹⁴);
R¹ and R² are each independently H, alkyl, or aralkyl; or R¹ and R² combine with the carbon that separates them to complete a cycloalkyl or heterocyclyl;
R³, R⁴, R⁵ R⁶, and R¹⁴ are each independently H, alkyl, or aryl;
R⁷ is H, alkyl, aralkyl, carabamate, or alkylacyl;
R⁸ is H, alkyl, cycloalkyl, aryl, heteroaryl, or heterocyclyl;
R¹³ is H, cycloalkyl, aryl, heteroaryl, or heterocyclyl; and
R⁴¹, R⁴², R⁴³, and R⁴⁴ are each independently H, alkyl, or aralkyl.

In another aspect, the present disclosure provides methods of treating neurodegenerative diseases and disorders.

### BRIEF DESCRIPTION OF THE DRAWINGS

FIG. 1 depicts how the expression of ApoE4 increases the risk for AD. ApoE4 affects cholesterol metabolism and the activity of the BACE enzyme, resulting increased production and reduced clearance of Aβ; ApoE4 is also associated with mitochondrial dysfunction and lysosomal leakage.

FIGS. 2A & B show that ApoE4 binds to the SirT1 promoter and reduces its activity.

FIG. 2A shows luciferase activity in SH-SY5Y cell lysates 24 hours after co-transfection with SIrT1-pGL3 reporter construct and ApoE isoforms (1:1) reveals both ApoE3 and 4 reduce SirT1 activity. FIB. 2B shows ChIP followed by PCR using SirT1-specific primers and probing with an anti-ApoE4 antibody showed ApoE4 binds to the SirT1 promoter in ApoE4-transfected SHSY5 cells.

FIG. 3 shows the structural differences between ApoE2, E3, & E4. A Cys-to-Arg substitution at aa 158 in ApoE2 makes it more stable than E3 or E4; the negative Cys at aa 112 in E2 & E3 reduces the likelihood of the domain interactions seen with E4.

FIGS. 4A-C shows the results of a HTS for SirT1 enhancers. FIG. 4A shows that the HTS in N2a-E4 cells of the UCLA compound library revealed SSRI & NMDA antagonist A03, but not SSRI fluoxetine or NMDA antagonist memantine increased SirT1. FIG. 4B shows that the SirT1 protein increased dose-response with A03 in N2a E4 cells. FIG. 4C shows that enantiomer 1 of A03 shows better activity than E2.

FIGS. 5A & B shows that A03 increases SirT1 and improves cognition. FIG. 5A shows that as a result of 56-day oral treatment of FAD-E4 mice at 40 mg/kg/day (20 mg/kg BID), A03 improved novel object recognition (NOR), increasing discrimination between familiar and new objects. FIG. 5B shows that the effects illustrated in FIG. 5A were accompanied by an increase in SirT1 in the hippocampi (Hip) of the A03-treated AD-E4 mice.

FIG. 6 shows PRMT8 in brain and arginine modulation by PRMT & PAD. The left panel depicts a northern blot analysis which shows that PRMT8 is a brain specific protein. The right panel shows that reactions catalyzed by PRMT and PAD on Arg could lead to alternations of ApoE4.

FIG. 7 shows A03 analog MP059 was conjugated to agarose beads which were then incubated with SH-SY5Y cell lysate. After purification and analysis, a large number of interacting protein were revealed.

FIG. 8 depicts a STRING analysis which suggests interaction between SirT1 and PRMT4. PRMT4 (CARM1) regulates the transcription of SirT1 and ReIA closely associated with SirT1. PRMT4 has strong sequence and functional homology to PRMT8 and PRMT5, which was found to be involved in A03 and its analogs' effects on ApoE4 binding to SirT1 promoter.

FIGS. 9A-C show that A03 inhibits PRMT9 activity but not protein levels. FIG. 9A shows that A03 inhibits PRMT8 but not 1 or 4 (MS23 control; a cell-free assay). FIG. 9B shows the dose-response curve for A03 PRMT8 inhibition. FIG. 9C shows that A03 does not reduce PRMT8 protein levels in E4-N2a cells at 10 μM.

FIGS. 10A & B depicts the interplay between PRMT8 inhibition and SirT1 enhancement.

FIG. 10A shows that A03 and analogs DDL209, 214 (MP48), and 216 show the greatest inhibition of PRMT8 at 0.25 μM. FIG. 10B show depicts that at a dose of 5 μM, linear regression analysis shows a correlation between cell-free PRMT8 inhibition and neuronal SirT1 enhancement.

FIGS. 11A-C show that PRMT8 knockdown increases SirT1. FIG. 11A shows blots for PRMT8, 1, and actin with scrambled or PRMT8 for 8 hour siRNA transfection. FIG. 11B shows that densitometry revealed that PRMT1 and 8 are knocked down with PRMT8 siRNA. FIG. 11C depicts that SirT1 alphaLISA shows SirT1 adjusted to protein is higher with PRMT8 siRNA compared to scrambled siRNA.

FIG. 12 shows that A03 decreases ApoE4 binding to SirT1 promotor. ChIP and real time PCR revealed 6 hour treatment of E4-N2a cells with A03 (50 μM) decreased ApoE4 and increased RNA polymerase binding to the SirT1 promoter as compared to DMSO control.

FIGS. 13A & B show the PRMT8-PRMT1 heterodimer and binding of A03 enantiomer at PRMT8 allosteric site. FIG. 13A shows the mode of PRMT8-PRMT1 interaction to form a heterodimer. FIG. 13B shows the modeled allosteric binding site for A03 enantiomer E1 on PRMT8.

FIG. 14 shows a putative model for SirT1 enhancement by A03 in the presence of ApoE4. ApoE4 binds to the SirT1 promoter, thereby reducing its expression. A03 interacts with PRMT8 and disrupts the PRMT8-PRMT1 heterodimer, thereby altering ApoE4 methylation and/or citrullination. As a result, efficient ApoE4 SirT1 promoter binding is prevented.

FIGS. 15A & B show that SirT1 is lower in E4-PS19 mice and E4 KI rats. FIG. 15A shows that SirT1 is significantly lower in the hippocampi of male E4 KI rats compared to age-matched SD rats. FIG. 15B shows that SirT1 is significantly lower in the hippocampi of E4-PS19 mice as compared to either PS19 or C57BI6J wildtype mice.

FIGS. 16A & B show N2a-E4 cells treatment and PK study of. FIG. 16A shows that MP-109 increases SirT1 in N2a-E4 cells. FIG. 16B shows the PK profile of MP-109 after oral administration.

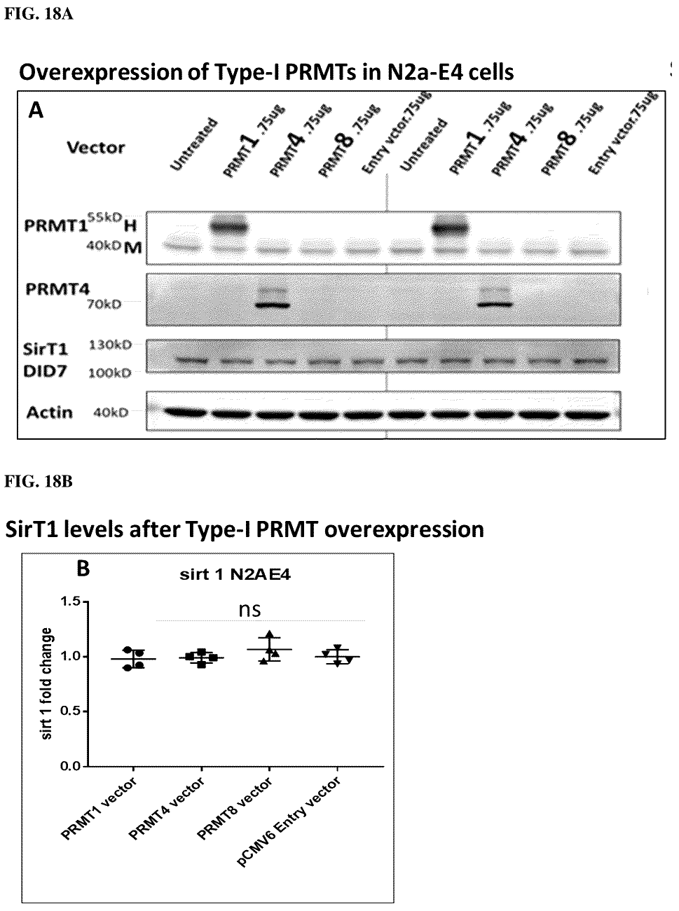

FIGS. 17 A & B show that the treatment of N2a-E4 cells resulted in decrease ApoE4 binding to the SirT1 promoter. FIG. 17A shows ChIP and real time PCR revealed 6 hour treatment of N2a-E4 cells with MP48 decreased ApoE4 and increased RNA polymerase binding to the SirT1 promoter as compared to DMSO control. FIG. 17B shows that the treatment of ApoE4:5×FAD-TR transgenic mice with MP48 resulted in increase SirT1 mRNA levels in the brain.

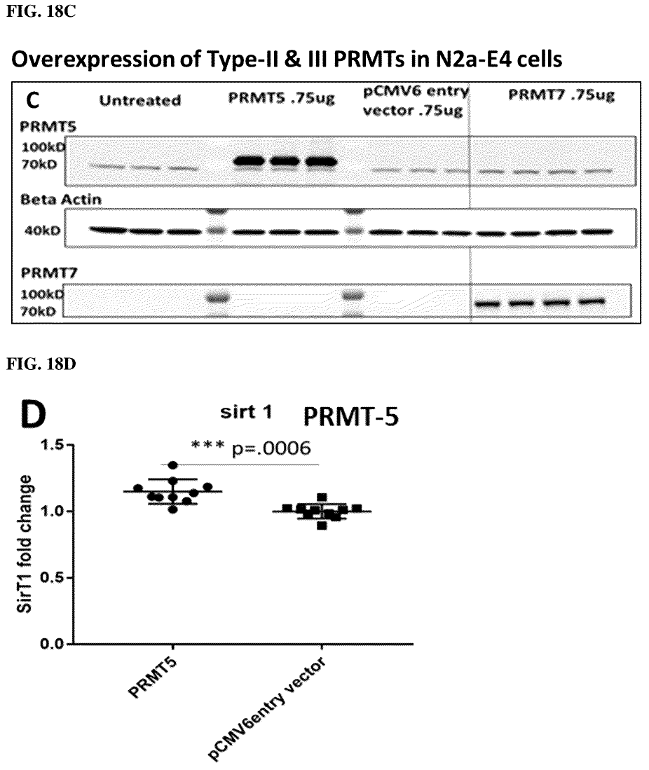

FIGS. 18A-E show that the overexpression of Type I PRMTs (PRMT-1, PRMT-4 and PRMT-8) in N2a-E4 cells do not result in increase in SirT1 levels. Overexpression of Type II PRMT (PRMT-5) results in highly significant increase SirT1 relative to pCMV vector. Overexpression of Type III PRMT (PRMT-7) results in small increase in SirT1 levels.

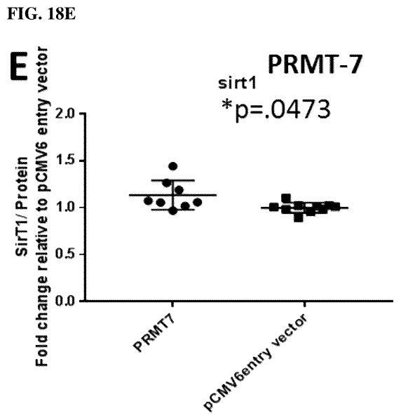

FIG. 19 shows that the overexpression of PRMT-5 in N2a-E4 cells resulted in decreased ApoE4 binding to the SirT1 promoter. ChIP and real time PCR revealed 6 h N2a-E4 cells with PRMT5 overexpression shows decreased ApoE4 and increased RNA polymerase binding to the SirT1 promoter as compared to pCMV control.

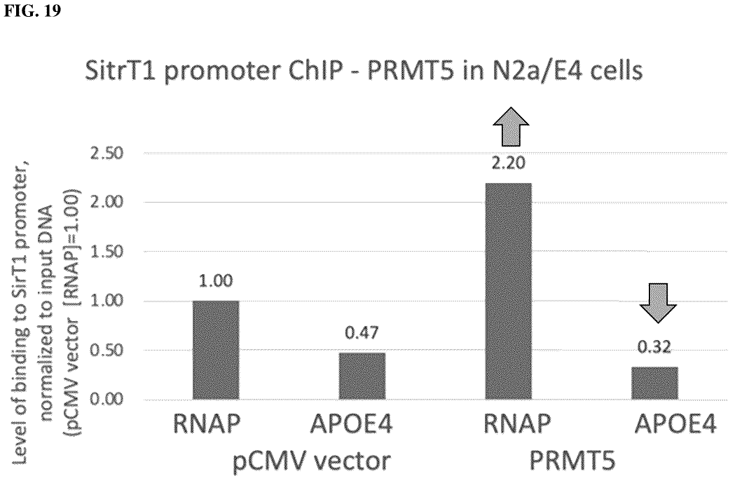

### DETAILED DESCRIPTION OF THE INVENTION

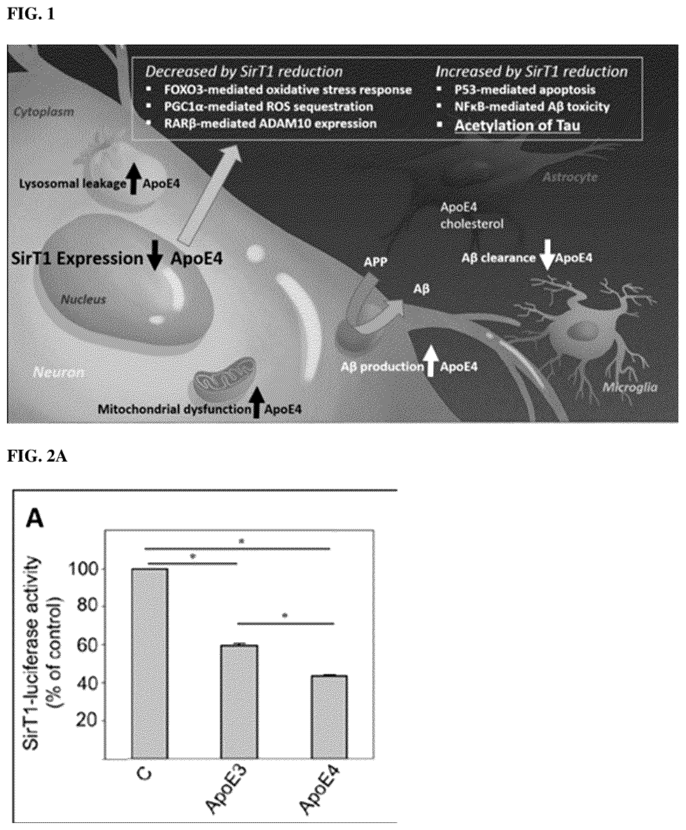

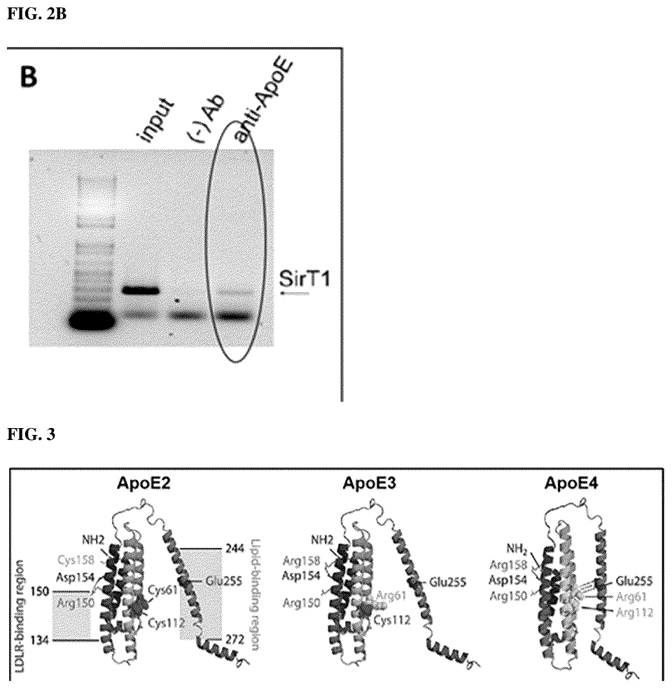

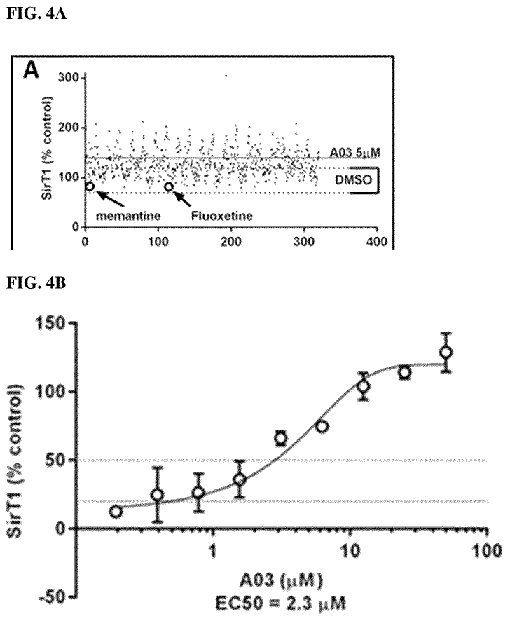

The dominant risk factor for sporadic, late-onset AD (LOAD) is ApoE4, which is present in about two-thirds of AD patients. Several mechanisms by which ApoE4 may increase AD risk have been reported, including increases in Aβ production, decreases in Aβ clearance, mitochondrial dysfunction and lysosomal leakage (FIG. 1). Studies herein have linked ApoE4 with major longevity determinants, the sirtuins; including binding of ApoE4 to the SirT1 promoter and a resultant decrease in expression of the tau deacetylase enzyme SirT1 as shown in FIG. 2. Disclosed herein are first candidate therapeutics that target this newly revealed mechanism for treatment of sporadic AD. Others have also reported a decrease in SirT1 expression in the presence of ApoE4. There are no published reports on the relationship of ApoE4 expression in humans and SirT1 levels, however the human SirT1 gene promoter does contain a putative binding site for ApoE4 (referred to as the CLEAR DNA sequence). In addition it has been reported that SirT1 is significantly lower in serum and post-mortem temporoparietal/parietal regions of AD brain compared to normal controls. These findings together explain why the ApoE e4 allele is the major susceptibility gene associated with AD and therefore should be considered a critical target for AD drug discovery.

Modulation of SirT1 expression is also an attractive target because decreases in SirT1 result in reduction of FOXO3-mediated oxidative stress response, PGC1α-mediated ROS sequestration, and RARβ-mediated ADAM10 expression; and, conversely, SirT1 decrease is associated with increases in p53-mediated apoptosis, NFκB-mediated Aβ toxicity, and the acetylation of tau (FIG. 1). The latter is particularly important, as increased tau acetylation is implicated, along with hyperphosphorylation, with the formation of the neurofibrillary tangles that are also characteristic of AD brain tissue and tied closely with cognitive decline.

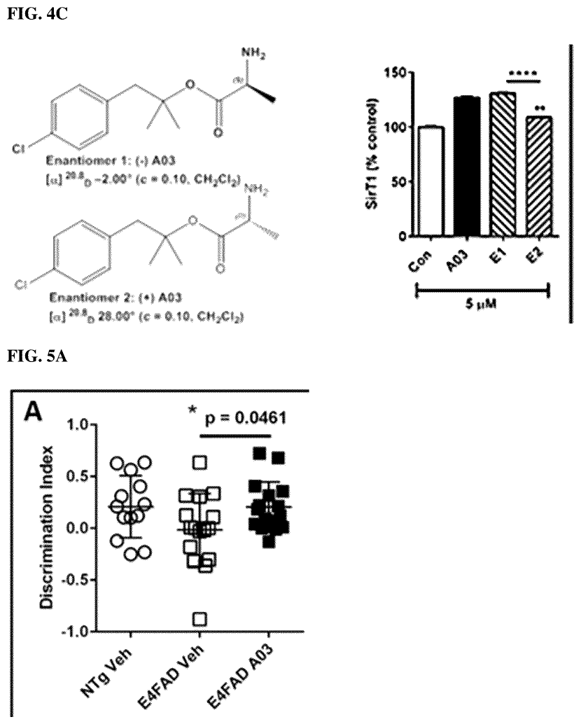

The increased risk for AD with expression of ApoE4 can be understood, in part, by its domain and structural differences from ApoE2 and ApoE3. As shown in FIG. 3, human ApoE contains amino- and carboxyl-terminal domains connected by a hinge region. The interaction of the domains is responsible for the preferential binding of very low-density lipoproteins by ApoE4, whereas ApoE2 and ApoE3 bind high-density lipoproteins. Arginine 61 (Arg 61) is critical for isoform preferences and is believed to interact with carboxyl terminus an acidic residue(s); this interaction is dependent upon the positive charge. A Cys-to-Arg substitution at amino acid 158 in ApoE2 makes it more stable than ApoE3 and ApoE4 and likely contributes to its protective effects. Key differences in ApoE4 vs ApoE3 or ApoE2 interactions are dependent on the carboxyl glutamic acid 255 residue and modifications such as acetylation/deacetylation may affect its domain interaction. There are 32 Arg residues in ApoE4 and it is known that Arg-112 is involved in ApoE4 binding to the CLEAR DNA sequence that is present in the human SirT1 promoter. In addition, ApoE4 has two GAR-motifs (residues Gly-31:Arg-32 and Gly-113:Arg-114); such motifs have been reported to be favored for methylation by PRMTs. The potential role of these residues in ApoE4 binding to the SirT1 promoter is not known and this question would be addressed in this grant.

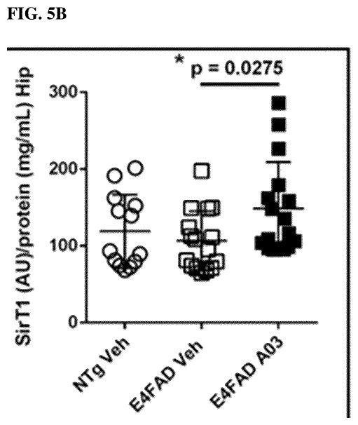

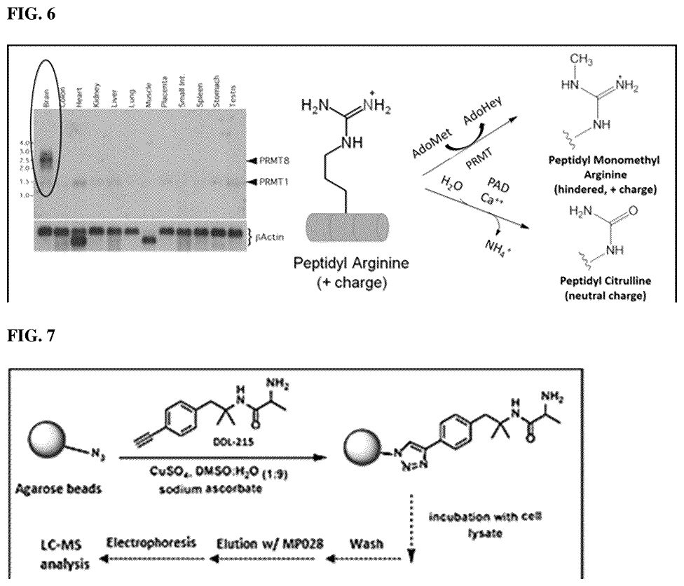

Prompted by the emerging importance of the associations between ApoE4 and the expression/protein levels of SirT1, high-throughput screening (HTS) of the UCLA compound library was performed using AlphaLISA to detect SirT1 levels in murine neuroblastoma cells stably transfected with ApoE4 (E4-N2a) to identify SirT1 enhancers. Screening revealed the hit A03 (alaproclate), a known potent selective serotonin reuptake inhibitory (SSRI) and weak N-methyl D-aspartyl (NMDA) receptor antagonist increased SirT1 in a dose dependent fashion with an EC₅₀˜2.3 μM. In HTS, other known SSRI fluoxetine and NMDA receptor antagonist memantine did not increase SirT1 (FIG. 4). A03 was also found to be efficacious in vivo, eliciting cognitive improvement and enhancing SirT1 in a murine ApoE4-expressing AD model. In the in vivo study, mice were treated orally at 40 mkd (20 mg/kg BID) for 56 days and underwent assessment of cognition in the Novel Object Recognition (NOR) testing paradigm. Mice that had received A03 had a significantly greater discrimination index than mice treated with vehicle. The performance of A03-treated E4-AD mice (FIG. 5A) improved to level of non-transgenic vehicle-treated mice. At the end of the study, mice were euthanized and brain regions dissected for SirT1 analysis; it was found that SirT1 was significantly higher in the hippocampi (Hip) of A03-treated E4-AD mice as compared to vehicle-treated E4-AD mice and furthermore mean SirT1 in A03-treated mice was higher than vehicle-treated NTg mice (FIG. 5B).

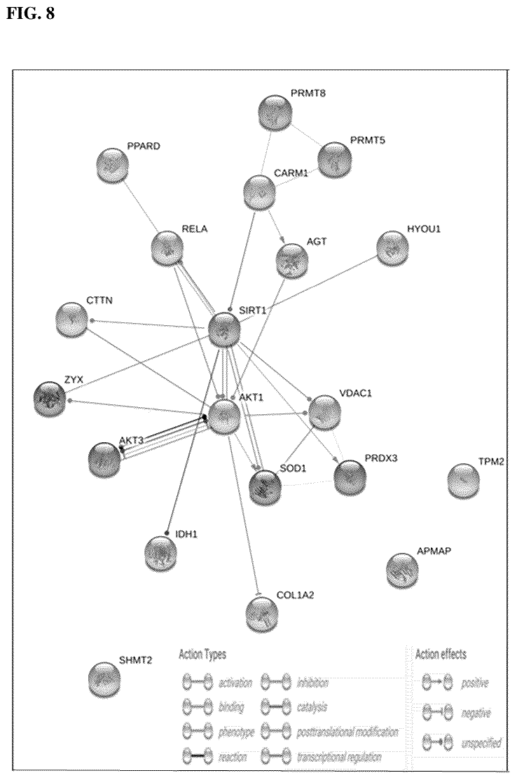

Mechanistic studies point to a member of the protein arginine methyltransferase (PRMT) family being involved as a target for A03/analogs. This is an intriguing finding as it has been reported that PRMT1 knockdown is associated with increase in SirT1 protein levels in retinal pigmental epithelial (RPE) cells. It was found that in neuronal cells, siRNA-induced knockdown of PRMT8 and PRMT1 is associated with an increase in SirT1 levels. PRMT8 is of particular interest because it is unique among PRMTs—which show high sequence homology and functional similarity—as it is expressed specifically in the brain (FIG. 6). It has also been reported that PRMT8 can be a membrane bound enzyme localized to the cytosolic compartment and is myristorylated at the N-terminus that anchors the enzyme to the membrane. The enzyme has high homology with PRMT1 and in neuronal cells the two enzymes can dimerize and exist as a transcriptional ‘rheostat’. Modulation of this dimer could result in inhibition of activity of these enzymes and transcriptional modulation or activation of other PRMTs such as CARM1 (PRMT4). PRMT4 is a known transcriptional activator of NF-kB signaling that preferentially methylates histones H3 that could reduce ApoE4 binding to the SirT1 promoter leading to enhancement of SirT1. Another transcriptional modulation could lead to activation of protein arginine deaminase (PAD) and citrullination of Arg in AD that in the case of ApoE4 could reduce binding to the SirT1 promoter and enhance SirT1 levels. In humans, there are five PAD isoforms and some PAD enzymes have been shown to be associated with AD. Mechanistic studies to be performed as part of the renewal grant have been designed to reveal if any such alterations in ApoE4 or its interactions with SirT1 promoter result from treatment with hits or analogs.

In one aspect, the present disclosure provides compounds represented formula Ia, Ib, Ic, Id, or Ie or a pharmaceutically acceptable salt thereof:

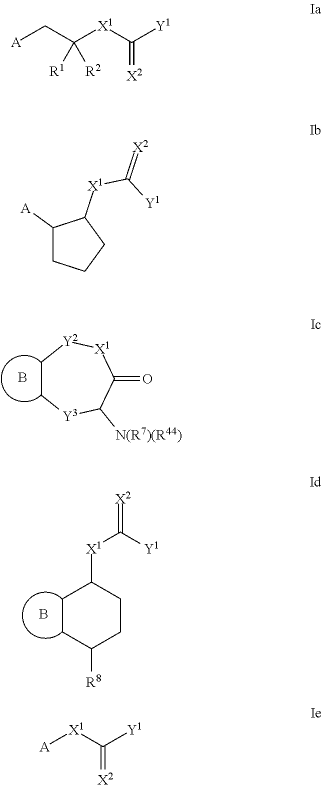

wherein
A and B are each independently, cycloalkyl, aryl, heteroaryl, or heterocyclyl;
X¹ is O, NR⁴¹, S, or C(R³)(R⁴);
X² is O, NR⁴², or S;
Y¹ is alkyl, aminoalkyl, amino, aminoaralkyl, or carbamate; or Y¹ combines with X¹ to form a heterocyclyl;
Y² is NR⁴³ or C(R⁵)(R⁶);
Y³ is a bond or C(R¹³)(R¹⁴);
R¹ and R² are each independently H, alkyl, or aralkyl; or R¹ and R² combine with the carbon that separates them to complete a cycloalkyl or heterocyclyl;
R³, R⁴, R⁵ R⁶, and R¹⁴ are each independently H, alkyl, or aryl;
R⁷ is H, alkyl, aralkyl, carabamate, or alkylacyl;
R⁸ is H, alkyl, cycloalkyl, aryl, heteroaryl, or heterocyclyl;
R¹³ is H, cycloalkyl, aryl, heteroaryl, or heterocyclyl; and
R⁴¹, R⁴², R⁴³, and R⁴⁴ are each independently H, alkyl, or aralkyl.

In certain embodiments, the compound of formula Ia, Ib, Ic, Id, or Ie is not

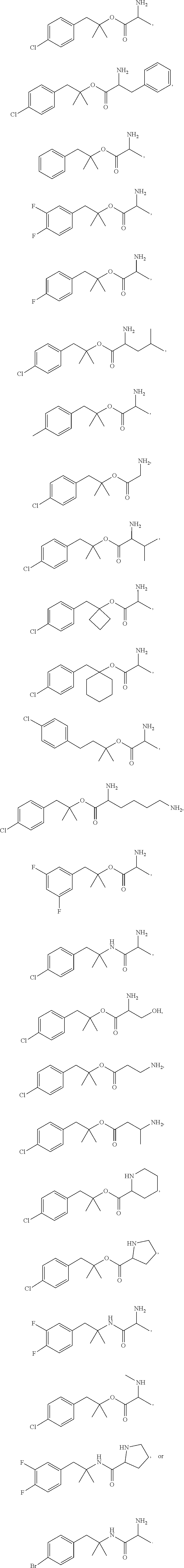

In other embodiments, the compound of formula Ia, Ib, Ic, Id, or Ie is not

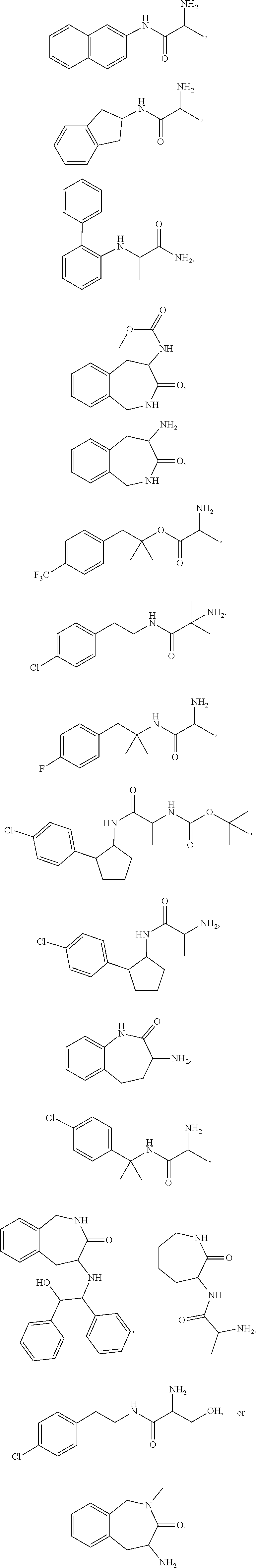

In certain embodiments, X¹ is 0. In other embodiments, X¹ is NR^{1′} and R^{1′} is H or alkyl. In yet other embodiments, X¹ is C(R³)(R⁴); R³ is alkyl (e.g., methyl); and R⁴ is H.

In certain embodiments, X² is O.

In certain embodiments, Y¹ is aminoalkyl (e.g., aminoethyl or amiopropyl), amino, carbamatealkyl (e.g., tert-butyl ethylcarbamate). In other embodiments, Y¹ combines with X¹ to form a heterocyclyl (e.g., imidazolidinyl). In certain embodiments, Y¹ is substituted with aryl (e.g., phenyl), carboxyalkyl, alkynylalkyl, or aminoalkylacyl.

In certain embodiments, the compound is represented by formula Ia, Ib, or Ie or a pharmaceutically acceptable salt thereof:

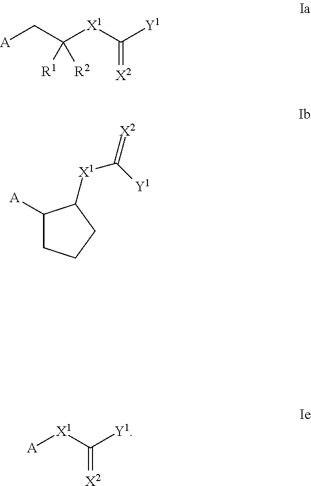

In certain embodiments of formulas Ia, Ib, or Ie, A is aryl (e.g., phenyl or naphthyl) or heteroaryl (e.g., pyridyl). In certain embodiments, A is substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, heterocyclylalkyl, heteroaralkyl, sulfonamide, aryl, heteroaryl, heterocyclyl, or aralkyl. In certain embodiments, A is substituted with halo (e.g., fluoro, chloro, or bromo), alkyl (e.g., trifluoromethyl or trifluoroethyl), alkynyl (e.g., ethynyl), acetyl, or aryl (e.g., phenyl or fluorophenyl).

In certain embodiments of formulas Ia, Ib, or Ie, R¹ and R² are both alkyl (e.g., methyl). In other embodiments, R¹ and R² are both H. In yet other embodiments, R¹ is alkyl (e.g., methyl) and R² is aryl (e.g., bromophenyl) or aralkyl (e.g., bromobenzyl or fluorobenzyl). In yet other embodiments, R¹ and R² combine to form a heterocyclyl (e.g., pyrrolidinyl or piperidinyl) or cycloalkyl (e.g., cyclopentyl).

In certain embodiments of formulas Ia and Ie, the compound is represented by formula IIa, IIb, IIc, or IIIa or a pharmaceutically acceptable salt thereof:

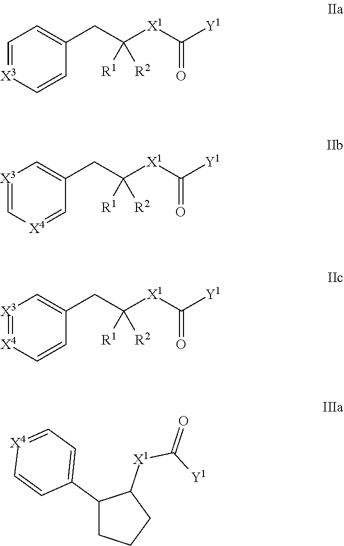

wherein,
each X³ is independently N or CR⁹
each X⁴ is each independently N or CR¹⁰; and
each R⁹ and R¹⁰ is selected from H, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, heterocyclylalkyl, heteroaralkyl, sulfonamide, aryl, heteroaryl, heterocyclyl, and aralkyl.

In certain embodiments, the compound is represented by formula IIa or a pharmaceutically acceptable salt thereof:

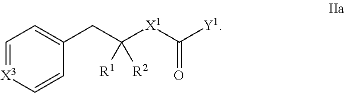

In certain embodiments, the compound is represented by formula IIa or a pharmaceutically acceptable salt thereof:

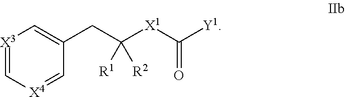

In certain embodiments, the compound is represented by formula IIc or a pharmaceutically acceptable salt thereof:

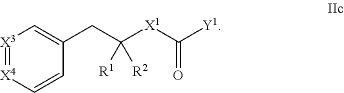

In certain embodiments of formulas IIa, IIb, and IIc, X³ is N. In other embodiments, X³ is CR⁹.

In certain embodiments, R⁹ is halo (e.g., fluoro, chloro, or bromo), alkyl (e.g., trifluoromethyl or trifluoroethyl), alkynyl (e.g., ethynyl), acetyl, or aryl (e.g., phenyl or fluorophenyl). In certain embodiments of formula Ib, the compound is represented by formula IIa or a pharmaceutically acceptable salt thereof:

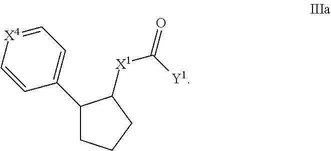

In certain embodiments of formulas IIa, IIb, IIc, or IIIa, X⁴ is N. In other embodiments, X⁴ is CR¹⁰. In certain embodiments, R¹⁰ is halo (e.g., fluoro, chloro, or bromo), alkyl (e.g., trifluoromethyl or trifluoroethyl), alkynyl (e.g., ethynyl), acetyl, or aryl (e.g., phenyl or fluorophenyl).

In certain embodiments, the compound is represented by formula Ia or Ib or a pharmaceutically acceptable salt thereof:

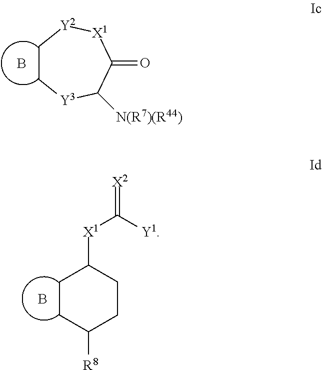

In certain embodiments of formulas Ic and Id, B is aryl (e.g., phenyl).

In certain embodiments of formulas Ic and Id, B is substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, heterocyclylalkyl, heteroaralkyl, sulfonamide, aryl, heteroaryl, heterocyclyl, or aralkyl.

In certain embodiments of formulas Ic and Id, the compound is represented by formula IVa or a pharmaceutically acceptable salt thereof:

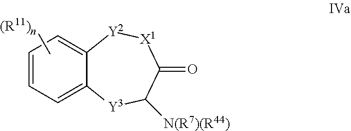

wherein,
each R¹¹ is independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, heterocyclylalkyl, heteroaralkyl, sulfonamide, aryl, heteroaryl, heterocyclyl, and aralkyl; and
n is 0, 1, 2, 3, or 4.

In certain embodiments of formula IVa, R¹¹ is halo (e.g., chloro).

In certain embodiments of formula IVa, n is 1. In other embodiments, n is 0.

In certain embodiments of formula IVa, R⁷ is H. In other embodiments, R⁷ is carbamate (e.g., alkylcarbamate or aralkylcarbamate).

In certain embodiments of formula IVa, R⁴ is H.

In certain embodiments of formula IVa, Y² is C(R⁵)(R⁶).

In certain embodiments of formula IVa, R⁵ is aryl (e.g., phenyl). In certain embodiments, R⁵ is substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, heterocyclylalkyl, heteroaralkyl, sulfonamide, aryl, heteroaryl, heterocyclyl, or aralkyl. In certain preferred embodiments, R⁵ is substituted with halo (e.g., chloro). In other embodiments, R⁵ is H.

In certain embodiments of formula IVa, R⁶ is H.

In certain embodiments of formula IVa, Y³ is a bond. In other embodiments, Y³ is a C(R¹³)(R¹⁴). In certain embodiments, R¹³ is aryl (e.g., phenyl). In certain embodiments, R¹³ is substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, heterocyclylalkyl, heteroaralkyl, sulfonamide, aryl, heteroaryl, heterocyclyl, or aralkyl. In certain preferred embodiments, R¹³ is substituted with halo (e.g., chloro). In other embodiments, R¹³ is H.

In certain embodiments of formula IVa, R¹⁴ is H.

In certain embodiments of formula Id, the compound is represented by formula Va or a pharmaceutically acceptable salt thereof:

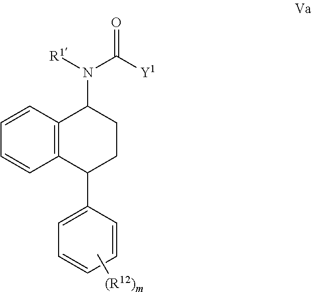

each R¹² is independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, cycloalkyl, heterocyclylalkyl, heteroaralkyl, sulfonamide, aryl, heteroaryl, heterocyclyl, and aralkyl; and
m is 0, 1, 2, 3, or 4.

In certain embodiments of formula Va, R¹² is halo (e.g., chloro). In certain embodiments, n is 2 and each R¹² is halo (e.g., chloro).

In certain embodiments of formulas IVa and Va, R^{1′} is H. In other embodiments, R^{1′} is alkyl (e.g., methyl).

In certain embodiments, the compound of formula Ia, Ib, Ic, Id, or Ie is selected from

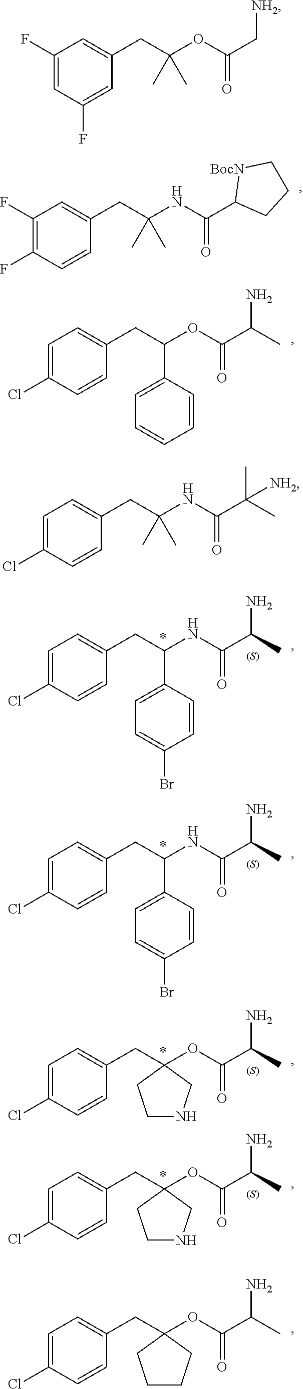

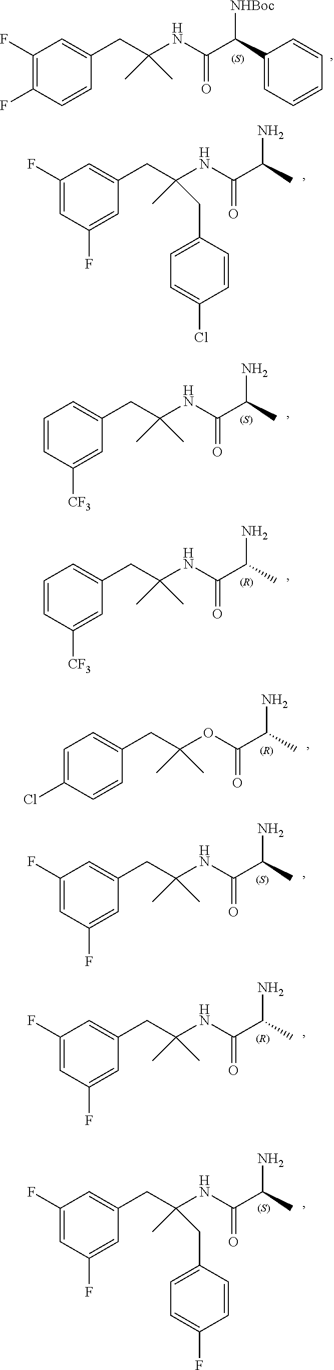

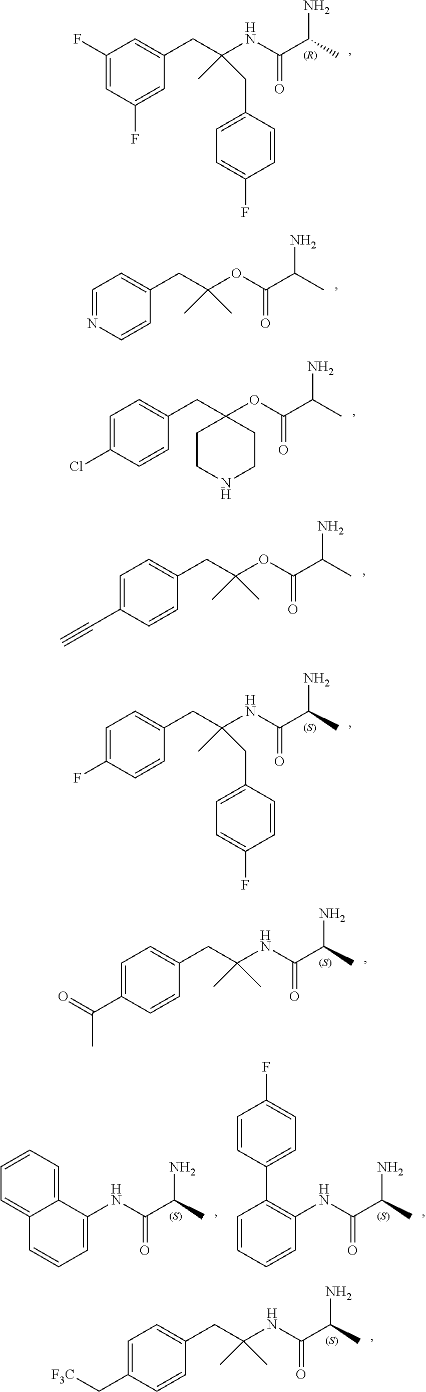

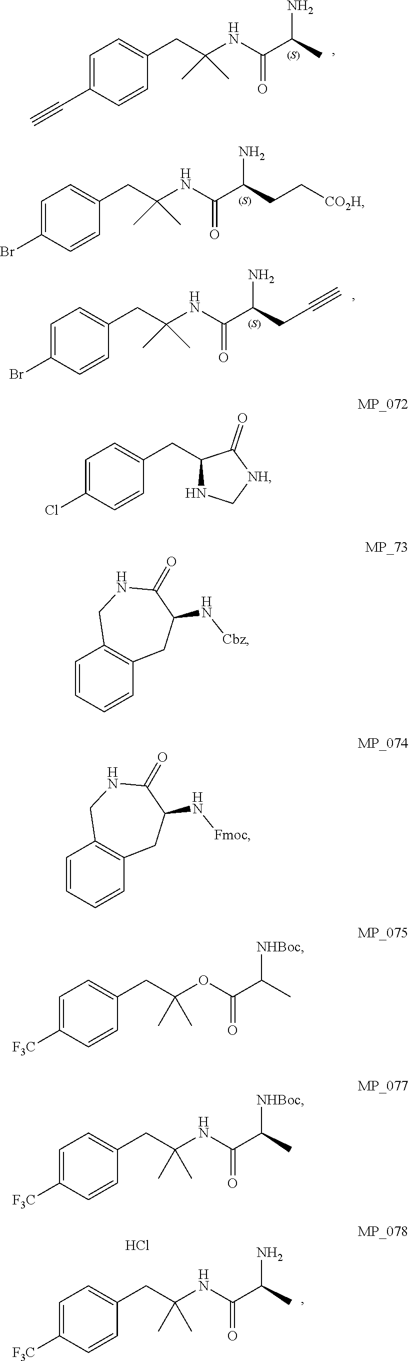

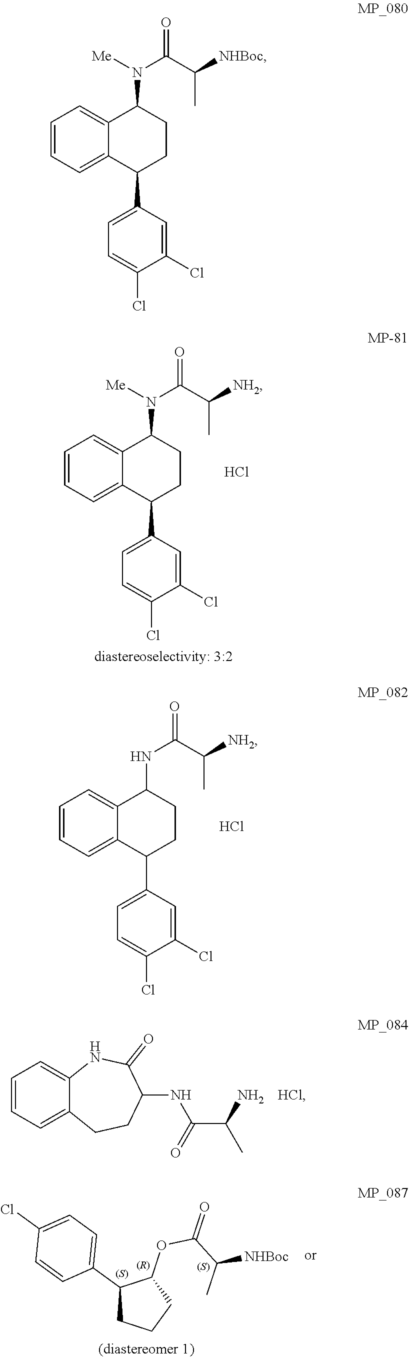

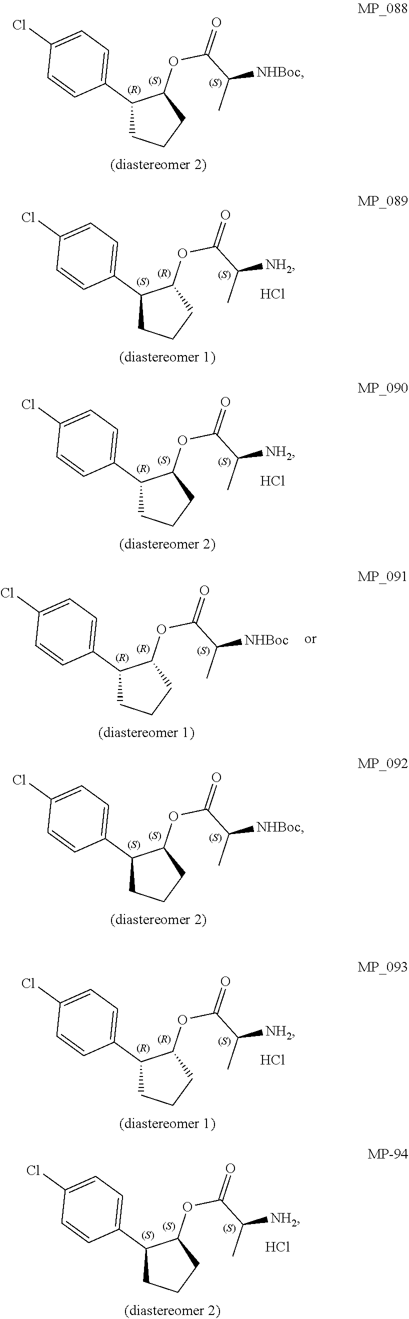

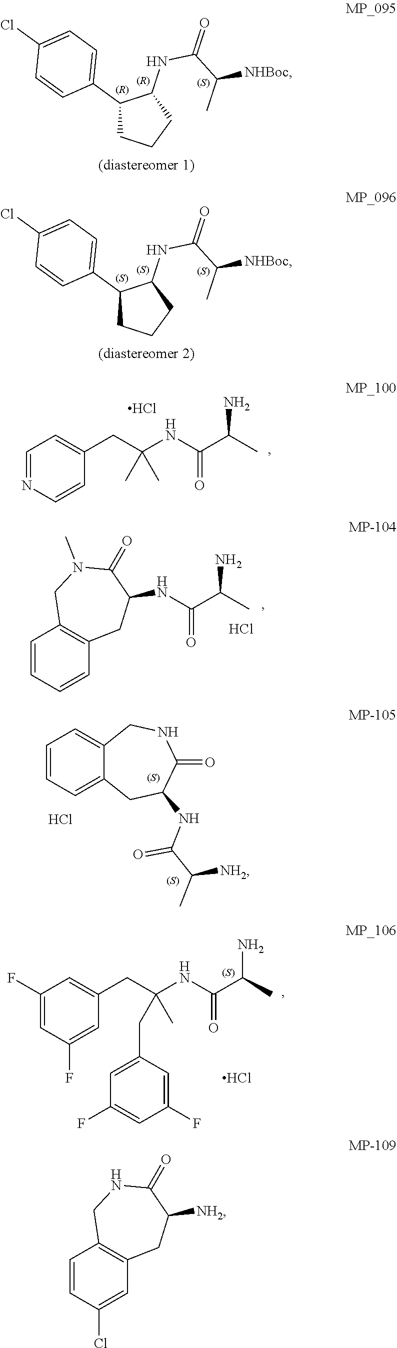

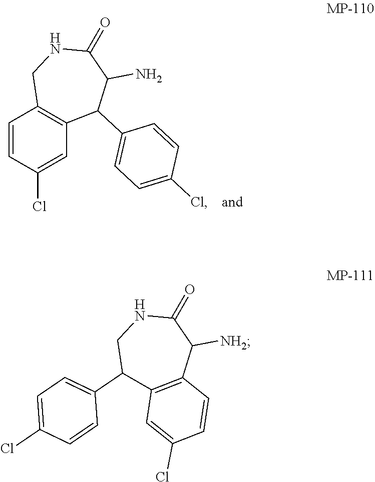 [SMILES: NC1C(=O)NCc2ccc(Cl)cc2C1c1ccc(Cl)cc1 ; "and" ; NC1C(=O)NCC(c2ccc(Cl)cc2)c2cc(Cl)ccc21]

or a pharmaceutically acceptable salt thereof.

In another aspect, the present disclosure provides a composition comprising a compound of the disclosure and a pharmaceutically acceptable excipient.

In yet another aspect, the present disclosure provides methods of treating a neurodegenerative disease or disorder in a subject in need thereof, comprising administering a compound of the disclosure or a pharmaceutically acceptable salt thereof to the subject.

In yet another aspect, the present disclosure provides methods of treating a neurodegenerative disease or disorder in a subject in need thereof, comprising administering a compound of the disclosure, or a pharmaceutically acceptable salt thereof, to the subject, wherein the compound is selected from

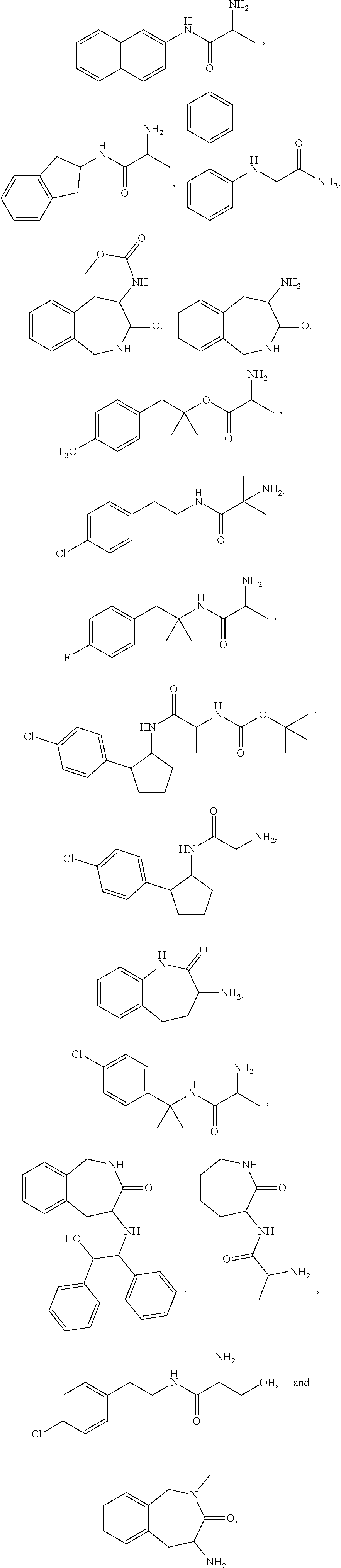

or a pharmaceutically acceptable salt thereof.

In certain embodiments, the neurodegenerative disease or disorder is associated with PRMT8. In certain embodiments, the neurodegenerative disease or disorder may be treated by inhibiting PRMT8. In certain embodiments, the neurodegenerative disease or disorder may be treated by upregulating SirT1. In certain embodiments, the neurodegenerative disease or disorder is Alzheimer's disease, Parkinson's disease, multiple sclerosis, stroke, amyotrophic lateral sclerosis, cerebellar ataxia, frontotemporal dementia, prion disease, Huntington's Disease, cerebral ischaemia, idiopathic Morbus Parkinson, Parkinson syndrome, Morbus Alzheimers, cerebral dementia syndrome, infection-induced neurodegeneration disorders, AIDS-encephalopathy, Creutzfeld-Jakob disease, encephalopathies induced by rubiola and herpes viruses and borrelioses, metabolic-toxic neurodegenerative disorders, hepatic-, alcoholic-, hypoxic-, hypo- or hyperglycemically-induced encephalopathies, encephalopathies induced by solvents or pharmaceuticals, degenerative retina disorders, trauma-induced brain damage, cerebral hyperexcitability symptoms, cerebral hyperexcitability states, neurodegenerative syndromes of the peripheral nervous system, peripheral nerve injury, or spinal cord injury. In certain embodiments, the neurodegenerative disease or disorder is Alzheimer's disease, Parkinson's disease, Huntington's disease, Lewy body dementia, frontotemporal dementia, amyotrophic lateral sclerosis, multiple sclerosis, progressive supranuclear palsy, or age related cognitive decline. In certain preferred embodiments, the neurodegenerative disease or disorder is Alzheimer's disease.

In yet another aspect, the present disclosure provides methods of treating a neurodegenerative disease or disorder in a subject in need thereof, comprising administering an inhibitor of PRMT8 to the subject. In certain embodiments, the PRMT8 inhibitor is a compound of the disclosure. In other embodiments, the PRMT8 inhibitor is selected from

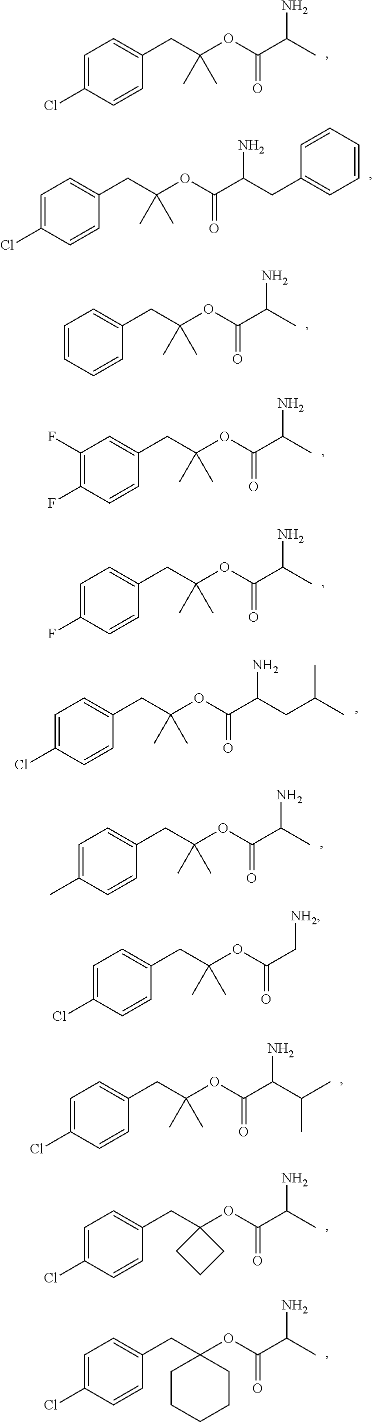

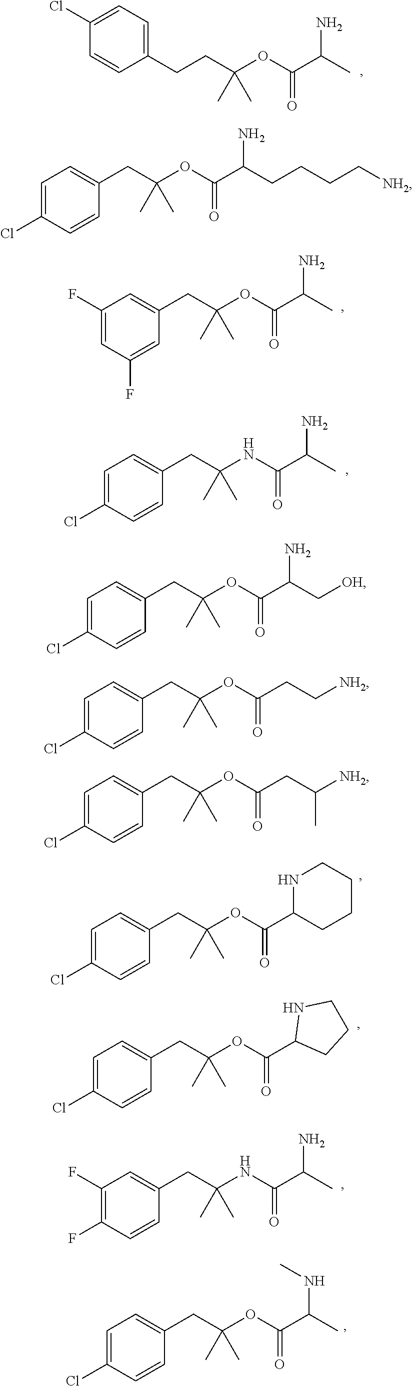

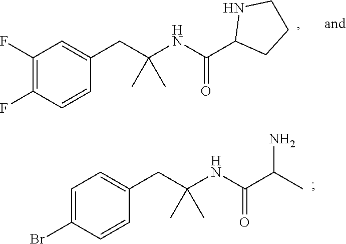 [SMILES: CC(C)(Cc1ccc(F)c(F)c1)NC(=O)C1CCCN1 ; CC(N)C(=O)NC(C)(C)Cc1ccc(Br)cc1]

or a pharmaceutically acceptable salt thereof. In yet other embodiments, the PRMT8 inhibitor is selected from

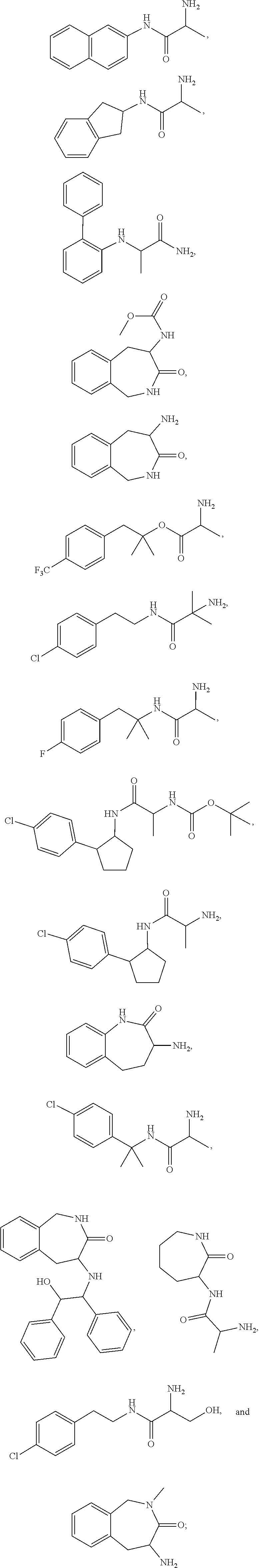

or a pharmaceutically acceptable salt thereof.

In certain preferred embodiments, the neurodegenerative disease or disorder is Alzheimer's disease.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier.

Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

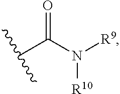

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

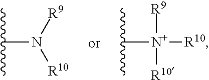

wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

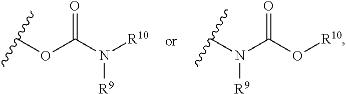

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic.

Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

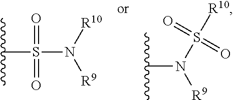

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

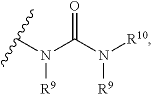

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “LogS” or “logS” as used herein is used in the art to quantify the aqueous solubility of a compound. The aqueous solubility of a compound significantly affects its absorption and distribution characteristics. A low solubility often goes along with a poor absorption. LogS value is a unit stripped logarithm (base 10) of the solubility measured in mol/liter.

### EXAMPLES

The invention now being generally described, it will be more readily understood by reference to the following examples which are included merely for purposes of illustration of certain aspects and embodiments of the present invention, and are not intended to limit the invention.

### Example 1: Preparation of Exemplary Compounds

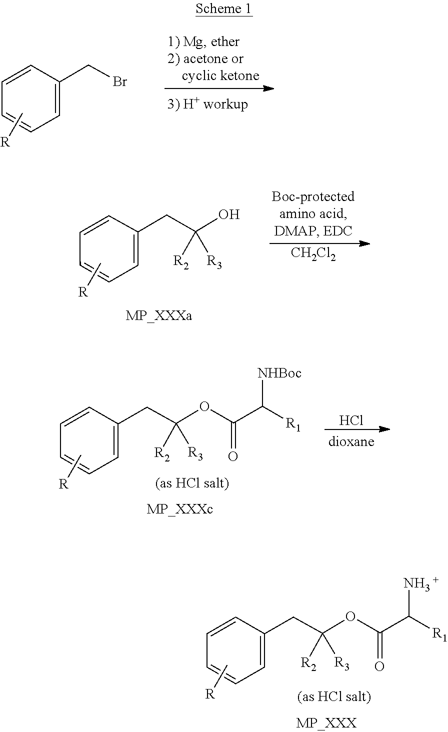

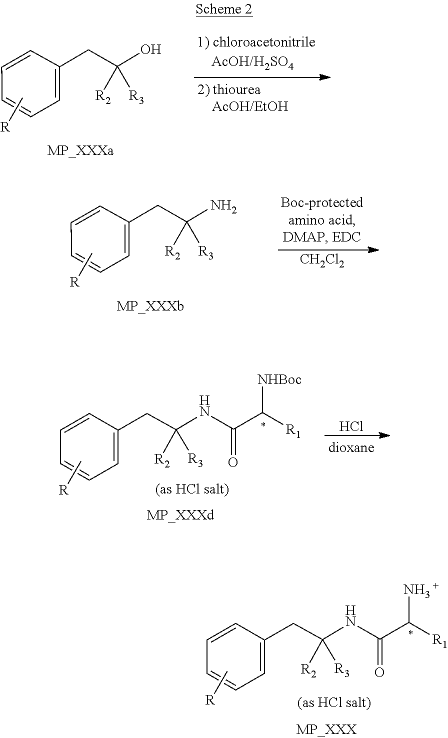

### Tertiary Alcohols (MP_XXXa); General Procedure

A dry 3-neck reaction flask equipped with a stir bar and reflux condenser was charged with magnesium turnings (22.6 mmol) and anhydrous Et₂O (20 mL). A solution of the alkyl bromide (15.1 mmol) in Et₂O (5 mL) was added dropwise from a pressure-equalizing additional funnel under argon dropwise for 10 min. The reaction was allowed to stir at room temperature for 30 min and became cloudy before refluxing for an additional 1 hr. The solution was then cooled to 0° C., and the ketone (18.1 mmol) was added dropwise in a span of 10 min. The solution was allowed to warm to room temperature and allowed to stir for 2 hours. The reaction mixture was quenched by addition of 20% aqueous ammonium chloride, and the organic layer was separated and the aqueous layer was extracted with diethyl ether (25 mL×2). Combined organic layers were washed with saturated sodium bicarbonate (50 mL) and brine (50 mL), dried over MgSO₄ and the solvent was evaporated under reduced pressure to afford the crude product. The tertiary alcohol was isolated by flash chromatography (EtOAc/Hexane=1:6).

### tert-Alkylamines (MP_XXXb); General Procedure

In a round bottom flask equipped with a stir bar, the tertiary alcohol (2.6 mmol) and was added 0.5 mL acetic acid and chloroacetonitrile (5.2 mmol) and allowed to stir at 0° C. The mixture was added 0.5 mL of H₂SO₄ (drop-wise). The solution was allowed to reach room temperature and stiffed for an additional 6 hr. The reaction mixture was poured into 10 mL of ice water and extracted with Et₂O (20 mL×2). The combined organic layers were washed with saturated sodium bicarbonate (40 mL) and brine (40 mL), dried over MgSO₄ and the solvent was evaporated under reduced pressure to afford the crude chloroacetamide product. The crude product was dried under high vacuum overnight and used for the next step without purification.

In a round bottom flask equipped with a stir bar and reflux condenser, the chloroacetamide (2.5 mmol) was added thiourea (3 mmol) along with 7 mL of ethanol and 1.5 mL of acetic acid. The reaction mixture was allowed to stir at reflux for 10 hr. The reaction mixture was cooled and poured into 40 mL of cold water. The solution was made alkaline with 4 M NaOH (pH˜11) and extracted with Et₂O (40 mL×2). The combined organic layers were washed with saturated sodium bicarbonate (80 mL) and brine (80 mL), dried over MgSO₄ and the solvent was evaporated under reduced pressure to afford the crude amine product. The crude amine product was placed under high vacuum overnight to remove any residual solvent. The crude amine product was used for the next step without purification.

### Boc Protected Esters (MP_XXXc); General Procedure

In a round bottom flask equipped with a stir bar, the tertiary alcohol (1.5 mmol) was dissolved with an appropriate amount of dichloromethane to give 2M concentration of the alcohol. The solution was added 4-dimethylaminopyridine (DMAP, 1.5 mmol) and the Boc-protected amino acid (3 mmol). The solution was allowed to stir for 10 min at 0° C. before adding N-(3-dimethylaminopropyl)-N′-ethylcarbodiimide hydrochloride (EDC HCl), 3 mmol). The reaction mixture was allowed to warm to room temperature and stirred overnight. The solution was concentrated under vacuum, and the product was isolated by flash chromatography (EtOAc/Hexanes=1:6).

### Boc-Protected Amides (MP_XXXd); General Procedure

In a round bottom flask equipped with a stir bar, the tertiary amine (1.5 mmol) was dissolved with an appropriate amount of dichloromethane to give 2M concentration of the alcohol. The solution was added 4-dimethylaminopyridine (DMAP, 0.2 mmol) and the Boc-protected amino acid (3 mmol). The solution was allowed to stir for 10 min on ice before adding N-(3-dimethylaminopropyl)-N′-ethylcarbodiimide hydrochloride (EDC HCl), 3 mmol). The reaction mixture was allowed to stir at 0° C. for 1 hr, and then warmed to room temperature to stir for an additional 2 hr. The solution was concentrated under vacuum, and the product was isolated by flash chromatography (EtOAc/Hexanes=1:6).

### Boc Deprotection and Generation of Compounds (MP_XXX); General Procedure

In a round bottom flask equipped with a stir bar, the N-Boc protected esters and amides (0.5 mmol) were cooled to 0° C. degrees and was added 1.25 mL of 4M HCl in dioxane and allowed to stir on ice for 1 hr. The solution was allowed to stir for an additional 1 hr at room temperature. The solution was concentrated under vacuum, and was added ether or hexanes. The hydrochloride salt was allowed to precipitate, and the product was filtered and dried. The hydrochloride salt product could also be triturated with diethyl ether. In the case where the hydrochloride salt product dissolves in ether, the ether was evaporated, and the product was added hexanes and sonicated for 10 min before decanting the hexane solvent, and dried under high vacuum.

### Synthesis of Ester Alkyne MP_050 by Sonogashira Coupling

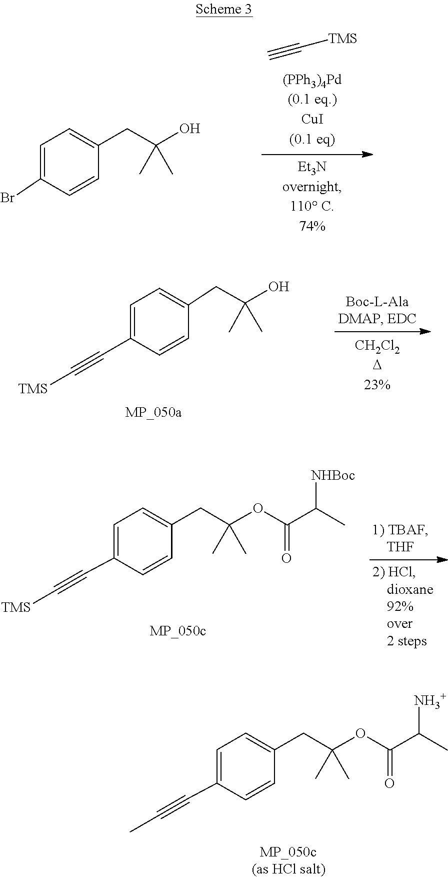

### Installing TMS-Alkyne

MP_050a: 2-methyl-1-(4-((trimethylsilyl)ethynyl)phenyl)propan-2-ol: In a 100 mL round bottom flask, 51 mg of CuI (0.27 mmol) and 151 mg of (PPh₃)PdCl₂ (0.21 mmol) was added and the flask was degassed and purged 3× with Ar gas. 501 mg of 1-(4-bromophenyl)-2-methylpropan-2-ol (2.2 mmol) in 8 mL of dry triethylamine was slowly added to the flask. Additional dry triethylamine (14 mL) was added to the reaction solution and stirred for 5 min. Trimethylsilylacetylene (0.39 mL, 2.8 mmol) was then added drop-wise to the reaction solution in a span of 1 hr. The reaction was stirred under Ar at room temperature for 1 hr then refluxed at 105° C. for 6 hr. The solution was allowed to cool and was then filtered through a bed of celite and concentrated under vacuum to give MP_50a as a yellow/brown oil. The product was isolated by flash chromatography (EtOAc/Hexanes=1:10 to 1:5) to give an off-yellow oil (294 mg, 59% yield). The 2-methyl-1-(4-((trimethylsilyl)ethynyl)phenyl)propan-2-ol was coupled with Boc-Ala-OH using the general procedure for synthesis of Boc-protected esters to give MP_50c.

### TMS Deprotection

In a 10 mL round bottom flask charged with 71 mg of 2-methyl-1-(4-((trimethylsilyl)ethynyl)phenyl)propan-2-yl (tert-butoxycarbonyl)alaninate (0.17 mmol), 1 mL dry THF was added and stirred at 0° C. for 3-5 min. Then 0.21 mL of TBAF (1M in THF) was drop-wise added to the solution at 0° C. The reaction solution was stirred under Ar at 0° C. for 10 min, then allowed to warm to room temperature and stirred an additional 40 min. At the end of the reaction, 50 mL H₂O was added and the organic product was extracted with ethyl acetate (3×). The organic layers were combined and washed with brine, dried with MgSO₄, and filtered. The organic solution was concentrated under vacuum to give yellow oil. The crude product was purified by flash chromography (EtOAc/Hexanes 1:7 then 1:5) to give 54 mg of the product. The Boc protected alkyne was carried out as described in the Boc deprotection procedure to give MP_050 (92% over two steps).

### Synthesis of TMS-Protected Alkyne Ester MP_059 by Sonogashira Coupling

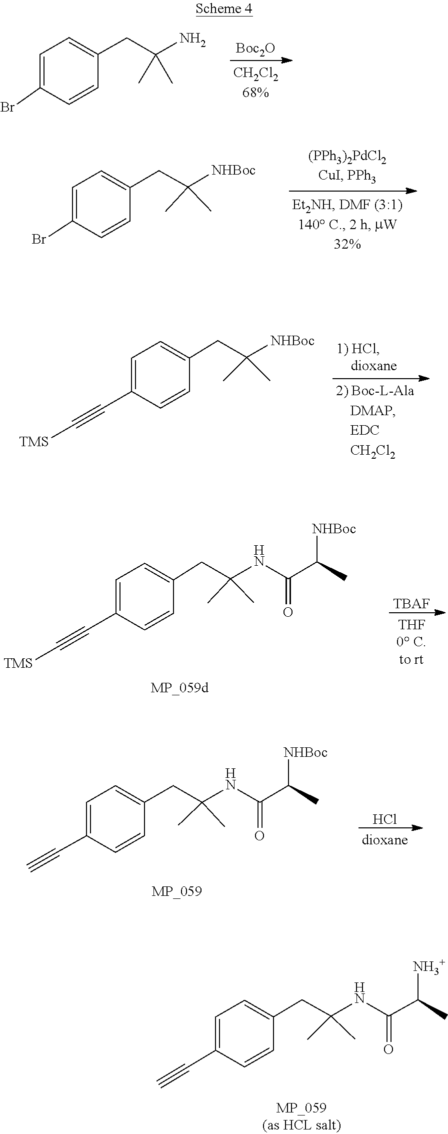

Boc protection of amine: A 25 mL round bottom flask charged with 308 mg (1.4 mmol) of crude 1-(4-bromophenyl)-2-methylpropan-2-amine was dissolved in 3 mL CH₂Cl₂ and stirred under Ar for 5 min. Boc₂O (320 mg, 1.5 mmol) was added dropwise to the solution and allowed to stir overnight. Imidazole (30 mg) was added to the reaction solution and stirred for 20 min. Aqueous HCl was added to the solution and extracted with diethyl ether (3×). The organics were combined, washed with brine and dried with MgSO₄. The crude product was purified by flash chromatography (EtOAc/Hexanes 1:10) to give a solid (300 mg, 68% yield).

### Synthesis of TMS-Protected Alkyne Amide MP_059 by Sonogashira Coupling

Sonogashira coupling: tert-butyl (2-methyl-1-(4-((trimethylsilyl)ethynyl)phenyl)propan-2-yl)carbamate: In a 25 mL, thick-walled reaction tube, 475 mg (1.5 mmol) tert-butyl (1-(4-bromophenyl)-2-methylpropan-2-yl)carbamate was dissolved with 3.75 mL dry diethylamine and 1.25 mL DMF. After dissolving the carbamate, 110 mg (PPh₃)₂PdCl₂ (0.1 mol equiv., 1.5 mmol), 63 mg CuI (0.22 mol equiv., 0.33 mmol), 83 mg (0.32 mmol) PPh₃ and 0.25 mL (1.2 mol equiv., 1.7 mmol) trimethylsilylacetylene were added immediately after and capped with a stir bar. The reaction was heated at 120-140° C. with stirring for 6 hours in a microwave. The reaction was filtered through a bed of celite, washed with CH₂Cl₂, concentrated under vacuum, and purified by flash chromatography (EtOAc/Hexanes 0:100 to 1/10) to give 159 mg of product (32% yield).

MP_059: Boc deprotection and amino acid coupling were followed according to the general procedures, and TBAF deprotection in the synthesis of MP_050 was carried out similarly for MP_059.

### NMR

### Alcohols

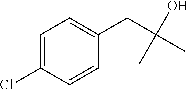

MP_001a (and other 4-chorophenylderivatives): 1-(4-chlorophenyl)-2-methylpropan-2-ol: ¹H NMR (500 MHz, CDCl₃) δ 7.27 (d, 2H, J=8.0 Hz), 7.15 (d, 2H, J=8.0 Hz), 2.73 (s, 2H), 1.28 (s, 1H), 1.21 (s, 6H).

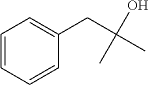

MP_002a: 2-methyl-1-phenylpropan-2-ol: ¹H NMR (400 MHz, CDCl₃) δ 7.33-7.21 (m, 6H), 2.77 (s, 2H), 1.36 (s, 1H), 1.28 (H, s), 1.23 (s, 6H).

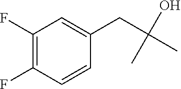

MP_003a (and other 3,3-difluorophenylderivatives): 1-(3,4-difluorophenyl)-2-methylpropan-2-ol: ¹H NMR (400 MHz, CDCl₃) δ 7.12-7.03 (m, 2H), 6.92 (m, 1H), 2.71 (s, 2H), 1.26 (s, 1H), 1.22 (s, 1H).

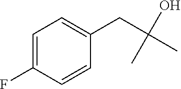

MP_004a: 1-(4-fluorophenyl)-2-methylpropan-2-ol: ¹H NMR (400 MHz, CDCl₃) δ 7.18 (m, 2H), 6.99 (m, 2H), 2.73 (s, 2H), 1.28 (s, 1H), 1.22 (s, 6H).

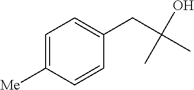

MP_006a: 2-methyl-1-p-tolylpropan-2-ol: ¹H NMR (400 MHz, CDCl₃) δ 7.13 (d, 2H, J=8.3 Hz), 7.10 (d, 2H, J=8.3 Hz), 2.73 (s, 2H), 2.34 (s, 3H), 1.36 (s, 1H), 1.22 (s, 6H).

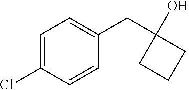 [SMILES: OC1(Cc2ccc(Cl)cc2)CCC1]

MP_009a: 1-(4-chlorobenzyl)cyclobutanol: ¹H NMR (400 MHz, CDCl₃) δ 7.28 (d, 2H, J=8.4 Hz), 7.19 (d, 2H, J=8.4 Hz), 2.87 (s, 2H), 2.18-2.11 (m, 2H), 2.03-1.95 (m, 2H), 1.81 (m, 1H), 1.64 (m, 1H), 1.59 (m, 1H).

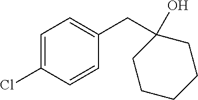 [SMILES: OC1(Cc2ccc(Cl)cc2)CCCCC1]

MP_010a: 1-(4-chlorobenzyl)cyclohexanol: ¹H NMR (400 MHz, CDCl₃) δ 7.26 (d, 2H, J=8.4 Hz), 7.14 (d, 2H, J=8.4 Hz), 2.71 (s, 2H), 1.89-1.41 (m, 10H).

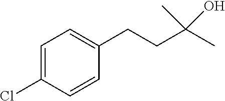

MP_012a: 4-(4-chlorophenyl)-2-methylbutan-2-ol: ¹H NMR (400 MHz, CDCl₃) δ 7.24 (d, 2H, J=8.4 Hz), 7.12 (d, 2H, J=8.4 Hz), 2.67 (m, 2H), 1.75 (m, 2H), 1.28 (s, 6H), 1.22 (bs, 1H).

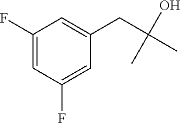

MP_014a (and other 3,5-difluorphenyl derivatives): 1-(3,5-difluorophenyl)-2-methylpropan-2-ol: ¹H NMR (400 MHz, CDCl₃) δ 6.72 (m, 3H), 2.74 (s, 2H), 1.30 (s, 1H), 1.23 (s, 6H).

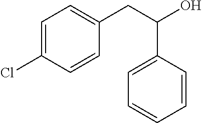 [SMILES: OC(Cc1ccc(Cl)cc1)c1ccccc1]

MP_026a: 2-(4-chlorophenyl)-1-phenylethan-1-ol: ¹H NMR (400 MHz, CDCl₃) δ 7.36-7.24 (m, 7H), 7.09 (d, 2H, J=8.3 Hz), 4.87 (td, 1H, J=6.6 Hz, 2.9 Hz), 2.99 (d, 2H, J=6.6 Hz), 1.88 (d, 2H, J=3.0 Hz).

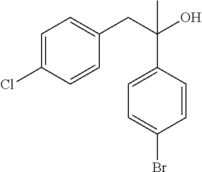 [SMILES: CC(O)(Cc1ccc(Cl)cc1)c1ccc(Br)cc1]

MP_030a (and MP_031a): 2-(4-bromophenyl)-1-(4-chlorophenyl)propan-2-ol: ¹H NMR (500 MHz, CDCl₃) δ 7.43 (d, 2H, J=8.6 Hz), 7.23 (d, 2H, J=8.6 Hz), 7.18 (d, 2H, J=8.4 Hz), 6.89 (d, 2H, J=8.4 Hz), 3.04 (d, 1H, J=8.6 Hz), 2.96 (d, 1H, J=8.6 Hz), 1.73 (bs, 1H), 1.54 (s, 3H).

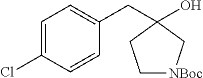 [SMILES: CC(C)(C)OC(=O)N1CCC(O)(Cc2ccc(Cl)cc2)C1]

MP_032a and MP_033a: tert-butyl 3-(4-chlorobenzyl)-3-hydroxypyrrolidine-1-carboxylate: ¹H NMR (500 MHz, CDCl₃) δ 7.29 (m, 2H), 7.17 (d, 2H, J=8.1 Hz), 3.54-3.24 (m, 4H), 2.98 (m, 2H), 1.91 (m, 1H), 1.79 (m, 1H), 1.48 (bs, 1H), 1.44 (s, 9H).

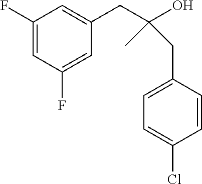 [SMILES: CC(O)(Cc1ccc(Cl)cc1)Cc1cc(F)cc(F)c1]

MP_038a: 1-(4-chlorophenyl)-3-(3,5-difluorophenyl)-2-methylpropan-2-ol: ¹H NMR (500 MHz, CDCl₃) δ 7.28 (d, 2H, J=8.4 Hz), 7.15 (d, 2H, J=8.5 Hz), 7.11-7.04 (m, 2H), 6.92 (m, 1H), 2.80-2.76 (m, 2H), 2.73-2.69 (m, 2H), 1.26 (s, 1H), 1.04 (s, 3H).

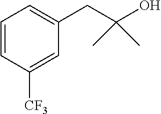

MP_039a: 2-methyl-1-(3-(trifluoromethyl)phenyl)propan-2-ol: ¹H NMR (500 MHz, CDCl₃) δ 7.50 (m, 2H), 7.41 (m, 2H), 2.82 (s, 2H), 1.26 (s, 1H), 1.24 (s, 6H).

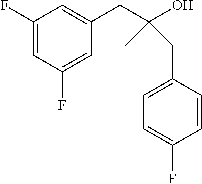 [SMILES: CC(O)(Cc1ccc(F)cc1)Cc1cc(F)cc(F)c1]

MP_045a and MP_046a: 1-(3,5-difluorophenyl)-3-(4-fluorophenyl)-2-methylpropan-2-ol: ¹H NMR (500 MHz, CDCl₃) δ 7.17 (m, 2H), 7.07 (m, 2H), 7.00 (m, 2H), 6.92 (m, 1H), 2.80-2.70 (m, 4H), 1.27 (s, 1H), 1.04 (s, 3H).

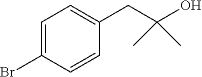

MP_047a (and for MP_061a and MP_062a): 1-(4-bromophenyl)-2-methylpropan-2-ol: ¹H NMR (500 MHz, CDCl₃) δ 7.43 (d, 2H, J=8.4 Hz), 7.09 (d, 2H, J=8.4 Hz), 2.72 (s, 2H), 1.26 (bs, 1H), 1.21 (s, 6H).

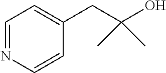

MP_048a: 2-methyl-1-(pyridin-4-yl)propan-2-ol: ¹H NMR (500 MHz, CDCl₃) δ 8.50 (d, 2H, J=6.0 Hz), 7.16 (d, 2H, J=6.0 Hz), 2.75 (s, 2H), 1.46 (s, 1H), 1.23 (s, 6H).

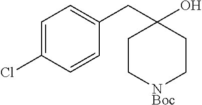 [SMILES: CC(C)(C)OC(=O)N1CCC(O)(Cc2ccc(Cl)cc2)CC1]

MP_049a: tert-butyl 4-(4-chlorobenzyl)-4-hydroxypiperidine-1-carboxylate: ¹H NMR (500 MHz, CDCl₃) δ 7.27 (d, 2H, J=8.4 Hz), 7.11 (d, 2H, J=8.5 Hz), 3.82 (bm, 2H), 3.07 (bm, 2H), 2.71 (s, 2H), 1.55 (m, 2H), 1.44 (m, 11H), 1.33 (s, 1H).

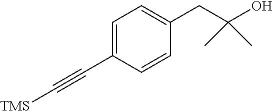 [SMILES: CC(C)(O)Cc1ccc(C#C[Si](C)(C)C)cc1]

MP_050a: 2-methyl-1-(4-((trimethylsilyl)ethynyl)phenyl)propan-2-ol: ¹H NMR (500 MHz, CDCl₃) δ 7.41 (d, 2H, J=8.2 Hz), 7.15 (d, 2H, J=8.2 Hz), 2.75 (s, 2H), 1.28 (s, 1H), 1.20 (s, 6H), 0.24 (s, 9H).

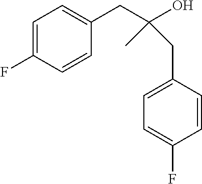 [SMILES: CC(O)(Cc1ccc(F)cc1)Cc1ccc(F)cc1]

MP_051a and 52a: 1,3-bis(4-fluorophenyl)-2-methylpropan-2-amine: ¹H NMR (500 MHz, CDCl₃) δ 7.18 (m, 4H), 6.99 (m, 4H), 2.76 (d, 2H, J=13.6 Hz), 2.74 (d, 2H, J=13.6 Hz), 1.28 (s, 1H), 1.04 (s, 3H).

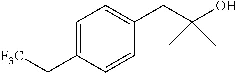 [SMILES: CC(C)(O)Cc1ccc(CC(F)(F)F)cc1]

MP_059a: 2-methyl-1-(4-(2,2,2-trifluoroethyl)phenyl)propan-2-ol: ¹H NMR (500 MHz, CDCl₃) δ 7.22 (m, 4H), 3.35 (q, 2H, J=10.8 Hz), 2.76 (s, 2H), 1.43 (bs, 1H), 1.23 (s, 6H).

### Amines

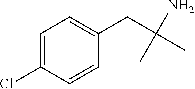

MP_001b (and other 2-chlorophenyl derivatives): 1-(4-chlorophenyl)-2-methylpropan-2-amine: ¹H NMR (400 MHz, CDCl₃) δ 7.26 (d, 2H, J=8.4 Hz), 7.12 (d, 2H, J=8.4 Hz), 2.62 (s, 2H), 1.10 (s, 6H), 1.05 (bs, 2H).

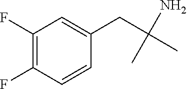

MP_022b (and other 3,4-difluorophenyl derivatives): 1-(3,4-difluorophenyl)-2-methylpropan-2-amine: ¹H NMR (500 MHz, CDCl₃) δ 7.05 (m, 2H), 6.89 (m, 1H), 2.63 (s, 2H), 1.25 (m, 2H), 1.13 (s, 6H).

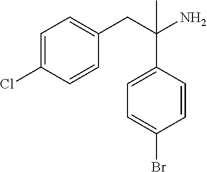 [SMILES: CC(N)(Cc1ccc(Cl)cc1)c1ccc(Br)cc1]

MP_030b (and MP_031b): 2-(4-bromophenyl)-1-(4-chlorophenyl)propan-2-amine: ¹H NMR (500 MHz, CDCl₃) δ 7.46 (d, 2H, J=8.6 Hz), 7.25 (apparent d, 2H), 7.15 (d, 2H, J=8.4 Hz), 6.79 (d, 2H, J=8.4 Hz), 2.96 (d, 1H, J=13.2 Hz), 2.88 (d, 1H, J=13.2 Hz), 1.50 (bs, 3H), 1.48 (s, 3H).

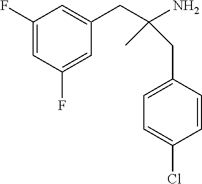 [SMILES: CC(N)(Cc1ccc(Cl)cc1)Cc1cc(F)cc(F)c1]

MP_038b: 1-(4-chlorophenyl)-3-(3,5-difluorophenyl)-2-methylpropan-2-amine: ¹H NMR (500 MHz, CDCl₃) δ 7.29-7.23 (m, 3H), 7.17-7.01 (m, 3H), 6.93-6.86 (m, 1H), 2.66 (m, 4H), 0.97 (s, 3H).

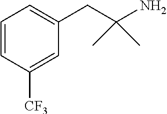

MP_039b: 2-methyl-1-(3-(trifluoromethyl)phenyl)propan-2-amine: ¹H NMR (500 MHz, CDCl₃) δ 7.57-7.37 (m, 4H), 2.72 (s, 2H), 1.58 (bs, 2H), 1.13 (s, 6H).

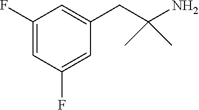

MP_043b (and MP_044b): 1-(3,5-difluorophenyl)-2-methylpropan-2-amine: ¹H NMR (500 MHz, CDCl₃) δ 6.70 (m, 3H), 2.66 (s, 2H), 1.80 (bs, 2H), 1.15 (s, 6H).

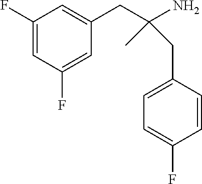 [SMILES: CC(N)(Cc1ccc(F)cc1)Cc1cc(F)cc(F)c1]

MP_045b and MP_046b: 1-(3,5-difluorophenyl)-3-(4-fluorophenyl)-2-methylpropan-2-amine: ¹H NMR (500 MHz, CDCl₃) δ 7.14-6.90 (m, 7H), 2.67 (m, 4H), 0.97 (s, 3H).

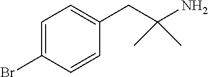

MP_047b (and JP_061b and JP_062b): 1-(4-bromophenyl)-2-methylpropan-2-amine: ¹H NMR (500 MHz, CDCl₃) δ 7.42 (d, 2H, J=8.3 Hz), 7.06 (d, 2H, J=8.3 Hz), 2.63 (s, 2H), 1.81 (bs, 2H), 1.12 (s, 6H).

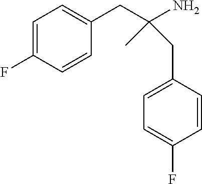 [SMILES: CC(N)(Cc1ccc(F)cc1)Cc1ccc(F)cc1]

MP_051c and 051b: 1,3-bis(4-fluorophenyl)-2-methylpropan-2-amine: ¹H NMR (500 MHz, CDCl₃) δ 7.15 (m, 4H), 6.89 (m, 4H), 2.70 (m, 4H), 1.54 (bs, 2H), 0.99 (s, 3H).

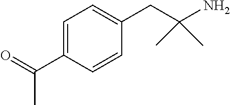

MP_053b: 1-(4-(2-amino-2-methylpropyl)phenyl)ethan-1-one: ¹H NMR (500 MHz, CDCl₃) δ 7.90 (d, 2H, J=8.3 Hz), 7.33 (d, 2H, J=8.2 Hz), 2.99 (s, 2H), 2.59 (s, 3H), 1.33 (s, 6H).

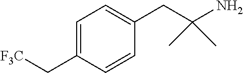 [SMILES: CC(C)(N)Cc1ccc(CC(F)(F)F)cc1]

MP_058b: 2-methyl-1-(4-(2,2,2-trifluoroethyl)phenyl)propan-2-amine: ¹H NMR (500 MHz, CDCl₃) δ 7.22 (d, 2H, J=8.0 Hz), 7.18 (d, 2H, J=8.1 Hz), 3.34 (q, 2H, J=10.9 Hz), 2.66 (s, 2H), 1.40 (bs, 3H), 1.12 (s, 6H).

### Boc-Protected Esters

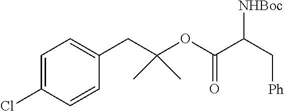 [SMILES: CC(C)(C)OC(=O)NC(Cc1ccccc1)C(=O)OC(C)(C)Cc1ccc(Cl)cc1]

MP_001c: 1-(4-chlorophenyl)-2-methylpropan-2-yl 2-(tert-butoxycarbonylamino)-3-phenylpropanoate: ¹H NMR (400 MHz, CDCl₃) δ 7.28-7.20 (m, 5H), 7.11 (d, 2H, J=8.3 Hz), 7.11 (d, 2H, J=8.3 Hz), 4.96 (1H, d, J=7.9 Hz), 4.44 (dd, 1H, J=7.9, 6.6 Hz), 3.00 (d, 2H, J=5.8 Hz), 2.96 (d, 1H, J=13.7 Hz), 2.82 (d, 1H, J=13.7 Hz), 1.43 (s, 9H), 1.42 (s, 3H), 1.37 (s, 3H).

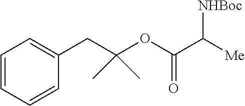 [SMILES: CC(NC(=O)OC(C)(C)C)C(=O)OC(C)(C)Cc1ccccc1]

MP_002c: 2-methyl-1-phenylpropan-2-yl 2-(tert-butoxycarbonylamino)propanoate: ¹H NMR (400 MHz, CDCl₃) δ 7.31-7.17 (m, 5H), 5.02 (bs, 1H), 4.19 (m, 1H), 3.05 s, 1H), 3.04 (s, 1H), 1.48 (s, 3H, 1.45 (s, 3H), 1.44 (s, 9H), 1.28 (d, 3H, J=7.12 Hz).

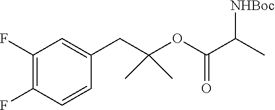 [SMILES: CC(NC(=O)OC(C)(C)C)C(=O)OC(C)(C)Cc1ccc(F)c(F)c1]

MP_003c: 1-(3,4-difluorophenyl)-2-methylpropan-2-yl 2-(tert-butoxycarbonylamino)propanoate: ¹H NMR (400 MHz, CDCl₃) δ 7.10-6.97 (m, 2H), 6.89 (m, 1H), 4.96 (bs, 1H), 4.17 (m, 1H), 3.04 (d, 1H, J=13.8 Hz), 2.98 (d, 1H, J=13.8 Hz), 1.47 (s, 3H), 1.45 (s, 9H), 1.44 (s, 3H), 1.28 (d, 2H, J=7.2 Hz).

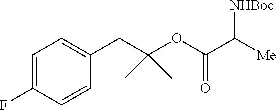 [SMILES: CC(NC(=O)OC(C)(C)C)C(=O)OC(C)(C)Cc1ccc(F)cc1]

MP_004c: 1-(4-fluorophenyl)-2-methylpropan-2-yl 2-(tert-butoxycarbonylamino)propanoate: ¹H NMR (400 MHz, CDCl₃) δ 7.14 (m, 2H), 6.97 (m, 2H), 4.98 (bs, 1H), 4.18 (m, 1H), 3.04 (d, 1H, J=13.8 Hz), 2.98 (d, 1H, J=13.8 Hz), 1.48 (s, 3H), 1.45 (s, 9H), 1.44 (s, 3H), 1.28 (d, 2H, J=7.2 Hz).

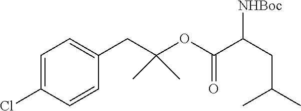 [SMILES: CC(C)CC(NC(=O)OC(C)(C)C)C(=O)OC(C)(C)Cc1ccc(Cl)cc1]

MP_005c: 1-(4-chlorophenyl)-2-methylpropan-2-yl 2-(tert-butoxycarbonylamino)-4-methylpentanoate: ¹H NMR (400 MHz, CDCl₃) δ 7.25 (d, 2H, J=8.3 Hz), 7.12 (d, 2H, J=8.3 Hz), 4.81 (d, 1H, J=8.8), 4.17 (m, 1H), 3.07 (d, 1H, J=13.8 Hz), 2.94 (d, 1H, J=13.8 Hz), 1.64 (m, 1H), 1.49 (s, 3H), 1.44 (s, 9H), 1.41 (s, 3H), 1.38 (m, 2H), 0.92 (d, 3H, J=6.6 Hz), 0.89 (d, 2H, J=6.6 Hz).

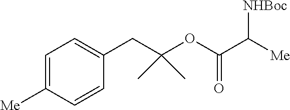 [SMILES: Cc1ccc(CC(C)(C)OC(=O)C(C)NC(=O)OC(C)(C)C)cc1]

MP_006c: 2-methyl-1-p-tolylpropan-2-yl 2-(tert-butoxycarbonylamino)propanoate: ¹H NMR (400 MHz, CDCl₃) δ 7.09 (d, 2H, J=8.3 Hz), 7.06 (d, 2H, J=8.3 Hz), 5.02 (bs, 1H), 4.19 (t, 1H, 7.9 Hz), 3.02 (d, 1H, J=13.7 Hz), 2.98 (d, 1H, J=13.7 Hz), 2.32 (s, 1H), 1.47 (s, 3H), 1.45 (s, 9H), 1.44 (s, 3H), 1.29 (d, 3H, J=7.2 Hz).

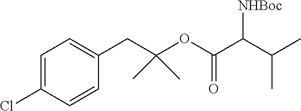 [SMILES: CC(C)C(NC(=O)OC(C)(C)C)C(=O)OC(C)(C)Cc1ccc(Cl)cc1]

MP_008c: 1-(4-chlorophenyl)-2-methylpropan-2-yl 2-(tert-butoxycarbonylamino)-3-methylbutanoate: ¹H NMR (400 MHz, CDCl₃) δ 7.25 (d, 2H, J=8.4 Hz), 7.12 (d, 2H, J=8.4 Hz), 4.94 (d, 1H, J=9.0 Hz), 4.10 (m, 1H), 3.07 (d, 1H, J=13.7 Hz), 2.92 (d, 1H, J=13.7 Hz), 2.07 (m, 1H), 1.49 (s, 3H), 1.45 (s, 9H), 1.43 (s, 3H), 0.93 (d, 2H, J=6.9 Hz), 0.84 (d, 3H, J=6.9 Hz).

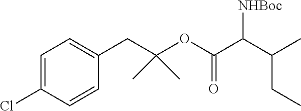 [SMILES: CCC(C)C(NC(=O)OC(C)(C)C)C(=O)OC(C)(C)Cc1ccc(Cl)cc1]

MP_011c: 1-(4-chlorophenyl)-2-methylpropan-2-yl 2-(tert-butoxycarbonylamino)-3-methylpentanoate: ¹H NMR (400 MHz, CDCl₃) δ 7.25 (d, 2H, J=8.2 Hz), 7.12 (d, 2H, J=8.3 Hz), 4.96 (d, 1H, J=9.7 Hz, diast. A), 4.89 (d, 1H, J=9.7 Hz, diast. B), 4.24 (dd, 1H, J=9.7, 3.5 Hz, diast. A), 4.13 (dd, 1H, J=9.4, 4.8 Hz, diast. B), 3.07 (d, 1H, J=13.7 Hz, diast. A), 3.06 (d, 1H, J=13.7 Hz, diast. B), 2.92 (d, 1H, J=13.7 Hz, diast. A), 2.91 (d, 1H, J=13.7 Hz, diast. B), 0.93 (t, 3H, J=7.4 Hz, diast. A), 0.89 (d, 3H, J=6.8 Hz, diast. B), 0.87 (t, 3H, J=7.4 Hz, diast. B), 0.77 (d, 3H, J=6.9 Hz, diast. A).

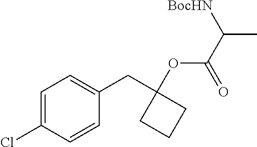 [SMILES: CC(NC(=O)OC(C)(C)C)C(=O)OC1(Cc2ccc(Cl)cc2)CCC1]

MP_009c: 1-(4-chlorobenzyl)cyclobutyl 2-(tert-butoxycarbonylamino)propanoate: ¹H NMR (400 MHz, CDCl₃) δ 7.25 (d, 2H, J=8.4 Hz), 7.10 (d, 2H, J=8.4 Hz), 4.97 (bs, 1H), 4.19 (m, 1H), 3.24 (d, 1H, J=14.3 Hz), 3.17 (d, 1H, J=14.3 Hz), 2.30 (m, 4H), 1.86 (m, 1H), 1.60 (s, 1H), 1.45 (s, 9H), 1.28 (d, 3H, J=7.2 Hz).

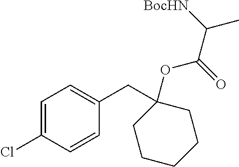 [SMILES: CC(NC(=O)OC(C)(C)C)C(=O)OC1(Cc2ccc(Cl)cc2)CCCCC1]

MP_010c: 1-(4-chlorobenzyl)cyclohexyl 2-(tert-butoxycarbonylamino)propanoate: ¹H NMR (400 MHz, CDCl₃) δ 7.24 (d, 2H, J=8.4 Hz), 7.05 (d, 2H, J=8.4 Hz), 4.98 (d, 1H, J=6.3 Hz), 4.21 (m, 1H), 3.23 (d, 1H, J=13.8 Hz), 3.09 (d, 1H, J=13.8 Hz), 2.30 (d, 1H, J=12.5 Hz), 2.14 (d, 1H, J=12.5 Hz), 1.59 (m, 1H), 1.47-1.18 (m, 7H), 1.45 (s, 9H), 1.32 (d, 3H, J=7.2 Hz).

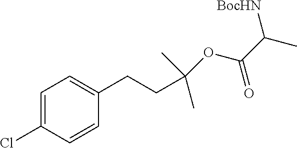 [SMILES: CC(NC(=O)OC(C)(C)C)C(=O)OC(C)(C)CCc1ccc(Cl)cc1]

MP_012c: 4-(4-chlorophenyl)-2-methylbutan-2-yl 2-(tert-butoxycarbonylamino)propanoate: ¹H NMR (400 MHz, CDCl₃) δ 7.24 (d, 2H, J=8.4 Hz), 7.10 (d, 2H, J=8.5 Hz), 4.99 (d, 1H, J=4.9 Hz), 4.21 (m, 1H), 2.60 (m, 2H), 2.04 (m, 2H), 1.51 (s, 3H), 1.49 (s, 3H, 1.44 (s, 9H), 1.36 (d, 3H, J=7.2 Hz).

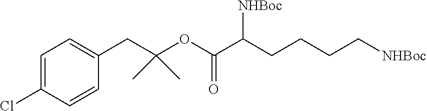 [SMILES: CC(C)(C)OC(=O)NCCCCC(NC(=O)OC(C)(C)C)C(=O)OC(C)(C)Cc1ccc(Cl)cc1]

MP_013c: 1-(4-chlorophenyl)-2-methylpropan-2-yl 2,6-bis(tert-butoxycarbonylamino)hexanoate: ¹H NMR (400 MHz, CDCl₃) δ 7.26 (d, 2H, J=8.4 Hz), 7.12 (d, 2H, J=8.5 Hz), 5.01 (d, 1H, J=7.9 Hz), 4.52 (bs, 1H), 4.14 (m, 1H), 3.06 (m, 3H), 2.94 (d, 1H, J=13.7 Hz), 1.68 (m, 1H), 1.56 (m, 1H), 1.50-1.39 (m, 2H), 1.48 (s, 3H), 1.45 (s, 9H), 1.44 (s, 9H). 1.43 (s, 3H), 1.29 (m, 2H).

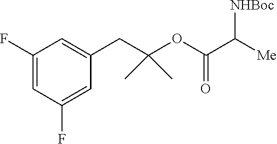 [SMILES: CC(NC(=O)OC(C)(C)C)C(=O)OC(C)(C)Cc1cc(F)cc(F)c1]

MP_014c: 1-(3,5-difluorophenyl)-2-methylpropan-2-yl 2-(tert-butoxycarbonylamino)propanoate: ¹H NMR (400 MHz, CDCl₃) δ 6.70 (m, 3H), 4.96 (bs, 1H), 4.20 (m, 1H), 3.03 (s, 2H), 1.48 (s, 3H), 1.46 (s, 3H), 1.44 (s, 9H), 1.29 (d, 3H, J=7.2 Hz).

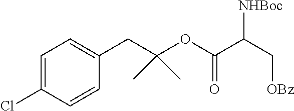

MP_016c1: 2-(tert-butoxycarbonylamino)-3-(1-(4-chlorophenyl)-2-methylpropan-2-yloxy)-3-oxopropyl benzoate: ¹H NMR (400 MHz, CDCl₃) δ 7.28 (m, 5H), 7.21 (d, 2H, J=8.4 Hz), 7.09 (d, 2H, J=8.4 Hz), 5.33 (d, 1H, J=8.7 Hz), 4.46 (d, 1H, J=12.1 Hz), 4.39 (d, 1H, J=12.0 Hz), 4.26 (m, 1H), 3.77 (dd, 1H, J=9.2, 3.1 Hz), 3.61 (dd, 1H, J=9.2, 3.1 Hz), 3.04 (d, 1H, J=13.7 Hz), 2.91 (d, 1H, J=13.7 Hz), 1.47 (s, 3H), 1.45 (s, 9H), 1.40 (s, 3H).

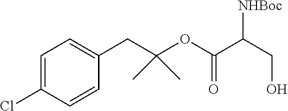 [SMILES: CC(C)(C)OC(=O)NC(CO)C(=O)OC(C)(C)Cc1ccc(Cl)cc1]

MP_016c2: 1-(4-chlorophenyl)-2-methylpropan-2-yl 2-(tert-butoxycarbonylamino)-3-hydroxypropanoate: ¹H NMR (400 MHz, CDCl₃) δ 7.26 (m, 5H), 7.11 (d, 2H, J=8.4 Hz), 5.38 (d, 1H, J=5.1 Hz), 4.24 (m, 1H), 3.83 (m, 2H), 3.02 (s, 2H), 2.29 (bs, 1H), 1.47 (s, 3H), 1.46 (s, 3H), 1.45 (s, 9H).

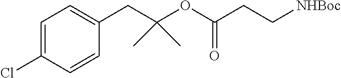 [SMILES: CC(C)(C)OC(=O)NCCC(=O)OC(C)(C)Cc1ccc(Cl)cc1]

MP_017c: 1-(4-chlorophenyl)-2-methylpropan-2-yl 3-(tert-butoxycarbonylamino)propanoate: ¹H NMR (500 MHz, CDCl₃) δ 7.25 (m, 2H), 7.09 (d, 2H, J=8.3 Hz), 4.92 (s, 1H), 3.33 (m, 2H), 3.01 (s, 2H), 2.41 (m, 2H), 1.43 (m, 15H).

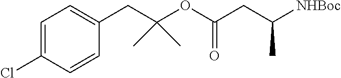 [SMILES: C[C@@H](CC(=O)OC(C)(C)Cc1ccc(Cl)cc1)NC(=O)OC(C)(C)C]

MP_018c: (S)-1-(4-chlorophenyl)-2-methylpropan-2-yl 3-(tert-butoxycarbonylamino)butanoate: ¹H NMR (500 MHz, CDCl₃) δ 7.25 (d, 2H, J=8.3 Hz), 7.11 (d, 2H, J=8.3 Hz), 4.85 (bs, 1H), 4.00 (m, 2H), 3.01 (s, 2H), 2.39 (m, 2H), 1.43 (bs, 15H), 1.17 (d, 3H, J=6.7 Hz).

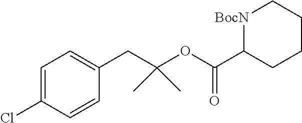 [SMILES: CC(C)(C)OC(=O)N1CCCCC1C(=O)OC(C)(C)Cc1ccc(Cl)cc1]

MP_019c: 1-tert-butyl 2-(1-(4-chlorophenyl)-2-methylpropan-2-yl) piperidine-1,2-dicarboxylate: ¹H NMR (500 MHz, MeOD) δ7.27 (d, 2H, J=8.4 Hz), 7.20 (d, 2H, J=8.5 Hz), 4.62 (m, 1H), 3.88 (m, 1H), 3.07 (m, 2H), 2.83 (m, 1H), 2.13 (m, 1H), 1.61 (m, 3H), 1.47-1.38 (m, 16H).

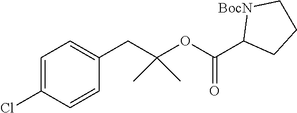 [SMILES: CC(C)(C)OC(=O)N1CCCC1C(=O)OC(C)(C)Cc1ccc(Cl)cc1]

MP_020c: 1-tert-butyl 2-(1-(4-chlorophenyl)-2-methylpropan-2-yl) pyrrolidine-1,2-dicarboxylate: ¹H NMR (500 MHz, CDCl₃) δ 7.24 (m, 2H), 7.12 (m, 2H), 4.19 (m, 1H, diastereomer B), 4.10 (m, 1H, diastereomer A), 3.44 (m, 2H), 3.10-2.94 (m, 2H), 2.13 (m, 1H), 1.81 (m, 3H), 1.46-1.38 (m, 15H).

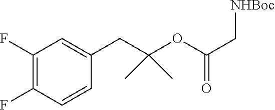 [SMILES: CC(C)(C)OC(=O)NCC(=O)OC(C)(C)Cc1ccc(F)c(F)c1]

MP_021c and MP_022c: 1-(3,4-difluorophenyl)-2-methylpropan-2-yl 2-(tert-butoxycarbonylamino)ethanoate: ¹H NMR (500 MHz, CDCl₃) δ 7.05 (m, 1H), 6.97 (m, 1H), 6.86 (m, 1H), 4.97 (bs, 1H), 3.78 (d, 2H, J=5.4 Hz), 2.97 (s, 2H), 1.44 (s, 6H), 1.43 (m, 9H).

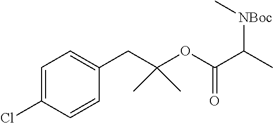 [SMILES: CC(C(=O)OC(C)(C)Cc1ccc(Cl)cc1)N(C)C(=O)OC(C)(C)C]

MP_023c: 1-(4-chlorophenyl)-2-methylpropan-2-yl 2-(tert-butoxycarbonyl(methyl)amino)propanoate: ¹H NMR (500 MHz, CDCl₃) δ 7.24 (d, 2H, J=8.0 Hz), 7.10 m, 2H), 4.70 (q, 1H, J=7.1 Hz, diastereomer A), 4.38 (q, 1H, J=7.0 Hz, diastereomer B), 2.97 (s, 3H, diastereomer B), 2.72 (s, 3H, diastereomer A), 1.44 (m, 15H), 1.30 (m, 3H).

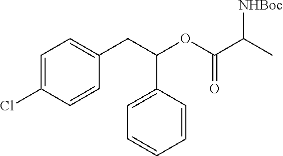 [SMILES: CC(NC(=O)OC(C)(C)C)C(=O)OC(Cc1ccc(Cl)cc1)c1ccccc1]

MP_026c: 2-(4-chlorophenyl)-1-phenylethyl (tert-butoxycarbonyl)alaninate: ¹H NMR (500 MHz, CDCl₃) δ 7.30-7.20 (m, 7H), 7.03 (d, 1H, J=8.2 Hz), 6.98 (d, 1H, J=7.9 Hz), 4.94 (m, 1H), 4.31 (m, 1H), 3.11 (m, 2H) 1.42 (d, 9H, J=13 Hz), 1.26 (m, 3H).

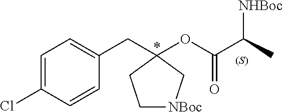

MP_032c (diastereomer 1): tert-butyl 3-(((tert-butoxycarbonyl)-L-alanyl)oxy)-3-(4-chlorobenzyl)pyrrolidine-1-carboxylate: ¹H NMR (500 MHz, CDCl₃) δ 7.26 (m, 2H), 7.07 (m, 2H), 4.91 (bs, 1H), 4.14 (bs, 1H), 3.78 (t, 1H, J=13.5 Hz), 3.50-3.29 (m, 5H), 2.41-2.19 (m, 1H), 1.99 (m, 1H), 1.95 (bs, 18H), 1.19 (bm, 3H).

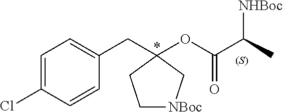

MP_033c (diastereomer 2): tert-butyl 3-(((tert-butoxycarbonyl)-L-alanyl)oxy)-3-(4-chlorobenzyl)pyrrolidine-1-carboxylate: ¹H NMR (500 MHz, CDCl₃) δ 7.25 (m, 2H), 7.06 (d, 2H, J=8.2 Hz), 4.90 (bd, 1H), 4.15 (bm, 1H), 3.79-3.60 (m, 1H), 3.50-3.19 (m, 5H), 2.48-2.37 (m, 1H), 2.03 (m, 1H), 1.93 (bs, 18H), 1.24 (bm, 3H).

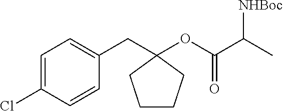 [SMILES: CC(NC(=O)OC(C)(C)C)C(=O)OC1(Cc2ccc(Cl)cc2)CCCC1]

MP_035c: 1-(4-chlorobenzyl)cyclopentyl (tert-butoxycarbonyl)alaninate: ¹H NMR (400 MHz, CDCl₃) δ 7.24 (d, 2H, J=10.6 Hz), 7.08 (d, 2H, J=10.6 Hz), 4.94 (bm, 1H), 4.14 (bm, 1H), 3.32 (d, 1H, J=14.0 Hz), 3.20 (d, 1H, J=13.9 Hz), 2.10 (m, 2H), 1.70 (m, 6H), 1.45 (s, 9H), 1.24 (d, 2H, J=7.2 Hz).

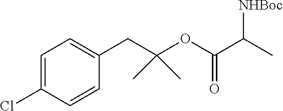 [SMILES: CC(NC(=O)OC(C)(C)C)C(=O)OC(C)(C)Cc1ccc(Cl)cc1]

MP_041c (and MP_042c): 1-(4-chlorophenyl)-2-methylpropan-2-yl (tert-butoxycarbonyl)alaninate: ¹H NMR (500 MHz, CDCl₃) δ 7.25 (d, 2H, J=8.3 Hz), 7.10 (d, 2H, J=8.4 Hz), 4.98 (bs, 1H), 4.17 (bm, 1H), 3.04 (d, 1H, J=13.8 Hz), 2.98 (d, 2H, J=13.8 Hz), 1.47-1.42 (m, 15H), 1.28 (d, 3H, J=7.2 Hz).

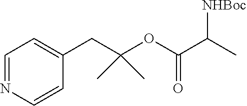 [SMILES: CC(NC(=O)OC(C)(C)C)C(=O)OC(C)(C)Cc1ccncc1]

MP_048c: 2-methyl-1-(pyridin-4-yl)propan-2-yl (tert-butoxycarbonyl)alaninate: ¹H NMR (500 MHz, CDCl₃) δ 8.52 (d, 2H, J=6.1 Hz), 7.14 (d, 2H, J=6.1 Hz), 4.98 (bd, 1H, J=6.8 Hz), 4.17 (bm, 1H), 3.09 (d, 1H, J=13.5 Hz), 3.02 (d, 1H, J=13.2 Hz), 1.43 (bs, 9H), 1.49 (bm, 6H), 1.27 (d, 3H, J=7.2 Hz)

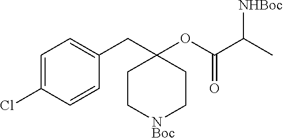 [SMILES: CC(NC(=O)OC(C)(C)C)C(=O)OC1(Cc2ccc(Cl)cc2)CCN(C(=O)OC(C)(C)C)CC1]

MP_049c: tert-butyl 4-(((tert-butoxycarbonyl)alanyl)oxy)-4-(4-chlorobenzyl)piperidine-1-carboxylate: ¹H NMR (500 MHz, CDCl₃) δ 7.24 (d, 2H, J=8.1 Hz), 7.03 (d, 2H, J=8.1 Hz), 4.91 (bm, 1H), 4.18 (m, 1H), 3.90 (bm, 2H), 3.28 (bd, 1H, J=13.8 Hz), 3.09 (bd, 1H, J=13.6 Hz), 2.89 (bm, 2H), 2.34 (bm, 1H), 2.12 (m, 1H), 1.42 (bs, 18H), 1.54 (m, 2H), 1.30 (d, 3H, J=7.3 Hz)

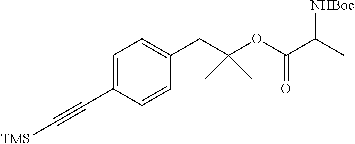 [SMILES: CC(NC(=O)OC(C)(C)C)C(=O)OC(C)(C)Cc1ccc(C#C[Si](C)(C)C)cc1]

MP_050c1: 2-methyl-1-(4-((trimethylsilyl)ethynyl)phenyl)propan-2-yl (tert-butoxycarbonyl)alaninate: ¹H NMR (500 MHz, CDCl₃) δ 7.38 (d, 2H, J=8.3 Hz), 7.11 (d, 2H, J=8.3 Hz), 4.78 (bd, 1H, J=6.8 Hz), 4.17 (m, 1H), 3.02 (m, 2H), 1.46 (s, 3H), 1.44 (s, 9H), 1.42 (s, 3H), 1.27 (d, 3H, J=7.2 Hz), 0.24 (s, 9H).

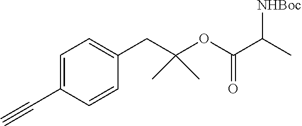 [SMILES: C#Cc1ccc(CC(C)(C)OC(=O)C(C)NC(=O)OC(C)(C)C)cc1]

MP_050c2: 1-(4-ethynylphenyl)-2-methylpropan-2-yl (tert-butoxycarbonyl)alaninate: ¹H NMR (500 MHz, CDCl₃) δ 7.41 (d, 2H, J=8.2 Hz), 7.14 (d, 2H, J=8.2 Hz), 4.99 (bd, 1H, J=5.9 Hz), 4.17 (m, 1H), 3.04 (m, 3H), 1.47 (s, 3H), 1.44 (m, 12H), 1.27 (d, 3H, J=7.1 Hz).

### Boc-Protected Amides

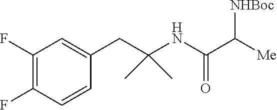 [SMILES: CC(NC(=O)OC(C)(C)C)C(=O)NC(C)(C)Cc1ccc(F)c(F)c1]

MP_022d: tert-butyl 1-(1-(3,4-difluorophenyl)-2-methylpropan-2-ylamino)-1-oxopropan-2-ylcarbamate: ¹H NMR (500 MHz, CDCl₃) δ 7.03 (dt, 1H, J=10.3, 8.4 Hz), 6.92 (ddd, 1H, J=11.5, 7.8, 2.1 Hz), 6.82 (m, 1H), 5.84 (bs, 1H), 4.85 (bs, 1H), 4.00 (m, 1H), 3.12 (d, 1H, J=13.4), 2.93 (d, 1H, J=13.4 Hz), 1.39 (s, 9H), 1.31 (d, 3H, J=7.1 Hz), 1.32 (s, 3H), 1.25 (s, 3H).

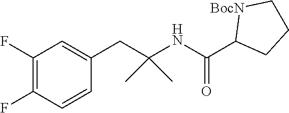 [SMILES: CC(C)(Cc1ccc(F)c(F)c1)NC(=O)C1CCCN1C(=O)OC(C)(C)C]

MP_024d: tert-butyl 2-(1-(3,4-difluorophenyl)-2-methylpropan-2-ylcarbamoyl)pyrrolidine-1-carboxylate: ¹H NMR (500 MHz, CDCl₃) δ 7.04-6.80 (m, 3H), 4.17 (m, 1H), 3.35 (m, 2H), 2.70-1.80 (m, 4H) 1.41-1.39 (m, 12H), 1.16 (s, 1H).

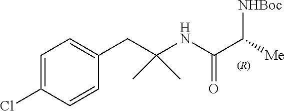

MP_027d: tert-butyl (R)-(1-((1-(4-chlorophenyl)-2-methylpropan-2-yl)amino)-1-oxopropan-2-yl)carbamate: ¹H NMR (500 MHz, CDCl₃) δ 7.22 (d, 2H, J=8.4 Hz), 7.04 (d, 2H, J=8.4 Hz), 5.78 (bs, 1H), 4.86 (bs, 1H), 3.99 (m, 1H), 3.11 (d, 1H, 13.3 Hz), 2.95 (d, 1H, 13.3 Hz), 1.40 (s, 9H), 1.32 (bs, 3H), 1.31 (d, 3H, J=7.1 Hz), 1.26 (bs, 3H).

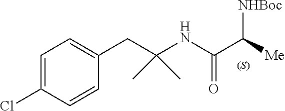

MP_028d: tert-butyl (S)-(1-((1-(4-chlorophenyl)-2-methylpropan-2-yl)amino)-1-oxopropan-2-yl)carbamate: ¹H NMR (500 MHz, CDCl₃) δ 7.23 (d, 2H, J=8.4 Hz), 7.04 (d, 2H, J=8.4 Hz), 5.79 (bs, 1H), 4.87 (bs, 1H), 4.00 (bm, 1H), 3.11 (d, 1H, 13.4 Hz), 2.95 (d, 1H, 13.4 Hz), 1.40 (s, 9H), 1.32 (bs, 3H), 1.31 (d, 3H, J=7.1 Hz), 1.26 (s, 3H).

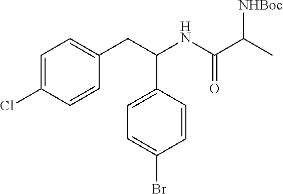 [SMILES: CC(NC(=O)OC(C)(C)C)C(=O)NC(Cc1ccc(Cl)cc1)c1ccc(Br)cc1]

MP_030d: tert-butyl (1-((1-(4-bromophenyl)-2-(4-chlorophenyl)ethyl)amino)-1-oxopropan-2-yl)carbamate: ¹H NMR (500 MHz, CDCl₃) δ 7.41 (m, 2H), 7.19 (m, 2H), 6.82 (m, 2H), 6.63 (bs, 0.5H, diastereomer), 6.50 (bs, 0.5H, diastereomer), 4.81 (m, 1H), 4.08 (m, 1H), 3.38 (m, 1H), 3.14 (d, 0.5H, J=13.1 Hz, diastereomer), 3.05 (d, 0.5H, J=13.2 Hz, diastereomer), 1.43 (m, 12H), 1.30 (d, 3H, J=7.0 Hz).

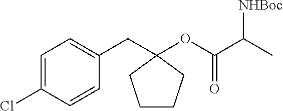 [SMILES: CC(NC(=O)OC(C)(C)C)C(=O)OC1(Cc2ccc(Cl)cc2)CCCC1]

MP_034d: 1-(4-chlorobenzyl)cyclopentyl (tert-butoxycarbonyl)alaninate: ¹H NMR (400 MHz, CDCl₃) δ 7.22-7.06 (m, 4H), 4.97 (bs, 1H), 4.14 (bm, 1H), 3.29-3.18 (m, 2H), 2.05 (m, 2H), 1.70 (m, 6H), 1.49 (s, 9H), 1.26 (d, 3H, J=7.2 Hz).

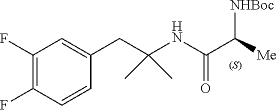

MP_035d: tert-butyl (S)-(1-((1-(3,4-difluorophenyl)-2-methylpropan-2-yl)amino)-1-oxopropan-2-yl)carbamate: ¹H NMR (500 MHz, CDCl₃) δ 7.03 (dt, 1H, J=10.3, 8.4 Hz), 6.92 (ddd, 1H, J=11.4, 7.8, 2.1 Hz), 6.81 (m, 1H), 5.84 (bs, 1H), 4.85 (bs, 1H), 4.00 (bm, 1H), 3.12 (d, 1H, J=13.5), 2.93 (d, 1H, J=13.5 Hz), 1.39 (s, 9H), 1.31 (d, 3H, J=7.1 Hz), 1.32 (s, 3H), 1.26 (s, 3H).

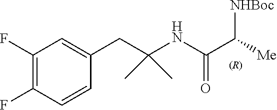

MP_036d: tert-butyl (R)-(1-((1-(3,4-difluorophenyl)-2-methylpropan-2-yl)amino)-1-oxopropan-2-yl)carbamate: ¹H NMR (500 MHz, CDCl₃) δ 7.04 (dt, 1H, J=10.3, 8.3 Hz), 6.92 (ddd, 1H, J=11.4, 7.7, 2.0 Hz), 6.82 (m, 1H), 5.84 (bs, 1H), 4.85 (bs, 1H), 4.00 (bm, 1H), 3.12 (d, 1H, J=13.5), 2.93 (d, 1H, J=13.5 Hz), 1.39 (s, 9H), 1.31 (d, 3H, J=7.1 Hz), 1.32 (s, 3H), 1.25 (s, 3H).

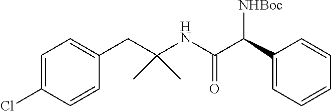 [SMILES: CC(C)(Cc1ccc(Cl)cc1)NC(=O)[C@@H](NC(=O)OC(C)(C)C)c1ccccc1]

MP_037d: tert-butyl (S)-(2-((1-(4-chlorophenyl)-2-methylpropan-2-yl)amino)-2-oxo-1-phenylethyl)carbamate: ¹H NMR (500 MHz, CDCl₃) δ 7.37-7.30 (m, 5H), 6.88 (m, 1H), 6.77 (m, 1H), 6.58 (m, 1H), 5.73 (bs, 1H), 4.94 (bs, 1H), 2.97 (d, 1H, J=13.6), 2.92 (d, 1H, J=13.5 Hz), 1.40 (s, 9H), 1.29 (s, 3H), 1.21 (s, 3H).

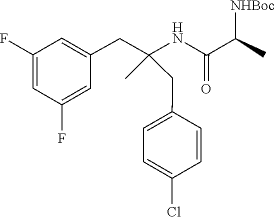 [SMILES: C[C@H](NC(=O)OC(C)(C)C)C(=O)NC(C)(Cc1ccc(Cl)cc1)Cc1cc(F)cc(F)c1]

MP_038d: tert-butyl ((2S)-1-((1-(4-chlorophenyl)-3-(3,5-difluorophenyl)-2-methylpropan-2-yl)amino)-1-oxopropan-2-yl)carbamate: ¹H NMR (500 MHz, CDCl₃) δ 7.25-7.22 (m, 2H), 7.07-7.02 (m, 3H), 6.95 (m, 1H) 6.82 (m, 1H), 5.72 (d, 1H, J=17.9 Hz), 4.77 (bs, 1H), 3.99 (m, 1H), 3.67-3.53 (m, 2H), 2.77 (m, 1H), 2.62 (m, 1H), 1.34-1.31 (m, 12H), 0.98 (bd, 3H).

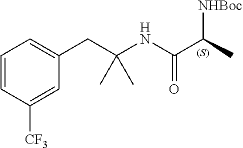

MP_039d: tert-butyl (S)-(1-((2-methyl-1-(3-(trifluoromethyl)phenyl)propan-2-yl)amino)-1-oxopropan-2-yl)carbamate: ¹H NMR (500 MHz, CDCl₃) δ 7.48 (bm, 1H), 7.37 (bm, 2H), 7.30 (bm, 1H), 5.85 (bs, 1H), 4.85 (bs, 1H), 4.00 (bm, 1H), 3.22 (d, 1H, J=13.1 Hz), 3.06 (d, 1H, J=13.3 Hz), 1.38 (s, 9H), 1.34 (bs, 3H), 1.31 (d, 3H, J=7.1 Hz), 1.28 (bs, 3H).

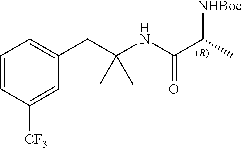

MP_040d: tert-butyl (R)-(1-((2-methyl-1-(3-(trifluoromethyl)phenyl)propan-2-yl)amino)-1-oxopropan-2-yl)carbamate: ¹H NMR (500 MHz, CDCl₃) δ 7.48 (bm, 1H), 7.38 (bm, 2H), 7.30 (bm, 1H), 5.85 (bs, 1H), 4.85 (bs, 1H), 4.00 (bm, 1H), 3.22 (d, 1H, J=13.1 Hz), 3.06 (d, 1H, J=13.3 Hz), 1.38 (s, 9H), 1.34 (bs, 3H), 1.31 (d, 3H, J=7.1 Hz), 1.28 (s, 3H).

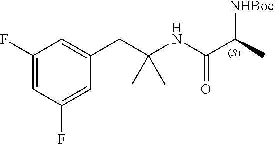

MP_043d: tert-butyl (S)-(1-((1-(3,5-difluorophenyl)-2-methylpropan-2-yl)amino)-1-oxopropan-2-yl)carbamate: ¹H NMR (500 MHz, CDCl₃) δ 6.65 (m, 3H), 5.89 (bs, 1H), 4.86 (bs, 1H), 4.01 (m, 1H), 3.15 (d, 1H, J=13.2 Hz), 2.98 (d, 1H, J=13.3 Hz), 1.40 (s, 9H), 1.34 (s, 3H), 1.31 (d, 3H, J=7.1 Hz), 1.27 (s, 3H).

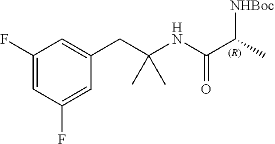

MP_044d: tert-butyl (R)-(1-((1-(3,5-difluorophenyl)-2-methylpropan-2-yl)amino)-1-oxopropan-2-yl)carbamate: ¹H NMR (500 MHz, CDCl₃) δ 6.68-6.63 (m, 3H), 5.88 (bs, 1H), 4.85 (bs, 1H), 4.00 (m, 1H), 3.15 (d, 1H, J=13.2 Hz), 2.98 (d, 1H, J=13.3 Hz), 1.40 (s, 9H), 1.33 (s, 3H), 1.31 (d, 3H, J=7.1 Hz), 1.27 (s, 3H).

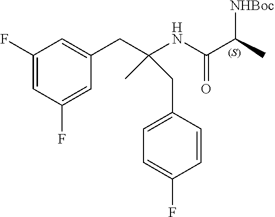

MP_045d: tert-butyl ((2S)-1-((1-(3,5-difluorophenyl)-3-(4-fluorophenyl)-2-methylpropan-2-yl)amino)-1-oxopropan-2-yl)carbamate: ¹H NMR (500 MHz, CDCl₃) δ 7.09-7.02 (m, 3H), 6.96 (m, 3H), 6.84 (m, 1H), 5.71 (s, 0.5H), 5.65 (s, 0.5H), 4.76 (s, 1H), 3.99 (m, 1H), 3.67-3.52 (m, 2H), 2.76 (m, 1H), 2.65 (d, 0.5H, J=13.6 Hz), 2.62 (d, 0.5H, J=13.6 Hz), 1.34-1.30 (m, 12H), 0.98 (bd, 3H, J=7.4 Hz).

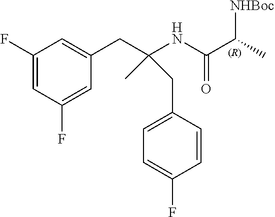

MP_046d: tert-butyl ((2R)-1-((1-(3,5-difluorophenyl)-3-(4-fluorophenyl)-2-methylpropan-2-yl)amino)-1-oxopropan-2-yl)carbamate: ¹H NMR (500 MHz, CDCl₃) δ 7.09-7.02 (m, 3H), 6.96 (m, 3H), 6.83 (m, 1H), 5.71 (s, 0.5H), 5.64 (s, 0.5H), 4.78 (s, 1H), 3.99 (m, 1H), 3.67-3.53 (m, 2H), 2.76 (m, 1H), 2.65 (d, 0.5H, J=13.6 Hz), 2.62 (d, 0.5H, J=13.6 Hz), 1.34-1.30 (m, 12H), 0.98 (bd, 3H, J=7.3 Hz).

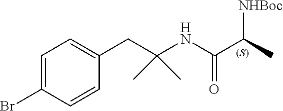

MP_047d: tert-butyl (S)-(1-((1-(4-bromophenyl)-2-methylpropan-2-yl)amino)-1-oxopropan-2-yl)carbamate: ¹H NMR (500 MHz, CDCl₃) δ 7.38 (d, 2H, J=8.4 Hz), 6.99 (d, 2H, J=8.4 Hz), 5.79 (bs, 1H), 4.85 (bs, 1H), 3.99 (m, 1H), 3.09 (d, 1H, J=13.4 Hz), 2.94 (d, 1H, J=13.5 Hz), 1.41 (s, 9H), 1.32 (s, 3H), 1.31 (d, 3H, J=7.1 Hz), 1.26 (s, 3H).

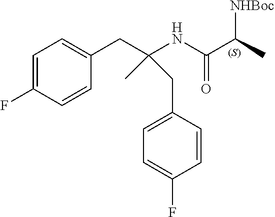

MP_051d: tert-butyl (S)-(1-((1,3-bis(4-fluorophenyl)-2-methylpropan-2-yl)amino)-1-oxopropan-2-yl)carbamate: ¹H NMR (500 MHz, CDCl₃) δ 7.08 (m, 4H), 6.95 (m, 4H), 5.58 (bs, 1H), 4.80 (bs, 1H), 3.99 (m, 1H), 3.60 (d, 1H, J=13.6 Hz), 3.55 (d, 1H, J=13.5 Hz), 2.78 (bd, 1H, J=12.9 Hz), 2.67 (d, 1H, J=13.5 Hz), 1.35 (s, 9H), 1.30 (d, 3H, J=7.1 Hz), 0.99 (s, 3H).

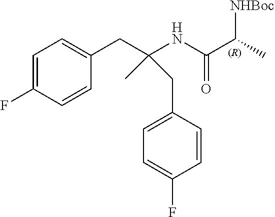

MP_052d: tert-butyl (R)-(1-((1,3-bis(4-fluorophenyl)-2-methylpropan-2-yl)amino)-1-oxopropan-2-yl)carbamate: ¹H NMR (500 MHz, CDCl₃) δ 7.08 (m, 4H), 6.95 (m, 4H), 5.58 (bs, 1H), 4.80 (bs, 1H), 3.98 (m, 1H), 3.60 (d, 1H, J=13.6 Hz), 3.55 (d, 1H, J=13.5 Hz), 2.78 (d, 1H, J=13.6 Hz), 2.67 (d, 1H, J=13.5 Hz), 1.35 (s, 9H), 1.30 (d, 3H, J=7.1 Hz), 0.99 (s, 3H).

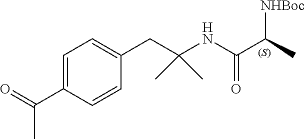

MP_053d: tert-butyl (S)-(1-((1-(4-acetylphenyl)-2-methylpropan-2-yl)amino)-1-oxopropan-2-yl)carbamate: ¹H NMR (500 MHz, CDCl₃) δ 7.86 (d, 2H, J=8.3 Hz), 7.21 (d, 2H, J=8.3 Hz), 5.81 (bs, 1H), 4.89 (bs, 1H), 4.01 (bm, 1H), 3.18 (d, 1H, J=12.8 Hz), 3.08 (d, 1H, J=13.0 Hz), 2.58 (s, 3H), 1.40 (s, 9H), 1.32-1.29 (m, 9H).

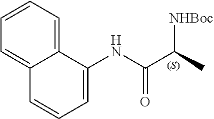

MP_054d: tert-butyl (S)-(1-(naphthalen-1-ylamino)-1-oxopropan-2-yl)carbamate: ¹H NMR (500 MHz, CDCl₃) δ 8.88 (bs, 1H), 8.07 (d, 1H, J=7.4 Hz), 7.97 (bm, 1H), 7.85 (m, 1H), 7.67 (d, 1H, J=8.2 Hz), 7.51-7.45 (m, 3H), 5.00 (bs, 1H), 4.46 (bm, 1H), 1.51 (m, 12H).

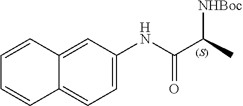

MP_055d: tert-butyl (S)-(1-(naphthalen-2-ylamino)-1-oxopropan-2-yl)carbamate: ¹H NMR (500 MHz, CDCl₃) δ 8.52 (bm, 1H), 8.22 (d, 1H, J=1.9 Hz), 7.77 (m, 3H), 7.47-7.38 (m, 3H), 4.95 (bs, 1H), 4.35 (bm, 1H), 1.47 (m, 12H).

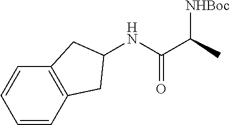 [SMILES: C[C@H](NC(=O)OC(C)(C)C)C(=O)NC1Cc2ccccc2C1]

MP_056d: tert-butyl (S)-(1-((2,3-dihydro-1H-inden-2-yl)amino)-1-oxopropan-2-yl)carbamate: ¹H NMR (500 MHz, CDCl₃) δ 7.21 (m, 2H), 7.16 (m, 2H), 6.31 (bs, 1H), 4.91 (bs, 1H), 4.71 (m, 1H), 4.06 (bm, 1H), 3.33 (dd, 1H, J=7.2 Hz), 3.29 (d, 1H, J=7.2 Hz), 2.78 (dt, 2H, J=16.1, 4.5 Hz), 1.39 (s, 9H), 1.33 (d, 3H, J=7.1 Hz).

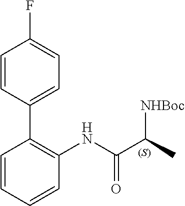

MP_057d: tert-butyl (S)-(1-((4′-fluoro-[1,1′-biphenyl]-2-yl)amino)-1-oxopropan-2-yl)carbamate: ¹H NMR (500 MHz, CDCl₃) δ 8.29 (d, 1H, J=8.2 Hz), 7.88 (s, 1H, J=7.9 Hz), 7.38-7.32 (m, 3H), 7.22-7.15 (m, 4H), 4.73 (bs, 1H), 4.14 (bm, 1H), 1.38 (s, 9H), 1.34 (d, 3H, J=7.2 Hz).

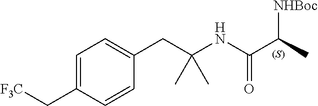

MP_058d: tert-butyl (S)-(1-((2-methyl-1-(4-(2,2,2-trifluoroethyl)phenyl)propan-2-yl)amino)-1-oxopropan-2-yl)carbamate: ¹H NMR (500 MHz, CDCl₃) δ 7.19 (d, 2H, J=7.9 Hz), 7.10 (d, 2H, J=8.0 Hz), 5.77 (bs, 1H), 4.92 (bs, 1H), 4.00 (bm, 1H), 3.32 (q, 2H, J=10.9 Hz), 3.04 (bm, 2H), 1.41 (s, 9H), 1.30 (m, 9H).

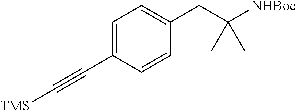 [SMILES: CC(C)(Cc1ccc(C#C[Si](C)(C)C)cc1)NC(=O)OC(C)(C)C]

MP_059d1: tert-butyl (2-methyl-1-(4-((trimethylsilyl)ethynyl)phenyl)propan-2-yl)carbamate: ¹H NMR (500 MHz, CDCl₃) δ 7.38 (d, 2H, J=8.2 Hz), 7.07 (d, 2H, J=8.2 Hz), 4.19 (s, 1H), 2.97 (s, 2H), 1.46, (bs, 9H), 1.23 (s, 6H), 0.24 (s, 9H).

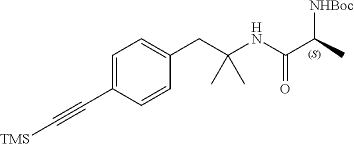

MP_059d2: tert-butyl (S)-(1-((2-methyl-1-(4-((trimethylsilyl)ethynyl)phenyl)propan-2-yl)amino)-1-oxopropan-2-yl)carbamate: ¹H NMR (500 MHz, CDCl₃) δ 7.36 (d, 2H, J=8.1 Hz), 7.04 (d, 2H, J=8.1 Hz), 5.77 (bs, 1H), 4.86 (bs, 1H), 3.99 (bm, 1H), 3.12 (d, 1H, J=13.2 Hz), 2.94 (d, 1H, J=13.2 Hz), 1.40 (s, 9H), 1.32-1.25 (m, 9H).

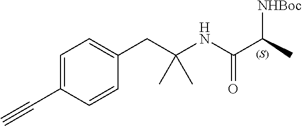

MP_059d3: tert-butyl (S)-(1-((1-(4-ethynylphenyl)-2-methylpropan-2-yl)amino)-1-oxopropan-2-yl)carbamate: ¹H NMR (500 MHz, CDCl₃) δ 7.39 (d, 2H, J=8.2 Hz), 7.07 (d, 2H, J=8.3 Hz), 5.74 (bs, 1H), 4.87 (bs, 1H), 4.00 (bm, 1H), 3.13 (d, 1H, J=13.2 Hz), 3.04 (s, 1H), 2.98 (d, 1H, J=13.2 Hz), 1.40 (s, 9H), 1.33 (bs, 3H), 1.31 (d, 3H, J=7.1 Hz), 1.27 (s, 3H).

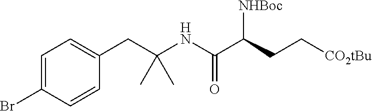 [SMILES: CC(C)(Cc1ccc(Br)cc1)NC(=O)[C@H](CCC(=O)OC(C)(C)C)NC(=O)OC(C)(C)C]

MP_061d: tert-butyl (S)-5-((1-(4-bromophenyl)-2-methylpropan-2-yl)amino)-4-((tert-butoxycarbonyl)amino)-5-oxopentanoate: ¹H NMR (500 MHz, CDCl₃) δ 7.34 (d, 2H, J=8.2 Hz), 6.99 (d, 2H, J=8.4 Hz), 5.92 (s, 1H), 5.16 (bm, 1H), 3.94 (bm, 1H), 3.09 (d, 1H, J=13.3 Hz), 2.93 (d, 1H, J=13.3 Hz), 2.39 (m, 1H), 2.27 (m, 1H), 2.01 (m, 1H), 1.85 (m, 1H), 1.44 (s, 9H), 1.41 (s, 9H), 1.32 (s, 3H), 1.26 (s, 3H).

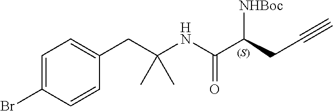

MP_062d: tert-butyl (S)-(1-((1-(4-bromophenyl)-2-methylpropan-2-yl)amino)-1-oxopent-4-yn-2-yl)carbamate: ¹H NMR (500 MHz, CDCl₃) δ 7.38 (d, 2H, J=8.0 Hz), 7.01 (d, 2H, J=8.0 Hz), 5.92 (bs, 1H), 5.18 (bs, 1H), 4.14 (bm, 1H), 3.11 (d, 1H, J=13.4 Hz), 2.91 (d, 1H, J=13.4 Hz), 2.77 (ddd, 1H, J=16.5, 7.3, 2.3 Hz), 2.55 (m, 1H), 2.05 (bs, 1H), 1.42 (s, 9H), 1.34 (s, 3H), 1.27 (s, 3H).

### Product Compounds (as HCl Salts)

### Except MP_024

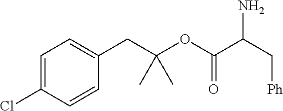 [SMILES: CC(C)(Cc1ccc(Cl)cc1)OC(=O)C(N)Cc1ccccc1]

MP_001: 1-(4-chlorophenyl)-2-methylpropan-2-yl 2-amino-3-phenylpropanoate: ¹H NMR (400 MHz, MeOD) □ 7.37-7.12 (m, 9H), 4.15 (t, 1H, J=7.0 Hz), 3.10 (d, 2H, J=7.0 Hz), 2.99 (d, 1H, J=13.7 Hz), 2.90 (d, 1H, J=13.7 Hz), 1.46 (s, 3H) 1.42 (s, 3H). ¹³C NMR (400 MHz, MeOD) □ 168.1, 135.2, 134.3, 131.9, 132.4, 129.1, 128.7, 127.8, 127.5, 85.2, 54.2, 45.6, 36.4, 24.4, 24.3. HRMS (ESI): calcd for C₁₉H22ClNO₂ [M+H]⁺: 332.1417, found: 332.1418.

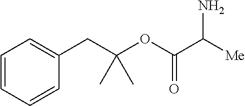 [SMILES: CC(N)C(=O)OC(C)(C)Cc1ccccc1]

MP_002: 2-methyl-1-phenylpropan-2-yl 2-aminopropanoate: ¹H NMR (400 MHz, MeOD) δ 7.27 (m, 5H), 3.95 (q, 1H, J=7.2 Hz), 3.12 (d, 2H, 2.9 Hz), 1.52 (s, 3H) 1.51 (s, 3H), 1.42 (d, 3H, J=7.2 Hz). ¹³C NMR (400 MHz, MeOD) δ 168.9, 136.5, 130.3, 127.7, 126.4, 85.3, 48.9, 45.8, 24.9, 24.5, 14.9. HRMS (ESI): calcd for C₁₃H₉NO₂ [M+H]⁺: 222.1494, found: 222.1484.

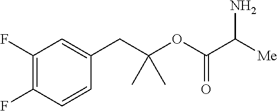 [SMILES: CC(N)C(=O)OC(C)(C)Cc1ccc(F)c(F)c1]

MP_003: 1-(3,4-difluorophenyl)-2-methylpropan-2-yl 2-aminopropanoate: ¹H NMR (500 MHz, MeOD) δ7.18 (m, 2H), 7.02 (m, 1H), 3.97 (q, 1H, J=7.2 Hz), 3.10 (d, 2H, J=3.5 Hz), 1.52 (s, 3H) 1.51 (s, 3H), 1.42 (d, 3H, J=7.0 Hz). ¹³C NMR (500 MHz, MeOD) δ 169.0, 149.7 (dd, J=246.5, 12.7 Hz), 149.3 (dd, J=245.9, 12.6 Hz), 134.0 (dd, J=5.7, 4.0 Hz), 129.8 (dd, J=6.2, 3.5 Hz), 119.0 (d, J=17.1 Hz), 116.4 (d, J=17.2 Hz), 84.8, 48.6, 44.9, 24.7, 24.4, 14.9. HRMS (ESI): calcd for C₁₃H₁₈ClF₂NO₂ [M+H]⁺: 258.1306, found: 258.1295.

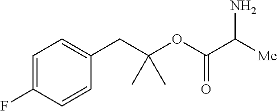 [SMILES: CC(N)C(=O)OC(C)(C)Cc1ccc(F)cc1]

MP_004: 1-(4-fluorophenyl)-2-methylpropan-2-yl 2-aminopropanoate: ¹H NMR (500 MHz, MeOD) δ7.23 (m, 2H), 7.02 (m, 2H), 3.95 (q, 1H, J=7.2 Hz), 3.10 (d, 2H, J=2.3 Hz), 1.52 (s, 3H) 1.49 (s, 3H), 1.42 (d, 3H, J=7.2 Hz). ¹³C NMR (500 MHz, MeOD) δ 169.0, 161.9 (d, J=243.6 Hz), 132.5 (d, J=3.3 Hz), 131.9 (d, J=7.9 Hz), 114.3 (d, J=21.4 Hz), 85.1, 48.9, 44.9, 24.7, 24.4, 14.9. HRMS (ESI): calcd for C₁₃H₁₉FNO₂ [M+H]⁺: 240.1400, found: 257.1389.

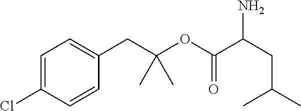 [SMILES: CC(C)CC(N)C(=O)OC(C)(C)Cc1ccc(Cl)cc1]

MP_005: 1-(4-chlorophenyl)-2-methylpropan-2-yl 2-amino-4-methylpentanoate: ¹H NMR (500 MHz, MeOD) δ7.30 (d, 2H, J=8.4 Hz), 7.22 (d, 2H, J=8.4 Hz), 3.86 (dd, 1H, J=7.6, 6.1 Hz), 3.12 (d, 1H, J=13.7 Hz), 3.07 (d, 1H, J=13.7 Hz), 1.65-1.50 (m, 9H), 0.92 (m, 6H). ¹³C NMR (500 MHz, MeOD) δ 169.0, 135.3, 132.5, 131.9, 127.8, 85.1, 51.4, 45.2, 39.4, 24.9, 24.4, 24.1, 21.2, 20.7. HRMS (DART): calcd for C₁₆H₂₄ClNO₂ [M+H]⁺: 298.1568, found: 284.1553.

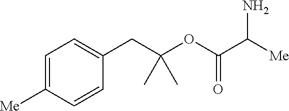 [SMILES: Cc1ccc(CC(C)(C)OC(=O)C(C)N)cc1]

MP_006: 2-methyl-1-p-tolylpropan-2-yl 2-aminopropanoate: ¹H NMR (500 MHz, MeOD) δ 7.10 (m, 4H), 3.94 (q, 1H, J=7.2 Hz), 3.09 (d, 1H, J=13.7 Hz), 3.06 (d, 1H, J=13.7 Hz), 2.30 (s, 3H), 1.51 (s, 3H), 1.50 (s, 3H), 1.43 (d, 3H, J=7.3 Hz). ¹³C NMR (500 MHz, MeOD) δ 168.9, 136.1, 133.3, 130.2, 128.3, 85.4, 48.9, 45.3, 24.8, 24.5, 19.7, 14.9. HRMS (DART): calcd for C₁₄H₂₁NO₂ [M+H]⁺: 236.1645, found: 236.1638.

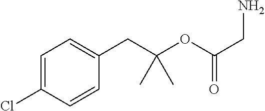 [SMILES: CC(C)(Cc1ccc(Cl)cc1)OC(=O)CN]

MP_007: 1-(4-chlorophenyl)-2-methylpropan-2-yl 2-aminoethanoate: ¹H NMR (500 MHz, MeOD) δ7.30 (d, 2H, J=8.4 Hz), 7.21 (d, 2H, J=8.4 Hz), 3.71 (s, 2H), 3.11 (s, 2H), 1.51 (s, 6H). ¹³C NMR (500 MHz, MeOD) δ 166.4, 135.4, 132.4, 131.8, 127.8, 85.0, 45.0, 40.2, 24.7. HRMS (DART): calcd for C₁₂H₁₆ClNO₂ [M+H]⁺: 242.0942, found: 242.0938.

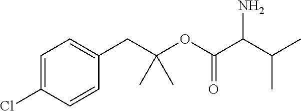 [SMILES: CC(C)C(N)C(=O)OC(C)(C)Cc1ccc(Cl)cc1]

MP_008: 1-(4-chlorophenyl)-2-methylpropan-2-yl 2-amino-3-methylbutanoate: ¹H NMR (500 MHz, MeOD) δ7.31 (d, 2H, J=8.4 Hz), 7.24 (d, 2H, J=8.4 Hz), 3.80 (d, 1H, J=4.4 Hz), 3.16 (d, 1H, J=13.7 Hz), 3.03 (d, 1H, J=13.7 Hz), 2.24 (qd, 1H, J=7.0, 4.3 Hz), 1.57 (s, 3H), 1.49 (s, 3H) 1.04 (d, 3H, J=7.0 Hz), 1.00 (d, 3H, J=7.0 Hz). ¹³C NMR (500 MHz, MeOD) δ 168.0, 135.2, 132.4, 132.1, 127.8, 85.4, 58.3, 45.7, 29.5, 24.6, 24.4, 16.9, 16.7. HRMS (DART): calcd for C₁₅H₂ClNO₂ [M+H]⁺: 284.1412, found: 284.1408.

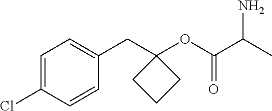 [SMILES: CC(N)C(=O)OC1(Cc2ccc(Cl)cc2)CCC1]

MP_009: 1-(4-chlorobenzyl)cyclobutyl 2-aminopropanoate: ¹H NMR (500 MHz, MeOD) δ7.33 (d, 2H, J=8.4 Hz), 7.23 (d, 2H, J=8.4 Hz), 4.02 (q, 1H, J=7.2 Hz), 3.30 (m, 2H), 2.41 (m, 4H), 1.92 (m, 1H), 1.73 (m, 1H), 1.44 (d, 3H, J=7.2 Hz). ¹³C NMR (500 MHz, MeOD) δ 168.4, 135.2, 132.3, 131.1, 127.9, 84.3, 48.5, 39.7, 33.1, 33.0, 14.7, 12.9. HRMS (ESI): calcd for C₁₄H₁₈ClNO₂ [M+H]⁺: 268.1104, found: 268.1098.

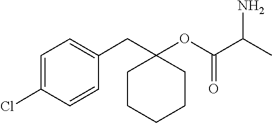 [SMILES: CC(N)C(=O)OC1(Cc2ccc(Cl)cc2)CCCCC1]

MP_010: 1-(4-chlorobenzyl)cyclohexyl 2-aminopropanoate: ¹H NMR (500 MHz, MeOD) δ7.29 (d, 2H, J=8.0 Hz), 7.15 (d, 2H, J=8.0 Hz), 3.99 (q, 1H, J=7.1 Hz), 3.24 (m, 2H), 2.25 (m, 2H), 1.68-1.40 (m, 10H), 1.32 (m, 1H). ¹³C NMR (500 MHz, MeOD) δ 169.1, 134.8, 132.4, 131.7, 127.8, 86.8, 48.6, 42.2, 33.8, 33.7 24.8, 21.3, 15.1. HRMS (DART): calcd for C₁₆H₂₂ClNO₂ [M+H]⁺: 296.14118, found: 296.13971.

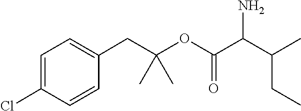 [SMILES: CCC(C)C(N)C(=O)OC(C)(C)Cc1ccc(Cl)cc1]

MP_011: 1-(4-chlorophenyl)-2-methylpropan-2-yl 2-amino-3-methylpentanoate: ¹H NMR (500 MHz, CDCl₃) δ 8.79 (bs, 3H), 7.26 (d, 2H, J=8.1 Hz), 7.13 (d, 2H, 8.1 Hz), 3.86 (m, 1H), 3.18-2.96 (m, 2H), 2.08 (m, 1H), 1.76-1.34 (m, 8H), 1.08-0.87 (m, 6H). ¹³C NMR (500 MHz, CDCl₃) δ 167.7 (diast. A), 167.3 (diast. B), 134.9, 132.7, 132.0, 128.3, 85.6 (diast. A), 85.8 (diast. B), 57.6 (diast. A), 57.8 (diast. B), 45.9 (diast. A), 45.8 (diast. B), 36.7 (diast. B), 36.4 (diast. A), 25.6, 25.5, 15.0, 14.4, 11.8 (diast. A), 11.7 (diast. B). HRMS (DART): calcd for C₁₆H₂₄ClNO₂ [M+H]⁺: 298.1568, found: 298.1555.

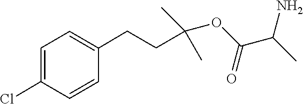 [SMILES: CC(N)C(=O)OC(C)(C)CCc1ccc(Cl)cc1]

MP_012: 4-(4-chlorophenyl)-2-methylbutan-2-yl 2-aminopropanoate: ¹H NMR (500 MHz, MeOD) δ7.26 (d, 2H, J=8.4 Hz), 7.18 (d, 2H, J=8.4 Hz), 3.97 (q, 1H, J=7.2 Hz), 2.66 (m, 2H), 2.11 (m, 2H), 1.56 (s, 6H), 1.51 (d, 3H, J=7.3 Hz). ¹³C NMR (500 MHz, MeOD) δ 168.8, 140.5, 131.3, 129.6, 128.1, 85.4, 48.9, 42.0, 29.2, 24.8, 24.7, 15.0. HRMS (DART): calcd for C₁₄H₂₀ClNO₂ [M+H]⁺: 270.1255, found: 270.1244.

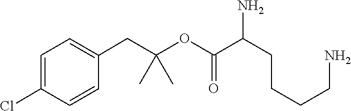 [SMILES: CC(C)(Cc1ccc(Cl)cc1)OC(=O)C(N)CCCCN]

MP_013: 1-(4-chlorophenyl)-2-methylpropan-2-yl 2,6-diaminohexanoate: ¹H NMR (500 MHz, MeOD) δ7.32 (d, 2H, J=8.4 Hz), 7.23 (d, 2H, J=8.4 Hz), 3.92 (t, 1H, J=6.5 Hz), 3.11 (s, 2H), 2.87 (t, 2H, J=7.9 Hz), 1.83 (m, 2H), 1.64 (m, 2H), 1.54 (s, 3H), 1.51 (s, 3H), 1.39 (m, 2H). ¹³C NMR (500 MHz, MeOD) δ 168.4, 135.3, 132.4, 132.0, 127.9, 85.4, 52.8, 45.2, 38.8, 29.8, 26.7, 24.9, 24.4, 21.4. HRMS (DART): calcd for C₁₆H25ClN₂O₂ [M+H]⁺: 313.1677, found: 313.1662.

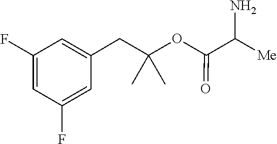 [SMILES: CC(N)C(=O)OC(C)(C)Cc1cc(F)cc(F)c1]

MP_014: 1-(3,5-difluorophenyl)-2-methylpropan-2-yl 2-aminopropanoate: ¹H NMR (500 MHz, CDCl₃) δ 8.78 (bs, 3H), 6.69 (m, 3H), 4.10 (q, 1H, J=7.1 Hz), 3.05 (m, 2H), 1.64 (d, 3H, J=7.2 Hz), 1.48 (s, 3H), 1.46 (s, 3H). ¹³C NMR (500 MHz, CDCl₃) δ 168.9, 162.6 (dd, J=248.1, 12.9 Hz), 140.2 (t, J=9.2 Hz), 113.4 (m), 102.3 (t, J=25.2 Hz), 84.9, 49.7, 46.3, 25.8, 25.5, 16.2. HRMS (DART): calcd for C₁₃H₁₇F₂NO₂ [M+H]⁺: 258.1300, found: 258.1290.

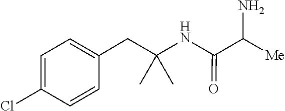 [SMILES: CC(N)C(=O)NC(C)(C)Cc1ccc(Cl)cc1]

MP_015: 2-amino-N-(1-(4-chlorophenyl)-2-methylpropan-2-yl)propanamide: ¹H NMR (500 MHz, MeOD) δ7.26 (d, 2H, J=8.3 Hz), 7.13 (d, 2H, J=8.3 Hz), 3.78 (t, 1H, J=7.0 Hz), 3.13 (s, 1H, J=13.7 Hz), 2.98 (d, 1H, J=13.7 Hz), 1.41 (d, 3H, J=7.0 Hz), 1.32 (m, 3H), 1.28 (s, 3H). ¹³C NMR (500 MHz, MeOD) δ 168.8, 136.6, 132.0, 131.8, 127.6, 53.9, 49.0, 42.9, 25.9, 16.7. HRMS (DART): calcd for C₁₃H₁₉ClN₂O [M+H]⁺: 255.1258, found: 255.1250.

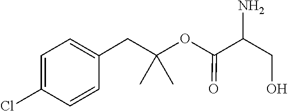 [SMILES: CC(C)(Cc1ccc(Cl)cc1)OC(=O)C(N)CO]

MP_016: 1-(4-chlorophenyl)-2-methylpropan-2-yl 2-amino-3-hydroxypropanoate: ¹H NMR (500 MHz, MeOD) δ7.29 (d, 2H, J=8.2 Hz), 7.22 (d, 2H, J=8.1 Hz), 3.97 (m, 1H), 3.86 (m, 2H), 3.10 (m, 2H), 1.52 (s, 3H), 1.49 (s, 3H). ¹³C NMR (500 MHz, MeOD) δ 166.8, 135.3, 132.3, 131.9, 127.8, 85.2, 59.3, 55.1, 45.1, 24.7, 24.5. HRMS (DART): calcd for C₁₃H₁₈ClNO₂ [M+H]⁺: 272.1048, found: 272.1041.

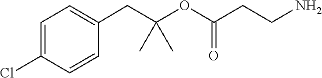 [SMILES: CC(C)(Cc1ccc(Cl)cc1)OC(=O)CCN]

MP_017: 1-(4-chlorophenyl)-2-methylpropan-2-yl 3-aminopropanoate: ¹H NMR (500 MHz, MeOD) δ7.28 (d, 2H, J=8.5 Hz), 7.19 (d, 2H, J=8.5 Hz), 3.13 (t, 1H, J=6.6 Hz), 3.00 (s, 2H), 2.64 (t, 2H, J=6.6 Hz), 1.47 (s, 6H). ¹³C NMR (500 MHz, MeOD) δ 170.2, 135.7, 132.2, 131.8, 127.7, 83.2, 45.2, 34.9, 31.8, 24.7. HRMS (DART): calcd for C₁₃H₁₈ClNO₂ [M+H]⁺: 256.1099, found: 256.1092.

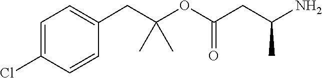 [SMILES: C[C@H](N)CC(=O)OC(C)(C)Cc1ccc(Cl)cc1]

MP_018: (S)-1-(4-chlorophenyl)-2-methylpropan-2-yl 3-aminobutanoate: ¹H NMR (500 MHz, CDCl₃) δ 8.51 (bs, 3H), 7.27 (d, 2H, J=8.3 Hz), 7.08 (d, 2H, J=8.3 Hz), 3.69 (m, 1H), 3.00 (s, 2H), 2.72 (m, 2H), 1.50 (d, 3H, J=5.6 Hz), 1.44 (s, 3H), 1.43 (s, 3H). ¹³C NMR (500 MHz, CDCl₃) δ 170.6, 135.1, 132.7, 131.8, 128.4, 84.3, 45.9, 45.3, 38.6, 25.9, 25.8, 18.4. HRMS (DART): calcd for C₁₄H₂₀ClNO₂ [M+H]⁺: 270.1255, found: 270.1242.

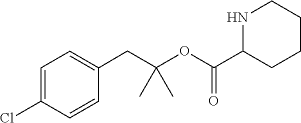 [SMILES: CC(C)(Cc1ccc(Cl)cc1)OC(=O)C1CCCCN1]

MP_019: 1-(4-chlorophenyl)-2-methylpropan-2-yl piperidine-2-carboxylate: ¹H NMR (500 MHz, CDCl₃) δ 10.25 (bs, 1H), 9.57 (bs, 1H), 7.27 (d, 2H, J=8.4 Hz), 7.14 (d, 2H, J=8.4 Hz), 3.83 (m, 1H), 3.58 (m, 1H), 3.07 (m, 3H), 2.14 (m, 1H), 2.02-1.85 (m, 3H), 1.68 (m, 1H), 1.56 (m, 1H), 1.49 (s, 3H), 1.45 (s, 3H). ¹³C NMR (500 MHz, CDCl₃) δ 167.4, 134.8, 132.8, 131.9, 128.4, 85.6, 56.5, 45.5, 43.3, 26.0, 25.9, 25.7, 21.7, 21.4. HRMS (DART): calcd for C₁₆H₂₂ClNO₂ [M+H]⁺: 296.1412, found: 296.1403.

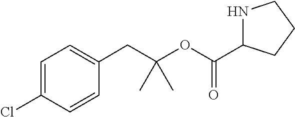 [SMILES: CC(C)(Cc1ccc(Cl)cc1)OC(=O)C1CCCN1]

MP_020: 1-(4-chlorophenyl)-2-methylpropan-2-yl pyrrolidine-2-carboxylate: ¹H NMR (500 MHz, MeOD) δ7.30 (d, 2H, J=8.3 Hz), 7.21 (d, 2H, J=8.3 Hz), 4.29 (m, 1H), 3.36 (m, 2H), 3.09 (s, 2H), 2.34 (m, 1H), 2.07-1.90 (m, 3H), 1.53 (s, 3H), 1.50 (s, 3H). ¹³C NMR (500 MHz, MeOD) δ 168.0, 135.3, 132.5, 131.9, 127.8, 85.5, 59.8, 45.7, 45.3, 28.0, 24.7, 24.4, 23.1. HRMS (DART): calcd for C₁₅H₂₀ClNO₂ [M+H]⁺: 282.1255, found: 282.1248.

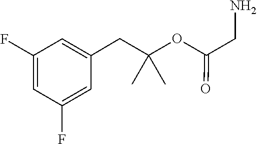 [SMILES: CC(C)(Cc1cc(F)cc(F)c1)OC(=O)CN]

MP_021: 1-(3,4-difluorophenyl)-2-methylpropan-2-yl 2-aminoethanoate: ¹H NMR (500 MHz, MeOD) δ7.17 (m, 2H), 7.02 (m, 1H), 3.72 (s, 2H), 3.09 (s, 2H), 1.50 (s, 6H). ¹³C NMR (500 MHz, CDCl₃) δ 166.4, 149.7 (d, J=246.4, 12.7 Hz), 149.3 (dd, J=245.3, 13.0 Hz), 134.1 (dd, J=5.7, 4.1 Hz), 126.7 (dd, J=6.3, 3.4 Hz), 118.9 (d, J=17.0 Hz), 116.4 (d, J=17.2 Hz), 84.9, 44.9, 40.1, 24.6.

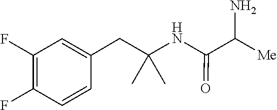 [SMILES: CC(N)C(=O)NC(C)(C)Cc1ccc(F)c(F)c1]

MP_022: -amino-N-(1-(3,4-difluorophenyl)-2-methylpropan-2-yl)propanamide: ¹H NMR (400 MHz, MeOD) δ7.13 (dt, 1H, J=10.7, 8.4 Hz), 7.05 (ddd, J=11.7, 7.9, 2.1 Hz), 6.92 (m, 1H), 3.75 (q, 1H, J=7.0 Hz), 3.15 (d, 1H, J=13.3 Hz), 2.94 (d, 2H, J=13.3 Hz), 1.39 (d, 3H, J=7.0 Hz), 1.32 (s, 3H), 1.26 (s, 3H). ¹³C NMR (500 MHz, MeOD) δ 168.8, 149.6 (dd, J=245.7, 12.7 Hz), 149.1 (dd, J=244.9, 12.6 Hz), 135.3 (dd, J=5.7, 4.2 Hz), 126.6 (dd, J=6.1, 3.5 Hz), 118.7 (d, J=17.1 Hz), 116.2 (d, J=17.1 Hz), 53.9, 49.0, 42.7, 25.9, 16.6. HRMS (ESI): calcd for C₁₃H₈F₂N₂O [M+H]⁺: 257.1460, found: 257.1454.

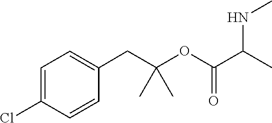 [SMILES: CNC(C)C(=O)OC(C)(C)Cc1ccc(Cl)cc1]

MP_023: 1-(4-chlorophenyl)-2-methylpropan-2-yl 2-(methylamino)propanoate: ¹H NMR (500 MHz, CDCl₃) δ 10.00 (bs, 1H), 9.69 (bs, 1H), 7.27 (m, 2H), 7.13 (d, 2H, J=7.6 Hz), 3.77 (m, 1H), 3.07 (m, 1H), 2.72 (s, 3H), 1.63 (s, 3H), 1.50 (s, 3H), 1.49 (s, 3H). ¹³C NMR (500 MHz, CDCl₃) δ 167.6, 134.7, 132.9, 131.9, 128.4, 85.9, 56.9, 45.7, 31.1, 25.9, 25.7, 14.8. HRMS (DART): calcd for C₁₄H₂₀ClNO₂ [M+H]⁺: 270.1255, found: 270.1242.

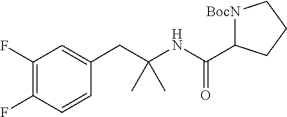 [SMILES: CC(C)(Cc1ccc(F)c(F)c1)NC(=O)C1CCCN1C(=O)OC(C)(C)C]

MP_024: tert-butyl 2-(1-(3,4-difluorophenyl)-2-methylpropan-2-ylcarbamoyl)pyrrolidine-1-carboxylate: ¹H NMR (500 MHz, CDCl₃) δ 7.04-6.80 (m, 3H), 4.17 (m, 1H), 3.35 (m, 2H), 2.70-1.80 (m, 4H) 1.41-1.39 (m, 12H), 1.16 (s, 1H). HRMS (DART): calcd for C₂₀H₂₈F₂N₂O₃ [M+H]⁺: 383.2141 found: 383.2134.

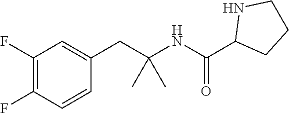 [SMILES: CC(C)(Cc1ccc(F)c(F)c1)NC(=O)C1CCCN1]

MP_025: N-(1-(3,4-difluorophenyl)-2-methylpropan-2-yl)pyrrolidine-2-carboxamide: ¹H NMR (500 MHz, MeOD) δ7.16 (m, 1H), 7.04 (m, 1H), 6.93 (m, 1H), 4.06 (m, 1H), 3.41 (m, 1H), 3.30 (m, 1H), 3.10 (d, 1H, J=13.4 Hz), 3.03 (d, 1H, J=13.4 Hz), 2.30 (m, 1H), 2.03 (m, 2H), 1.89 (m, 1H), 1.32 (s, 3H), 1.31 (s, 3H). ¹³C NMR (500 MHz, MeOD) δ 167.7, 150.3 (dd, J=71.7, 13.3 Hz), 148.3 (dd, J=71.3, 12.8 Hz), 135.4 (m), 126.0 (m), 118.6 (d, J=16.8 Hz), 116.3 (d, J=17.2 Hz), 60.0, 54.1, 45.9, 42.8, 29.9, 25.9, 25.8, 23.8. HRMS (DART): calcd for C₁₅H₂₀F₂N₂O [M+H]⁺: 283.1617, found: 283.1606.

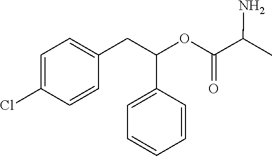 [SMILES: CC(N)C(=O)OC(Cc1ccc(Cl)cc1)c1ccccc1]

MP_026: ¹H NMR (400 MHz, CDCl₃) δ 8.65 (bs, 3H), 7.21-7.07 (m, 7H), 6.92 (d, 1H, J=7.8 Hz), 6.87 (d, 1H, 7.6 Hz), 5.85 (m, 1H), 4.03 (m, 1H), 3.10 (m, 2H), 1.50 (m, 3H). ¹³C NMR (400 MHz, CDCl₃) δ 169.1, 138.1, 134.4, 134.3, 132.7, 132.6, 131.1, 130.9, 128.54, 128.50, 128.46, 128.4, 126.6, 126.5, 78.9, 49.3, 41.9, 41.8, 30.9, 15.9. HRMS (DART): calcd for C₁₇H₁₈ClNO₂ [M+H]⁺: 304.1099, found: 304.1089.

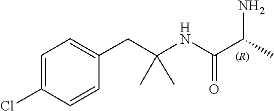

MP_027: (R)-2-amino-N-(1-(4-chlorophenyl)-2-methylpropan-2-yl)propanamide: ¹H NMR (500 MHz, MeOD) δ7.26 (d, 2H, J=8.1 Hz), 7.14 (d, 2H, J=8.0 Hz), 3.79 (m, 1H), 3.14 (d, 1H, J=13.3 Hz), 2.99 (d, 1H, J=13.3 Hz), 1.42 (d, 3H, J=6.9 Hz), 1.33 (s, 3H), 1.29 (s, 3H). ¹³C NMR (500 MHz, MeOD) δ 168.8, 136.6, 132.0, 131.8, 127.6, 53.9, 49.1, 42.9, 26.0, 25.9, 16.7. HRMS (DART): calcd for C₁₃H₁₉ClN₂O [M+H]⁺: 255.1259, found: 255.1251.

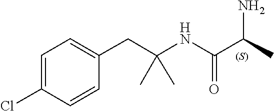

MP_028: (S)-2-amino-N-(1-(4-chlorophenyl)-2-methylpropan-2-yl)propanamide: ¹H NMR (500 MHz, MeOD) δ7.26 (d, 2H, J=8.3 Hz), 7.14 (d, 2H, J=8.3 Hz), 3.75 (q, 1H, J=7.1 Hz), 3.14 (d, 1H, J=13.3 Hz), 2.99 (d, 1H, J=13.3 Hz), 1.41 (d, 3H, J=7.1 Hz), 1.32 (s, 3H), 1.29 (s, 3H). ¹³C NMR (500 MHz, MeOD) δ 168.9, 136.6, 132.0, 131.8, 127.6, 53.9, 49.1, 43.0, 26.0, 25.9, 16.8. HRMS (DART): calcd for C₁₃H₁₉ClN₂O [M+H]⁺: 255.1259, found: 255.1253.

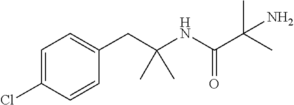 [SMILES: CC(C)(Cc1ccc(Cl)cc1)NC(=O)C(C)(C)N]

MP_029: 2-amino-N-(1-(4-chlorophenyl)-2-methylpropan-2-yl)-2-methylpropanamide: ¹H NMR (500 MHz, MeOD) δ 7.26 (d, 2H, J=8.5 Hz), 7.14 (d, 2H, J=8.5 Hz), 3.08 (s, 2H), 1.47 (s, 3H), 1.33 (s, 3H). ¹³C NMR (500 MHz, MeOD) δ 171.0, 136.7, 132.0, 131.8, 127.5, 57.0, 42.6, 26.0, 22.7. HRMS (DART): calcd for C₁₄H₂₁ClN₂O [M+H]⁺: 269.1415, found: 269.1408.

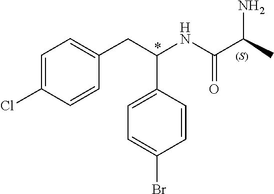

MP_030: (2S)-2-amino-N-(1-(4-bromophenyl)-2-(4-chlorophenyl)ethyl)propanamide: ¹H NMR (500 MHz, MeOD) δ7.45 (d, 2H, J=8.4 Hz), 7.23 (m, 4H), 6.97 (d, 2H, J=8.4 Hz), 3.89 (m, 1H, J=7.0 Hz), 3.50 (d, 1H, J=12.9 Hz), 3.21 (d, 1H, J=12.9 Hz), 1.52 (bm, 6H). ¹³C NMR (500 MHz, MeOD) δ 168.4, 144.7, 135.2, 132.4, 132.0, 130.8, 127.6, 127.3, 120.1, 58.5, 48.9, 43.6, 24.9, 16.5. HRMS (DART): calcd for C₁₈H₂₀BrClN₂O [M+H]⁺: 395.0520, found: 395.0508.

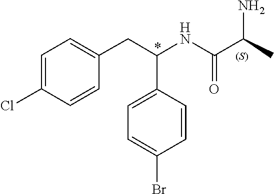

MP_031: (2S)-2-amino-N-(1-(4-bromophenyl)-2-(4-chlorophenyl)ethyl)propanamide: ¹H NMR (500 MHz, MeOD) δ7.44 (d, 2H, J=8.6 Hz), 7.22 (m, 4H), 6.94 (d, 2H, J=8.4 Hz), 3.92 (m, 1H, J=7.0 Hz), 3.52 (d, 1H, J=13.0 Hz), 3.20 (d, 1H, J=13.0 Hz), 1.55 (s, 3H), 1.43 (d, 3H, J=7.0 Hz). ¹³C NMR (500 MHz, MeOD) δ 168.3, 144.6, 135.0, 132.4, 132.1, 130.8, 127.5, 127.3, 120.1, 58.5, 48.9, 44.0, 25.2, 16.4. HRMS (ESI): calcd for C₁₈H₂₀BrClN₂O [M+H]⁺: 395.0520, found: 395.0536.

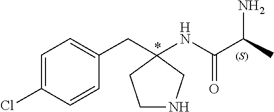

MP_032 (diastereomer 1): 3-(4-chlorobenzyl)pyrrolidin-3-yl L-alaninate: ¹H NMR (500 MHz, MeOD) δ7.35 (d, 2H, J=8.4 Hz), 7.23 (d, 2H, J=8.4 Hz), 4.15 (q, 1H, J=7.3 Hz), 4.01 (dd, 1H, J=13.3, 1.4 Hz), 3.67-3.65 (m, 1H), 3.56-3.44 (m, 3H), 3.32-3.29 (m, 1H), 2.46-2.34 (m, 2H), 1.37 (d, 3H, J=7.3 Hz). ¹³C NMR (500 MHz, MeOD) δ 169.5, 133.9, 133.2, 133.1, 128.5, 90.6, 51.9, 49.0, 43.7, 37.6, 34.9, 14.4.

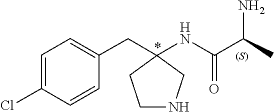

MP_033 (diastereomer 2): 3-(4-chlorobenzyl)pyrrolidin-3-yl L-alaninate: ¹H NMR (500 MHz, MeOD) δ7.36 (d, 2H, J=8.5 Hz), 7.23 (d, 2H, J=8.5 Hz), 4.09 (q, 1H, J=7.3 Hz), 3.77 (dd, 1H, J=13.3, 1.8 Hz), 3.57-3.43 (m, 5H), 2.67 (m, 1H), 2.34 (m, 1H), 1.39 (d, 3H, J=7.3 Hz). ¹³C NMR (500 MHz, MeOD) δ 169.1, 133.7, 133.2, 131.3, 128.5, 90.8, 53.1, 48.9, 43.7, 37.9, 33.9, 14.6.

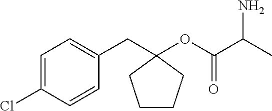 [SMILES: CC(N)C(=O)OC1(Cc2ccc(Cl)cc2)CCCC1]

MP_034: 1-(4-chlorobenzyl)cyclopentyl alaninate: ¹H NMR (500 MHz, MeOD) δ 7.29 (d, 2H, J=8.5 Hz), 7.19 (d, 2H, J=8.5 Hz), 3.92 (q, 1H, J=7.3 Hz), 3.34 (m, 2H), 2.13 (m, 2H), 1.88 (m, 2H), 1.74 (m, 4H), 1.37 (d, 3H, J=7.2 Hz). ¹³C NMR (500 MHz, MeOD) δ 169.3, 135.9, 132.3, 131.3, 127.9, 95.6, 48.8, 40.8, 36.6, 36.5, 22.8, 22.7, 14.8. HRMS (ESI): calcd for C₁₅H20ClNO₂ [M+H]⁺: 282.1253, found: 282.1268.

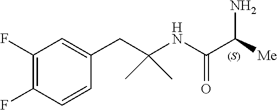

MP_035: (S)-2-amino-N-(1-(3,4-difluorophenyl)-2-methylpropan-2-yl)propanamide: ¹H NMR (500 MHz, MeOD) δ7.15 (dt, 1H, J=10.5, 8.4 Hz), 7.05 (ddd, 1H, J=11.5, 7.8, 2.0 Hz), 6.95 (m, 1H), 3.76 (q, 1H, J=7.0 Hz), 3.18 (d, 1H, J=13.4 Hz), 2.97 (d, 1H, J=13.4 Hz), 1.41 (d, 3H, J=7.1 Hz), 1.34 (s, 3H), 1.28 (s, 3H). ¹³C NMR (500 MHz, MeOD) δ 168.8, 149.6 (dd, J=245.5, 12.6 Hz), 149.1 (dd, J=245.7, 12.3 Hz), 135.4 (dd, J=5.5, 4.0 Hz), 126.6 (dd, J=6.1, 3.4 Hz), 118.7 (d, J=16.8 Hz), 116.2 (d, J=17.0 Hz), 53.9, 49.0, 42.7, 25.9, 16.6. HRMS (DART): calcd for C₁₃H₈F₂N₂O [M+H]⁺: 257.1460, found: 257.1454.

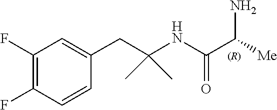

MP_036: (R)-2-amino-N-(1-(3,4-difluorophenyl)-2-methylpropan-2-yl)propanamide: ¹H NMR (500 MHz, MeOD) δ7.15 (dt, 1H, J=10.7, 8.4 Hz), 7.05 (ddd, 1H, J=11.7, 7.8, 2.1 Hz), 6.95 (m, 1H), 3.77 (q, 1H, J=7.0 Hz), 3.18 (d, 1H, J=13.4 Hz), 2.96 (d, 1H, J=13.4 Hz), 1.41 (d, 3H, J=7.1 Hz), 1.34 (s, 3H), 1.28 (s, 3H). ¹³C NMR (500 MHz, MeOD) δ 168.8, 149.6 (dd, J=245.5, 12.7 Hz), 149.1 (dd, J=245.5, 12.6 Hz), 135.4 (dd, J=5.6, 4.1 Hz), 126.6 (dd, J=6.1, 3.3 Hz), 118.7 (d, J=16.8 Hz), 116.2 (d, J=17.0 Hz), 53.9, 49.0, 42.7, 25.9, 16.6. HRMS (DART): calcd for C₁₃H₈F₂N₂O [M+H]⁺: 257.1460, found: 257.1453.

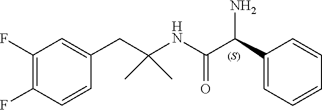

MP_037: tert-butyl (S)-(2-((1-(3,4-difluorophenyl)-2-methylpropan-2-yl)amino)-2-oxo-1-phenylethyl)carbamate: ¹H NMR (500 MHz, MeOD) δ 7.54-7.47 (m, 5H), 6.87-6.81 (, 2H), 6.61 (m, 1H), 4.79 (s, 1H), 3.11 (d, 1H, J=13.5 Hz), 2.88 (d, 1H, J=13.5 Hz), 1.33 (s, 3H), 1.19 (s, 3H). ¹³C NMR (500 MHz, MeOD) δ 166.6, 149.5 (d, J=246.6 Hz), 149.4 (d, J=247.3 Hz), 148.9 (d, J=244.4 Hz), 148.8 (d, J=244.9 Hz), 135.0 (m), 133.4, 129.7, 129.1, 127.6, 126.2 (m), 118.6 (d, J=16.6 Hz), 116.6 (d, J=17.1 Hz), 55.62, 55.60, 54.1, 42.7, 26.0, 25.7. HRMS (DART): calcd for C₁₈H₂₀F₂N₂O [M+H]⁺: 319.1617, found: 319.1609.

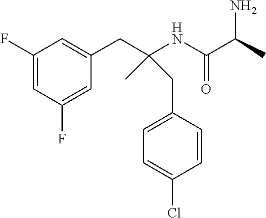 [SMILES: C[C@H](N)C(=O)NC(C)(Cc1ccc(Cl)cc1)Cc1cc(F)cc(F)c1]

MP_038: (2S)-2-amino-N-(1-(4-chlorophenyl)-3-(3,5-difluorophenyl)-2-methylpropan-2-yl)propanamide: ¹H NMR (500 MHz, MeOD) δ7.29-7.26 (m, 2H), 7.19-7.05 (m, 4H), 6.98 (m, 1H), 3.76-3.72 (m, 1H), 3.70-3.65 (m, 1H), 3.60-3.56 (m, 1H), 2.80-2.70 (m, 2H), 1.34 (d, 3H, J=7.1 Hz). 1.02 (d, 3H, J=3.8 Hz). ¹³C NMR (500 MHz, MeOD) δ 169.17, 169.09, 169.02, 150.64, 150.59, 150.5, 150.2, 150.1, 150.0, 148.7, 148.6, 148.5, 148.21, 148.16, 148.12, 148.1, 136.1, 136.0, 135.01, 134.98, 134.97, 134.93, 134.86, 134.83, 134.82, 134.78, 132.2, 132.1, 132.04, 131.96, 127.8, 127.6, 126.8 (m), 119.1, 119.0, 118.92, 118.87, 116.4, 116.3, 116.1, 72.1, 71.2, 60.8, 57.1, 57.0, 54.6, 49.02, 48.98, 42.5, 42.4, 42.2, 28.1, 22.6 (m), 16.73, 16.67. HRMS (DART): calcd for C₁₉H₂₁ClF₂N₂O [M+H]⁺: 367.1383, found: 367.1375.

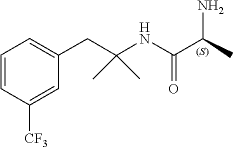

MP_039: (S)-2-amino-N-(2-methyl-1-(3-(trifluoromethyl)phenyl)propan-2-yl)propanamide: ¹H NMR (500 MHz, MeOD) δ7.54 (bm, 1H), 7.48 (m, 1H), 7.43 (bm, 2H), 3.76 (q, 1H, J=7.0 Hz), 3.38 (d, 1H, J=13.3 Hz), 3.00 (d, 1H, J=13.3 Hz), 1.41 (d, 1H, J=7.0 Hz), 1.39 (s, 3H), 1.27 (s, 3H). ¹³C NMR (500 MHz, MeOD) δ 168.9, 139.2, 134.1, 129.9 (q, J=31.9 Hz), 128.4, 126.5 (aq, J=3.8 Hz), 124.4 (aq, J=270.9 Hz), 122.9 (aq, J=3.9 Hz), 53.9, 49.1, 43.2, 26.0, 25.9, 16.6. HRMS (DART): calcd for C₁₄H₁₉F₃N₂O [M+H]⁺: 289.1522, found: 289.1515.

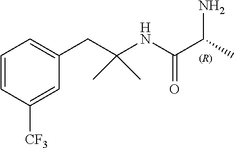

MP_040: (R)-2-amino-N-(2-methyl-1-(3-(trifluoromethyl)phenyl)propan-2-yl)propanamide: ¹H NMR (500 MHz, MeOD) δ7.53 (bm, 1H), 7.48 (m, 1H), 7.43 (bm, 2H), 3.77 (q, 1H, J=7.0 Hz), 3.37 (d, 1H, J=13.3 Hz), 3.00 (d, 1H, J=13.3 Hz), 1.41 (d, 1H, J=7.0 Hz), 1.39 (s, 3H), 1.27 (s, 3H). ¹³C NMR (500 MHz, MeOD) δ 168.9, 139.2, 134.1, 129.8 (q, J=31.9 Hz), 128.4, 126.5 (aq, J=3.7 Hz), 124.4 (aq, J=272.0 Hz), 122.9 (aq, J=3.9 Hz), 53.9, 49.1, 43.0, 26.1, 25.9, 16.6. HRMS (DART): calcd for C₁₄H₁₉F₃N₂O [M+H]⁺: 289.1522, found: 289.1515.

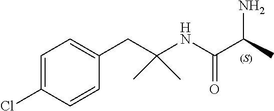

MP_041: 1-(4-chlorophenyl)-2-methylpropan-2-yl L-alaninate: ¹H NMR (500 MHz, CDCl₃) δ 7.25 (d, 2H, J=8.4 Hz), 7.11 (d, 2H, J=8.5 Hz), 3.40 (q, 1H, J=7.0 Hz), 3.02 (s, 2H), 1.46 (bs, 6H), 1.44 (s, 3H), 1.24 (d, 3H, J=7.1 Hz). ¹³C NMR (500 MHz, CDCl₃) δ 176.1, 135.4, 132.6, 131.8, 128.1, 82.2, 50.7, 46.0, 25.9, 25.7, 20.8. HRMS (DART): calcd for C₁₃H₁₈ClNO₂ [M+H]⁺: 256.1099, found: 256.1097.

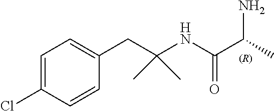

MP_042: 1-(4-chlorophenyl)-2-methylpropan-2-yl D-alaninate: ¹H NMR (500 MHz, CDCl₃) δ 7.25 (d, 2H, J=8.4 Hz), 7.11 (d, 2H, J=8.5 Hz), 3.40 (q, 1H, J=7.0 Hz), 3.02 (s, 2H), 1.46 (bs, 6H), 1.44 (s, 3H), 1.24 (d, 3H, J=7.1 Hz). ¹³C NMR (500 MHz, CDCl₃) δ 176.1, 135.4, 132.6, 131.8, 128.1, 82.2, 50.7, 46.0, 25.9, 25.7, 20.8. HRMS (DART): calcd for C₁₃H₁₈ClNO₂ [M+H]⁺: 256.1099, found: 256.1097.

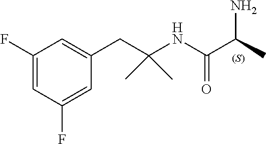

MP_043: (S)-2-amino-N-(1-(3,5-difluorophenyl)-2-methylpropan-2-yl)propanamide: ¹H NMR (500 MHz, MeOD) δ 6.79 (m, 3H), 3.77 (q, 1H, J=7.1 Hz), 3.26 (d, 1H, J=13.2 Hz), 2.96 (d, 1H, J=13.2 Hz), 1.41 (d, 3H, J=7.1 Hz), 1.37 (s, 3H), 1.29 (s 3H). ¹³C NMR (500 MHz, MeOH) δ 168.8, 162.8 (d, J=245.9 Hz), 162.6 (J=247.1 Hz), 142.3 (t, J=9.2 Hz), 112.9 (m), 101.2 (t, J=25.7 Hz), 53.8, 49.0, 43.2, 26.1, 26.0, 16.6. HRMS (DART): calcd for C₁₃H₈F₂N20 [M+H]⁺: 256.1460, found: 256.1455.

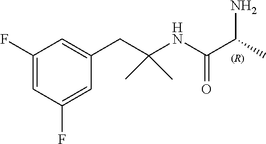

MP_044: (R)-2-amino-N-(1-(3,5-difluorophenyl)-2-methylpropan-2-yl)propanamide: ¹H NMR (500 MHz, MeOD) δ 6.79 (m, 3H), 3.78 (q, 1H, J=7.0 Hz), 3.26 (d, 1H, J=13.3 Hz), 2.96 (d, 1H, J=13.3 Hz), 1.41 (d, 3H, J=7.0 Hz), 1.37 (s, 3H), 1.29 (s 3H). ¹³C NMR (500 MHz, MeOH) δ 168.8, 162.8 (d, J=245.9 Hz), 162.6 (J=246.8 Hz), 142.3 (t, J=9.1 Hz), 112.9 (m), 101.2 (t, J=25.8 Hz), 53.8, 49.0, 43.2, 26.1, 26.0, 16.6. HRMS (DART): calcd for C₁₃H₈F₂N20 [M+H]⁺: 256.1460, found: 256.1457.

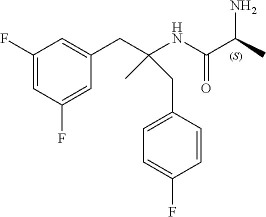

MP_045: (2S)-2-amino-N-(1-(3,5-difluorophenyl)-3-(4-fluorophenyl)-2-methylpropan-2-yl)propanamide: ¹H NMR (500 MHz, MeOD) δ 7.19-6.98 (m, 7H), 3.72-3.56 (m, 3H), 2.79 (dd, 1H, J=13.4, 2.1 Hz), 2.72 (dd, 1H, J=13.4, 8.7 Hz), 1.32 (d, 3H, J=7.1 Hz), 1.02 (d, 3H, J=2.7 Hz). ¹³C NMR (500 MHz, MeOH) δ 169.0, 162.8, 160.9, 133.3, 133.1, 132.1, 133.0, 131.9, 126.8, 119.0, 118.9, 118.8, 116.3, 116.2, 116.1, 114.3, 114.2, 114.1, 57.1, 49.0, 48.9, 42.4, 42.3, 42.2, 42.1, 22.5, 16.7, 16.6. HRMS (ESI): calcd for C₁₉H₂₁F₃N₂O [M+H]⁺: 351.1679, found: 351.1674.

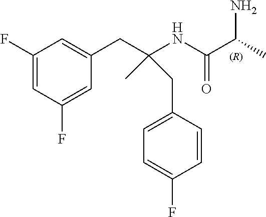

MP_046: (2R)-2-amino-N-(1-(3,5-difluorophenyl)-3-(4-fluorophenyl)-2-methylpropan-2-yl)propanamide: ¹H NMR (500 MHz, MeOD) δ7.19-6.98 (m, 7H), 3.72-3.56 (m, 3H), 2.79 (dd, 1H, J=13.4, 2.1 Hz), 2.72 (dd, 1H, J=13.4, 8.7 Hz), 1.32 (d, 3H, J=7.1 Hz), 1.02 (d, 3H, J=2.7 Hz). ¹³C NMR (500 MHz, MeOH) δ 169.02, 168.94, 162.8, 160.9, 133.2 (m), 133.1 (m), 132.0 (m), 126.8 (m), 118.9 (m), 116.4, 116.3, 116.1, 114.4, 114.2, 114.1, 57.1, 49.02, 48.98, 42.4, 42.3, 42.4, 42.1, 22.53, 22.50, 16.71, 16.65. HRMS (ESI): calcd for C₁₉H₂₁F₃N₂O [M+H]⁺: 351.1679, found: 351.1673.

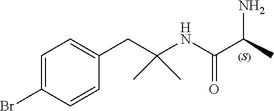

MP_047: (S)-2-amino-N-(1-(4-bromophenyl)-2-methylpropan-2-yl)propanamide: ¹H NMR (500 MHz, MeOD) δ7.41 (d, 2H, J=8.4 Hz), 7.07 (d, 2H, J=8.4 Hz), 3.75 (q, 1H, J=7.0 Hz), 3.12 (d, 1H, J=13.3 Hz), 2.98 (d, 1H, J=13.3 Hz), 1.41 (d, 3H, J=7.0 Hz), 1.33 (s, 3H), 1.29 (s, 3H). ¹³C NMR (500 MHz, MeOD) δ 168.6, 137.1, 132.1, 130.6, 119.9, 53.9, 49.0, 43.0, 25.9, 25.93, 16.7. HRMS (ESI): calcd for C₁₃H₁₉BrNO₂ [M+H]⁺: 299.0810, found: 299.0809.

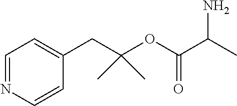 [SMILES: CC(N)C(=O)OC(C)(C)Cc1ccncc1]

MP_048: 2-methyl-1-(pyridin-4-yl)propan-2-yl alaninate: ¹H NMR (500 MHz, MeOD) δ8.81 (d, 2H, J=4.9 Hz), 8.04 (d, 2H, J=5.8 Hz), 4.08 (q, 1H, J=7.2 Hz), 3.48 (d, 1H, J=13.4 Hz), 3.44 (d, 1H, J=13.3 Hz), 1.62 (s, 3H), 1.59 (s, 3H), 1.48 (d, 3H, J=7.2 Hz). ¹³C NMR (500 MHz, MeOD) δ 169.1, 158.6, 140.9, 129.3, 83.9, 48.8, 46.0, 26.5, 24.61, 24.56, 15.0. HRMS (ESI): calcd for C₁₂H₁₈N₂O₂ [M+H]⁺: 223.1441, found: 223.1450.

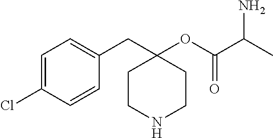 [SMILES: CC(N)C(=O)OC1(Cc2ccc(Cl)cc2)CCNCC1]

MP_049: 4-(4-chlorobenzyl)piperidin-4-yl alaninate: ¹H NMR (500 MHz, MeOD) δ7.35 (d, 2H, J=8.5 Hz), 7.19 (d, 2H, J=8.5 Hz), 4.18 (q, 1H, J=7.3 Hz), 3.43 (d, 1H, J=14.1 Hz), 3.35-3.31 (m, 2H), 3.27 (d, 1H, J=14.1 Hz), 3.19-3.11 (m, 2H), 2.63 (dd, 1H, J=15.3, 2.8 Hz), 2.44 (dd, 1H, J=15.2, 2.9 Hz), 1.96 (m, 2H), 1.45 (d, 3H, J=7.3 Hz). ¹³C NMR (500 MHz, MeOD) δ 169.3, 133.2, 133.1, 131.8, 128.3, 81.5, 48.8, 41.4, 39.5, 30.6, 29.8, 14.9. HRMS (ESI): calcd for C₁₅H₂₁ClN₂O₂ [M+H]⁺: 297.1379, found: 297.1364.

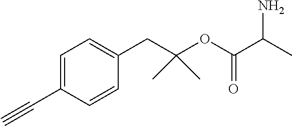 [SMILES: C#Cc1ccc(CC(C)(C)OC(=O)C(C)N)cc1]

MP_050: 1-(4-ethynylphenyl)-2-methylpropan-2-yl alaninate: ¹H NMR (500 MHz, MeOD) δ 7.40 (d, 2H, J=8.2 Hz), 7.22 (d, 2H, J=8.2 Hz), 3.95 (q, 1H, J=7.2 Hz), 3.46 (s, 1H), 3.15 (d, 1H, J=13.7 Hz), 3.11 (d, 1H, J=13.7 Hz), 1.52 (s, 3H), 1.51 (s, 3H), 1.42 (d, 3H, J=7.2 Hz). ¹³C NMR (500 MHz, MeOD) δ 169.0, 137.4, 131.3, 130.4, 120.9, 85.0, 82.8, 77.3, 48.8, 45.6, 24.8, 24.5, 14.9. HRMS (ESI): calcd for C₁₅H₁₉NO₂ [M+H]⁺: 246.1494, found: 246.1484.

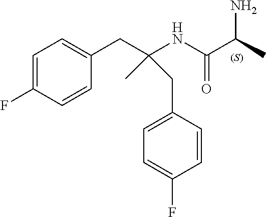

MP_051: (S)-2-amino-N-(1,3-bis(4-fluorophenyl)-2-methylpropan-2-yl)propanamide: ¹H NMR (500 MHz, MeOD) δ7.18 (m, 4H), 6.99 (m, 4H), 3.71 (q, 1H, J=7.1 Hz), 3.68 (d, 1H, J=13.4 Hz), 3.58 (d, 1H, J=13.4 Hz), 2.79 (d, 1H, J=13.5 Hz), 2.73 (d, 1H, J=13.4 Hz), 1.33 (d, 3H, J=7.1 Hz), 1.01 (s, 3H). ¹³C NMR (500 MHz, MeOD) δ 168.9, 161.9 (d, J=243.3 Hz), 133.4 (dd, J=18.8, 3.2 Hz), 132.03 (dd, J=7.9, 1.8 Hz), 114.2, (m), 57.2, 49.0, 48.2, 42.3, 42.2, 22.5, 16.7. HRMS (ESI): calcd for C₁₉H₂₂F₂N₂O [M+H]⁺: 332.1773, found: 333.1771.

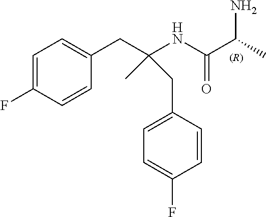

MP_052: (R)-2-amino-N-(1,3-bis(4-fluorophenyl)-2-methylpropan-2-yl)propanamide: ¹H NMR (500 MHz, MeOD) δ7.18 (m, 4H), 6.99 (m, 4H), 3.71 (q, 1H, J=7.1 Hz), 3.68 (d, 1H, J=13.4 Hz), 3.58 (d, 1H, J=13.4 Hz), 2.79 (d, 1H, J=13.5 Hz), 2.73 (d, 1H, J=13.4 Hz), 1.33 (d, 3H, J=7.1 Hz), 1.01 (s, 3H). ¹³C NMR (500 MHz, MeOD) δ 168.9, 161.9 (d, J=243.3 Hz), 133.4 (dd, J=18.8, 3.2 Hz), 132.03 (dd, J=8.0, 1.5 Hz), 114.2, (m), 57.2, 49.0, 48.2, 42.3, 42.2, 22.5, 16.7. HRMS (ESI): calcd for C₁₉H₂₂F₂N₂O [M+H]⁺: 332.1773, found: 333.1771.

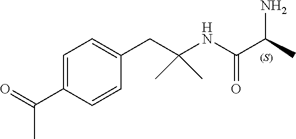

MP_053: (S)—N-(1-(4-acetylphenyl)-2-methylpropan-2-yl)-2-aminopropanamide: ¹H NMR (500 MHz, MeOD) δ7.91 (d, 2H, J=8.4 Hz), 7.30 (d, 2H, J=8.4 Hz), 3.76 (q, 1H, J=7.0 Hz), 3.25 (d, 1H, J=13.0 Hz), 3.09 (d, 1H, J=13.1 Hz), 2.58 (s, 3H), 1.42 (d, 3H, J=7.1 Hz), 1.35 (s, 3H), 1.31 (s, 3H). ¹³C NMR (500 MHz, MeOD) δ 198.9, 168.9, 168.8, 144.0, 135.3, 130.6, 127.7, 54.09, 53.98, 49.1, 43.6, 26.13, 26.11, 26.08, 26.04, 25.2, 16.7. HRMS (ESI): calcd for C₁₅H22N20 [M+H]⁺: 263.1754, found: 263.1756.

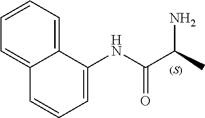

MP_054: (S)-2-amino-N-(naphthalen-1-yl)propanamide: ¹H NMR (500 MHz, MeOD) δ7.98 (m, 1H), 7.93 (m, 1H), 7.83 (d, 1H, J=8.3 Hz), 7.63 (m, 1H), 7.58-7.49 (m, 3H), 4.30 (q, 1H, J=7.1 Hz), 1.75 (d, 3H, J=7.1 Hz). ¹³C NMR (500 MHz, MeOD) δ 169.2, 134.4, 131.7, 128.5, 128.1, 126.8, 126.2, 125.9, 125.0, 122.6, 121.6, 49.4, 16.5. HRMS (ESI): calcd for C₁₃H₁₄N₂O [M+H]⁺: 215.1179, found: 215.1183.

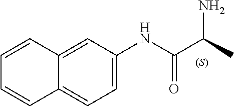

MP_055: (S)-2-amino-N-(naphthalen-2-yl)propanamide: ¹H NMR (500 MHz, MeOD) δ8.25 (d, 1H, J=1.9 Hz), 7.84-7.77 (m, 3H), 7.59 (dd, 1H, J=8.8, 2.1 Hz), 7.48-7.39 (m, 2H), 4.13 (q, 1H, J=7.1 Hz), 1.64 (d, 3H, J=7.1 Hz). ¹³C NMR (500 MHz, MeOD) δ 167.9, 135.2, 133.8, 130.9, 128.4, 127.2, 127.1, 126.3, 124.9, 119.5, 116.6, 49.6, 16.3. HRMS (ESI): calcd for C₁₃H₁₄N₂O [M+H]⁺: 215.1179, found: 215.1181.

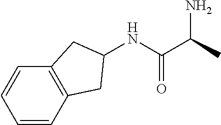

MP_056: (S)-2-amino-N-(2,3-dihydro-1H-inden-2-yl)propanamide: ¹H NMR (500 MHz, MeOD) δ7.21 (m, 2H), 7.14 (m, 2H), 4.59 (m, 1H), 3.83 (q, 1H, J=7.0 Hz), 3.27 (m, 2H), 2.85 (m, 2H), 1.45 (d, 3H, J=7.1 Hz). ¹³C NMR (500 MHz, MeOD) δ 169.9, 140.6, 140.5, 126.5, 124.2, 50.8, 48.8, 39.9, 38.8, 16.3. HRMS (ESI): calcd for C₁₃H₁₄N₂O [M+H]⁺: 205.1335, found: 205.1337.

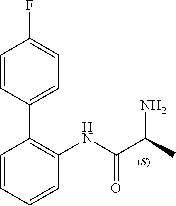

MP_057: (S)-2-amino-N-(4′-fluoro-[1,1′-biphenyl]-2-yl)propanamide: ¹H NMR (500 MHz, MeOD) δ7.49 (m, 1H), 7.42-7.36 (m, 5H), 7.16 (t, 2H, J=8.8 Hz), 3.95 (q, 1H, J=7.1 Hz), 1.39 (d, 3H, J=7.1 Hz). ¹³C NMR (500 MHz, MeOD) δ 168.6, 162.5, (d, J=245.5 Hz), 137.1, 135.1 (d, J=3.4 Hz), 133.1, 130.7 (d, J=8.1 Hz), 130.4, 127.9, 127.0, 126.9, 114.9 (d, J=21.7 Hz), 48.9, 15.9. HRMS (ESI): calcd for C₁₅H₁₅FN₂O [M+H]⁺: 259.1241, found: 259.1243.

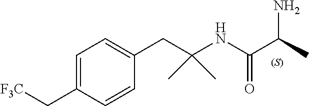

MP_058: (S)-2-amino-N-(2-methyl-1-(4-(2,2,2-trifluoroethyl)phenyl)propan-2-yl)propanamide: ¹H NMR (500 MHz, MeOD) δ7.24 (d, 2H, J=7.9 Hz), 7.16 (d, 2H, J=8.0 Hz), 3.74 (q, 1H, J=7.0 Hz), 3.43 (q, 2H, J=11.2 Hz), 3.16 (d, 1H, J=13.2 Hz), 2.99 (d, 1H, J=13.2 Hz), 1.40 (d, 3H, J=7.1 Hz), 1.35 (s, 3H), 1.30 (s, 3H). ¹³C NMR (500 MHz, MeOD) δ 168.7, 137.8, 130.4, 129.5, 128.6, 127.7, 125.0, 53.9, 49.1, 43.3, 38.8 (d, J=29.5 Hz), 26.06, 26.02, 16.7. HRMS (ESI): calcd for C₁₅H₂₁F₃N₂O [M+H]⁺: 303.1671, found: 303.1678.

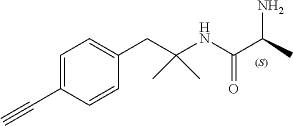

MP_059: (S)-2-amino-N-(1-(4-ethynylphenyl)-2-methylpropan-2-yl)propanamide: ¹H NMR (500 MHz, MeOD) δ7.37 (d, 2H, J=8.2 Hz), 7.14 (d, 2H, J=8.3 Hz), 3.76 (q, 1H, J=7.0 Hz), 3.43 (s, 1H), 3.17 (d, 3H, J=13.2 Hz), 3.00 (d, 1H, J=13.2 Hz), 1.41 (d, 3H, J=7.1 Hz), 1.34 (s, 3H), 1.29 (s, 3H). ¹³C NMR (500 MHz, MeOD) δ 168.7, 138.8, 131.2, 130.3, 120.5, 82.9, 77.1, 54.0, 49.0, 43.5, 26.0, 16.7. HRMS (ESI): calcd for C₁₅H₂₀N₂O [M+H]⁺: 245.1648, found: 245.1649.

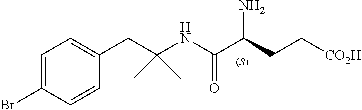

MP_061: (S)-4-amino-5-((1-(4-bromophenyl)-2-methylpropan-2-yl)amino)-5-oxopentanoic acid: ¹H NMR (500 MHz, MeOD) δ7.42 (d, 2H, J=8.2 Hz), 7.09 (d, 2H, J=8.2 Hz), 3.75 (t, 1H, J=6.3 Hz), 3.07 (d, 1H, J=13.3 Hz), 3.04 (d, 2H, J=13.4 Hz), 2.39 (m, 2H), 2.05 (m, 2H), 1.33 (s, 3H), 1.31 (s, 3H). ¹³C NMR (500 MHz, MeOD) δ 169.1, 158.6, 140.9, 129.3, 83.9, 48.8, 46.0, 26.5, 24.6, 15.0. HRMS (ESI): calcd for C₁₅H₂₁BrN₂O₃ [M+H]⁺: 357.0808, found: 357.0803.

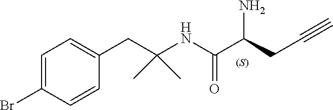

MP_062: (S)-2-amino-N-(1-(4-bromophenyl)-2-methylpropan-2-yl)pent-4-ynamide: ¹H NMR (500 MHz, MeOD) δ7.42 (d, 2H, J=8.3 Hz), 7.10 (d, 2H, J=8.2 Hz), 3.85 (t, 1H, J=5.9 Hz), 3.08 (d, 1H, J=13.4 Hz), 3.02 (d, 1H, J=13.4 H3), 2.78-2.68 (m, 3H), 1.31 (s, 3H), 1.30 (s, 3H). ¹³C NMR (500 MHz, MeOD) δ 169.2, 137.0, 132.2, 130.7, 120.0, 75.9, 73.9, 54.2, 51.5, 43.1, 25.86, 25.82, 21.4. HRMS (ESI): calcd for C₁₅H₁₉BrN₂O [M+H]⁺: 323.0753, found: 323.0766.

### Example 2: Further Preparation of Exemplary Compounds

General Information

THF and DCM were subjected to distillation from sodium and benzophenone immediately before use. All commercial reagents were purchased from commercial suppliers and used as received without further purification. Analytical thin-layer chromatography (TLC) was carried out using 0.25 mm commercial silica gel plates. Visualization was accomplished with UV light followed by dipping in a ceric ammonium molybdate or ninhydrin solutions followed by heating. Purification of reactions products was carried out by flash column chromatography using 40-63 m silica gel.

### Detailed Procedures and Spectral Data for Synthesis of (S)-4-amino-4,5-dihydro-1H-benzo[c]azepin-3(2H)-one hydrochloride (MP_071)

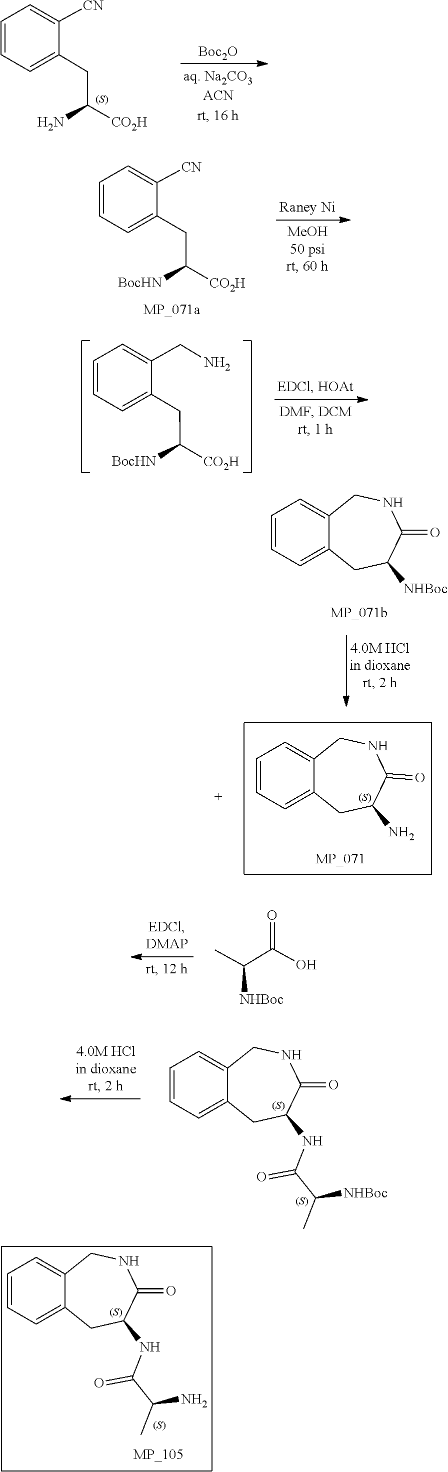

### Preparation of(S)—N-tert-butyloxycarbonyl(2-cyanophenyl)alanine (MP_071a)¹

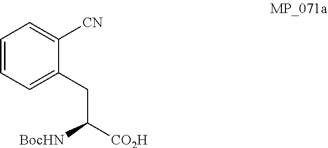 [SMILES: CC(C)(C)OC(=O)N[C@@H](Cc1ccccc1C#N)C(=O)O]

To a solution of (S)-L-2-cyanophenylalanine (380 mg, 2 mmol) in 6 ml of acetonitrile was added di-tert butyl dicarbonate (655 mg, 3 mmol), followed by saturated solution of sodium carbonate (6 mL) at room temperature. After 16 h, the acetonitrile was removed under reduced pressure, and the aqueous layer was extracted with diethyl ether (10 mL, three times). The combined organic layers were washed sequentially with 0.5 M aqueous hydrochloric acid (10 mL) and brine (50 mL), dried over anhydrous sodium sulfate and concentrated under vacuum to provide the desired compound MP_071a (480 mg, 1.65 mmol) in 83% yield. MP_071a: (S)—N-tert-butyloxycarbonyl(2-cyanophenyl)alanine: ¹H NMR (500 MHz, CDCl₃) δ 11.55 (s, 2H), 7.67-7.45 (m, 4H), 7.45-7.27 (m, 4H), 6.88 (s, 1H), 5.26 (d, J=6.3 Hz, 1H), 4.61 (d, J=37.8 Hz, 2H), 3.48 (dd, J=20.3, 14.4 Hz, 2H), 3.21-2.98 (m, 2H), 1.32 (d, J=3.2 Hz, 9H), 1.22-1.15 (m, 8H). ¹³C NMR (126 MHz, CDCl₃) δ 174.85, 174.44, 156.52, 155.21, 140.79, 140.68, 132.88, 132.77, 131.24, 130.60, 127.51, 127.43, 117.93, 117.63, 113.41, 81.94, 80.89, 80.22, 60.55, 54.89, 54.08, 38.35, 37.19, 28.61, 28.21, 27.87

### Preparation of (S)-4-(tert-butyloxycarbonylamino)-2,3,4,5-tetrahydro-2-benzazepin-3(1H)-one (MP_071b)¹

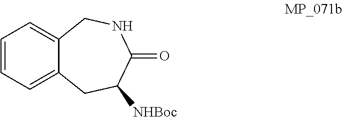 [SMILES: CC(C)(C)OC(=O)N[C@H]1Cc2ccccc2CNC1=O]

A mixture of (S)—N-tert-butyloxycarbonyl(2-cyanophenyl)alanine MP_071a (480 mg, 1.65 mmol) and Raney® Nickel 4200 slurry in H₂O (1.0 mL) in MeOH (20 mL) was hydrogenated in a Parr apparatus at 50 psi for 60 h at room temperature. The catalyst was filtered and rinsed with methanol (5 mL, three times). The combined filtrate and rinses were evaporated under vacuum, and the resulting residue was triturated with EtOAc to obtain crude product of (S)—N-tert-butyloxycarbnoyl(2-aminomethylphenyl)alanine (247 mg, 0.84 mmol), which was used directly in the next step. To a solution of (S)—N-tert-butyloxycarbnoyl(2-aminomethylphenyl)alanine (247 mg, 0.84 mmol) in N,N-dimethylformamide (5 mL) in dichloromethane (25 mL) was added 1-hydroxy-7-azabenzotriazole (176 mg, 1.29 mmol), followed by 1-Ethyl-3-(3-dimethylaminopropyl)carbodiimide hydrochloride (101 mg, 0.65 mmol) was stirred at room temperature for 1 h. The solvent was removed under reduced pressure and the residue was dissolved in EtOAc (50 mL). This solution was washed sequentially with 1.0 M aqueous hydrochloric acid (20 mL twice), 1.0 M aqueous sodium hydroxide (20 mL), water (20 mL), and brine (20 mL), dried over anhydrous sodium sulfate, concentrated under vacuum, and purified by flash column chromatography (EtOAc:hexane=100:0) to furnish the desired compound MP_071b as a white solid in 15% yield (35 mg, 0.15 mmol); TLC (SiO₂) R_{f}=0.5 (100% of EtOAc, visualized by Ninhydrin); ¹H NMR (400 MHz, CDCl₃) δ 7.18 (d, J=7.3 Hz, 1H), 7.13-7.06 (m, 2H), 7.00 (d, J=7.6 Hz, 1H), 5.86 (d, J=6.0 Hz, 1H), 5.09-4.93 (m, 1H), 4.84 (dd, J=16.5, 4.5 Hz, 1H), 3.96 (dd, J=16.6, 7.1 Hz, 1H), 3.41 (dd, J=16.9, 3.5 Hz, 1H), 2.97 (dd, J=16.6, 13.0 Hz, 1H), 1.46 (s, 9H). ¹³C NMR (101 MHz, CDCl₃) δ 174.28, 155.19, 135.53, 133.97, 131.08, 128.21, 127.82, 126.26, 79.75, 49.22, 45.75, 36.99, 28.41.

### Preparation of (S)-4-amino-4,5-dihydro-1H-benzo[c]azepin-3(2H)-one hydrochloride (MP_071)

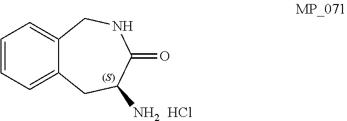

To a solution of (S)-4-(tert-butyloxycarbonylamino)-2,3,4,5-tetrahydro-2-benzazepin-3(1H)-one MP_073 (35 mg, 0.15 mmol) in dichloromethane (1 mL) was added 4.0 M hydrochloric acid in dioxane at room temperature. After 2 h stirring the precipitate slowly formed and the solvent was evaporated under vacuum. The white solid was washed with Et₂O to give product MP_071 (32 mg, 0.15 mmol) in 99% yield. ¹H NMR (400 MHz, DMSO) δ 8.66 (t, J=5.8 Hz, 1H), 8.52-8.42 (m, 2H), 7.27-7.18 (m, 1H), 7.17-7.10 (m, 3H), 4.81 (d, J=4.6 Hz, 1H), 4.80-4.76 (m, 1H), 3.93 (d, J=7.0 Hz, 1H), 3.89 (d, J=6.9 Hz, 1H), 3.35 (d, J=4.3 Hz, 1H), 3.07 (d, J=13.1 Hz, 1H), 3.03 (d, J=13.1 Hz, 1H). ¹³C NMR (101 MHz, DMSO) δ 170.81, 135.87, 134.21, 131.01, 129.05, 128.05, 126.92, 48.86, 44.30, 33.42. HRMS (DART, m/z): Calculated for C₁₀H₁₂N₂OH [M+H]⁺=177.1028; found 177.1020

### Detailed Procedures and Spectral Data for Synthesis of (S)-5-(4-chlorobenzyl)imidazolidin-4-one (MP_072)

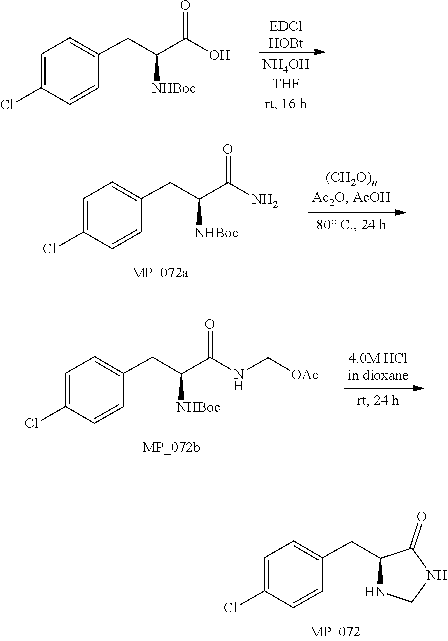 [SMILES: CC(C)(C)OC(=O)N[C@@H](Cc1ccc(Cl)cc1)C(=O)O ; "EDClHOBt" ; CC(C)(C)OC(=O)N[C@@H](Cc1ccc(Cl)cc1)C(N)=O ; CC(=O)OCNC(=O)[C@H](Cc1ccc(Cl)cc1)NC(=O)OC(C)(C)C ; O=C1NCN[C@H]1Cc1ccc(Cl)cc1]

### Preparation of(S)-tert-butyl (1-amino-3-(4-chlorophenyl)-1-oxopropan-2-yl)carbamate (MP_072a)

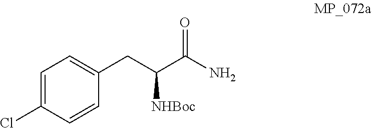 [SMILES: CC(C)(C)OC(=O)N[C@@H](Cc1ccc(Cl)cc1)C(N)=O]

To a solution of Boc-4-chloro-L-phenylalanine (893 mg, 3 mmol) in THF (40 mL) was added 1-hydroxybenzotriazole hydrate (459.4 mg, 3 mmol) followed by 1-Ethyl-3-(3-dimethylaminopropyl)carbodiimide hydrochloride (661 mg, 3.45 mmol). After 30 min, concentrated NH₄OH (239 μL, 6 mmol) was added to the solution and the reaction mixture was stirred for 16 h. The solution was concentrated and the reaction residue was diluted with EtOAc. The suspension was washed with saturated NaHCO₃(aq) and brine, dried over Na₂SO₄, filtered and concentrated to afford the desired product MP_072a (880 mg, 2.94 mmol) in 98% yield. ¹H NMR (400 MHz, DMSO) δ 7.28 (d, J=8.4 Hz, 2H), 7.23 (d, J=8.4 Hz, 2H), 6.97 (s, 1H), 6.78 (d, J=8.8 Hz, 1H), 4.02 (ddd, J=14.2, 11.1, 5.8 Hz, 1H), 2.90 (dd, J=13.7, 4.3 Hz, 1H), 2.67 (dd, J=13.6, 10.4 Hz, 1H), 1.26 (s, 9H). ¹³C NMR (101 MHz, DMSO) δ 173.83, 155.68, 137.86, 131.52, 128.37, 78.39, 55.89, 37.33, 28.57.

### Preparation of (S)-(2-((tert-butoxycarbonyl)amino)-3-(4-chlorophenyl)propanamido)methyl acetate (MP_72b)

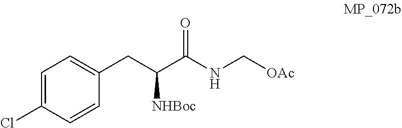 [SMILES: CC(=O)OCNC(=O)[C@H](Cc1ccc(Cl)cc1)NC(=O)OC(C)(C)C]

To a (S)-tert-butyl (1-amino-3-(4-chlorophenyl)-1-oxopropan-2-yl)carbamate MP_072a (880 mg, 2.95 mmol) were added paraformaldehyde (90 mg) in an acetic anhydride (2.83 mL, 30 mmol) and acetic acid (9 mL). The reaction mixture was heated to 80° C. for 16 h. After cooling the solution to room temperature, the reaction mixture was treated with NaHCO₃ and organic layer was extracted with EtOAc, dried over anhydrous Na₂SO₄ and filtered. The filtrate was concentrated under reduced pressure and the residue was purified by flash column chromatography to furnish the desired product MP_72b as an white solid in 37% yield (400 mg, 1.08 mmol). TLC (SiO₂) R_{f}=0.4 (EtOAc:hexane=1:2, visualized by ceric ammonium molybdate)¹H NMR (400 MHz, CDCl₃) δ 7.23 (d, J=8.4 Hz, 10H), 7.10 (d, J=8.4 Hz, 7H), 5.24-5.11 (m, 8H), 4.35 (s, 3H), 3.00 (ddd, J=20.3, 13.8, 7.0 Hz, 6H), 2.01 (d, J=6.2 Hz, 12H), 1.37 (s, 31H). ¹³C NMR (126 MHz, CDCl₃) δ 172.11, 171.54, 155.45, 134.89, 132.96, 130.78, 130.54, 128.81, 80.58, 63.98, 55.53, 37.72, 28.29, 28.20, 20.86.

### Preparation of of (S)-5-(4-chlorobenzyl)imidazolidin-4-one (MP_072)

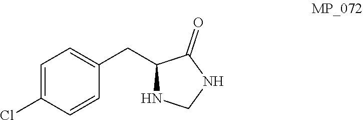 [SMILES: O=C1NCN[C@H]1Cc1ccc(Cl)cc1]

To a solution of (S)-(2-((tert-butoxycarbonyl)amino)-3-(4-hlorophenyl)propanamido)methyl acetate MP_72b (120 mg, 0.32 mmol) in dichloromethane (1 mL) was added 4.0 M hydrochloric acid in dioxane (4 mL) at room temperature. After 18 h stirring the precipitate slowly formed and the solvent was evaporated under vacuum. The white solid was washed with Et₂O to give product MP_072 (52 mg, 0.25 mmol) in 78% yield. ¹H NMR (500 MHz, DMSO) δ 9.16 (d, J=6.2 Hz, 1H), 8.27 (d, J=3.5 Hz, 3H), 7.34 (dd, J=8.6, 2.0 Hz, 2H), 7.29 (d, J=8.5 Hz, 2H), 4.55 (dd, J=9.9, 6.3 Hz, 1H), 4.48 (dd, J=10.0, 6.1 Hz, 1H), 3.94 (s, 1H), 3.09 (dd, J=14.0, 5.7 Hz, 1H), 2.98 (dd, J=14.0, 7.4 Hz, 1H) ¹³C NMR (126 MHz, DMSO) δ 168.38, 134.33, 132.36, 132.04, 128.88, 62.92, 53.80, 36.36. HRMS (DART, m/z): Calculated for C₁₀H₁₁ClN₂OH [M+H]⁺=211.0638; found 211.0619

### Detailed Procedures and Spectral Data for Synthesis of (S)-methyl (3-oxo-2,3,4,5-tetrahydro-1H-benzo[c]azepin-4-yl)carbamate (MP_073)

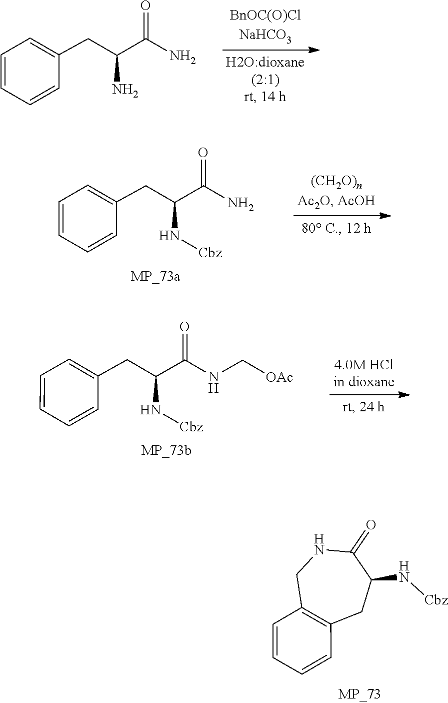 [SMILES: NC(=O)[C@@H](N)Cc1ccccc1 ; NC(=O)[C@H](Cc1ccccc1)NC(=O)OCc1ccccc1 ; CC(=O)OCNC(=O)[C@H](Cc1ccccc1)NC(=O)OCc1ccccc1 ; O=C(N[C@H]1Cc2ccccc2CNC1=O)OCc1ccccc1]

### Preparation of of (S)-benzyl(1-amino-1-oxo-3-phenylpropane-2-yl)carbamate (MP_073a)5

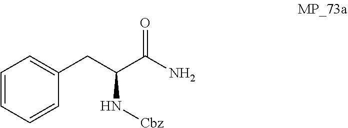 [SMILES: NC(=O)[C@H](Cc1ccccc1)NC(=O)OCc1ccccc1]

(S)-2-amino-3-phenylpropanamide HCl (1.15 g, 7 mmol) and sodium bicarbonate (706 mg, 8.4 mmol) were dissolved in a 2:1 mixture of H₂O/dioxane (14 mIJ7 mL). The mixture was cooled to 0° C. and benzyl chloroformate (1.99 mL, 8.4 mmol) was added dropwise to the solution. The mixture was warmed to room temperature and stirred for 14 h. The mixture was diluted with EtOAc and washed with water and brine. The organic layers were combined, dried over Na₂SO₄, filtered and concentrated under reduced pressure to give the crude product as a white solid. Purification by recrystallization in MeOH provided product MP_073a as a white solid (1.49 g, 5 mmol) in 71% yield. ¹H NMR (400 MHz, DMSO) δ 7.50-7.09 (m, 13H), 7.04 (s, 1H), 4.90 (s, 2H), 4.27-4.03 (m, 1H), 3.09-2.83 (m, 1H), 2.71 (dd, J=13.7, 10.5 Hz, 1H).

### Preparation of of (S)-(2-(((benzyloxy)carbonyl)amino)-3-phenylpropanamido)methyl acetate (MP_073b)

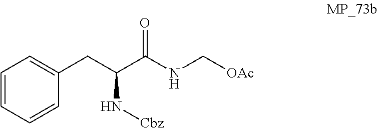 [SMILES: CC(=O)OCNC(=O)[C@H](Cc1ccccc1)NC(=O)OCc1ccccc1]

To a (S)-benzyl(1-amino-1-oxo-3-phenylpropane-2-yl)carbamate MP_073a (1.11 g, 5 mmol) were added paraformaldehyde (180 mg) in an acetic anhydride (5.67 mL, 60 mmol) and acetic acid (20 mL). The reaction mixture was heated to 80° C. for 12 h. After cooling the solution to room temperature, the reaction mixture was treated with NaHCO₃ and organic layers were extracted with EtOAc, dried over anhydrous Na₂SO₄ and filtered. The filtrate was concentrated under reduced pressure and the residue was recrystallized with EtOAc and hexane to furnish the desired product MP_73b as a white solid in 42% yield (780 mg, 2.11 mmol). ¹H NMR (400 MHz, DMSO) δ 9.07 (s, 1H), 7.53 (d, J=8.6 Hz, 1H), 7.35-7.16 (m, 11H), 5.07 (dd, J=6.9, 3.9 Hz, 2H), 4.90 (s, 2H), 4.22 (dd, J=5.3, 3.3 Hz, 1H), 2.93 (dd, J=13.7, 4.2 Hz, 1H), 2.70 (dd, J=13.6, 10.7 Hz, 1H), 1.95 (d, J=5.6 Hz, 3H).

### Preparation of of (S)-benzyl (3-oxo-2,3,4,5-tetrahydro-1H-benzo[c]azepin-4-yl)carbamate (MP_073)

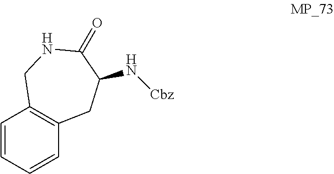 [SMILES: O=C(N[C@H]1Cc2ccccc2CNC1=O)OCc1ccccc1]

To a solution of (S)-(2-(((benzyloxy)carbonyl)amino)-3-phenylpropanamido)methyl acetate (MP_073b) (207 mg, 0.67 mmol) in dichloromethane (2 mL) was added 4.0 M hydrochloric acid in dioxane (4 mL) at room temperature. After 24 h stirring, the mixture was quenched with saturated NaHCO₃(aq). The organic layers were extracted with DCM, dried over Na₂SO₄, filtered and concentrated under reduced pressure to give the crude product. Purification by flash chromatography with EtOAc and hexane provided product MP_073 as a white solid (48 mg, 0.2 mmol) in 31% yield. TLC (SiO₂) R_{f}=0.4 (EtOAc:hexane=3:1, visualized by ceric ammonium molybdate)¹H NMR (400 MHz, CDCl₃) δ 7.37-7.20 (m, 9H), 7.17 (d, J=6.9 Hz, 2H), 5.54 (d, J=6.1 Hz, 1H), 5.02 (dd, J=30.3, 12.3 Hz, 2H), 4.76 (s, 1H), 4.49-4.14 (m, 2H), 3.04 (ddd, J=43.4, 13.8, 7.3 Hz, 2H). ¹³C NMR (126 MHz, CDCl₃) δ 172.72, 156.34, 136.18, 135.95, 129.26, 128.73, 128.55, 128.25, 128.02, 127.12, 68.40, 67.22, 56.44, 38.00, 29.72. HRMS (DART, m/z): Calculated for C₁₈H₁₈N₂O₃H [M+H]⁺=311.1396; found 311.1369.

### Detailed Procedures and Spectral Data for Synthesis of (S)-(9H-fluoren-9-yl)methyl (3-oxo-2,3,4,5-tetrahydro-1H-benzo[c]azepin-4-yl)carbamate (MP_074)

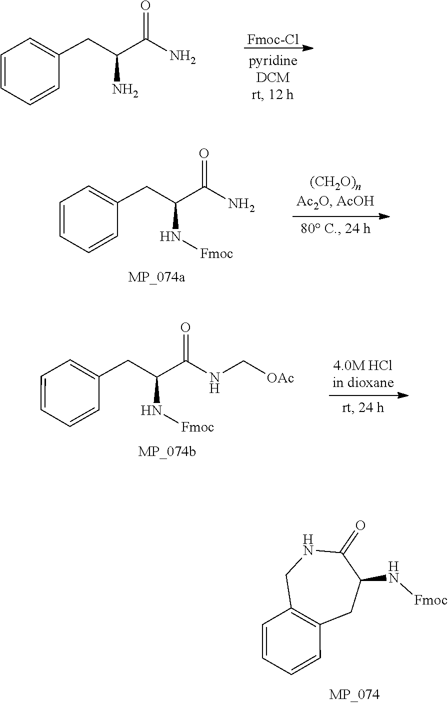

### Preparation of (S)-(9H-fluoren-9-yl)methyl (1-amino-1-oxo-3-phenylpropan-2-yl)carbamate (MP_74a)

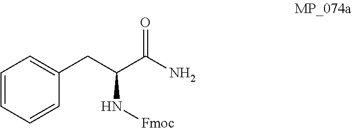

To a solution of (S)-2-amino-3-phenylpropanamide HCl (1.15 g, 7 mmol) in DCM (35 mL) was added pyridine (676 μL, 8.4 mmol) at 0° C. After 5 min stirring, 9-Fluorenylmethoxycarbonyl chloride was added dropwise to the reaction mixture at same temperature. The suspension was warmed to room temperature and stirred for 12 h. The mixture was quenched with saturated NH₄Cl (aq) and the organic layers were extracted by DCM. The combined organic layers were washed with brine, dried over Na₂SO₄, filtered and concentrated under reduced pressure to give the product MP_74a (1.93 g, 5 mmol) in 71% yield. ¹H NMR (500 MHz, DMSO) δ 7.85 (d, J=7.5 Hz, 2H), 7.65-7.14 (m, 12H), 7.08 (s, 1H), 4.29-4.00 (m, 4H), 3.64 (s, 1H), 3.00 (dd, J=13.6, 4.0 Hz, 1H), 2.79 (dd, J=13.4, 10.8 Hz, 1H). ¹³C NMR (126 MHz, DMSO) δ 173.97, 156.28, 144.26, 144.23, 141.12, 138.82, 129.69, 129.40, 128.50, 128.09, 127.77, 127.52, 126.68, 125.84, 125.76, 121.86, 120.54, 120.50, 66.07, 56.57, 47.04, 37.99.

### Preparation of (S)-(2-((((9H-fluoren-9-yl)methoxy)carbonyl)amino)-3-phenylpropanamido)methyl acetate (MP_74b)

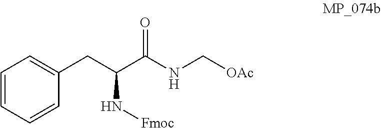

To a (S)-(9H-fluoren-9-yl)methyl (1-amino-1-oxo-3-phenylpropan-2-yl)carbamate MP_074a (1.93 g, 5 mmol) were added paraformaldehyde (180 mg) in an acetic anhydride (5.67 mL, 60 mmol) and acetic acid (20 mL). The reaction mixture was heated to 80° C. for 12 h. After cooling the solution to room temperature, the reaction mixture was treated with NaHCO₃ and organic layers were extracted with EtOAc, dried over anhydrous Na₂SO₄ and filtered. The filtrate was concentrated under reduced pressure and the residue was recrystallized with Et₂O to furnish the desired product MP_74b (620 mg, 1.35 mmol) in 27% yield. ¹H NMR (400 MHz, DMSO) δ 7.83 (d, J=7.6 Hz, 2H), 7.61 (t, J=7.6 Hz, 1H), 7.47 (d, J=8.8 Hz, 1H), 7.40 (d, J=5.9 Hz, 1H), 7.36 (ddd, J=7.5, 5.4, 2.4 Hz, 2H), 7.31-7.19 (m, 6H), 7.15 (d, J=7.1 Hz, 1H), 7.03 (s, 1H), 4.12 (dd, J=14.5, 8.2 Hz, 4H), 3.30 (s, 2H), 2.98 (dd, J=13.7, 4.2 Hz, 1H), 2.76 (dd, J=13.6, 10.6 Hz, 1H), 2.45 (s, 1H). ¹³C NMR (126 MHz, CDCl₃) δ 173.48, 169.75, 156.00, 143.69, 143.65, 141.33, 129.31, 129.22, 128.95, 128.88, 128.79, 128.73, 127.78, 127.43, 127.18, 127.11, 125.03, 120.03, 79.18, 67.23, 66.99, 47.16, 38.35, 20.75.

### Preparation of (S)-(9H-fluoren-9-yl)methyl (3-oxo-2,3,4,5-tetrahydro-1H-benzo[c]azepin-4-yl)carbamate (MP_074)

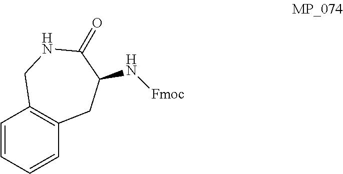

To a solution of (S)-(2-((((9H-fluoren-9-yl)methoxy)carbonyl)amino)-3-phenylpropanamido)methyl acetate MP_74b (280 mg, 0.61 mmol) in DCM (2 mL) was added 4.0 M hydrochloric acid in dioxane (2 mL) at room temperature. After 24 h stirring, the mixture was quenched with saturated NaHCO₃(aq). The organic layers were extracted with DCM, dried over Na₂SO₄, filtered and concentrated under reduced pressure. The product MP_074 (130 mg, 0.32 mmol) was purified by flash chromatography with EtOAc and hexane provided product in 53% yield. ¹H NMR (500 MHz, CDCl₃) δ 7.76 (d, J=7.5 Hz, 2H), 7.53 (t, J=7.5 Hz, 2H), 7.43-7.03 (m, 8H), 5.74 (s, 1H), 5.56-5.34 (m, 1H), 4.50-4.28 (m, 2H), 4.16 (dd, J=19.5, 12.9 Hz, 1H), 3.07 (s, 2H). ¹³C NMR (126 MHz, CDCl₃) δ 173.10, 156.00, 143.69, 143.65, 141.34, 136.31, 129.33, 128.81, 128.72, 127.78, 127.23, 127.19, 127.12, 125.02, 120.03, 66.97, 55.78, 47.17, 47.12, 38.37.

### Detailed Procedures and Spectral Data for Synthesis of CF3-substituted compound (MP_075, 076, 077, 078)

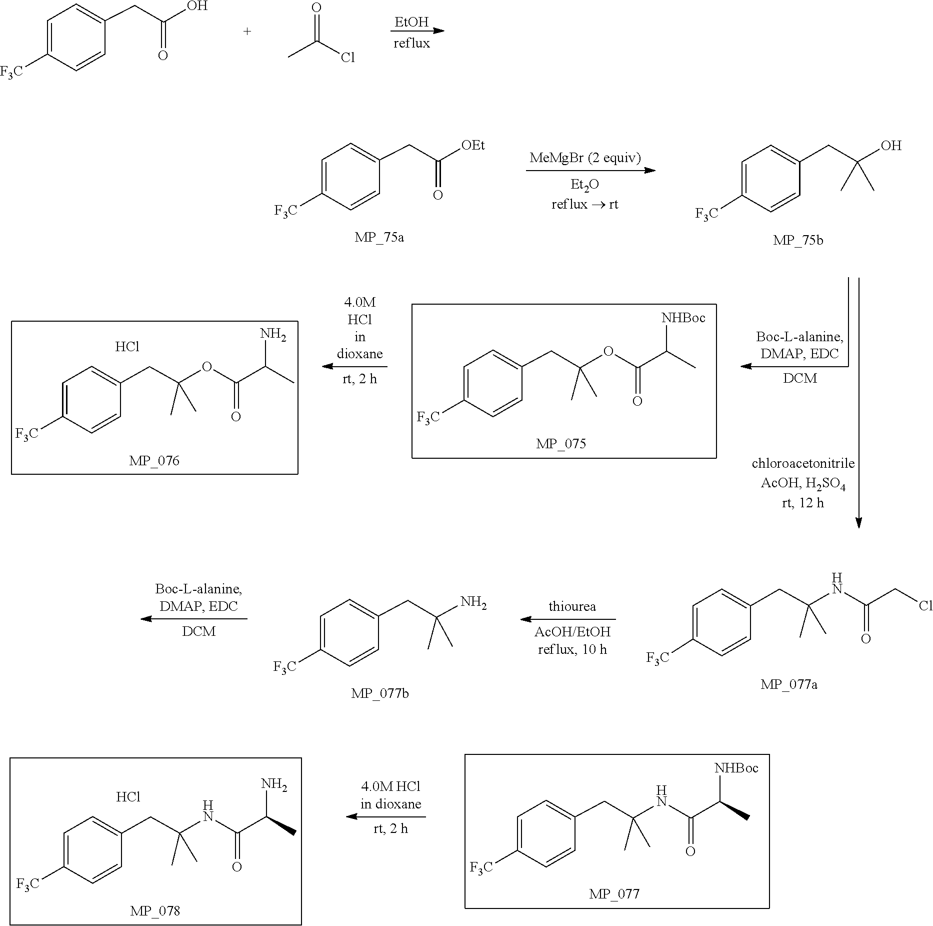

### Preparation of ethyl 2-(4-(trifluoromethyl)phenyl)acetate (MP_75a)

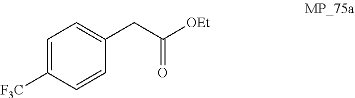 [SMILES: CCOC(=O)Cc1ccc(C(F)(F)F)cc1]

To a solution of 4-(trifluoromethyl)phenylacetic acid (204 mg, 1 mmol) in EtOH (2 mL) was added acetyl chloride (85 μl, 1.2 mmol) and the reaction mixture was refluxed at 50° C. for 1.5 h. The reaction was allowed to cool to ambient temperature, and solvent was removed under reduced pressure. The residue was taken up in Et₂O and washed with sat. NaHCO₃(aq), brine, dried over Na₂SO₄ and concentrated to afford the product MP_75a (234 mg, 0.99 mmol) in 99% yield. ¹H NMR (400 MHz, CDCl₃) δ 7.57 (d, J=8.1 Hz, 2H), 7.40 (d, J=8.0 Hz, 2H), 4.16 (q, J=7.1 Hz, 2H), 3.66 (s, 2H), 1.24 (t, J=7.1 Hz, 3H). ¹³C NMR (101 MHz, CDCl₃) δ 170.74, 138.19 (d, J=1.2 Hz), 129.39 (q, J=32.4 Hz), 125.41 (q, J=3.8 Hz), 124.19 (q, J=271.9 Hz), 61.09, 41.04, 14.02.

### Preparation of 2-methyl-1-(4-(trifluoromethyl)phenyl)propan-2-ol (MP_75b)8

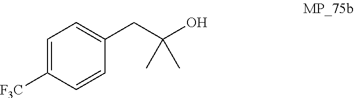

A three-necked flask, equipped with a magnetic stir bar, an addition funnel, and a reflux condenser, was flame-dried then placed under argon. To the flask were added ethyl 2-(4-(trifluoromethyl)phenyl)acetate MP_75a (232 mg, 1 mmol) and anhydrous diethyl ether (10 mL). A methylmagnesium bromide solution in Et₂O (2.7 mmol, 3 M, 0.9 mL) was added at a rate such that the reaction mixture refluxed smoothly. When the Grignard reagent had been fully added, the reaction mixture was heated at reflux for 6 hours. The reaction mixture was cooled to 0° C. in an ice-water bath, and crushed ice was added carefully. The resulting magnesium salt precipitate was dissolved by adding 1.0 N hydrochloric acid solution. The organic layer was separated, and the aqueous layer was further extracted with diethyl ether (2×50 mL). The combined organic layers were dried over Na₂SO₄. The solution was filtered, and the solvent was removed in vacuum to give the crude product which was purified by flash column chromatography on silica gel using EtOAc/hexane to afford product MP_75b (190 mg, 0.87 mmol) in 87% yield. TLC (SiO₂) R_{f}=0.3 (EtOAc:hexane=1:4)¹H NMR (400 MHz, CDCl₃) δ 7.52 (s, 1H), 7.33 (s, 1H), 2.79 (s, 1H), 1.20 (s, 3H). ¹³C NMR (101 MHz, CDCl₃) δ 142.23, 130.78, 128.68 (q, J=32.2 Hz), 124.88 (q, J=3.8 Hz), 124.40 (q, J=271.8 Hz), 70.73, 49.44, 29.19.

### Preparation of 2-methyl-1-(4-(trifluoromethyl)phenyl)propan-2-yl 2-((tert-butoxycarbonyl)amino)propanoate (MP_75)

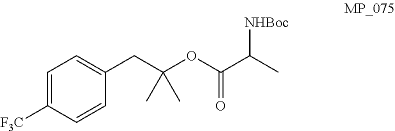 [SMILES: CC(NC(=O)OC(C)(C)C)C(=O)OC(C)(C)Cc1ccc(C(F)(F)F)cc1]

To a solution of 2-methyl-1-(4-(trifluoromethyl)phenyl)propan-2-ol MP_75b (185 mg, 0.85 mmol) in DCM was added the solution of 4-dymethylaminopyridine (21 mg, 0.17 mmol) in DCM followed by N-(tert-butoxycarbonyl)-L-alanine (241 mg, 1.27 mmol), and the mixture was allowed to stir for 10 min at 0° C. Then, N-(3-dimethylaminopropyl)-N′-ethylcarbodiimide hydrochloride (150 mg, 0.78 mmol) was added to the solution and the mixture was stirred for 1 h. After being stirred for 12 h at room temperature, the reaction mixture was quenched with water. The organic layer was extracted by ethyl acetate (10 mL×2). The combined organic layers were washed with brine (5 mL), dried over Na₂SO₄, filtered and concentrated under reduced pressure. The residue was subjected to flash column chromatography using EtOAc/hexane to afford product MP_75 (51 mg, 0.13 mmol) in 13% yield. TLC (SiO₂) R_{f}=0.7 (EtOAc:hexane=1:4, visualized by ceric ammonium molybdate) ¹H NMR (500 MHz, CDCl₃) δ 7.53 (d, J=8.0 Hz, 2H), 7.29 (d, J=8.0 Hz, 2H), 5.00 (d, J=6.4 Hz, 1H), 4.29-4.12 (m, 1H), 3.10 (dd, J=34.4, 13.6 Hz, 2H), 1.48 (s, 3H), 1.43 (d, J=4.0 Hz, 12H), 1.27 (d, J=7.2 Hz, 3H). ¹³C NMR (126 MHz, CDCl₃) δ 172.65, 155.10, 140.93, 130.90, 128.98 (q, J=32.4 Hz), 124.91 (q, J=3.7 Hz), 124.25 (q, J=271.9 Hz), 82.84, 79.72, 49.84, 46.47, 28.31, 25.90, 25.72, 18.65.

### Preparation of 2-methyl-1-(4-(trifluoromethyl)phenyl)propan-2-yl 2-aminopropanoate hydrochloride (MP_76)

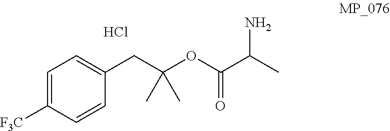 [SMILES: CC(N)C(=O)OC(C)(C)Cc1ccc(C(F)(F)F)cc1 ; Cl]

To 2-methyl-1-(4-(trifluoromethyl)phenyl)propan-2-yl 2-((tert-butoxycarbonyl)amino)propanoate MP_75 (51 mg, 0.13 mmol) was added 4.0 M hydrochloric acid in dioxane at room temperature. After being stirred for 8 h, the mixture was concentrated under reduced pressure. Et₂O was added to the mixture and concentrated under reduced pressure (×2). By adding Et₂O, white solid was precipitated and filtered out with Et₂O to afford product MP_76 (26 mg, 0.08 mmol) in 61% yield. ¹H NMR (500 MHz, MeOD) δ 7.61 (d, J=8.0 Hz, 2H), 7.44 (d, J=8.0 Hz, 2H), 3.97 (q, J=7.2 Hz, 1H), 3.22 (s, 2H), 1.54 (d, J=10.3 Hz, 6H), 1.43 (d, J=7.2 Hz, 3H). ¹³C NMR (126 MHz, MeOD) δ 169.06, 141.19, 131.02, 128.73 (q, J=32.3 Hz), 124.57 (q, J=3.8 Hz), 124.40 (q, J=270.9 Hz), 84.80, 48.88, 45.54, 24.79, 24.53, 14.93. HRMS (DART, m/z): Calculated for C₁₄H₁₈F₃NO₂H [M+H]⁺=290.1367; found 290.1345.

### Preparation of 2-chloro-N-(2-methyl-1-(4-(trifluoromethyl)phenyl)propan-2-yl)acetamide (MP_077a)

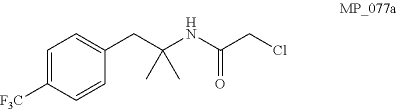 [SMILES: CC(C)(Cc1ccc(C(F)(F)F)cc1)NC(=O)CCl]

To the the 2-methyl-1-(4-(trifluoromethyl)phenyl)propan-2-ol MP_75b (872 mg, 4 mmol) was added 1 mL of acetic acid and chloroacetonitrile (510 μL, 8 mmol) and allowed to stir at 0° C. The mixture was added 1.0 mL of H₂SO₄ dropwise. The solution was allowed to reach room temperature and stiffed for an additional 12 h. the reaction mixture was poured into 20 mL of ice water and extracted with Et₂O (30 mL×2). The combined organic layers were washed with saturated NaHCO₃ and brine, dried over Na₂SO₄ and the solvent was evaporated under reduced pressure to afford the crude chloroacetamide product MP_077a (955 mg, 3.42 mmol) in 86% yield. ¹H NMR (400 MHz, CDCl₃) δ 7.51 (d, J=8.0 Hz, 2H), 7.21 (d, J=8.0 Hz, 2H), 6.32-5.93 (m, 1H), 3.90 (s, 2H), 3.10 (s, 2H), 1.32 (s, 6H). ¹³C NMR (101 MHz, CDCl₃) δ 165.48, 141.64 (d, J=1.1 Hz), 128.85 (q, J=32.4 Hz), 124.94 (q, J=3.7 Hz), 124.28 (q, J=271.9 Hz), 54.43, 44.26, 42.88, 26.90.

### Preparation of 2-methyl-1-(4-(trifluoromethyl)phenyl)propan-2-amine (MP_077b)

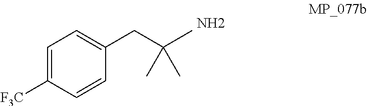

In a round bottom flask equipped with a stir bar and reflux condenser, the chloroacetamide MP_077a (955 mg, 3.42 mmol) was added thiourea (326 mg, 4.28 mmol) along with 10 mL of ethanol and 2 mL of acetic acid. The reaction mixture was allowed to stir at reflux for 10 h. The reaction mixture was cooled and poured into 40 mL of cold water. The solution was made alkaline with 4 M NaOH (pH 11) and extracted with EtOAc (40 mL×2). The combined organic layers were washed with saturated NaHCO₃ and brine, dried over Na₂SO₄ and the solvent was evaporated under reduced pressure to afford the crude amine product. The crude amine product was placed under high vacuum overnight to remove any residual solvent. The crude amine product (470 mg, 2.31 mmol) was used for the next step without purification. ¹H NMR (400 MHz, DMSO) δ 7.58 (d, J=7.9 Hz, 2H), 7.38 (d, J=7.9 Hz, 2H), 3.86 (s, 1H), 2.64 (s, 2H), 0.96 (s, 6H). ¹³C NMR (101 MHz, DMSO) δ 188.19, 183.26, 144.19, 127.14 (q, J=31.8 Hz), 124.87 (q, J=3.7 Hz), 125.00 (q, J=271.7 Hz), 50.53, 50.04, 30.20.

### Preparation of (S)-tert-butyl (1-((2-methyl-1-(4-(trifluoromethyl)phenyl)propan-2-yl)amino)-1-oxopropan-2-yl)carbamate (MP077)

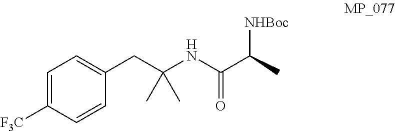 [SMILES: C[C@H](NC(=O)OC(C)(C)C)C(=O)NC(C)(C)Cc1ccc(C(F)(F)F)cc1]

To a solution of tertiary amine MP_077b (470 mg, 2.31 mmol) in 10 ml of DCM was added 4-dimethylaminopyridine (56 mg, 0.46 mmol) and N-(tert-butoxycarbonyl)-L-alanine (657 mg, 3.47 mmol). The mixture was allowed to stir for 10 min at 0° C. Then, N-(3-dimethylaminopropyl)-N′-ethylcarbodiimide hydrochloride (657 mg, 2.31 mmol) was added to the solution and the mixture was stirred for 1 h at the same temperature. After being stirred for 12 h at room temperature, reaction mixture was quenched with water. The organic layer was extracted by ethyl acetate (40 mL×2), washed with brine (20 mL), dried over Na₂SO₄, filtered and concentrated under reduced pressure. The residue was subjected to flash column chromatography using EtOAc/hexane to afford product MP_77 (642 mg, 1.65 mmol) in 72% yield. TLC (SiO₂) R_{f}=0.4 (EtOAc:hexane=1:4, visualized by ceric ammonium molybdate)¹H NMR (400 MHz, CDCl₃) δ 7.50 (d, J=8.0 Hz, 2H), 7.26-7.13 (m, 2H), 5.91 (s, 1H), 4.95 (s, 1H), 4.01 (s, 1H), 3.19 (d, J=13.2 Hz, 1H), 3.06 (d, J=13.2 Hz, 1H), 1.38 (s, 9H), 1.32 (d, J=2.1 Hz, 4H), 1.28 (d, J=9.8 Hz, 4H). ¹³C NMR (101 MHz, CDCl₃) δ 172.28, 155.64, 142.06, 130.80, 128.67 (q, J=32.7 Hz), 124.80 (q, J=3.7 Hz), 124.33 (q, J=271.9 Hz), 80.18, 53.79, 53.42, 44.09, 28.20, 27.33, 17.87.

### Preparation of (S)-2-amino-N-(2-methyl-1-(4-(trifluoromethyl)phenyl)propan-2-yl)propanamide hydrochloride (MP_078)

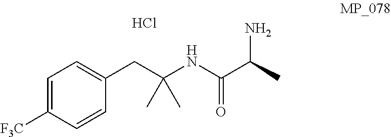 [SMILES: Cl ; C[C@H](N)C(=O)NC(C)(C)Cc1ccc(C(F)(F)F)cc1]

In a round bottom flask equipped with a stir bar, the (S)-tert-butyl (1-((2-methyl-1-(4-(trifluoromethyl)phenyl)propan-2-yl)amino)-1-oxopropan-2-yl)carbamate MP077 (300 mg, 0.77 mmol) was cooled to 0° C. and was added 4M HCl in dioxane (1.5 mL). After being stirred for 1 h at 0° C., the reaction mixture was allowed to stir for an additional 17 h at room temperature. The solution was concentrated under vacuum, and was added Et₂O. The hydrochloride salt was allowed to precipitate and the product MP_078 (138 mg, 0.48 mmol) was filtered and dried in high vacuum in 62% yield. The hydrochloride salt product could also be triturated with Et₂O. ¹H NMR (500 MHz, DMSO) δ 8.27 (s, 3H), 7.86 (s, 1H), 7.62 (d, J=8.1 Hz, 2H), 7.34 (d, J=8.0 Hz, 2H), 3.71 (s, 1H), 3.08 (q, J=12.9 Hz, 2H), 1.29 (d, J=7.0 Hz, 3H), 1.22 (d, J=11.2 Hz, 5H). ¹³C NMR (126 MHz, DMSO) δ 169.67, 143.29, 131.68, 127.39 (q, J=31.6 Hz), 125.07 (d, J=3.6 Hz), 124.96 (q, J=271.8 Hz), 53.96, 48.80, 43.56, 27.24, 18.02. HRMS (DART, m/z): Calculated for C₁₄H₁₉F₃N₂OH [M+H]⁺=289.1528; found 289.1502 Detailed Procedures and Spectral Data for Synthesis of 2-amino-N-(4-chlorophenethyl)-2-methylpropanamide (MP_079)

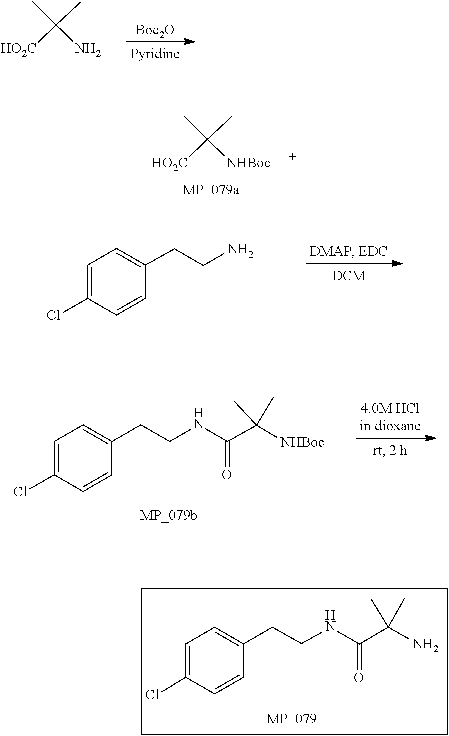

### Preparation of 2-((tert-butoxycarbonyl)amino)-2-methylpropanoic acid (MP_079a)

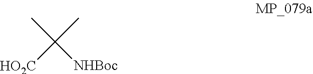

A solution of 2-aminoisobutyric acid (1.03 g, 10 mmol) in 1.0 M NaOH (10 mL), H₂O (20 mL) and dioxane (20 mL) cooled to 0° C. was treated with di-tert-butyl dicarbonate (2.18 g, 11 mmol) and stirred at room temperature for 10 h. The reaction mixture was concentrated under reduced pressure and acidified to pH 2.0 with 1.0 M NaHSO₄. The organic layers were extracted with EtOAc, dried over MgSO₄, filtered and concentrated under reduced pressure to give product MP_079a (921 mg, 4.04) as a white solid in 40% yield. ¹H NMR (400 MHz, DMSO) δ 12.11 (s, 1H), 6.97 (s, 1H), 1.32 (s, 9H), 1.25 (s, 6H). ¹³C NMR (101 MHz, DMSO) δ 176.56 (s), 154.96 (s), 146.66 (s), 86.04 (s), 78.17 (s), 28.67 (s), 27.32 (s), 25.61 (s).

### Preparation of tert-butyl (1-((4-chlorophenethyl)amino)-2-methyl-1-oxopropan-2-yl)carbamate (MP_079b)

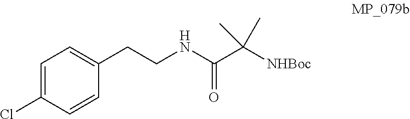 [SMILES: CC(C)(C)OC(=O)NC(C)(C)C(=O)NCCc1ccc(Cl)cc1]

To a solution of 2-(4-chlorophenyl)ethanamine (140 μL, 1 mmol) in 5 ml of DCM were added 4-dimethylaminopyridine (24 mg, 0.2 mmol) and 2-((tert-butoxycarbonyl)amino)-2-methylpropanoic acid MP_079a (305 mg, 1.5 mmol) and the mixture was allowed to stir for 10 min at 0° C. Then, N-(3-dimethylaminopropyl)-N′-ethylcarbodiimide hydrochloride (288 mg, 1.5 mmol) was added to the solution and the mixture was stirred for 1 h. After being stirred for 12 h at room temperature, the reaction mixture was quenched with water. The organic layer was extracted by ethyl acetate (10 mL×2), washed with brine (5 mL), dried over Na₂SO₄, filtered and concentrated under reduced pressure. The residue was subjected to flash column chromatography using EtOAc/hexane to afford product MP_79b (323 mg, 0.95 mmol) in 95% yield. TLC (SiO₂) R_{f}=0.3 (EtOAc:hexane=1:2, visualized by ninhydrin)¹H NMR (400 MHz, CDCl₃) δ 7.29-7.21 (m, 2H), 7.13 (d, J=8.5 Hz, 2H), 6.53 (s, 1H), 4.82 (s, 1H), 3.48 (dd, J=13.0, 7.0 Hz, 2H), 2.78 (t, J=7.1 Hz, 2H), 1.44 (s, 6H), 1.41 (s, 9H). ¹³C NMR (101 MHz, CDCl₃) δ 174.63, 154.79, 137.50, 132.25, 130.13, 128.68, 56.83, 40.74, 35.07, 28.30, 25.78.

### Preparation of 2-amino-N-(4-chlorophenethyl)-2-methylpropanamide (MP_079)

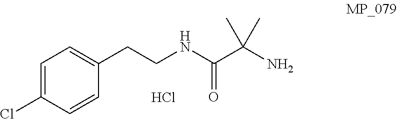 [SMILES: CC(C)(N)C(=O)NCCc1ccc(Cl)cc1 ; Cl]

In a round bottom flask equipped with a stir bar, tert-butyl (1-((4-chlorophenethyl)amino)-2-methyl-1-oxopropan-2-yl)carbamate MP_079b (85 mg, 0.25 mmol) was cooled to 0° C. and was added 4M HCl in dioxane (1 mL). After being stirred for 1 h at 0° C., the reaction mixture was allowed to stir for an additional 2 h at room temperature. The solution was concentrated under vacuum, and was added Et₂O. The hydrochloride salt was allowed to precipitate, and the product (54 mg, 0.2 mmol) was filtered and dried in high vacuum (78% yield). The hydrochloride salt product could also be triturated with Et₂O. ¹H NMR (500 MHz, DMSO) δ 8.40 (s, 2H), 8.20 (s, 5H), 7.32 (d, J=8.2 Hz, 3H), 7.21 (d, J=8.2 Hz, 3H), 3.34 (d, J=6.5 Hz, 3H), 2.74 (t, J=6.7 Hz, 3H), 1.38 (s, 9H). ¹³C NMR (126 MHz, DMSO) δ 171.85, 138.74, 131.28, 131.20, 128.62, 56.79, 40.79, 34.37, 24.06. HRMS (DART, m/z) Calculated for C₁₂H₁₇ClN₂OH [M+H]⁺=241.1108, found 241.1102.

Detailed Procedures and Spectral Data for Synthesis of MP compound from Sertraline (MP080, 081)

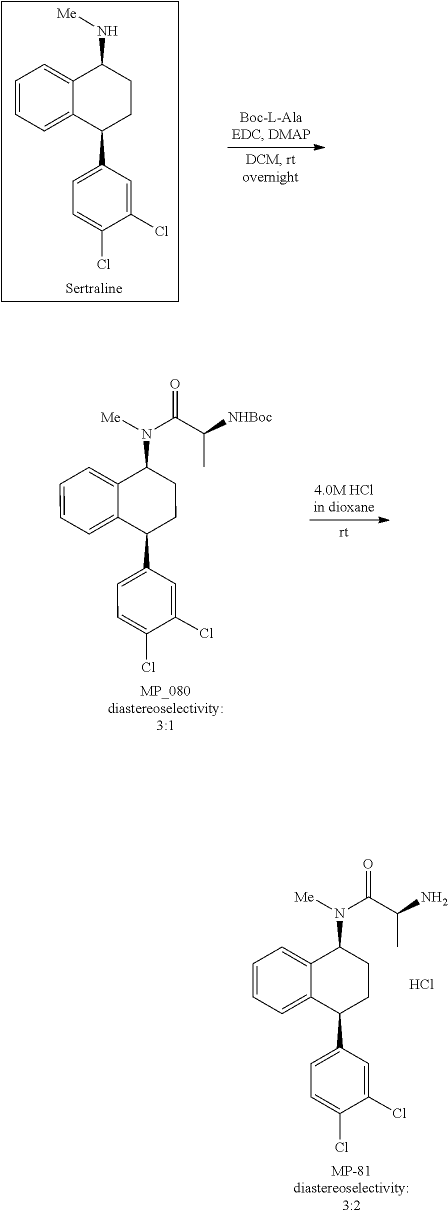 [SMILES: CN[C@H]1CC[C@@H](c2ccc(Cl)c(Cl)c2)c2ccccc21 ; C[C@H](NC(=O)OC(C)(C)C)C(=O)N(C)[C@H]1CC[C@@H](c2ccc(Cl)c(Cl)c2)c2ccccc21 ; C[C@H](N)C(=O)N(C)[C@H]1CC[C@@H](c2ccc(Cl)c(Cl)c2)c2ccccc21 ; Cl]

### Preparation of tert-butyl ((S)-1-(((1S,4R)-4-(3,4-dichlorophenyl)-1,2,3,4-tetrahydronaphthalen-1-yl)(methyl)amino)-1-oxopropan-2-yl)carbamate (MP_080)

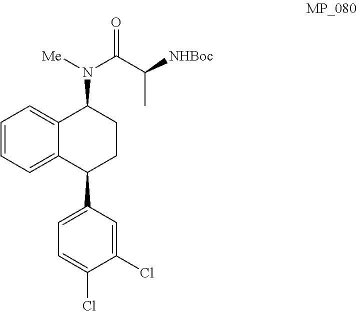 [SMILES: C[C@H](NC(=O)OC(C)(C)C)C(=O)N(C)[C@H]1CC[C@@H](c2ccc(Cl)c(Cl)c2)c2ccccc21]

To a solution of Sertraline (103 mg, 0.3 mmol) in 1.5 ml of DCM were added 4-dimethylaminopyridine (7 mg, 0.06 mmol) and N-(tert-butoxycarbonyl)-L-alanine (85 mg, 0.45 mmol), and the mixture was allowed to stir for 10 min at 0° C. Then, N-(3-dimethylaminopropyl)-N′-ethylcarbodiimide hydrochloride (86 mg, 0.45 mmol) was added to the solution and the mixture was stirred for 1 h. After being stirred for 12 h at room temperature, the reaction mixture was quenched with water. The organic layer was extracted by ethyl acetate (10 mL×2), washed with brine (5 mL), dried over Na₂SO₄, filtered and concentrated under reduced pressure. The residue was subjected to flash column chromatography using EtOAc/hexane to afford product mixture of MP_080 (diastereoselectivity=3:1, 67 mg, 0.14 mmol) in 47% yield. TLC (SiO₂) R_{f}=0.3 (EtOAc:hexane=1:4, visualized by ninhydrin)¹H NMR (400 MHz, CDCl₃) δ 7.33 (d, J=8.3 Hz, 1H), 7.30-7.11 (m, 4H), 7.08 (dd, J=5.0, 1.9 Hz, 1H), 6.99 (dd, J=16.6, 7.5 Hz, 2H), 6.82 (dd, J=8.3, 1.6 Hz, 1H), 5.87 (dd, J=9.9, 6.8 Hz, 1H), 5.57 (d, J=7.8 Hz, 1H), 4.74-4.58 (m, 1H), 4.29-4.15 (m, 1H), 2.80 (s, 3H), 2.29 (dd, J=8.4, 4.7 Hz, 1H), 1.81-1.61 (m, 3H), 1.45 (d, J=5.8 Hz, 9H), 1.41 (s, 3H). ¹³C NMR (101 MHz, CDCl₃) δ 173.88, 155.14, 146.86, 138.39, 135.73, 135.13, 132.39, 131.02, 130.62, 130.22, 130.17, 127.98, 127.85, 127.58, 127.52, 126.94, 79.56, 60.40, 56.39, 52.94, 46.85, 43.02, 42.78, 30.70, 30.21, 29.95, 29.38, 28.40, 28.35, 22.93, 21.29, 20.10, 19.26. HRMS (DART, m/z): Calculated for C₂₅H₃₀Cl₂N₂O₃H [M+H]⁺=477.1712; found 477.1682.

### Preparation of(S)-2-amino-N-((1S,4R)-4-(3,4-dichlorophenyl)-1,2,3,4-tetrahydronaphthalen-1-yl)-N-methylpropanamide hydrochloride (MP_081)

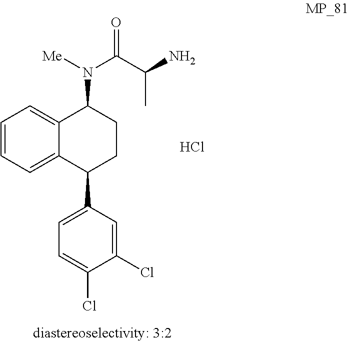 [SMILES: C[C@H](N)C(=O)N(C)[C@H]1CC[C@@H](c2ccc(Cl)c(Cl)c2)c2ccccc21 ; Cl]

In a round bottom flask equipped with a stir bar, tert-butyl ((S)-1-(((1S,4R)-4-(3,4-dichlorophenyl)-1,2,3,4-tetrahydronaphthalen-1-yl)(methyl)amino)-1-oxopropan-2-yl)carbamate MP_080 (60 mg, 0.13 mmol) was cooled to 0° C. and was added 4M HCl in dioxane (1 mL). After being stirred for 1 h at 0° C., the reaction mixture was allowed to stir for an additional 2 h at room temperature. The solution was concentrated under vacuum, and was added Et₂O. The hydrochloride salt was allowed to precipitate, and the product MP_081 (34 mg, 0.08 mmol) was filtered and dried in high vacuum (65% yield). The hydrochloride salt product could also be triturated with Et₂O. ¹H NMR (500 MHz, DMSO) δ 8.37 (m, 4H), 7.53 (dd, J=8.3, 1.9 Hz, 2H), 7.31-7.27 (m, 2H), 7.26-7.21 (m, 2H), 7.00-6.91 (m, 3H), 6.88 (dd, J=8.4, 1.7 Hz, 1H), 5.65 (s, 1H), 5.25-5.14 (m, 1H), 4.33 (dd, J=15.0, 10.3 Hz, 2H), 2.77 (s, 3H), 2.59 (d, J=2.6 Hz, 2H), 2.36 (d, J=3.8 Hz, 1H), 2.28-2.19 (m, 1H), 1.97 (d, J=13.5 Hz, 2H), 1.75-1.56 (m, 3H), 1.47 (d, J=6.8 Hz, 3H), 1.36 (d, J=6.8 Hz, 2H). ¹³C NMR (126 MHz, DMSO) δ 170.96, 170.61, 148.24, 148.20, 138.87, 138.62, 135.95, 135.70, 131.38, 131.35, 131.26, 130.93, 130.90, 130.82, 129.35, 129.27, 129.15, 128.26, 127.98, 127.92, 127.89, 127.17, 126.81, 66.82, 65.39, 55.60, 46.90, 46.74, 42.41, 42.21, 29.79, 29.62, 29.32, 22.57, 21.29, 17.85, 16.54, 15.64. HRMS (DART, m/z): Calculated for C₂₀H₂₂Cl₂N₂OH [M+H]⁺=377.1187; found 377.0800.

Detailed Procedures and Spectral Data for Synthesis of Sertraline Moiety MP Compound (MP 082)

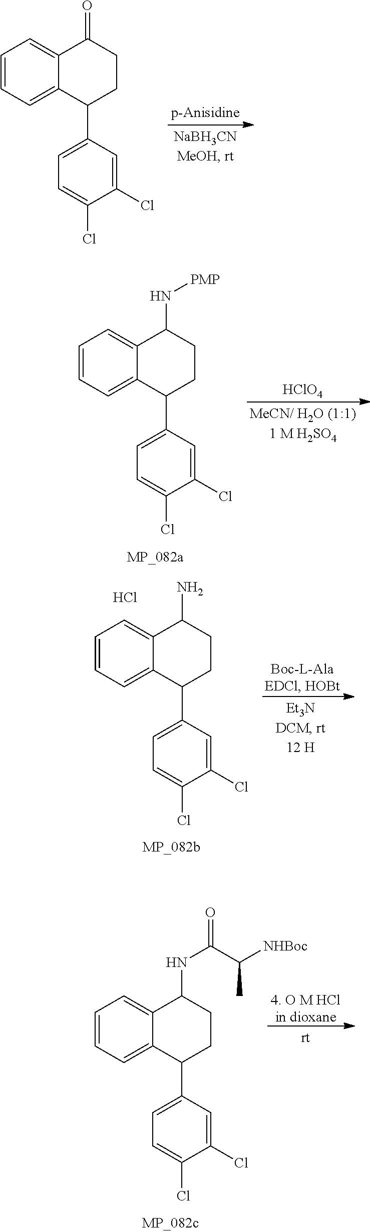

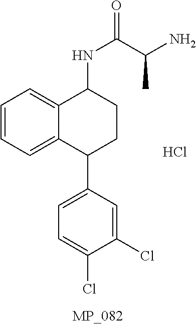 [SMILES: C[C@H](N)C(=O)NC1CCC(c2ccc(Cl)c(Cl)c2)c2ccccc21 ; Cl]

### Preparation of 4-(3,4-dichlorophenyl)-N-(4-methoxyphenyl)-1,2,3,4-tetrahydronaphthalen-1-amine (MP_082a)

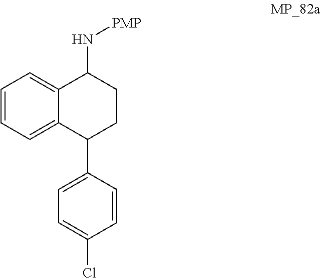

To a solution of p-anisidine (3.69 g, 30 mmol) in absolute methanol (12 mL) was added 6 mmol of 3 N HCl-methanol (methanolic HCl), followed by 4-(3,4-dichlorophenyl)-3,4-dihydronaphthalen-1(2H)-one (874 mg, 3 mmol) and sodium cyanoborohydride (170 mg, 2.7 mmol). The solution was stirred at room temperature for 72 h. Concentrated HCl was added until Ph<2, and the MeOH was removed in reduced pressure. The residue was taken up in 20 mL of H₂O and extracted with Et₂O. The aqueous solution was brought to pH>10 with saturated KOH and brine, and extracted with EtOAC. The combined reaction mixture was dried over MgSO₄ and evaporated under reduced pressure to give crude product. Purification by flash chromatography with EtOAc and hexane provided product MP_082a (843 mg, 2.12 mmol) in 71% yield. TLC (SiO₂) R_{f}=0.6 (EtOAc:hexane=1:9, visualized by ninhydrin). ¹H NMR (500 MHz, CDCl₃) δ 7.47 (d, J=7.7 Hz, 1H), 7.39 (d, J=8.2 Hz, 1H), 7.28-7.12 (m, 3H), 7.00 (dd, J=8.3, 2.1 Hz, 1H), 6.89-6.78 (m, 3H), 6.75-6.65 (m, 2H), 4.60 (t, J=4.1 Hz, 1H), 4.01 (dd, J=8.8, 5.3 Hz, 1H), 3.78 (s, 3H), 2.20-1.87 (m, 4H). ¹³C NMR (126 MHz, CDCl₃) δ 152.17, 147.02, 141.61, 138.83, 138.74, 132.44, 130.70, 130.42, 130.28, 130.10, 129.71, 129.69, 128.31, 128.28, 127.63, 127.01, 115.10, 114.41, 55.89, 52.13, 45.16, 28.89, 26.76.

### Preparation of 4-(3,4-dichlorophenyl)-1,2,3,4-tetrahydronaphthalen-1-amine hydrochloride (MP_082b)

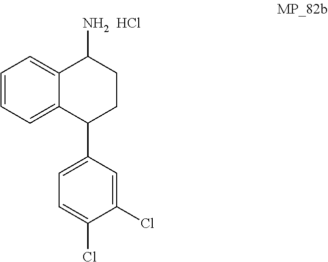

To a solution of 4-(3,4-dichlorophenyl)-N-(4-methoxyphenyl)-1,2,3,4-tetrahydronaphthalen-1-amine MP_082a (330 mg, 0.83 mmol) in MeCN/H₂O (20 mL, 1:1) were added periodic acid (189 mg, 0.83 mmol) and 1 M aqueous H₂SO₄ (1 mL). The mixture was stirred for 16 h at room temperature and then washed with CH₂Cl₂(3×50 mL). The resulting aqueous phase was subsequently brought to pH 10.5 through addition of 5 M aqueous KOH and extracted with EtOAc (4×50 mL). The combined organic layers were brought to pH 1 via addition of 4.0 M HCl in dioxane, and then stirred for 1 h. The residue was concentrated to afford the HCl salt of 4-(3,4-dichlorophenyl)-1,2,3,4-tetrahydronaphthalen-1-amine MP_082b (185 mg, 0.57 mmol) in 68% yield. ¹H NMR (500 MHz, DMSO) δ 8.67 (s, 3H), 7.70-7.47 (m, 3H), 7.36-7.12 (m, 3H), 6.71 (d, J=7.6 Hz, 1H), 4.47 (s, 1H), 4.11 (s, 1H), 2.18-1.83 (m, 4H). ¹³C NMR (126 MHz, DMSO) δ 147.59, 139.91, 133.36, 131.47, 131.29, 130.87, 130.11, 129.92, 129.48, 129.09, 127.16, 66.82, 48.02, 44.21, 27.20, 25.93.

### Preparation of tert-butyl ((2S)-1-((4-(3,4-dichlorophenyl)-1,2,3,4-tetrahydronaphthalen-1-yl)amino)-1-oxopropan-2-yl)carbamate (MP_082c)

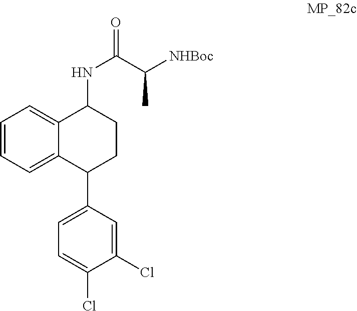 [SMILES: C[C@H](NC(=O)OC(C)(C)C)C(=O)NC1CCC(c2ccc(Cl)c(Cl)c2)c2ccccc21]

To a solution of 4-(3,4-dichlorophenyl)-1,2,3,4-tetrahydronaphthalen-1-amine hydrochloride MP_082b (164 mg, 0.5 mmol) in 2 ml of DCM was added 1-Hydroxybenzotriazole hydrate (20 mg, 0.15 mmol) and N-(tert-butoxycarbonyl)-L-alanine (142 mg, 0.75 mmol). Followed by trimethylamine (105 μL, 0.75 mmol) and the mixture was allowed to stir for 10 min at 0° C. Then, N-(3-dimethylaminopropyl)-N′-ethylcarbodiimide hydrochloride (144 mg, 0.75 mmol) was added to the solution and the mixture was stirred for 1 h. After being stirred for 12 h at room temperature, the reaction mixture was quenched with water. The organic layer was extracted by EtOAc (50 mL×3), washed with brine (5 mL), dried over Na₂SO₄, filtered and concentrated under reduced pressure. The residue was subjected to flash column chromatography using EtOAc/hexane to afford product mixture of MP_082c (diastereoselectivity=1:4, 24 mg, 0.05 mmol) in 10% yield. TLC (SiO₂) R_{f}=0.5 (EtOAc: hexane=1:2, visualized by ceric ammonium molybdate) MP_082c—diastereomer 1 (upper spot, 5 mg)¹H NMR (500 MHz, CDCl₃) δ 7.36 (d, J=8.2 Hz, 1H), 7.32-7.25 (m, 2H), 7.23-7.08 (m, 3H), 6.93 (dd, J=8.3, 2.0 Hz, 1H), 6.81 (d, J=7.6 Hz, 1H), 6.67 (s, 1H), 5.17 (dt, J=8.3, 5.5 Hz, 1H), 4.97 (s, 1H), 4.13 (dt, J=14.3, 6.8 Hz, 1H), 4.08-3.98 (m, 1H), 2.21-2.07 (m, 1H), 1.96 (ddd, J=24.5, 13.0, 10.7 Hz, 1H), 1.91-1.80 (m, 2H), 1.39 (d, J=9.9 Hz, 12H). ¹³C NMR (126 MHz, CDCl₃) δ 171.86, 146.79, 138.41, 137.00, 132.46, 130.59, 130.34, 129.98, 128.93, 128.25, 127.81, 127.24, 80.38, 47.32, 44.55, 29.33, 28.25, 27.27. MP_082c—diastereomer 2 (lower spot, 19 mg)¹H NMR (400 MHz, CDCl₃) δ 7.41-7.06 (m, 5H), 6.94 (dd, J=8.3, 2.0 Hz, 1H), 6.82 (d, J=7.6 Hz, 1H), 6.65 (s, 1H), 5.15 (d, J=7.9 Hz, 1H), 4.98 (s, 1H), 4.23-4.08 (m, 1H), 4.03 (t, J=6.4 Hz, 1H), 2.12 (s, 1H), 2.02-1.81 (m, 3H), 1.39 (t, J=4.9 Hz, 12H). ¹³C NMR (101 MHz, CDCl₃) δ 171.76, 146.76, 138.48, 136.90, 132.43, 130.65, 130.34, 129.99, 129.00, 128.27, 127.84, 127.27, 80.34, 47.51, 44.58, 29.21, 28.28, 27.24. HRMS (DART, m/z): Calculated for C₂₄H₂₈Cl₂N₂O₃H [M+H]⁺=463.1555; found 463.1519 Preparation of (2S)-2-amino-N-(4-(3,4-dichlorophenyl)-1,2,3,4-tetrahydronaphthalen-1-yl)propanamide (MP_082) (from MP_082c-diastereomer 2)

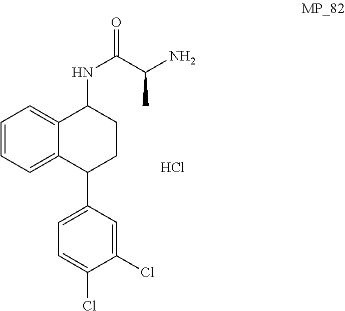 [SMILES: C[C@H](N)C(=O)NC1CCC(c2ccc(Cl)c(Cl)c2)c2ccccc21 ; Cl]

In a round bottom flask equipped with a stir bar, tert-butyl ((2S)-1-((4-(3,4-dichlorophenyl)-1,2,3,4-tetrahydronaphthalen-1-yl)amino)-1-oxopropan-2-yl)carbamate MP_082c (19 mg, 0.04 mmol) was cooled to 0° C. and was added 4M HCl in dioxane (0.5 mL). After being stirred for 1 h at 0° C., the reaction mixture was allowed to stir for an additional 2 h at room temperature. The solution was concentrated under vacuum, and was added Et₂O. The hydrochloride salt was allowed to precipitate, and the product MP_082 (10 mg, 0.03 mmol) was filtered and dried in high vacuum (63% yield). The hydrochloride salt product could also be triturated with Et₂O. ¹H NMR (500 MHz, MeOD) δ 7.44 (d, J=8.3 Hz, 1H), 7.31 (d, J=2.1 Hz, 1H), 7.24 (dd, J=6.2, 1.1 Hz, 2H), 7.20-7.11 (m, 2H), 6.84 (d, J=7.8 Hz, 2H), 5.20-5.01 (m, 2H), 4.15 (t, J=6.0 Hz, 1H), 3.92 (dd, J=14.1, 7.0 Hz, 1H), 3.75-3.71 (m, 0.5H), 3.69-3.62 (m, 1H), 3.57 (dd, J=5.5, 4.1 Hz, 0.5H), 2.15 (ddd, J=10.8, 7.8, 4.7 Hz, 1H), 2.01-1.92 (m, 3H), 1.57 (d, J=7.0 Hz, 3H), 1.49 (s, 0.4H). ¹³C NMR (126 MHz, MeOD) δ 168.89, 147.53, 138.75, 136.39, 131.81, 130.53, 129.97, 129.81, 129.71, 128.64, 128.35, 127.52, 126.82, 49.00, 44.25, 28.92, 26.71, 16.41. HRMS (DART, m/z): Calculated for C₁₉H₂₁C₁₂N₂OH [M+H]⁺=363.1031; found 363.0822

Detailed Procedures and Spectral Data for Synthesis of MP Compound from Sertraline (MP-083)

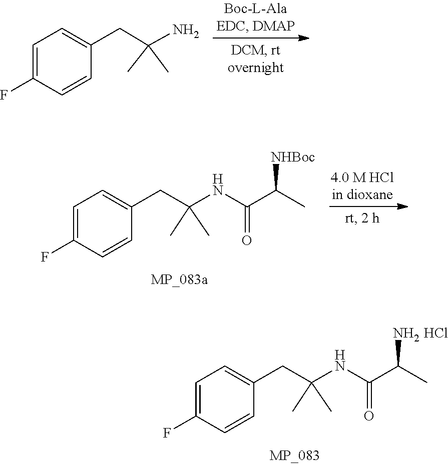 [SMILES: CC(C)(N)Cc1ccc(F)cc1 ; C[C@H](NC(=O)OC(C)(C)C)C(=O)NC(C)(C)Cc1ccc(F)cc1 ; C[C@H](N)C(=O)NC(C)(C)Cc1ccc(F)cc1 ; Cl]

### Preparation of (S)-tert-butyl (1-((1-(4-fluorophenyl)-2-methylpropan-2-yl)amino)-1-oxopropan-2-yl)carbamate (MP_083a)

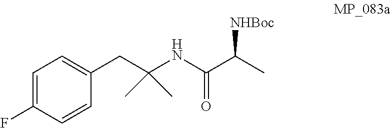 [SMILES: C[C@H](NC(=O)OC(C)(C)C)C(=O)NC(C)(C)Cc1ccc(F)cc1]

To a solution of 1-(4-fluorophenyl)-2-methylpropan-2-amine (335 mg, 2 mmol) in 10 ml of DCM was added 4-dimethylaminopyridine (49 mg, 0.4 mmol) and N-(tert-butoxycarbonyl)-L-alanine (568 mg, 3 mmol), and the mixture was allowed to stir for 10 min at 0° C. Then, N-(3-dimethylaminopropyl)-N′-ethylcarbodiimide hydrochloride (575 mg, 3 mmol) was added to the solution and the mixture was stirred for 1 h. After being stirred for 12 h at room temperature, the reaction mixture was quenched with water. The organic layer was extracted by ethyl acetate (50 mL×2), washed with brine (30 mL), dried over Na₂SO₄, filtered and concentrated under reduced pressure. The residue was subjected to flash column chromatography using EtOAc/hexane to afford product MP_083a (438 mg, 1.29 mmol) in 65% yield. TLC (S102) R_{f}=0.3 (EtOAc: hexane=1:4)¹H NMR (500 MHz, CDCl₃) δ 7.11-7.01 (m, 2H), 6.93 (dd, J=9.8, 7.7 Hz, 2H), 5.81 (s, 1H), 4.96 (s, 1H), 4.00 (s, 1H), 3.07 (d, J=13.4 Hz, 1H), 2.94 (d, J=13.4 Hz, 1H), 1.39 (s, 9H), 1.29 (d, J=7.1 Hz, 3H), 1.26 (s, 3H). ¹³C NMR (126 MHz, CDCl₃) δ 172.09, 162.65, 161.68 (d, J=244.3 Hz), 155.58133.49 (d, J=3.1 Hz), 131.85 (d, J=7.8 Hz), 114.73 (d, J=21.0 Hz), 80.08, 53.85, 50.48, 43.68, 28.25, 27.18, 18.01.

### Preparation of (S)-2-amino-N-(1-(4-fluorophenyl)-2-methylpropan-2-yl)propanamide hydrochloride (MP_083)

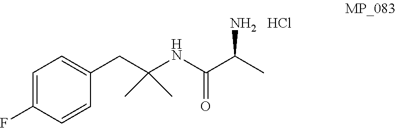

In a round bottom flask equipped with a stir bar, (S)-tert-butyl (1-((1-(4-fluorophenyl)-2-methylpropan-2-yl)amino)-1-oxopropan-2-yl)carbamate MP_083a (68 mg, 0.2 mmol) was cooled to 0° C. and was added 4M HCl in dioxane (1 mL). After being stirred for 1 h at 0° C., the reaction mixture was allowed to stir for an additional 2 h at room temperature. The solution was concentrated under reduced pressure, and was added DCM. The hydrochloride salt was allowed to precipitate, and the product MP_083 (45 mg, 0.16 mmol) was filtered and dried in high vacuum (82% yield). The hydrochloride salt product could also be triturated with DCM. ¹H NMR (500 MHz, DMSO) δ 8.30 (s, 3H), 7.88 (s, 1H), 7.14 (dd, J=8.5, 5.8 Hz, 2H), 7.06 (t, J=8.8 Hz, 2H), 3.84-3.66 (m, 1H), 2.95 (q, J=13.1 Hz, 2H), 1.29 (d, J=6.9 Hz, 3H), 1.19 (d, J=1.9 Hz, 6H). ¹³C NMR (126 MHz, DMSO) δ 169.56, 161.45 (d, J=241.7 Hz), 134.44 (d, J=3.0 Hz), 132.60 (d, J=7.9 Hz), 114.93 (d, J=20.8 Hz), 54.00, 48.79, 43.13, 27.13, 27.12, 18.04. HRMS (DART, m/z) Calculated for C₁₃H₁₉FN₂OH [M+H]⁺=239.1560, found 239.0684

Detailed Procedures and Spectral Data for Synthesis of MP compound from Sertraline (MP-04)

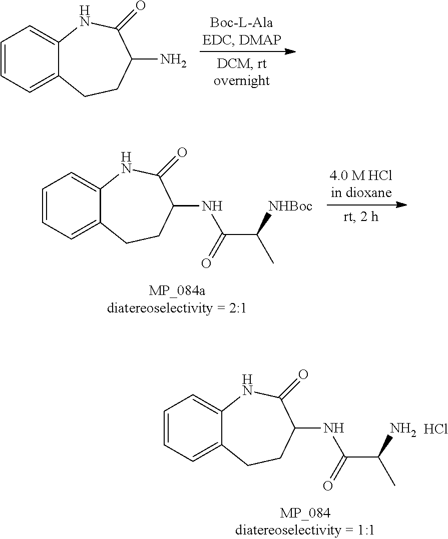 [SMILES: NC1CCc2ccccc2NC1=O ; C[C@H](NC(=O)OC(C)(C)C)C(=O)NC1CCc2ccccc2NC1=O ; C[C@H](N)C(=O)NC1CCc2ccccc2NC1=O ; Cl]

### Preparation of tert-butyl ((2S)-1-oxo-1-((2-oxo-2,3,4,5-tetrahydro-1H-benzo[b]azepin-3-yl)amino)propan-2-yl)carbamate (MP_084a)

(lab note volume 3 page 39, y-245)

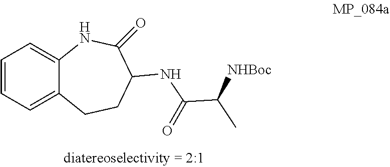 [SMILES: C[C@H](NC(=O)OC(C)(C)C)C(=O)NC1CCc2ccccc2NC1=O]

To a solution of 3-amino-4,5-dihydro-1H-benzo[b]azepin-2(3H)-one (264 mg, 1.5 mmol) in 10 ml of DCM was added 4-dimethylaminopyridine (37 mg, 0.3 mmol) and N-(tert-butoxycarbonyl)-L-alanine (426 mg, 2.3 mmol), and the mixture was allowed to stir for 10 min at 0° C. Then, N-(3-dimethylaminopropyl)-N′-ethylcarbodiimide hydrochloride (431 mg, 2.3 mmol) was added to the solution and the mixture was stirred for 1 h. After being stirred for 12 h at room temperature, the reaction mixture was quenched with water. The organic layer was extracted by ethyl acetate (50 mL×2), washed with brine (30 mL), dried over Na₂SO₄, filtered and concentrated under reduced pressure. The residue was subjected to flash column chromatography using EtOAc/hexane to afford product MP_084a (105 mg, 0.3 mmol) in 20% yield.

TLC (SiO₂) R_{f}=0.2 (EtOAc:hexane=1:1)

¹H NMR (500 MHz, CDCl₃) δ 9.03 (s, 0.3H), 8.64 (s, 0.6H), 7.40 (dd, J=15.5, 5.4 Hz, 1H), 7.19 (dd, J=11.0, 4.6 Hz, 2H), 7.12 (dd, J=10.9, 3.8 Hz, 1H), 6.96 (dd, J=9.3, 4.7 Hz, 1H), 5.57 (d, J=7.0 Hz, 0.3H), 5.31 (d, J=7.3 Hz, 0.6H), 4.49 (dt, J=11.2, 7.6 Hz, 1H), 4.39 (s, 3H), 4.25 (d, J=5.9 Hz, 5H), 3.03-2.81 (m, 1H), 2.81-2.65 (m, 11H), 2.65-2.49 (m, 1H), 1.95 (dd, J=20.0, 11.5 Hz, 1H), 1.39 (s, 9H), 1.28 (dd, J=13.5, 7.1 Hz, 3H).

¹³C NMR (126 MHz, CDCl₃) δ 172.82, 172.76, 172.40, 172.15, 155.66, 155.30, 136.31, 136.15, 133.94, 129.97, 129.94, 127.82, 127.75, 126.52, 126.39, 122.55, 122.41, 79.79, 60.46, 50.00, 49.56, 36.32, 36.18, 28.60, 28.40, 19.13, 18.89.

### Preparation of (2S)-2-amino-N-(2-oxo-2,3,4,5-tetrahydro-1H-benzo[b]azepin-3-yl)propanamide hydrochloride (MP_084)

(lab note volume 3 page 59, y-255)

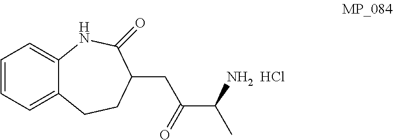

In a round bottom flask equipped with a stir bar, tert-butyl ((2S)-1-oxo-1-((2-oxo-2,3,4,5-tetrahydro-1H-benzo[b]azepin-3-yl)amino)propan-2-yl)carbamate MP_084a (45 mg, 0.13 mmol) was cooled to 0° C. and was added 4M HCl in dioxane (1 mL). After being stirred for 1 h at 0° C., the reaction mixture was allowed to stir for an additional 2 h at room temperature. The solution was concentrated under reduced pressure, and was added DCM. The hydrochloride salt was allowed to precipitate, and the product MP_084 (20 mg, 0.07 mmol) was filtered and dried in high vacuum (54% yield). The hydrochloride salt product could also be triturated with DCM. ¹H NMR (500 MHz, DMSO) δ 9.91 (dd, J=22.9, 10.1 Hz, 1H), 8.68 (dd, J=17.0, 7.8 Hz, 1H), 8.16 (s, 3H), 7.35-6.89 (m, 4H), 4.19 (dt, J=18.8, 9.4 Hz, 1H), 3.84 (s, 1H), 2.82-2.58 (m, 2H), 2.37-2.21 (m, 1H), 2.12-1.89 (m, 1H), 1.40-1.24 (m, 3H). ¹³C NMR (126 MHz, DMSO) δ 171.27, 171.21, 169.47, 138.04, 133.92, 133.86, 130.07, 128.02, 125.78, 125.72, 122.54, 49.58, 48.49, 48.39, 35.40, 35.32, 28.51, 28.43, 17.90, 17.71. HRMS (DART, m/z): Calculated for C₁₃H₁₈ClN₃O₂H [M+H]⁺=248.1399; found 248.1380.

### Detailed Procedures and Spectral Data for Synthesis of (S)-2-amino-N-((1S,2R)-2-(4-chlorophenyl)cyclopentyl)propanamide hydrochloride (MP_085, 086)

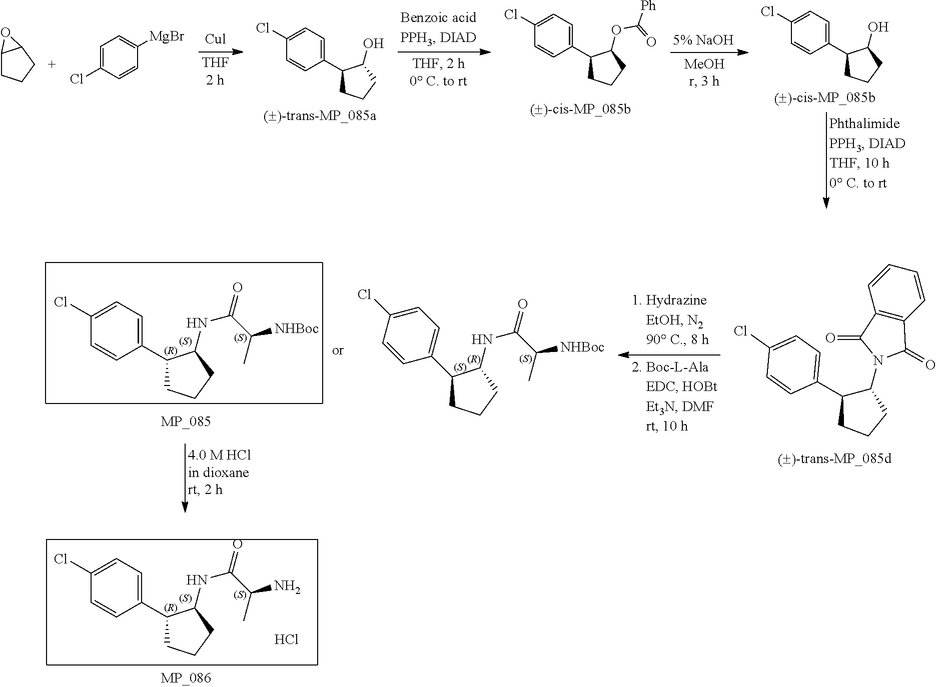

### Preparation of (±)trans-2-(4-chlorophenyl)cyclopentanol (MP 0.085a)¹¹

(lab note volume 3 page 75, y-264)

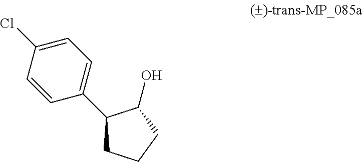 [SMILES: O[C@@H]1CCC[C@H]1c1ccc(Cl)cc1]

A three-necked round-bottom flask equipped with a magnetic stirrer and reflux condenser was charged with 1 M tetrahydrofuran (THF) solution of 4-chlorophenylmagnesium bromide (20 ML, 20 mmol) and copper iodide (380 mg, 2 mmol). To this reaction mixture was then added cyclopentene oxide (1.74 ml, 20 mmol) dissolved in THF (20 mL) dropwise over a period of 60 min (reaction was quite exothermic, reaching THF reflux by the end of addition). The reaction mixture was then stirred to room temperature and quenched with a 25% solution of ammonium chloride (100 mL). Ether was added (100 mL), and the upper organic layer was separated. The organic layer was washed with 25% ammonium chloride solution, dried over MgSO₄, filtered, and concentrated. The residue was subjected to flash column chromatography using EtOAc/hexane to afford product MP_085a (1.74 g, 8.85 mmol) in 44% yield.

TLC (SiO₂) R_{f}=0.5 (EtOAc:hexane=1:4)

¹H NMR (500 MHz, CDCl₃) δ 7.27 (d, J=8.3 Hz, 1H), 7.17 (d, J=8.5 Hz, 1H), 4.10-4.01 (m, 1H), 2.82 (dt, J=16.2, 8.0 Hz, 1H), 2.10 (dddd, J=27.9, 14.8, 8.2, 5.6 Hz, 2H), 1.90-1.58 (m, 2H).

¹³C NMR (126 MHz, CDCl₃) δ 141.81, 131.88, 128.64, 128.47, 80.15, 53.51, 33.96, 31.70, 21.58.

### Preparation of (±)-cis-2-(4-chlorophenyl)cyclopentyl benzoate (MP_085b)¹¹

(lab note volume 3 page 77, y-265)

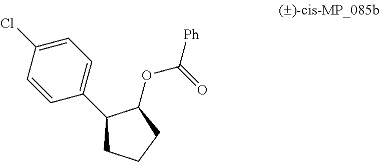 [SMILES: O=C(O[C@H]1CCC[C@H]1c1ccc(Cl)cc1)c1ccccc1]

Into a round bottom fitted with a stirrer were placed diisopropyl azodicarboxylate (1.74 mL, 8.85 mmol) and benzoic acid (1.19 g, 9.73 mmol) in THF (20 mL). A total of (±)-trans-2-(4-chlorophenyl)cyclopentanol MP_085a (1.74 g, 8.85 mmol) and triphenylphosphine (2.55 g, 9.73 mmol) in THF (20 mL) were added dropwise while stirring at 0° C. under argon atmosphere.

After 2 h at this temperature, the TLC showed that the reaction was complete. The solution was allowed to warm to room temperature and then concentrated under reduced vacuum. The residue was subjected to flash column chromatography using EtOAc/hexane to afford product MP_085b (1.66 g, 5.52 mmol) in 62% yield.

TLC (SiO₂) R_{f}=0.4 (EtOAc:hexane=1:15)

¹H NMR (500 MHz, CDCl₃) δ 7.93-7.68 (m, 2H), 7.50 (dd, J=10.5, 4.4 Hz, 1H), 7.36 (dd, J=10.9, 4.8 Hz, 2H), 7.30-7.14 (m, 4H), 5.60 (td, J=5.1, 1.7 Hz, 1H), 3.34-3.15 (m, 1H), 2.31-2.11 (m, 3H), 2.11-1.96 (m, 2H), 1.82 (ddd, J=14.3, 6.9, 3.6 Hz, 1H).

¹³C NMR (126 MHz, CDCl₃) δ 165.75, 138.09, 132.63, 131.96, 130.45, 129.70, 129.21, 128.14, 128.02, 78.45, 49.27, 32.70, 29.23, 22.31.

### Preparation of (±)-cis-2-(4-chlorophenyl)cyclopentanol (MP_085c)¹¹

(lab note volume 3 page 79, y-266)

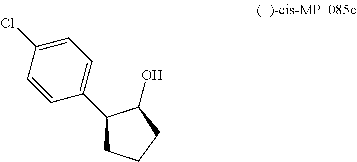 [SMILES: O[C@H]1CCC[C@H]1c1ccc(Cl)cc1]

To (±)-cis-2-(4-chlorophenyl)cyclopentyl benzoate MP_085b (1.66 g, 5.52 mmol) was added 5% NaOH/MeOH (30 mL, excess) and stiffed at room temperature for 3 h. The reaction mixture was then concentrated under reduced pressure. This material was taken into EtOAc and washed once with water, dried over MgSO₄ and concentrated under reduced vacuum. The residue was subjected to flash column chromatography using EtOAc/hexane to afford product MP_085c (872 mg, 4.43 mmol) in 80% yield as a transparent oil.

TLC (SiO₂) R_{f}=0.3 (EtOAc:hexane=1:9)

¹H NMR (500 MHz, CDCl₃) δ 7.27 (d, J=8.6 Hz, 1H), 7.19 (d, J=8.3 Hz, 1H), 4.18 (td, J=4.4, 1.2 Hz, 1H), 2.94 (ddd, J=11.2, 7.2, 4.3 Hz, 1H), 2.05-1.86 (m, 2H), 1.80-1.64 (m, 1H).

¹³C NMR (126 MHz, CDCl₃) δ 138.72, 132.23, 130.04, 128.45, 75.55, 51.31, 34.06, 27.74, 22.33.

### Preparation of (±)-trans-2-(2-(4-chlorophenyl)cyclopentyl)isoindoline-1,3-dione (MP_085d)

(lab note volume 3 page 81, y-267)¹¹

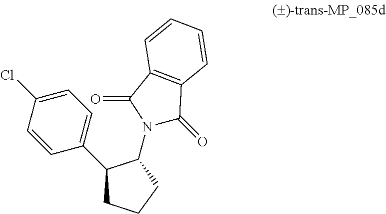 [SMILES: O=C1c2ccccc2C(=O)N1[C@@H]1CCC[C@H]1c1ccc(Cl)cc1]

Into a round bottom fitted with a magnetic stirrer, diisopropyl azodicarboxylate (872 μL, 4.43 mmol) in THF (10 mL) was added dropwise to triphenylphosphine (1.16 g, 4.43 mmol) in THF (10 mL) while stirring at 0° C. under argon atmosphere. At this same temperature, phthalimide (652 mg, 4.43 mmol) in THF (5 mL) was added dropwise followed by adding the solution of (±)-cis-2-(4-chlorophenyl)cyclopentanol MP_085c (872 mg, 4.43 mmol) in THF (5 mL) at 0° C. The reaction was then stirred at this temperature for 4 h and then allowed to warm to room temperature. The reaction mixture was quenched with water. The organic layer was washed once with water, dried over MgSO₄, and concentrated under reduced vacuum. The residue was subjected to flash column chromatography using EtOAc/hexane to afford product MP_085d (480 mg, 2.68 mmol) in 61% yield as a pale yellow oil.

TLC (SiO₂) R_{f}=0.4 (EtOAc:hexane=1:4)

¹H NMR (500 MHz, CDCl₃) δ 7.75 (dd, J=5.5, 3.0 Hz, 2H), 7.65 (dd, J=5.5, 3.0 Hz, 2H), 7.18 (dd, J=6.5, 4.2 Hz, 2H), 7.15 (d, J=8.6 Hz, 2H), 4.71-4.60 (m, 1H), 3.88 (td, J=11.1, 7.6 Hz, 1H), 2.36-2.19 (m, 2H), 2.14-2.02 (m, 2H), 1.96-1.86 (m, 1H), 1.80-1.71 (m, 1H).

¹³C NMR (126 MHz, CDCl₃) δ 168.38, 140.41, 133.88, 132.19, 131.82, 129.97, 128.68, 128.64, 128.58, 123.13, 57.35, 46.72, 33.50, 28.42, 22.34.

### Preparation of tert-butyl ((S)-1-(((1S,2R)-2-(4-chlorophenyl)cyclopentyl)amino)-1-oxopropan-2-yl)carbamate (MP_085)

(lab note volume 3 page 87, y-270 and page 89, y-271)

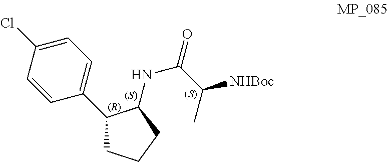

Into a round bottom fitted with a magnetic stirrer and reflux condenser was placed (±)-trans-2-(2-(4-chlorophenyl)cyclopentyl)isoindoline-1,3-dione MP_085d (231 mg, 1.29 mmol) in EtOH (5 mL). To the mixture was added dropwise hydrazine (0.78 mL, 25 mmol) in EtOH (5 mL) while it was stirred at room temperature under argon atmosphere. The reaction was then heated at 90° C. After 8 h the reaction was cooled to room temperature. The precipitate that had formed was filtered. The filtrate was dried over MgSO₄ and concentrated under reduced vacuum. The crude amine product was used for the next step without purification.

To a solution of amine of previous step in 10 ml of DMF was added 1-hydroxybenzotriazole hydrate (270 mg, 2 mmol) and N-(tert-butoxycarbonyl)-L-alanine (284 mg, 1.5 mmol). Followed by trimethylamine (167 μL, 1.2 mmol) and the mixture was allowed to stir for 10 min at 0° C. Then, N-(3-dimethylaminopropyl)-N′-ethylcarbodiimide hydrochloride (288 mg, 1.5 mmol) was added to the solution and the mixture was stirred for 1 h. After being stirred for 12 h at room temperature, the reaction mixture was quenched with water. The organic layer was extracted by EtOAc (50 mL×3), washed with brine (10 mL), dried over MgSO₄, filtered and concentrated under reduced pressure. The residue was subjected to flash column chromatography using EtOAc/hexane to afford product MP_085 and its diastereomer (total: 95 mg, 0.26 mmol) in total 20% yield. In the mixture of two diastereomers, MP_085 (28 mg, 0.08 mmol) was recrystallized by Et₂O as a white solid and filtered (stereochemistry of MP_085 was confirmed by X-ray).

TLC (SiO₂) R_{f}=0.4 (EtOAc:hexane=1:1, visualized by ninhydrin)

¹H NMR (500 MHz, CDCl₃) δ 7.21 (d, J=8.5 Hz, 2H), 7.13 (d, J=8.4 Hz, 2H), 6.33 (s, 1H), 4.89 (s, 1H), 4.34-4.13 (m, 1H), 4.03 (s, 1H), 2.82 (dd, J=17.9, 9.5 Hz, 1H), 2.23 (td, J=13.7, 7.4 Hz, 1H), 2.11 (ddd, J=12.7, 10.2, 6.3 Hz, 1H), 1.92-1.75 (m, 2H), 1.74-1.59 (m, 1H), 1.54-1.45 (m, 1H), 1.38 (s, 9H), 1.24 (d, J=6.3 Hz, 3H).

¹³C NMR (126 MHz, CDCl₃) δ 172.22, 155.68, 140.96, 132.06, 128.60, 128.55, 80.13, 56.72, 51.38, 49.91, 32.84, 32.42, 28.26, 22.22, 17.67.

HRMS (DART, m/z): Calculated for C₁₉H₂₇ClN₂O₃H [M+H]⁺=367.1789; found 367.1761

### Preparation of (S)-2-amino-N-((1S,2R)-2-(4-chlorophenyl)cyclopentyl)propanamide (MP_086)

(lab note volume 3 page 99, y-277 or volume 5 page 27, y-449)

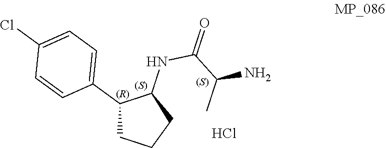

In a round bottom flask equipped with a stir bar, tert-butyl ((S)-1-(((1S,2R)-2-(4-chlorophenyl)cyclopentyl)amino)-1-oxopropan-2-yl)carbamate MP_085 (22 mg, 0.06 mmol) was cooled to 0° C. and was added 4M HCl in dioxane (1 mL). After being stirred for 1 h at 0° C., the reaction mixture was allowed to stir for an additional 2 h at room temperature. The solution was concentrated under reduced pressure, and was added Et₂O. The hydrochloride salt was allowed to precipitate, and the product MP_086 (14 mg, 0.05 mmol) was filtered and dried in high vacuum (88% yield). The hydrochloride salt product could also be triturated with Et₂O.

¹H NMR (500 MHz, DMSO) δ 8.66 (d, J=7.8 Hz, 1H), 8.10 (s, 3H), 7.30 (s, 4H), 4.16-3.97 (m, 1H), 3.73-3.65 (m, 1H), 3.03-2.95 (m, 1H), 2.10-1.99 (m, 2H), 1.79-1.70 (m, 2H), 1.69-1.60 (m, 1H), 1.50-1.42 (m, 1H), 1.30 (d, J=7.0 Hz, 3H).

¹³C NMR (126 MHz, DMSO) δ 169.37, 142.70, 131.13, 129.74, 128.64, 57.54, 49.82, 48.50, 33.04, 32.79, 22.75, 17.72.

HRMS (DART, m/z): Calculated for C₁₄H₁₉ClN₂OH [M+H]⁺=267.1264; found 267.1249

### Detailed Procedures and Spectral Data for Synthesis of (±)-Trans-Cyclopentane Ester Compound (MP_087, 088, 089, 090)

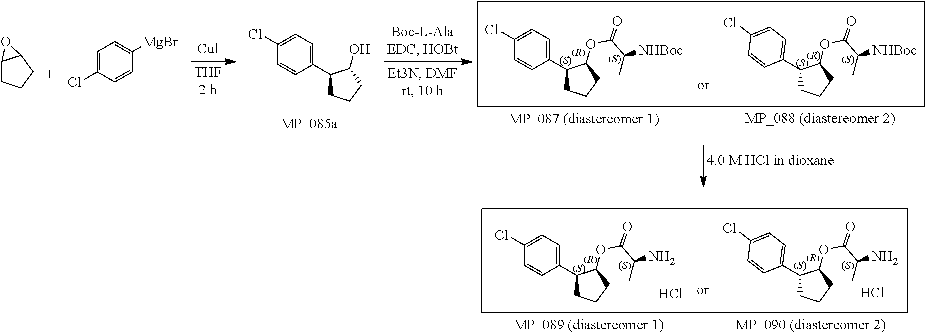

### Preparation of (S)-(±)-trans-2-(4-chlorophenyl)cyclopentyl 2-((tert-butoxycarbonyl)amino)propanoate (MP_087, 088) (lab note volume 3 page 101, y-278)

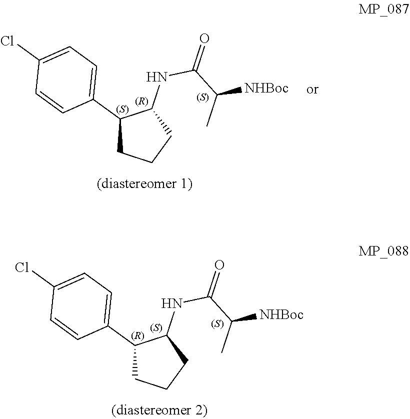

To a solution of (±)-trans-2-(4-chlorophenyl)cyclopentanol MP_085a (393 mg, 2 mmol) in 10 ml of DMF was added 4-dimethylaminopyridine (49 mg, 0.4 mmol) and N-(tert-butoxycarbonyl)-L-alanine (568 mg, 3 mmol), and the mixture was allowed to stir for 10 min at 0° C. Then, N-(3-dimethylaminopropyl)-N′-ethylcarbodiimide hydrochloride (575 mg, 3 mmol) was added to the solution and the mixture was stirred for 1 h. After being stirred for 12 h at room temperature, the reaction mixture was quenched with water. The organic layer was extracted by ethyl acetate (50 mL×3), washed with brine (30 mL), dried over Na₂SO₄, filtered and concentrated under reduced pressure. The residue was subjected to flash column chromatography using EtOAc/hexane to afford product MP_087 (diastereomer 1, 97 mg, 0.26 mmol) and MP_088 (diastereomer 2, 39 mg, 0.11 mmol) in 18% yield.

TLC (SiO₂) R_{f}=0.4 (Et₂O: hexane=1:4)

MP_087 (diastereomer 1, upper spot in TLC)

¹H NMR (400 MHz, CDCl₃) δ 7.27-7.21 (m, 2H), 7.16-7.10 (m, 2H), 5.08 (td, J=6.9, 4.9 Hz, 1H), 5.02 (d, J=5.8 Hz, 1H), 4.24 (d, J=7.0 Hz, 1H), 3.11 (dd, J=15.6, 8.3 Hz, 1H), 2.22-2.09 (m, 2H), 1.90-1.80 (m, 2H), 1.79-1.65 (m, 2H), 1.41 (s, 9H), 1.27 (d, J=7.2 Hz, 3H).

¹³C NMR (101 MHz, CDCl₃)¹³C NMR (101 MHz, CDCl₃) δ 173.14, 155.07, 140.87, 132.22, 128.58, 82.72, 79.76, 50.65, 49.24, 31.68, 31.62, 28.32, 22.75, 18.70.

HRMS (DART, m/z): Calculated for C₁₉H₂₆ClNO₄H [M+H]⁺=368.1629; found 368.1603

MP_088 (diastereomer 2, lower spot in TLC)

¹H NMR (400 MHz, CDCl₃) δ 7.25 (dd, J=7.1, 1.3 Hz, 2H), 7.18-7.07 (m, 2H), 5.09 (td, J=6.9, 5.0 Hz, 1H), 5.00 (d, J=6.6 Hz, 1H), 4.33-4.18 (m, 1H), 3.12 (dd, J=15.8, 8.1 Hz, 1H), 2.22-2.10 (m, 2H), 1.85 (tt, J=7.7, 5.4 Hz, 2H), 1.79-1.68 (m, 2H), 1.42 (s, 9H), 1.27 (d, J=7.2 Hz, 3H).

¹³C NMR (101 MHz, CDCl₃) δ 173.07, 155.03, 140.99, 132.21, 128.60, 82.83, 79.77, 50.52, 49.26, 31.96, 31.89, 30.33, 29.71, 28.32, 22.98, 18.69.

### Preparation of (S)-(1R,2S)-2-(4-chlorophenyl)cyclopentyl 2-aminopropanoate hydrochloride (MP_089, 090)

(lab note volume 3 page 105, y-280 for MP_089 and y-281 for MP_090)

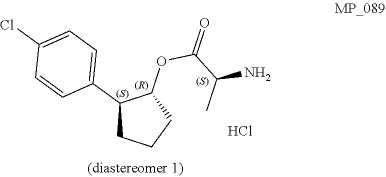

In a round bottom flask equipped with a stir bar, (S)-(±)-trans-2-(4-chlorophenyl)cyclopentyl 2-((tert-butoxycarbonyl)amino)propanoate MP_087 (diastereomer 1, upper spot in TLC) (72 mg, 0.20 mmol) was cooled to 0° C. and was added 4M HCl in dioxane (1 mL). After being stirred for for 1 h at 0° C., the reaction mixture was allowed to stir for an additional 2 h at room temperature. The solution was concentrated under reduced pressure, and was added Et₂O. The hydrochloride salt was allowed to precipitate, and the product MP_089 (47 mg, 0.18 mmol) was filtered and dried in high vacuum (90% yield). The hydrochloride salt product could also be triturated with Et₂O.

¹H NMR (500 MHz, DMSO) δ 8.44 (s, 3H), 7.36-7.31 (m, 2H), 7.29 (d, J=8.5 Hz, 2H), 5.08 (td, J=7.1, 5.0 Hz, 1H), 4.00 (q, J=7.2 Hz, 1H), 3.14 (dt, J=9.5, 7.5 Hz, 1H), 2.19-2.00 (m, 2H), 1.86-1.74 (m, 2H), 1.74-1.61 (m, 2H), 1.29 (d, J=7.2 Hz, 3H).

¹³C NMR (126 MHz, DMSO) δ 170.23, 141.75, 131.50, 129.67, 128.79, 83.43, 50.58, 48.27, 32.10, 31.65, 22.96, 16.16.

HRMS (DART, m/z): Calculated for C₁₄H₁₈ClNO₂H [M+H]⁺=268.1104; found 268.1087

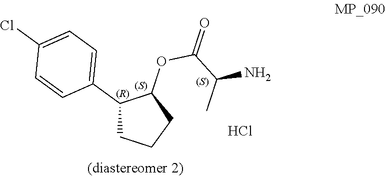

In a round bottom flask equipped with a stir bar, (S)-(±)-trans-2-(4-chlorophenyl)cyclopentyl 2-((tert-butoxycarbonyl)amino)propanoate MP_087 (diastereomer 2, lower spot in TLC) (30 mg, 0.08 mmol) was cooled to 0° C. and was added 4M HCl in dioxane (1 mL). After being stirred for 1 h at 0° C., the reaction mixture was allowed to stir for an additional 2 h at room temperature.

The solution was concentrated under reduced pressure, and was added Et₂O. The hydrochloride salt was allowed to precipitate, and the product MP_090 (20 mg, 0.07 mmol) was filtered and dried in high vacuum (99% yield). The hydrochloride salt product could also be triturated with Et₂O.

¹H NMR (500 MHz, CDCl₃) δ 8.36 (s, 3H), 7.40-7.34 (m, 2H), 7.34-7.29 (m, 2H), 5.10 (td, J=6.9, 4.6 Hz, 1H), 4.03 (q, J=7.2 Hz, 1H), 3.23-3.15 (m, 1H), 2.16 (ddd, J=20.2, 11.2, 6.3 Hz, 2H), 1.85-1.77 (m, 2H), 1.69 (ddd, J=12.5, 9.3, 4.7 Hz, 2H), 1.38 (d, J=7.2 Hz, 3H). ¹³C NMR (126 MHz, CDCl₃) δ 175.01, 174.99, 146.63, 136.25, 134.45, 134.41, 133.57, 133.56, 88.33, 55.10, 53.05, 37.32, 36.73, 27.92, 20.91.

HRMS (DART, m/z): Calculated for C₁₄H₁₈ClNO₂H [M+H]⁺=268.1104; found 268.1086 Detailed Procedures and Spectral Data for Synthesis of (±)-cis-cyclopentane ester compound (MP_091, 092, 093, 094)

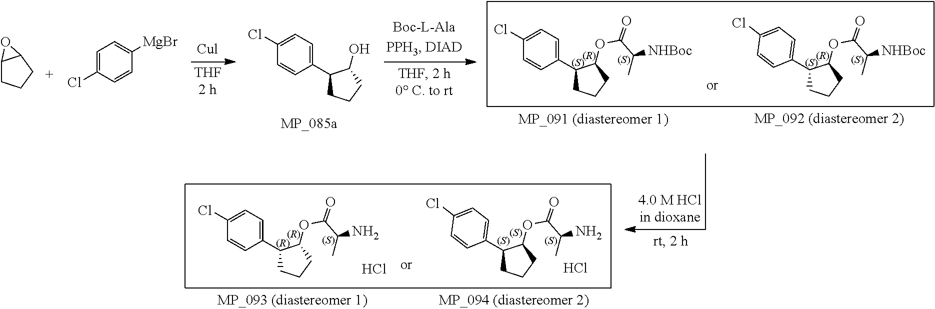

### Preparation of tert-butyl ((2S)-1-(((±)-cis-2-(4-chlorophenyl)cyclopentyl)amino)-1-oxopropan-2-yl)carbamate (MP_091, 092)

(lab note volume 3 page 103, y-279)

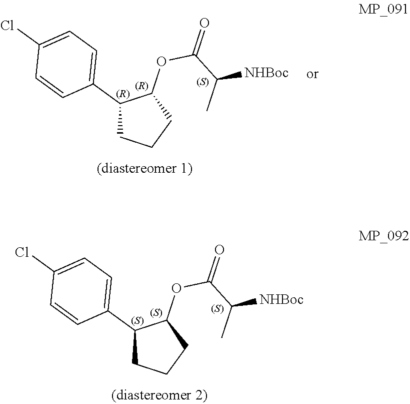

Into a round bottom fitted with a stirrer were placed diisopropyl azodicarboxylate (197 μL, 1 mmol) and Boc-L-Alanine (208 mg, 1.1 mmol) in THF (5 mL). A total of (±)-trans-2-(4-chlorophenyl)cyclopentanol MP_085a (157 mg, 1 mmol) and triphenylphosphine (262 mg, 1 mmol) in THF (5 mL) were added dropwise to the solution while stirring at 0° C. under argon atmosphere. After 2 h at this temperature, the TLC showed that the reaction was complete. The solution was allowed to warm to room temperature and then concentrated under reduced vacuum. The residue was subjected to flash column chromatography using EtOAc/hexane to afford product MP_091 (diastereomer 1, 65 mg, 5.52 mmol) and MP_092 (diastereomer 2, 32 mg, 5.52 mmol) in 26% yield.

TLC (SiO₂) R_{f}=0.3 (EtOAc:hexane=1:2)

MP_091 (diastereomer 1, upper spot in TLC)

¹H NMR (400 MHz, CDCl₃) δ 7.25-7.20 (m, 2H), 7.19-7.14 (m, 2H), 5.43 (td, J=5.3, 2.0 Hz, 1H), 4.87 (d, J=6.3 Hz, 1H), 4.16-3.95 (m, 1H), 3.23-3.02 (m, 1H), 2.21-1.82 (m, 5H), 1.73 (ddd, J=13.5, 8.9, 6.0 Hz, 1H), 1.41 (s, 9H), 0.86 (d, J=7.2 Hz, 3H).

¹³C NMR (101 MHz, CDCl₃) δ 172.62, 154.97, 137.79, 132.18, 129.84, 128.09, 79.69, 78.68, 49.11, 32.60, 28.84, 28.31, 22.34, 18.31.

HRMS (DART, m/z): Calculated for C₁₉H₂₆ClNO₄H [M+H]⁺=368.1629; found 368.1610

MP_092 (diastereomer 2, lower spot in TLC))

¹H NMR (500 MHz, CDCl₃) δ 7.27-7.21 (m, 2H), 7.16 (d, J=8.4 Hz, 2H), 5.36 (t, J=4.1 Hz, 1H), 4.76 (d, J=5.1 Hz, 1H), 4.14-4.01 (m, 1H), 3.20-3.01 (m, 1H), 2.17-2.01 (m, 3H), 1.95 (ddd, J=12.1, 5.3, 3.5 Hz, 1H), 1.85 (ddd, J=14.5, 5.9, 1.7 Hz, 1H), 1.78-1.67 (m, 1H), 1.43 (s, 9H), 1.18 (d, J=6.7 Hz, 3H).

¹³C NMR (126 MHz, CDCl₃) δ 172.30, 154.90, 137.77, 132.28, 130.08, 129.84, 129.81, 128.29, 128.19, 128.09, 79.71, 78.80, 49.33, 49.27, 32.53, 29.24, 28.32, 22.32, 18.54.

HRMS (DART, m/z): Calculated for C₁₉H₂₆ClNO₄H [M+H]⁺=368.1629; found 368.1611

### Preparation of (S)-(±)-cis-2-(4-chlorophenyl)cyclopentyl 2-((tert-butoxycarbonyl)amino)propanoate (MP_093, diastereomer 1)

(Lab Note Volume 3 Page 115, y-286)

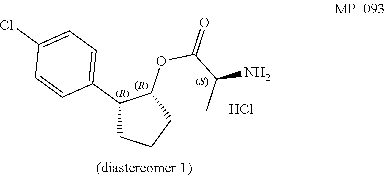

In a round bottom flask equipped with a stir bar, (S)-(±)-cis-2-(4-chlorophenyl)cyclopentyl 2-((tert-butoxycarbonyl)amino)propanoate MP_091 (diastereomer 1, upper spot in TLC) (60 mg, 0.16 mmol) was cooled to 0° C. and was added 4M HCl in dioxane (1 mL). After being stirred for 1 h at 0° C., the reaction mixture was allowed to stir for an additional 2 h at room temperature. The solution was concentrated under reduced pressure, and was added Et₂O. The hydrochloride salt was allowed to precipitate, and the product MP_093 (43 mg, 0.16 mmol) was filtered and dried in high vacuum (99% yield). The hydrochloride salt product could also be triturated with Et₂O.

¹H NMR (400 MHz, DMSO) δ 8.22 (s, 3H), 7.41-7.25 (m, 4H), 5.42 (td, J=5.2, 1.4 Hz, 1H), 3.84 (q, J=7.2 Hz, 1H), 3.28-3.24 (m, 1H), 2.14 (ddd, J=14.9, 9.9, 4.7 Hz, 1H), 2.07-1.96 (m, 2H), 1.94-1.84 (m, 1H), 1.84-1.64 (m, 2H), 0.92 (d, J=7.2 Hz, 3H).

¹³C NMR (126 MHz, DMSO) δ 169.98, 138.54, 131.41, 130.71, 128.32, 80.06, 48.61, 48.25, 32.43, 28.50, 22.32, 15.82.

HRMS (DART, m/z): Calculated for C₁₄H₁₈ClNO₂H [M+H]⁺=268.1104; found 268.1093

### Preparation of (S)-(±)-cis-2-(4-chlorophenyl)cyclopentyl 2-((tert-butoxycarbonyl)amino)propanoate (MP_094, diastereomer 2)

(lab note volume 3 page 115, y-286)

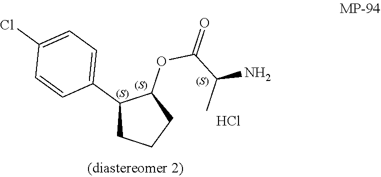

In a round bottom flask equipped with a stir bar, (S)-(±)-cis-2-(4-chlorophenyl)cyclopentyl 2-((tert-butoxycarbonyl)amino)propanoate MP092 (diastereomer 2, lower spot in TLC) (24 mg, 0.07 mmol) was cooled to 0° C. and was added 4M HCl in dioxane (1 mL). After being stirred for 1 h at 0° C., the reaction mixture was allowed to stir for an additional 2 h at room temperature. The solution was concentrated under reduced pressure, and was added Et₂O. The hydrochloride salt was allowed to precipitate, and the product MP_094 (17 mg, 0.06 mmol) was filtered and dried in high vacuum (91% yield). The hydrochloride salt product could also be triturated with Et₂O.

¹H NMR (400 MHz, DMSO) δ 8.41 (s, 3H), 7.33 (s, 4H), 5.33 (t, J=4.3 Hz, 1H), 3.73 (q, J=7.1 Hz, 1H), 3.27-3.19 (m, 1H), 2.16-1.97 (m, 3H), 1.92-1.83 (m, 1H), 1.77 (ddd, J=27.7, 14.9, 7.5 Hz, 2H), 1.29 (d, J=7.2 Hz, 3H).

¹³C NMR (126 MHz, DMSO) δ 169.48, 138.61, 131.45, 130.87, 128.41, 80.26, 48.76, 48.31, 32.53, 28.93, 22.14, 16.11.

HRMS (DART, m/z): Calculated for C₁₄H₁₈ClNO₂H [M+H]⁺=268.1104; found 268.1095

### Detailed Procedures and Spectral Data for Synthesis of (±)-cis-cyclopentane amide compound (MP_095, 096, 097, 098)

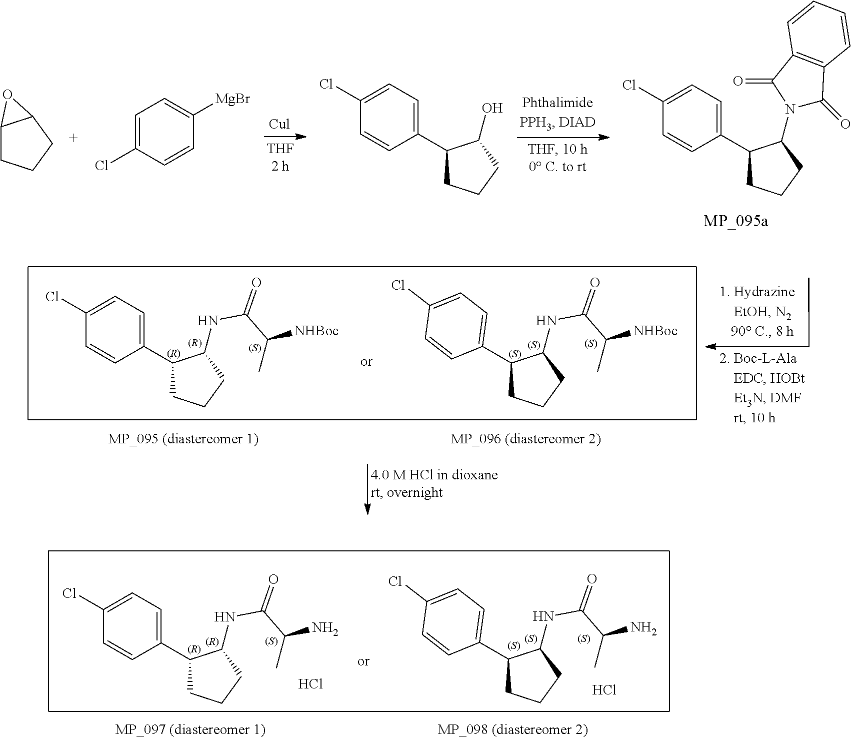

### Preparation of (±)-cis-2-(2-(4-Chlorophenyl)isoindole-1,3-dione (MP_095a)¹¹

(lab note volume 3 page 137, y-298)

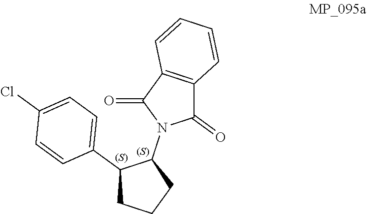

In a 500 mL round-bottom flask equipped with a magnetic stirrer, argon was charged with triphenylphosphine (1.83 g, 7 mmol) and THF 20 mL. To the solution at 0° C. was added dropwise a solution of diisopropyl azodicarboxylate (1.38 mL, 7 mmol) dissolved in THF (20 mL) over a period of 10 min. A massive precipitate formed immediately after addition. To the slurry was then added solid phthalimide (1.03 g, 7 mmol), followed by a solution of of (±)-trans-2-(4-chlorophenyl)cyclopentanol MP_085a (1.38 g, 7 mmol) dissolved in THF (30 mL) over a period of 20 min, maintaining temperature at 0° C. The reaction was then stirred at 0° C. for 4 h and brought to room temperature overnight for convenience. The reaction was quenched with water (100 mL), and the organics were extracted with EtOAc (100 mL). The organic layer was washed with water (100 mL) and dried with anhydrous MgSO₄. Subsequent filtration and concentration under reduced pressure afforded an oil that solidified on equilibrating to room temperature. To the precipitate was then added hexane (100 mL) with vigorous stirring. The triphenylphosphine oxide precipitate was filtered off, and the filtrate was concentrated to an oil. The residue was subjected to flash column chromatography using EtOAc/hexane to afford product MP_095a (1.26 g, 3.87 mmol) in 55% yield.

TLC (SiO₂) R_{f}=0.7 (EtOAc:hexane=1:4)

¹H NMR (400 MHz, CDCl₃) δ 7.51 (dd, J=5.5, 3.1 Hz, 2H), 7.43 (dd, J=5.5, 3.1 Hz, 2H), 6.99 (d, J=8.5 Hz, 2H), 6.92 (d, J=8.5 Hz, 2H), 4.94 (td, J=9.1, 6.2 Hz, 1H), 3.29 (ddd, J=12.3, 9.1, 6.7 Hz, 1H), 2.52-2.26 (m, 2H), 2.22-2.01 (m, 2H), 1.94 (dd, J=12.4, 6.4 Hz, 1H), 1.68-1.46 (m, 1H).

### Preparation of tert-butyl-((S)-1-(((±)-cis-2-(4-chlorophenyl)cyclopentyl)amino)-1-oxopropan-2-yl)carbamate (MP_095a, diastereomer 1)

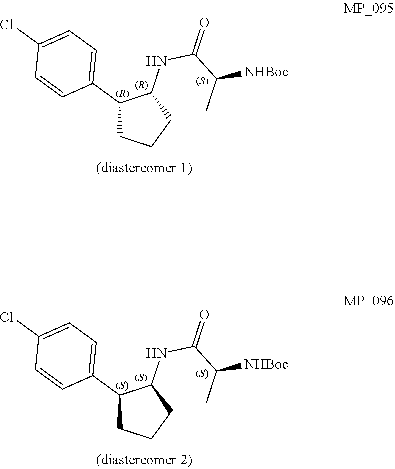

(lab note volume 3 page 143-145, y-301-302)

Into a round bottom fitted with a magnetic stirrer and reflux condenser was placed (±)-cis-2-(2-(4-Chlorophenyl)isoindole-1,3-dione MP_095a (1.26 g, 3.88 mmol) in EtOH (20 mL). To the mixture was added dropwise hydrazine (1.21 mL, 38.8 mmol) in EtOH (5 mL) while it was stirred at room temperature under argon atmosphere. The reaction was then heated at 90° C. for 8 h. The reaction was cooled to room temperature ant the precipitate that had formed was filtered with EtOH. The filtrate was dried over MgSO₄ and concentrated under reduced vacuum. The crude amine product was used for the next step without purification.

To a solution of amine of previous step in 10 ml of DMF was added 1-hydroxybenzotriazole hydrate (1.15 g, 8.52 mmol) and N-(tert-butoxycarbonyl)-L-alanine (1.21 g, 6.39 mmol). Followed by trimethylamine (1.19 mL, 8.52 mmol) and the mixture was allowed to stir for 10 min at 0° C. Then, N-(3-dimethylaminopropyl)-N′-ethylcarbodiimide hydrochloride (1.22 g, 6.39 mmol) was added to the solution and the mixture was stirred for 1 h at the same temperature. After being stirred for 12 h at room temperature, the reaction mixture was quenched with water. The organic layer was extracted by EtOAc (50 mL×3), washed with brine (30 mL), dried over MgSO₄, filtered and concentrated under reduced pressure. The residue was subjected to flash column chromatography using EtOAc/hexane to afford product MP095 (diastereomer 1) and MP_096 (diastereomer 2) (total: 496 mg, 1.35 mmol) in total 35% yield. In the mixture of two diastereomers, MP_095 (diastereomer 1) (211 mg, 0.58 mmol) was recrystallized by Et₂O as a white solid and filtered. The filtratre was concentrated under reduced pressure to give MP_096 (diastereomer 2) (285 mg, 0.78 mmol).

TLC (SiO₂) R_{f}=0.3 (EtOAc:hexane=1:3)

MP_095, diastereomer 1 (upper spot in TLC)

¹H NMR (400 MHz, CDCl₃) δ 7.25 (dd, J=6.9, 1.5 Hz, 2H), 7.16-7.04 (m, 2H), 5.68 (s, 1H), 4.90 (s, 1H), 4.51 (dq, J=13.8, 6.8 Hz, 1H), 3.82 (dd, J=14.2, 7.1 Hz, 1H), 3.32 (q, J=7.6 Hz, 1H), 2.21-2.03 (m, 2H), 1.94-1.84 (m, 2H), 1.80-1.67 (m, 1H), 1.63-1.50 (m, 1H), 1.39 (s, 9H), 1.01 (d, J=7.0 Hz, 3H).

¹³C NMR (101 MHz, CDCl₃) δ 171.81, 139.30, 132.27, 129.77, 128.35, 79.92, 53.36, 49.83, 47.13, 32.03, 29.33, 28.29, 22.19, 18.13

HRMS (DART, m/z): Calculated for C₁₉H₂₇ClN₂O₃H [M+H]⁺=367.1789; found 367.1769

MP_096, diastereomer 2 (lower spot in TLC)

¹H NMR (400 MHz, CDCl₃) δ 7.26-7.23 (m, 2H), 7.13-7.03 (m, 2H), 5.84 (s, 1H), 4.59 (d, J=6.3 Hz, 1H), 4.49 (dt, J=14.6, 7.3 Hz, 1H), 3.92 (s, 1H), 3.32 (q, J=7.4 Hz, 1H), 2.19-2.04 (m, 2H), 1.95-1.79 (m, 2H), 1.79-1.67 (m, 1H), 1.59 (td, J=8.3, 2.7 Hz, 1H), 1.48-1.29 (m, 12H)

¹³C NMR (101 MHz, CDCl₃) δ 171.92, 155.45, 139.34, 132.19, 129.81, 129.77, 128.34, 128.26, 80.00, 53.38, 53.29, 47.12, 31.86, 29.32, 28.26, 22.20, 17.90.

HRMS (DART, m/z): Calculated for C₁₉H₂₇ClN₂O₃H [M+H]⁺=367.1789; found 367.1773

### Preparation of (S)-2-amino-N-((±)-cis-2-(4-chlorophenyl)cyclopentyl)propanamide (MP_097, diastereomer 1)

(lab note volume 3 page 153, y-306)

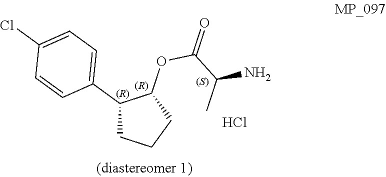

In a round bottom flask equipped with a stir bar, tert-butyl-((S)-1-(((±)-cis-2-(4-chlorophenyl)cyclopentyl)amino)-1-oxopropan-2-yl)carbamate MP_095 (diastereomer 1, upper spot in TLC) (78 mg, 0.21 mmol) was cooled to 0° C. and was added 4M HCl in dioxane (2 mL). After being stirred for 1 h at 0° C., the reaction mixture was allowed to stir overnight at room temperature. The solution was concentrated under reduced pressure, and was added Et₂O. The hydrochloride salt was allowed to precipitate, and the product MP_097 (36 mg, 0.13 mmol) was filtered and dried in high vacuum (64% yield). The hydrochloride salt product could also be triturated with Et₂O.

¹H NMR (500 MHz, DMSO) δ 8.16 (d, J=9.0 Hz, 1H), 7.96 (s, 3H), 7.27 (d, J=8.4 Hz, 2H), 7.17 (d, J=8.5 Hz, 2H), 4.48-4.41 (m, 1H), 3.58 (q, J=6.9 Hz, 1H), 3.27-3.14 (m, 1H), 2.09-1.98 (m, 1H), 1.96-1.83 (m, 3H), 1.67-1.50 (m, 2H), 0.78 (d, J=7.0 Hz, 3H).

¹³C NMR (126 MHz, DMSO) δ 168.67, 140.17, 131.03, 130.86, 128.00, 53.04, 48.30, 48.28, 32.15, 29.41, 22.76, 17.35.

HRMS (DART, m/z): Calculated for C₁₄H₁₉ClN₂OH [M+H]⁺=267.1264; found 267.1251

### Preparation of (S)-2-amino-N-((±)-cis-2-(4-chlorophenyl)cyclopentyl)propanamide (MP_098, diastereomer 2)

(lab note volume 3 page 153, y-307)

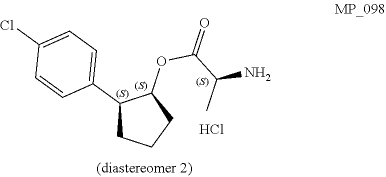

In a round bottom flask equipped with a stir bar, tert-butyl-((S)-1-(((±)-cis-2-(4-chlorophenyl)cyclopentyl)amino)-1-oxopropan-2-yl)carbamate MP_096 (diastereomer 2, lower spot in TLC) (78 mg, 0.21 mmol) was cooled to 0° C. and was added 4M HCl in dioxane (2 mL).

After being stirred for 1 h at 0° C., the reaction mixture was allowed to stir overnight at room temperature. The solution was concentrated under reduced pressure, and was added Et₂O. The hydrochloride salt was allowed to precipitate, and the product MP_097 (45 mg, 0.17 mmol) was filtered and dried in high vacuum (80% yield). The hydrochloride salt product could also be triturated with Et₂O.

¹H NMR (500 MHz, DMSO) δ 8.27-8.06 (m, 4H), 7.30-7.22 (m, 4H), 4.34 (dd, J=8.6, 4.7 Hz, 1H), 3.54 (s, 2H), 3.18 (dd, J=16.5, 10.8 Hz, 1H), 2.05-1.88 (m, 4H), 1.65-1.52 (m, 2H), 1.25-1.24 (m, 3H).

¹³C NMR (126 MHz, DMSO) δ 169.16, 140.17, 131.02, 130.89, 130.81, 128.25, 127.97, 66.82, 53.90, 48.59, 47.60, 31.94, 29.15, 21.88, 17.88.

HRMS (DART, m/z): Calculated for C₁₄H₁₉ClN₂OH [M+H]⁺=267.1264; found 267.1252

### Detailed Procedures and Spectral Data for Synthesis of (S)-2-amino-N-(2-methyl-1-(pyridin-4-yl)propan-2-yl)propanamide hydrochloride (MP 100)

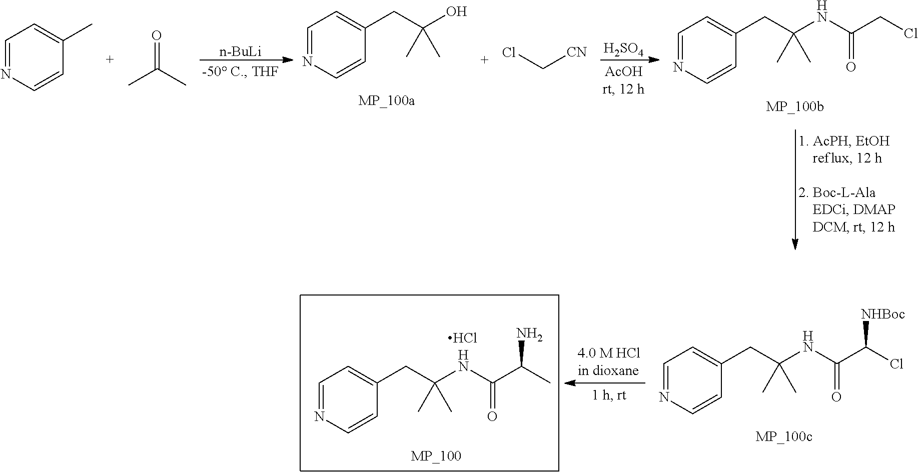

### Preparation of 1,1′-Dimethyl-2-(4-pyridyl)-ethanol (MP_100a)¹²

(lab note volume 4 page 21, y-342)

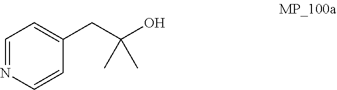

To a stirred solution of 4-picoline (1.46 mL, 15 mmol) in THF (80 ml) was added n-BuLi (6.6 mL, 2.5 M in hexane) dropwise at −50° C. After being stirred for 15 min at the same temperature, dried acetone (1.5 ml, 20 mmol) was slowly added and reaction mixture was stirred for an additional 30 min at −50° C. The reaction was quenched with sat. NH₄Cl and extracted with EtOAc (3×100 mL). The EtOAc layer was washed with water (50 mL) brine (50 mL), dried over Na₂SO₄ and concentrated under reduced pressure. The residue was subjected to flash column chromatography using EtOAc/hexane to afford product MP_100a (1.15 g, 7.58 mmol) in 51% yield.

TLC (SiO₂) R_{f}=0.2 (EtOAc:hexane=1:1)

¹H NMR (500 MHz, CDCl₃) δ 8.32 (d, J=6.0 Hz, 2H), 7.10 (dd, J=4.5, 1.5 Hz, 2H), 3.90 (s, 1H), 2.68 (s, 2H), 1.16 (d, J=5.3 Hz, 6H).

¹³C NMR (126 MHz, CDCl₃) δ 148.66, 147.80, 126.04, 70.18, 49.17, 29.33.

### Preparation of 2-Chloro-N-[1,1-dimethyl-2-(4-pyridinyl)ethyl]acetamide (MP_100b)¹²

(lab note volume 4 page 27, y-345)

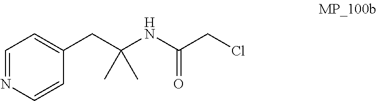

To a stirred solution of 1,1′-Dimethyl-2-(4-pyridyl)-ethanol MP_100a (453 mg, 3 mmol) in acetic acid (4 ml) was added chloroacetonitrile (570 μL, 9 mmol), followed by the addition of sulfuric acid (2 ml). The reaction mixture was stirred at room temperature for 12 hr and quenched with cold water. The solution was basified with saturated NaHCO₃(aq) and extracted with EtOAc (3×25 mL). The EtOAc layer was washed with water (20 mL), dried over Na₂SO₄ and concentrated under reduced pressure. The residue was subjected to flash column chromatography using EtOAc/hexane to afford product MP_100b (371 mg, 1.64 mmol) in 55% yield.

TLC (SiO₂) R_{f}=0.15 (only Ethyl acetate)

¹H NMR (400 MHz, CDCl₃) δ 8.48 (d, J=6.0 Hz, 2H), 7.04 (d, J=6.0 Hz, 2H), 3.94 (s, 2H), 3.08 (s, 2H), 1.34 (s, 6H).

¹³C NMR

### Preparation of (S)-tert-butyl (1-((2-methyl-1-(pyridin-4-yl)propan-2-yl)amino)-1-oxopropan-2-yl)carbamate (MP_100c)¹²

(lab note volume 4 page 33, y-348 and page 45, y-354)

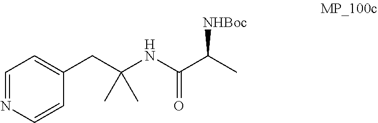 [SMILES: C[C@H](NC(=O)OC(C)(C)C)C(=O)NC(C)(C)Cc1ccncc1]

To a solution of 2-Chloro-N-[1,1-dimethyl-2-(4-pyridinyl)ethyl]acetamide MP_100b (370 mg, 1.64 mmol) in 10 mL of EtOH was added thiourea (149 mg, 1.96 mmol) along with 10 mL of EtOH and 1.5 mL of acetic acid. The resulting mixture was allowed to stir at reflux for 10 h. The reaction mixture was cooled and poured into 40 mL of cold water. The solution was made alkaline with 4 M NaOH (pH 11) and extracted with EtOAc (40 mL×2). The combined organic layers were washed with saturated NaHCO₃(aq) and brine, dried over Na₂SO₄ and the solvent was evaporated under reduced pressure to afford the crude amine product. The crude amine product was placed under high vacuum overnight to remove any residual solvent. The crude amine product was used for the next step without purification.

To a solution of N-(tert-butoxycarbonyl)-L-alanine (227 mg, 1.2 mmol) was added 1-hydroxybenzotrialzole hydrate (184 mg, 1.2 mmol) in 10 ml of DCM. Followed by crude amine from previous step and trimethylamine (306 μl, 2.2 mmol), the mixture was allowed to stir for 10 min at 0° C. Then, N-(3-dimethylaminopropyl)-N′-ethylcarbodiimide hydrochloride (230 mg, 1.2 mmol) was added to the solution and the mixture was stirred for 1 h at 0° C. After being stirred for 12 h at room temperature, the reaction mixture was quenched with water. The organic layer was extracted by ethyl acetate (40 mL×2), washed with brine (20 mL), dried over Na₂SO₄, filtered and concentrated under reduced pressure. The residue was subjected to flash column chromatography using EtOAc/hexane to afford product MP_100c (67 mg, 0.21 mmol) in 13% yield.

TLC (SiO₂) R_{f}=0.3 (EtOAc: Acetonitrile=14:1, visualized by ninhydrin)

¹H NMR (500 MHz, CDCl₃) δ 8.44 (d, J=5.0 Hz, 2H), 7.03 (d, J=5.7 Hz, 2H), 6.04 (s, 1H), 5.19-4.97 (m, 1H), 4.00 (s, 1H), 3.15 (d, J=12.8 Hz, 1H), 2.99 (d, J=12.9 Hz, 1H), 1.38 (s, 9H), 1.34-1.27 (m, 6H), 1.25 (s, 3H).

¹³C NMR (126 MHz, CDCl₃) δ 172.37, 155.68, 149.14, 147.11, 125.91, 80.18, 53.49, 50.49, 43.62, 28.24, 27.40, 17.81.

### Preparation of (S)-2-amino-(2-methyl-1-(pyridin-4-yl)propan-2-yl)propanamide hydrochloride (MP_100)

(lab note volume 4 page 67, y-366)

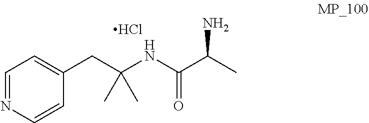 [SMILES: C[C@H](N)C(=O)NC(C)(C)Cc1ccncc1 ; Cl]

In a round bottom flask equipped with a stir bar, (S)-tert-butyl (1-((2-methyl-1-(pyridin-4-yl)propan-2-yl)amino)-1-oxopropan-2-yl)carbamate MP_100c (20 mg, 0.06 mmol) was cooled to 0° C. and was added 4M HCl in dioxane (1 mL). The reaction mixture was allowed to stir 1 h at room temperature. The solution was concentrated under reduced pressure, and was added Et₂O. The hydrochloride salt was allowed to precipitate, and the product MP_100 (10 mg, 0.04 mmol) was filtered and dried in high vacuum (65% yield). The hydrochloride salt product could also be triturated with Et₂O.

¹H NMR (500 MHz, DMSO) δ 8.78 (d, J=5.7 Hz, 2H), 8.31 (s, 3H), 8.04 (s, 1H), 7.78 (d, J=5.8 Hz, 2H), 3.78-3.71 (m, 2H), 3.39 (d, J=12.3 Hz, 4H), 3.18 (d, J=12.2 Hz, 2H), 1.39-1.24 (m, 6H), 1.19 (s, 3H).

¹³C NMR (126 MHz, DMSO) δ 170.11, 157.84, 142.04, 128.97, 54.02, 48.76, 43.68, 27.38, 27.15, 17.95.

HRMS (DART, m/z): Calculated for C₁₂H₁₉N₃OH [M+H]⁺=221.1606; found 222.1122

### Detailed Procedures and Spectral Data for Synthesis of (S)-2-amino-N-(2-(4-chlorophenyl)propan-2-yl)propanamide hydrochloride (MP_101)

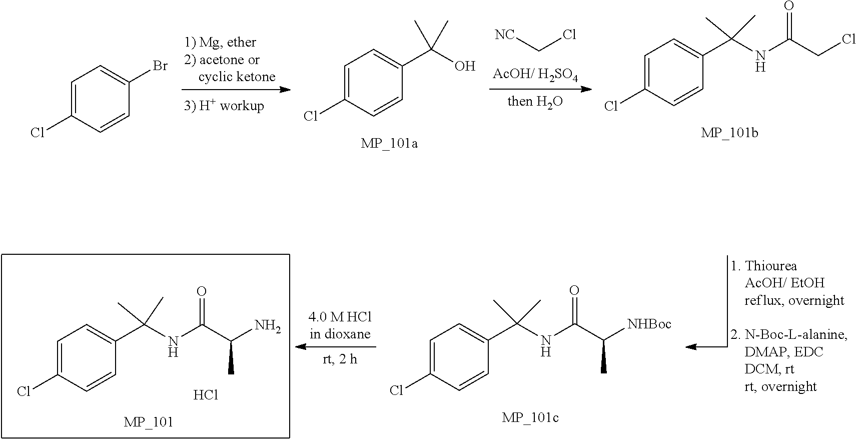

### Preparation of 2-(4-chlorophenyl)propan-2-ol (MP101a)¹³

(lab note volume 4 page 83, y-376)

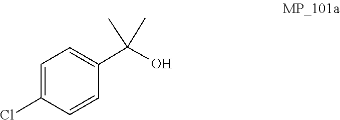

A dry 3-neck reaction flask equipped with a stir bar and reflux condenser was charged with anhydrous Et₂O (30 mL). A solution of the 1.0 M 4-chlorophenylmagnesium bromide solution in diethyl ether (10 mL, 10 mmol) was added dropwise to the solution under argon atmosphere for 10 min. The solution was then cooled to 0° C. and the solution of acetone (741 μL, 18.1 mmol) in Et₂O (20 mL) was added dropwise in a span of 10 min. The solution was allowed to warm to room temperature and allowed to stir for 2 h. The reaction mixture was quenched by addition of 20% aqueous NH₄Cl, and the organic layer was separated and the aqueous layer was extracted with Et₂O. The combined organic layers were washed with saturated NaHCO₃(aq) and brine, dried over MgSO₄ and the solvent was evaporated under reduced pressure to afford the crude product. The tertiary alcohol MP_101a (1.34 g, 7.85 mmol) was isolated by flash chromatography in 79% yield.

TLC (SiO₂) R_{f}=0.5 (EtOAc: Acetonitrile=1:4)

¹H NMR (300 MHz, CDCl₃) δ 7.45 (d, J=8.8 Hz, 2H), 7.33 (d, J=8.8 Hz, 2H), 1.90 (s, 1H), 1.60 (s, 6H).

¹³C NMR (126 MHz, CDCl₃) δ 147.63, 132.42, 128.26, 126.01, 72.29, 31.71.

### Preparation of 2-chloro-N-(2-(4-chlorophenyl)propan-2-yl)acetamide (MP_101b) (lab note volume 4 page 89, y-379)

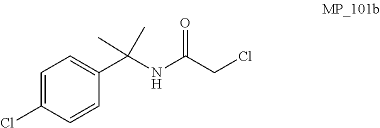

To a stirred solution of 2-(4-chlorophenyl)propan-2-ol MP_101a (510 mg, 3 mmol) in acetic acid (4 ml) was added chloroacetonitrile (570 μL, 9 mmol), followed by the addition of sulfuric acid (2 ml). The reaction mixture was stirred at room temperature for 12 h and quenched with cold water. The solution was basified with saturated NaHCO₃(aq) and organic layers were extracted with EtOAc (3×50 mL). The EtOAc layer was washed with water (50 mL), dried over Na₂SO₄ and concentrated under reduced pressure. The residue was subjected to flash column chromatography using EtOAc/hexane to afford product MP_101b (224 mg, 0.88 mmol) in 30% yield.

TLC (SiO₂) R_{f}=0.3 (EtOAc: Acetonitrile=1:4)

¹H NMR (500 MHz, CDCl₃) δ 7.29 (d, J=1.9 Hz, 4H), 6.88 (s, 1H), 3.93 (s, 2H), 1.68 (s, 6H).

¹³C NMR (126 MHz, CDCl₃) δ 164.84, 144.57, 132.63, 128.59, 126.26, 55.84, 42.87, 28.94.

### Preparation of (S)-tert-butyl (1-((2-(4-chlorophenyl)propan-2-yl)amino)-1-oxopropan-2-yl)carbamate (MP_101c)

(lab note volume 4 page 89, y-379 and page 97, y-382)

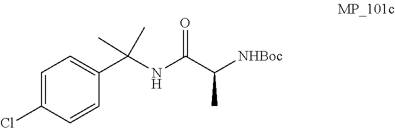 [SMILES: C[C@H](NC(=O)OC(C)(C)C)C(=O)NC(C)(C)c1ccc(Cl)cc1]

To a solution of 2-chloro-N-(2-(4-chlorophenyl)propan-2-yl)acetamide MP_101b (2240 mg, 0.88 mmol) in 10 mL of ethanol was added thiourea (84 mg, 1.11 mmol) along with 1 mL of acetic acid. The reaction mixture was allowed to stir at reflux overnight. The reaction mixture was cooled and poured into 40 mL of cold water. The solution was made alkaline with 4 M NaOH (pH 11) and extracted with EtOAc (40 mL×2). The combined organic layers were washed with saturated NaHCO₃ and brine, dried over Na₂SO₄ and the solvent was evaporated under reduced pressure to afford the crude amine product. The crude amine product was placed under high vacuum overnight to remove any residual solvent. The crude amine product was used for the next step without purification.

To a solution of N-(tert-butoxycarbonyl)-L-alanine (218 mg, 1.15 mmol) in 10 mL of DCM and 5 mL of DMF was added 1-hydroxybenzotrialzole hydrate (124 mg, 0.92 mmol). The crude amine from previous step was added to the mixture followed by trimethylamine (128 μl, 0.92 mmol), the mixture was allowed to stir for 10 min at 0° C. Then, N-(3-dimethylaminopropyl)-N′-ethylcarbodiimide hydrochloride (220 mg, 1.15 mmol) was added to the solution and the mixture was stirred for 1 h at 0° C. After being stirred for 12 h at room temperature, the reaction mixture was quenched with water. The organic layer was extracted by ethyl acetate (40 mL×2), washed with brine (20 mL), dried over Na₂SO₄, filtered and concentrated under reduced pressure. The residue was subjected to flash column chromatography using EtOAc/hexane to afford product MP_101c (161 mg, 0.47 mmol) in 54% yield.

TLC (SiO₂) R_{f}=0.3 (EtOAc: Acetonitrile=1:4, visualized by ninhydrin)

¹H NMR (500 MHz, CDCl₃) δ 7.41-7.20 (m, 4H), 6.83 (s, 1H), 5.26 (d, J=6.6 Hz, 1H), 4.14 (dd, J=14.1, 7.0 Hz, 1H), 1.63 (d, J=10.9 Hz, 6H), 1.49 (s, 9H), 1.30 (d, J=7.0 Hz, 3H).

¹³C NMR (126 MHz, CDCl₃) δ 171.59, 155.84, 145.34, 132.23, 128.37, 126.21, 80.12, 55.18, 50.41, 29.61, 28.92, 28.34, 17.65.

HRMS (DART, m/z): Calculated for C₁₇H₂₅ClN₂O₃H [M+H]⁺=341.1632; found 341.0592

### Preparation of (S)-2-amino-N-(2-(4-chlorophenyl)propan-2-yl)propanamide hydrochloride (MP_101)

(lab note volume 4 page 103, y-386)

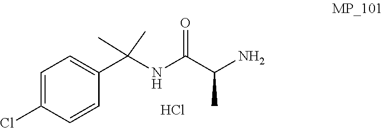 [SMILES: C[C@H](N)C(=O)NC(C)(C)c1ccc(Cl)cc1 ; Cl]

In a round bottom flask equipped with a stir bar, (S)-tert-butyl (1-((2-(4-chlorophenyl)propan-2-yl)amino)-1-oxopropan-2-yl)carbamate MP_101c (48 mg, 0.14 mmol) was cooled to 0° C. and was added 4M HCl in dioxane (2 mL). The reaction mixture was allowed to stir 1 h at room temperature. The solution was concentrated under reduced pressure, and was added Et₂O. The hydrochloride salt was allowed to precipitate, and the product MP_101 (25 mg, 0.09 mmol) was filtered and dried in high vacuum (64% yield). The hydrochloride salt product could also be triturated with Et₂O.

¹H NMR (500 MHz, DMSO) δ 8.81 (s, 1H), 8.12 (s, 3H), 7.31 (dd, J=19.8, 8.7 Hz, 4H), 3.86 (q, J=6.9 Hz, 1H), 1.53 (d, J=19.2 Hz, 6H), 1.35 (d, J=6.9 Hz, 3H).

¹³C NMR (126 MHz, DMSO) δ 168.87, 146.68, 131.09, 128.30, 127.28, 55.26, 48.71, 29.63, 29.31, 17.74.

HRMS (DART, m/z): Calculated for C₁₂H₁₇ClN₂OH [M+H]⁺=241.1108; found 241.1087

### Detailed Procedures and Spectral Data for Synthesis of (S)-4-(((1S,2R)-2-hydroxy-1,2-diphenylethyl)amino)-4,5-dihydro-1H-benzo[c]azepin-3(2H)-one (MP_102)

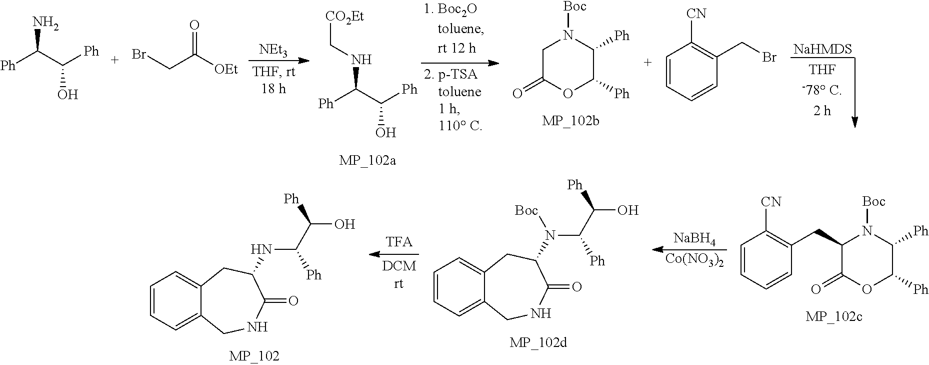

### Preparation of ethyl 2-(((1R,2S)-2-hydroxy-1,2-diphenylethyl)amino)acetate (MP_102a)¹⁴

(lab note volume 3 page 63, y-257)

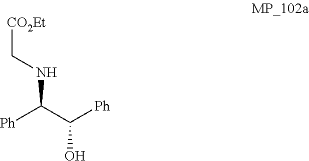 [SMILES: CCOC(=O)CN[C@H](c1ccccc1)[C@@H](O)c1ccccc1]

To a suspension of (1S,2R)-2-amino-1,2-diphenylethanol (5.33 g, 25 mmol) in dry THF (70 mL) was added ethyl bromoacetate (4.15 mL, 37.5 mmol) followed by addition of triethylamine (6.97 mL, 50 mmol). After being stirred vigorously for 18 h, the mixture was filtered to remove Et₃N—HBr. The filtrate was evaporated under vacuum to remove excess Et₃N, THF, and ethyl bromoacetate. The solid residue was washed with cold water in a large filter funnel and the product recrystallized from absolute ethanol. The crystals were collected to give MP_102a (3.78 g, 12.63 mmol) in 50% yield.

¹H NMR (500 MHz, CDCl₃) δ 7.34-7.10 (m, 10H), 4.80 (d, J=6.0 Hz, 1H), 4.11 (q, J=7.1 Hz, 2H), 3.95 (d, J=6.0 Hz, 1H), 3.28 (d, J=17.5 Hz, 1H), 3.16 (d, J=17.5 Hz, 1H), 1.20 (t, J=7.1 Hz, 3H).

¹³C NMR (126 MHz, CDCl₃) δ 172.29, 140.19, 138.53, 128.47, 128.36, 128.17, 127.92, 126.97, 68.28, 60.79, 48.41, 14.16.

### Preparation of ethyl 2-((tert-butoxycarbonyl)((1R,2S)-2-hydroxy-1,2-diphenylethyl)amino)acetate (MP_102b)^{14,15}

(lab note volume 3 page 69, y-260)

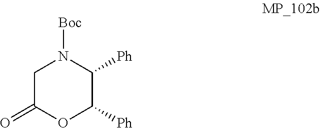 [SMILES: CC(C)(C)OC(=O)N1CC(=O)O[C@@H](c2ccccc2)[C@H]1c1ccccc1]

A solution of ethyl 2-(((1R,2S)-2-hydroxy-1,2-diphenylethyl)amino)acetate MP_102a (3.78 g, 12.62 mmol) in toluene (60 mL) was refluxed. The solution of di-t-butyl dicarbonate (3.74 g, 17.16 mol) in toluene (30 mL) was added dropwise and the reaction mixture was heated at 110° C. for 12 h. Toluene was distilled from the reaction mixture.

To the reaction mixture was added 30 mL of fresh toluene, followed by p-TsOH·H₂O (240 mg, 1.26 mol). The reaction residue was heated at 110° C. for 1 h. Toluene was distilled from the mixture over 2 h, followed by cooling to the room temperature. The resulting solid was filtered and crystallized from hot EtOH to afford product MP_102b.

¹H NMR (500 MHz, DMSO) δ 7.49-6.95 (m, 8H), 6.58 (d, J=7.4 Hz, 2H), 6.19 (d, J=2.1 Hz, 1H), 5.13 (d, J=64.9 Hz, 1H), 4.51 (q, J=17.8 Hz, 2H), 1.48-0.99 (m, 9H).

¹³C NMR (126 MHz, DMSO) δ 168.74, 168.32, 153.42, 153.04, 137.65, 136.77, 135.27, 135.02, 128.65, 128.42, 128.07, 127.98, 127.82, 127.66, 126.70, 80.81, 80.41, 79.72, 79.28, 60.70, 59.26, 46.29, 45.28, 28.39, 28.01.

### Preparation of (3R,5R,6S)-tert-butyl 3-(2-cyanobenzyl)-2-oxo-5,6-diphenylmorpholine-4-carboxylate (MP_102c)^{16,17}

(lab note volume 3 page 85, y-269)

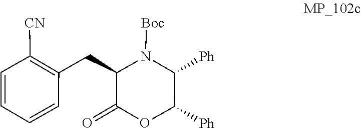 [SMILES: CC(C)(C)OC(=O)N1[C@H](Cc2ccccc2C#N)C(=O)O[C@@H](c2ccccc2)[C@H]1c1ccccc1]

To a stirred solution of 2-((tert-butoxycarbonyl)((1R,2S)-2-hydroxy-1,2-diphenylethyl)amino)acetate MP_102b (353 mg, 1 mmol) in THF (5 mL) was added 1M sodium bis(trimethylsilyl)amide in THF (1 mL, 1 mmol) dropwise via syringe at −78° C. After 40 min, 2-cyanobenzyl bromide (216 mg, 1.1 mmol) was added to the reaction mixture. The resulting solution was stirred additional 1.5 h at −78° C. and poured into water. The aqueous layer was extracted three times with ethyl acetate. The combined organic extracts were dried over anhydrous magnesium sulfate, filtered, concentrated, and separated by flash column chromatography on silica gel to afford MP_102c (100 mg, 0.21 mmol) in 21% yield as a white solid.

TLC (SiO₂) R_{f}=0.4 (Et₂O/hexanes=1:1.5, visualized by ceric ammonium molybdate)

H NMR indicates this compound exists as an approximately 5:1 mixture of rotamers.

¹H NMR (400 MHz, CDCl₃) major rotamer; δ 7.71-7.57 (m, 3H), 7.27-6.99 (m, 7H), 6.93 (d, J=7.0 Hz, 2H), 6.54 (t, J=7.3 Hz, 2H), 5.81 (d, J=3.1 Hz, 1H), 5.41 (dd, J=8.8, 5.5 Hz, 1H), 5.09 (d, J=3.0 Hz, 1H), 3.63 (dd, J=13.6, 5.5 Hz, 1H), 3.53 (dd, J=13.6, 8.9 Hz, 1H), 0.93 (s, 9H).

¹³C NMR (101 MHz, CDCl₃) major rotamer; δ 168.96, 153.74, 140.23, 136.69, 134.24, 133.42, 132.52, 131.47, 128.48, 128.05, 127.99, 127.86, 127.78, 127.65, 127.51, 127.43, 127.18, 126.54, 118.39, 113.52, 81.15, 78.86, 61.49, 56.76, 39.18, 27.59.

### Preparation of 4(R)-t-butoxycarbonyl(1R,2S)-1,2-diphenyl-2-hydroxyethylamino-2,3,4,5-tetrahydro-1H-2-benzazepin-3-one (MP_102d)¹⁷

(lab note volume 3 page 113, y-285)

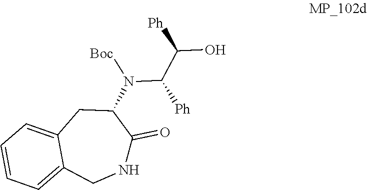 [SMILES: CC(C)(C)OC(=O)N([C@H]1Cc2ccccc2CNC1=O)[C@@H](c1ccccc1)[C@H](O)c1ccccc1]

To a stirred solution of (3R,5R,6S)-tert-butyl 3-(2-cyanobenzyl)-2-oxo-5,6-diphenylmorpholine-4-carboxylate MP_102c (363 mg, 0.78 mmol) and cobalt (II) nitrate hexahydrate (227 mg, 0.78 mmol) in methanol-THF (10:1=20 mL methanol and 2 mL THF) at room temperature was added solid sodium borohydride (295 mg, 7.8 mmol) in ten approximately equal portions over 1 h (Caution: each addition is accompanied by vigorous effervescence). The mixture was stirred at room temperature for 20 h and a further addition of sodium borohydride was made (148 mg, 3.9 mmol) in five approximately equal portions. The mixture was stirred at ambient temperature for another 24 h then acidified with 2N HCl and concentrated under reduced pressure to remove most of the methanol. The residue was basified with concentrated aqueous ammonium hydroxide and extracted with ethyl acetate (5×50 mL). The combined organic extracts were washed with water, brine, dried over anhydrous MgSO₄, filtered, concentrated under reduced pressure. The residue was subjected to flash column chromatography using EtOAc/hexane to afford product MP_102d (53 mg, 0.88 mmol) in 18% yield.

TLC (SiO₂) R_{f}=0.2 (EtOAc:hexane=1:3, visualized by ceric ammonium molybdate)

H NMR indicates this compound exists as an approximately 1:1 mixture of rotamers.

¹H NMR (500 MHz, CDCl₃) two rotamers; δ 7.74 (t, J=5.8 Hz, 4H), 7.49 (d, J=8.1 Hz, 2H), 7.42 (d, J=8.0 Hz, 2H), 7.32-7.01 (m, 21H), 6.62 (d, J=3.4 Hz, 1H), 6.20 (d, J=7.2 Hz, 1H), 6.10 (d, J=7.3 Hz, 1H), 5.71 (d, J=3.0 Hz, 1H), 5.65 (t, J=3.0 Hz, 1H), 5.58 (t, J=2.9 Hz, 1H), 5.49 (d, J=3.0 Hz, 1H), 4.91 (dd, J=14.5, 2.5 Hz, 1H), 4.71 (dd, J=14.6, 2.1 Hz, 1H), 4.09 (dd, J=12.7, 3.8 Hz, 1H), 4.04 (dd, J=12.6, 4.4 Hz, 1H), 3.85-3.80 (m, 1H), 3.80-3.76 (m, 1H), 3.66 (t, J=13.1 Hz, 1H), 3.53 (t, J=13.2 Hz, 1H), 1.85 (dd, J=13.8, 3.8 Hz, 1H), 1.76 (dd, J=13.7, 4.4 Hz, 1H), 1.66 (s, 9H), 1.62 (s, 9H).

¹³C NMR (126 MHz, CDCl₃) two rotamers; δ 176.62, 176.05, 154.42, 154.26, 141.08, 140.97, 138.23, 137.27, 137.19, 137.10, 135.75, 135.56, 131.99, 131.87, 129.09, 129.04, 128.77, 128.51, 128.22, 127.92, 127.86, 127.76, 127.31, 127.22, 127.19, 127.07, 126.36, 126.20, 125.97, 125.77, 81.81, 81.22, 75.35, 74.51, 64.96, 64.04, 58.36, 58.35, 45.29, 45.17, 35.42, 34.33, 28.72, 28.67.

### Preparation of 4(R)-(1R,2S)-1,2-diphenyl-2-hydroxyethylamino-2,3,4,5-tetrahydro-1H-2-benzazepin-3-one (MP_102)¹⁷

(lab note volume 4 page 131, y-400)

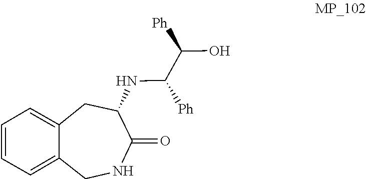 [SMILES: O=C1NCc2ccccc2C[C@@H]1N[C@@H](c1ccccc1)[C@H](O)c1ccccc1]

A solution of 4(R)-t-butoxycarbonyl(1R,2S)-1,2-diphenyl-2-hydroxyethylamino-2,3,4,5-tetrahydro-1H-2-benzazepin-3-one MP_102d (240 mg, 0.5 mmol) in 5 mL of methylene chloride at room temperature was treated with trifluoroacetic acid (570 μL, 7.5 mmol). The mixture was stirred at room temperature for 3 h then all volatiles removed under vacuum. The residue was dissolved in 5 mL of water and the solution made basic (pH 10-11) by the addition of solid sodium carbonate. The mixture was extracted with chloroform (5×) and the combined extracts dried over Na₂SO₄, filtered and solvents removed under vacuum to give MP_102 (179 mg, 0.48 mmol) in 96% yield.

¹H NMR (500 MHz, DMSO) δ 7.33-7.02 (m, 14H), 6.95 (d, J=7.3 Hz, 1H), 4.96 (d, J=4.8 Hz, 1H), 4.37 (dd, J=16.1, 4.4 Hz, 1H), 4.13 (d, J=4.8 Hz, 1H), 3.88 (dd, J=16.2, 6.3 Hz, 1H), 3.75 (dd, J=11.9, 4.0 Hz, 1H), 3.19 (dd, J=16.6, 3.5 Hz, 1H), 2.99 (dd, J=16.4, 12.2 Hz, 1H).

¹³C NMR (126 MHz, DMSO) δ 176.24, 140.52, 138.86, 136.35, 134.87, 130.74, 128.17, 128.16, 127.88, 127.81, 127.54, 127.37, 126.79, 126.33, 76.14, 66.40, 54.40, 45.66, 36.86. (one low-field carbon is not observed but it seems like hidden in δ 128.15).

HRMS (DART, m/z): Calculated for C₂₄H₂₄N₂O₂H [M+H]⁺=373.1916; found 373.1923

### Detailed Procedures and Spectral Data for Synthesis of (S)-2-amino-N—((S)-2-oxoazepan-3-yl)propanamide hydrochloride (MP_103)

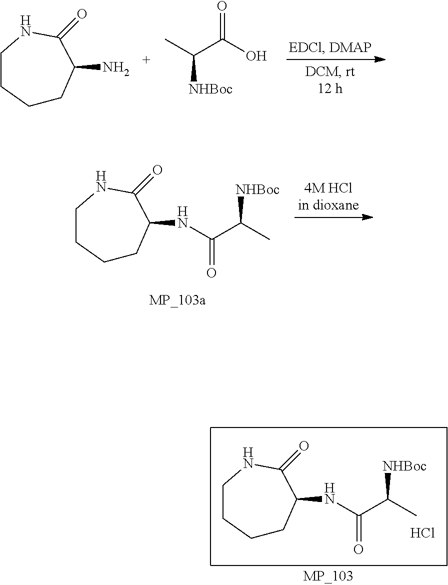 [SMILES: N[C@H]1CCCCNC1=O ; C[C@H](NC(=O)OC(C)(C)C)C(=O)O ; C[C@H](NC(=O)OC(C)(C)C)C(=O)N[C@H]1CCCCNC1=O ; C[C@H](NC(=O)OC(C)(C)C)C(=O)N[C@H]1CCCCNC1=O ; Cl]

### Preparation of tert-butyl ((S)-1-oxo-1-(((S)-2-oxoazepan-3-yl)amino)propan-2-yl)carbamate (MP_103a)

(lab note volume 4 page 149, y-411)

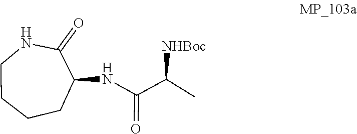 [SMILES: C[C@H](NC(=O)OC(C)(C)C)C(=O)N[C@H]1CCCCNC1=O]

To a solution of (S)-3-aminoazepan-2-one (64 mg, 0.5 mmol) in DCM (2 mL) was added N-(tert-butoxycarbonyl)-L-alanine (189 mg, 1 mmol) followed by dimethylaminopyridine (24 mg, 0.2 mmol). The reaction mixture was allowed to stir for 10 min at 0° C. Then, N-(3-dimethylaminopropyl)-N′-ethylcarbodiimide hydrochloride (192 mg, 1 mmol) was added to the solution and the mixture was stirred for 1 h at 0° C. After being stirred for 12 h at room temperature, the reaction mixture was quenched with water. The organic layer was extracted by ethyl acetate (40 mL×2), washed with brine (20 mL), dried over Na₂SO₄, filtered and concentrated under reduced pressure. The residue was subjected to flash column chromatography using EtOAc/hexane to afford product MP_103a (101 mg, 0.34 mmol) in 67% yield.

TLC (SiO₂) R_{f}=0.3 (EtOAc/ACN/H₂O/MeOH=70:5:2.5:2.5, visualized by ninhydrin)

¹H NMR (400 MHz, CDCl₃) δ 7.61 (s, 1H), 7.04 (s, 1H), 5.45 (d, J=7.5 Hz, 1H), 4.50-4.37 (m, 1H), 4.22 (s, 1H), 3.25-3.11 (m, 2H), 1.99-1.87 (m, 2H), 1.81-1.70 (m, 2H), 1.36 (s, 9H), 1.28 (d, J=7.0 Hz, 3H).

¹³C NMR (101 MHz, CDCl₃) δ 175.61, 172.08, 155.23, 79.58, 52.16, 52.06, 50.09, 41.95, 31.53, 28.78, 28.31, 27.91, 19.04.

### Preparation of (S)-2-amino-N—((S)-2-oxoazepan-3-yl)propanamide hydrochloride (MP_103) (lab note volume 4 page 155, y-414)

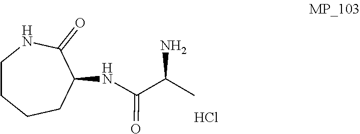

In a round bottom flask equipped with a stir bar, tert-butyl ((S)-1-oxo-1-(((S)-2-oxoazepan-3-yl)amino)propan-2-yl)carbamate MP_103a (31 mg, 0.10 mmol) was cooled to 0° C. and was added 4M HCl in dioxane (1 mL). The reaction mixture was allowed to stir 1 h at room temperature. The solution was concentrated under reduced pressure, and was added Et₂O. The hydrochloride salt was allowed to precipitate, and the product MP_103 (25 mg, 0.09 mmol) was filtered and dried in high vacuum (85% yield). The hydrochloride salt product could also be triturated with Et₂O.

¹H NMR (400 MHz, DMSO) δ 8.45 (d, J=7.2 Hz, 1H), 8.29 (s, 3H), 7.83-7.75 (m, 1H), 4.37 (dd, J=10.1, 7.4 Hz, 1H), 3.94-3.85 (m, 1H), 3.19-3.08 (m, 1H), 3.07-2.95 (m, 1H), 1.89-1.78 (m, 1H), 1.78-1.51 (m, 3H), 1.46-1.34 (m, 1H), 1.30 (d, J=6.9 Hz, 3H), 1.22-1.08 (m, 1H).

¹³C NMR (101 MHz, DMSO) δ 174.07, 168.98, 52.19, 48.33, 41.01, 31.32, 29.26, 28.05, 17.76.

HRMS (DART, m/z): Calculated for C₉H₁₇N₃O₂H [M+H]⁺=200.1399; found 200.1388

### Detailed Procedures and Spectral Data for Synthesis of (S)-2-amino-N—((S)-2-methyl-3-oxo-2,3,4,5-tetrahydro-1H-benzo[c]azepin-4-yl)propanamide hydrochloride (MP_104)

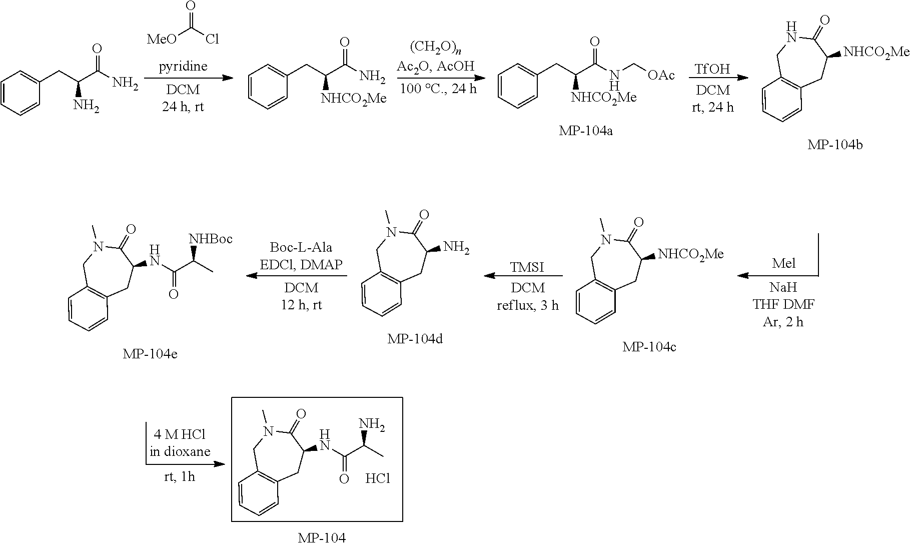

### Preparation of (S)-methyl (1-amino-1-oxo-3-phenylpropan-2-yl)carbamate (MP_104a)^{3,4}

(lab note volume 3 page 121, y-290)

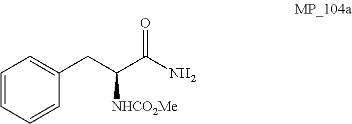

In a round bottom flask with a magnetic stir bar (S)-2-amino-3-phenylpropanamide (9.04 g, 55.09 mmol) in DCM (200 mL) was cooled to 0° C. and pyridine (7.54 mL, 93.65 mmol) was added. After being stirred for 5 min, methylchloroformate (5.11 mL, 66.11 mmol) was added to the mixture. It was allowed to stir overnight at room temperature. 1 N HCl was added to the reaction mixture to acidify to pH 2-3 and filtered with DCM. After evaporating the solvent, the reaction mixture was treated with brine and saturated NaHCO₃(aq). The organic layer was extracted by ethyl acetate (40 mL×2), washed with brine (20 mL), dried over Na₂SO₄, filtered, and concentrated under reduced pressure to afford white solid product MP_104a (1.7 g, 7.66 mmol) in 14% yield.

¹H NMR (500 MHz, DMSO) δ 7.45 (s, 1H), 7.27 (d, J=4.4 Hz, 4H), 7.22-7.15 (m, 1H), 7.05 (s, 1H), 4.14 (td, J=10.4, 4.2 Hz, 1H), 3.44 (s, 3H), 2.97 (dd, J=13.7, 4.1 Hz, 1H), 2.73 (dd, J=13.7, 10.6 Hz, 1H).

¹³C NMR (126 MHz, DMSO) δ 174.01, 156.88, 138.81, 129.62, 128.50, 126.68, 56.52, 51.75, 37.95.

### Preparation of (S)-(2-((methoxycarbonyl)amino)-3-phenylpropanamido)methyl acetate (MP_104b)^{3,4}

(lab note volume 3 page 125, y-292)

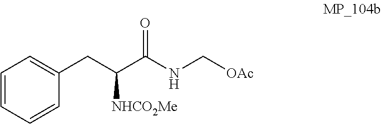

To an Ar flushed round bottom flask were added paraformaldehyde (230 mg, 7.65 mmol) and acetic anhydride (7.21 mL, 76.5 mmol), followed by a solution of (S)-methyl (1-amino-1-oxo-3-phenylpropan-2-yl)carbamate MP_104a (1.7 g, 7.65 mmol) in acetic acid (5 mL). The reaction mixture was refluxed at 80° C. for 16 h. After being cooled to room temperature, the reaction mixture was concentrated under reduced pressure to give the crude amide compound. Neutralized the crude amide with saturated NaHCO₃(aq) and organic layers were extracted with EtOAc. The residue was dried over MgSO₄ and concentrated under vacuum. The MP_104b (1.23 g, 4.18 mmol) was crystalized by Et₂O as a white solid in 55% yield

¹H NMR (400 MHz, CDCl₃) δ 7.33-7.22 (m, 3H), 7.20-7.10 (m, 2H), 7.05 (t, J=6.7 Hz, 1H), 5.28 (d, J=6.8 Hz, 1H), 5.15 (dd, J=7.2, 3.0 Hz, 2H), 4.42 (d, J=6.5 Hz, 1H), 3.64 (s, 3H), 3.06 (t, J=6.2 Hz, 2H), 2.02 (s, 3H).

¹³C NMR (101 MHz, CDCl₃) δ 171.86, 171.52, 135.93, 129.27, 128.77, 127.22, 83.95, 63.86, 56.07, 52.56, 38.43, 20.84.

### Preparation of (S)-methyl (3-oxo-2,3,4,5-tetrahydro-1H-benzo[c]azepin-4-yl)carbamate (MP_104c)^{3,4}

(lab note volume 3 page 155, y-308)

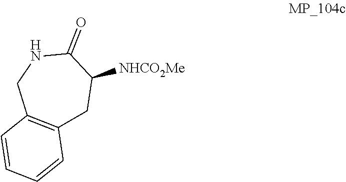

To a solution of (S)-(2-((methoxycarbonyl)amino)-3-phenylpropanamido)methyl acetate MP_104b (926 mg, 3.15 mmol) in DCM (30 mL) was added trifluoromethanesulfonic acid (2 mL, 22.6 mmol) at 0° C. The reaction mixture was allowed to warm to room temperature and stirred for 48 h. The residue was quenched by ice water and suspension was collected with DCM.

The organic layers were dried over MgSO₄, concentrated under reduced pressure and separated by flash column chromatography on silica gel to afford MP_104c (184 mg, 0.79 mmol) in 25% yield.

TLC (SiO₂) R_{f}=0.3 (EtOAc/hexanes=3:1, visualized by ceric ammonium molybdate)

¹H NMR (500 MHz, DMSO) δ 8.67 (t, J=5.8 Hz, 1H), 7.32 (d, J=8.8 Hz, 1H), 7.26-7.22 (m, 2H), 7.15 (t, J=7.0 Hz, 1H), 4.44 (t, J=5.8 Hz, 1H), 4.25-4.14 (m, 1H), 3.40 (s, 3H), 2.90 (dd, J=13.7, 3.6 Hz, 1H), 2.67 (dd, J=13.6, 11.0 Hz, 1H).

¹³C NMR (126 MHz, DMSO) δ 172.74, 156.84, 138.62, 129.68, 128.49, 128.44, 126.70, 56.52, 51.76, 43.92, 37.97.

### Preparation of (S)-methyl (2-methyl-3-oxo-2,3,4,5-tetrahydro-1H-benzo[c]azepin-4-yl)carbamate (MP_104d)¹⁸

(lab note volume 4 page 163, y-418)

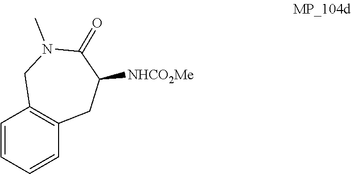

A solution of (S)-methyl (3-oxo-2,3,4,5-tetrahydro-1H-benzo[c]azepin-4-yl)carbamate MP_104c (117 mg, 0.5 mmol) dissolved in a mixture of 2 mL of dry THF and 2 mL of dry DMF was added dropwise at room temperature to a suspension of 60% NaH dispersed in oil (24 mg, 0.6 mmol), suspended in 2 mL of anhydrous THF under an inert atmosphere. At the end of the addition, methyliodide (46.5 μL, 0.75 mmol) were added. The reaction mixture was then stirred at room temperature for 2 hours and 10 ml of saturated NH₄Cl solution (aq) were then added, while cooling the reaction medium in an ice bath. The mixture is extracted with ethyl acetate (3×10 mL). The organic phases are combined, washed with brine, dried over MgSO₄ and evaporated to dryness under reduced pressure. The oily residue obtained was separated by flash column chromatography on silica gel to afford MP_104d (131 mg, mixed with DMF).

TLC (SiO₂) R_{f}=0.6 (DCM/MeOH=9:1)

¹H NMR (400 MHz, CDCl₃) δ 7.20-7.00 (m, 4H), 6.16 (d, J=6.0 Hz, 1H), 5.15 (d, J=15.9 Hz, 2H), 3.76 (d, J=16.6 Hz, 1H), 3.64 (s, 3H), 3.45 (dd, J=17.2, 4.7 Hz, 1H), 3.01 (s, 3H).

¹³C NMR (101 MHz, CDCl₃) δ 171.21, 156.27, 135.78, 132.82, 130.98, 130.94, 128.99, 128.69, 128.25, 128.13, 127.78, 126.09, 53.81, 52.16, 49.72, 36.95, 35.08.

### Preparation of (S)-4-amino-2-methyl-4,5-dihydro-1H-benzo[c]azepin-3(2H)-one (MP_104e)¹⁸

(lab note volume 4 page 179, y-426)

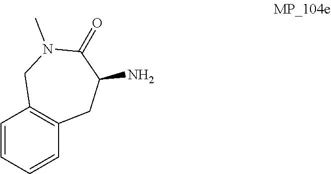

Trimethylsilyl iodide (0.33 ml, 2.3 mmol) was added to a solution of (S)-methyl (2-methyl-3-oxo-2,3,4,5-tetrahydro-1H-benzo[c]azepin-4-yl)carbamate MP_104d in 10 ml of DCM, and the mixture was refluxed for 3 h. After cooling to room temperature and adding 50 ml of ethyl acetate, the organic phase was extracted with 1 N hydrochloric acid solution (2×25 ml). The aqueous phases were combined, cooled, and basified by addition of 5 N sodium hydroxide. The basic aqueous phases were treated with brine, and then extracted with 3×25 ml of ethyl acetate. The organic phases were combined, dried over MgSO₄, and evaporated under reduced pressure to give an oily residue. The oily residue was separated by flash column chromatography on silica gel to afford MP_104e (38 mg, 0.2 mmol) in 38% yield.

TLC (SiO₂) R_{f}=0.2 (DCM/MeOH=9:1)

¹H NMR (500 MHz, CDCl₃) δ 7.22-7.02 (m, 4H), 5.15 (d, J=16.5 Hz, 1H), 4.52 (s, 1H), 3.77 (d, J=16.5 Hz, 1H), 3.41 (s, 2H), 3.32 (dd, J=17.0, 3.6 Hz, 1H), 3.02 (s, 3H), 2.99-2.89 (m, 1H).

¹³C NMR (126 MHz, CDCl₃) δ 174.26, 136.08, 133.30, 130.88, 128.73, 128.11, 126.06, 53.75, 49.97, 38.79, 35.42.

### Preparation of tert-butyl ((S)-1-(((S)-2-methyl-3-oxo-2,3,4,5-tetrahydro-1H-benzo[c]azepin-4-yl)amino)-1-oxopropan-2-yl)carbamate (MP_104f) (lab note volume 5 page 1, y-437)

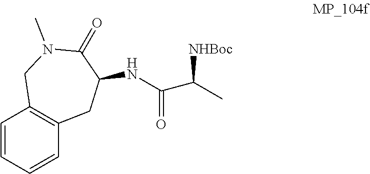 [SMILES: C[C@H](NC(=O)OC(C)(C)C)C(=O)N[C@H]1Cc2ccccc2CN(C)C1=O]

To a solution of N-(tert-butoxycarbonyl)-L-alanine (114 mg, 0.6 mmol) in DCM (3 mL) was added (S)-4-amino-2-methyl-4,5-dihydro-1H-benzo[c]azepin-3(2H)-one MP_104e (95 mg, 0.5 mmol) at room temperature. N-(3-dimethylaminopropyl)-N′-ethylcarbodiimide hydrochloride (115 mg, 0.6 mmol) and dimethylaminopyridine (24 mg, 0.2 mmol) were added to the reaction mixture. After being stirred for 2 days at room temperature, the reaction mixture was quenched with water. The organic layer were extracted by ethyl acetate (30 mL×3), washed with brine (20 mL), dried over Na₂SO₄, filtered and concentrated under reduced pressure. The residue was subjected to flash column chromatography using EtOAc/hexane to afford product MP_104f (73 mg, 0.2 mmol) in 40% yield.

TLC (SiO₂) R_{f}=0.4 (EtOAc:hexane=3:1, visualized by ninhydrin) ¹H NMR (500 MHz, CDCl₃) δ 7.47 (s, 1H), 7.17 (t, J=7.5 Hz, 1H), 7.05 (dt, J=17.9, 7.6 Hz, 3H), 5.40-5.28 (m, 2H), 5.19 (d, J=16.5 Hz, 1H), 4.25 (d, J=6.1 Hz, 1H), 3.76 (d, J=16.7 Hz, 1H), 3.46 (dd, J=17.1, 4.1 Hz, 1H), 3.03 (s, 3H), 2.83 (dd, J=16.3, 13.5 Hz, 1H), 1.42 (s, 9H), 1.36 (d, J=7.1 Hz, 3H).

¹³C NMR (126 MHz, CDCl₃) δ 172.11, 170.98, 155.25, 135.70, 132.74, 130.95, 128.71, 128.15, 126.09, 79.76, 53.81, 50.23, 48.32, 36.24, 35.09, 28.36, 19.11.

### Preparation of (S)-2-amino-N—((S)-2-methyl-3-oxo-2,3,4,5-tetrahydro-1H-benzo[c]azepin-4-yl)propanamide hydrochloride (MP_104)

(lab note volume 5 page 7, y-440)

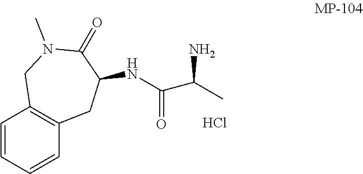 [SMILES: C[C@H](N)C(=O)N[C@H]1Cc2ccccc2CN(C)C1=O ; Cl]

In a round bottom flask equipped with a stir bar, tert-butyl ((S)-1-(((S)-2-methyl-3-oxo-2,3,4,5-tetrahydro-1H-benzo[c]azepin-4-yl)amino)-1-oxopropan-2-yl)carbamate MP_104f (43 mg, 0.12 mmol) was cooled to 0° C. and was added 4M HCl in dioxane (2 mL). The reaction mixture was allowed to stir 2 h at room temperature. The solution was concentrated under reduced pressure, and was added Et₂O. The hydrochloride salt was allowed to precipitate, and the product MP_104 (22 mg, 0.07 mmol) was filtered and dried in high vacuum (62% yield). The hydrochloride salt product could also be triturated with Et₂O.

¹H NMR (500 MHz, DMSO) δ 8.70 (d, J=7.3 Hz 1H), 8.32 (br s, 3H), 7.26-7.19 (m, 2H), 7.16-7.10 (m, 2H), 5.39-5.30 (m, 1H), 5.22 (d, J=16.5 Hz, 1H), 4.00 (d, J=16.5 Hz, 1H), 3.97 (m, 1H), 3.22 (dd, J=17.2, 4.9 Hz, 1H), 2.90 (s, 3H), 2.83 (dd, J=17.2, 13.1 Hz, 1H), 1.37 (d, J=7.0 Hz, 3H).

¹³C NMR (126 MHz, DMSO) δ 170.83, 169.57, 135.69, 134.65, 131.01, 129.49, 128.22, 126.47, 52.63, 48.63, 48.42, 35.56, 34.93, 17.81.

HRMS (DART, m/z): Calculated for C₁₄H₁₉N₃O₂H [M+H]⁺=262.1555; found 262.1552

### Detailed Procedures and Spectral Data for Synthesis of (S)-2-amino-N—((S)-3-oxo-2,3,4,5-tetrahydro-1H-benzo[c]azepin-4-yl)propanamide hydrochloride (MP_105)

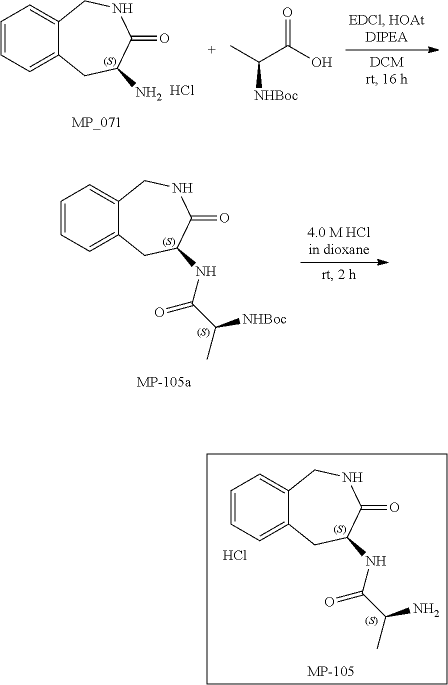

### Preparation of tert-butyl ((S)-1-oxo-1-(((S)-3-oxo-2,3,4,5-tetrahydro-1H-benzo[c]azepin-4-yl)amino)propan-2-yl)carbamate (MP_105a)

(lab note volume 5 page 77, y-474)

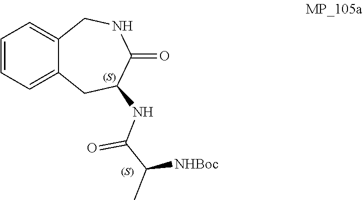

To a solution of N-(tert-butoxycarbonyl)-L-alanine (48 mg, 0.25 mmol) and 1-hydroxy-7-azabenzotriazole (35 mg, 0.25 mmol) in DCM (5 mL) was added N-(3-dimethylaminopropyl)-N′-ethylcarbodiimide hydrochloride (53 mg, 0.28 mmol) in DCM (3 mL). After 10 min, (S)-4-amino-4,5-dihydro-1H-benzo[c]azepin-3(2H)-one hydrochloride MP_071 (45 mg, 0.21 mmol) was added to the mixture followed by N,N-diisopropylethylamine (59 μL, 0.34 mmol). After being stirred for 16 h at room temperature, the resulting solution was evaporated under vacuum. The residue was washed with EtOAc and 1.0 M aqueous HCl (×2), then subsequently washed with 1.0 M aqueous NaOH, H₂O and brine. The organic layers were dried over Na₂SO₄ and evaporated under vacuum to provide crude product. The residue was subjected to flash column chromatography using EtOAc/hexane to afford product MP_105a (45 mg, 0.13 mmol) in 61% yield.

TLC (SiO₂) R_{f}=0.4 (only EtOAc, visualized by ninhydrin)

¹H NMR (500 MHz, CDCl₃) δ 7.53 (s, 1H), 7.17 (dd, J=14.3, 6.7 Hz, 2H), 7.12-7.03 (m, 2H), 7.01 (d, J=7.4 Hz, 1H), 5.34 (d, J=6.8 Hz, 1H), 5.26-5.14 (m, 1H), 4.84 (dd, J=16.5, 4.6 Hz, 1H), 4.29 (s, 1H), 3.97 (dd, J=16.7, 7.1 Hz, 1H), 3.42 (dd, J=16.9, 3.9 Hz, 1H), 2.92 (dd, J=16.6, 12.9 Hz, 1H), 1.44 (s, 9H), 1.37 (d, J=7.0 Hz, 3H).

¹³C NMR (126 MHz, CDCl₃) δ 173.68, 172.30, 155.31, 135.30, 133.88, 131.05, 128.29, 127.90, 126.34, 79.88, 50.26, 48.24, 45.66, 36.11, 28.37, 18.99.

### Preparation of (S)-2-amino-N—((S)-3-oxo-2,3,4,5-tetrahydro-1H-benzo[c]azepin-4-yl)propanamide hydrochloride (MP_105)

(lab note volume 5 page 81, y-476)

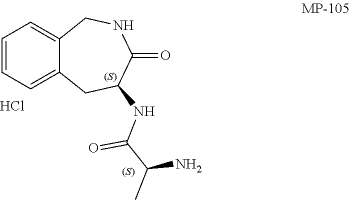

In a round bottom flask equipped with a stir bar, tert-butyl ((S)-1-oxo-1-(((S)-3-oxo-2,3,4,5-tetrahydro-1H-benzo[c]azepin-4-yl)amino)propan-2-yl)carbamate MP_105a (45 mg, 0.13 mmol) was cooled to 0° C. and was added 4M HCl in dioxane (2 mL). The reaction mixture was allowed to stir 2 h at room temperature. The solution was concentrated under reduced pressure, and was added Et₂O. The hydrochloride salt was allowed to precipitate, and the product MP_105 (26 mg, 0.09 mmol) was filtered and dried in high vacuum (71% yield). The hydrochloride salt product could also be triturated with Et₂O.

¹H NMR (400 MHz, DMSO) δ 8.64 (d, J=7.2 Hz, 1H), 8.37-8.32 (m, 1H), 8.25 (s, 3H), 7.21-7.08 (m, 4H), 5.16-5.06 (m, 1H), 4.78 (dd, J=16.4, 4.6 Hz, 1H), 3.97-3.85 (m, 2H), 3.15 (dd, J=16.8, 4.2 Hz, 1H), 2.92 (dd, J=16.7, 12.7 Hz, 1H), 1.34 (d, J=6.9 Hz, 3H).

¹³C NMR (126 MHz, DMSO) δ 172.55, 169.52, 136.22, 135.59, 131.01, 128.89, 127.84, 126.63, 48.64, 48.47, 44.70, 35.79, 17.82.

HRMS (DART, m/z): Calculated for C₁₃H₁₇N₃O₂H [M+H]⁺=248.1399; found 248.1388

### Detailed Procedures and Spectral Data for Synthesis of (S)-2-amino-N-(1,3-bis(3,5-difluorophenyl)-2-methylpropan-2-yl)propanamide hydrochloride (MP_106)

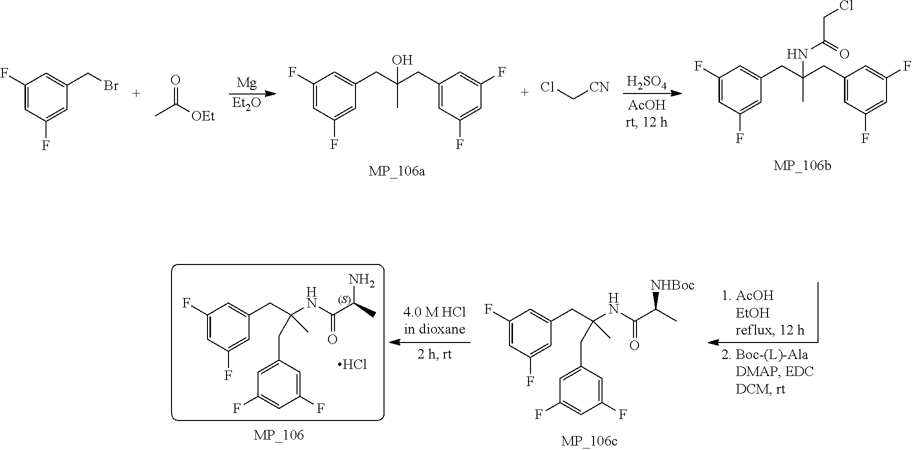

### Preparation of 1,3-bis(3,5-difluorophenyl)-2-methylpropan-2-ol (MP_106a)

(lab note volume 5 page 45, y-459)

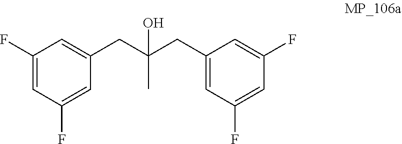 [SMILES: CC(O)(Cc1cc(F)cc(F)c1)Cc1cc(F)cc(F)c1]

To a flame dried round bottom flask with reflux condenser were added Mg (363 mg, 15 mmol), 15 mL Et₂O and 0.1 mL of dibromoethane. The reaction mixture was refluxed for 30 min. After reaction was allowed to cool to 0° C., 3,5-difluorobenzylbromide (1.55 mL, 12 mmol) was added dropwise. After 30 min, EtOAc (586 μL, 6 mmol) was added dropwise at the same temperature and the solution was stirred at room temperature for 12 h. The reaction mixture was quenched with saturated NH₄Cl (aq) and brine. The organic layers were extracted with Et₂O, dried over Na₂SO₄, filtered, and concentrated under reduced pressure. The residue was subjected to flash column chromatography using EtOAc/hexane to afford product MP_106a (1.17 g, 3.93 mmol) in 33% yield.

TLC (SiO₂) R_{f}=0.4 (EtOAc:hexane=1:9)

¹H NMR (400 MHz, CDCl₃) δ 6.81-6.73 (m, 4H), 6.69 (tt, J=9.0, 2.3 Hz, 2H), 2.80 (d, J=13.4 Hz, 2H), 2.71 (d, J=13.4 Hz, 2H), 1.07 (s, 3H).

¹³C NMR (101 MHz, CDCl₃) δ 162.71 (dd, J=247.9, 12.9 Hz), 141.07 (t, J=9.1 Hz), 113.49 (d, J=6.5 Hz), 113.31 (d, J=6.5 Hz), 102.07 (t, J=25.3 Hz), 72.26, 48.05, 26.28.

### Preparation of N-(1,3-bis(3,5-difluorophenyl)-2-methylpropan-2-yl)-2-chloroacetamide (MP_106b)

(lab note volume 4 page 135, y-402)

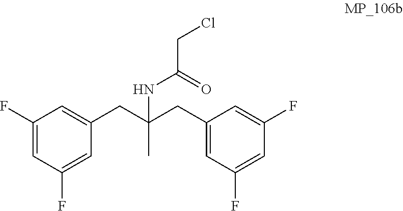 [SMILES: CC(Cc1cc(F)cc(F)c1)(Cc1cc(F)cc(F)c1)NC(=O)CCl]

To a stirred solution of 2-(4-chlorophenyl)propan-2-ol MP_106a (373 mg, 1.25 mmol) in acetic acid (3 ml) was added chloroacetonitrile (158 μL, 2.5 mmol) followed by the addition of sulfuric acid (0.5 mL). The reaction mixture was stirred at room temperature for 13 hr and quenched with cold water. The solution was basified with saturated NaHCO₃(aq) and organic layers were extracted with EtOAc (3×50 mL). The EtOAc layer was washed with water (50 mL), dried over Na₂SO₄ and concentrated under reduced pressure. The residue was subjected to flash column chromatography using EtOAc/hexane to afford product MP_106b (50 mg, 0.13 mmol) in 11% yield.

TLC (SiO₂) R_{f}=0.6 (EtOAc: Acetonitrile=1:4)

¹H NMR (400 MHz, CDCl₃) δ 6.75-6.63 (m, 6H), 5.92 (s, 1H), 3.98 (s, 2H), 3.58 (d, J=13.4 Hz, 2H), 2.80 (d, J=13.4 Hz, 2H), 1.13 (s, 3H).

¹³C NMR (101 MHz, CDCl₃) δ 166.22, 162.80 (dd, J=248.5, 12.9 Hz), 140.39 (t, J=9.1 Hz), 113.39 (d, J=6.5 Hz), 113.21 (d, J=6.5 Hz), 102.46 (t, J=25.2 Hz), 57.23, 43.36, 42.81, 24.21.

### Preparation of (S)-tert-butyl (1-((1,3-bis(3,5-difluorophenyl)-2-methylpropan-2-yl)amino)-1-oxopropan-2-yl)carbamate (MP_106c)

(lab note volume 4 page 137, y-40 and page 139, y-404)

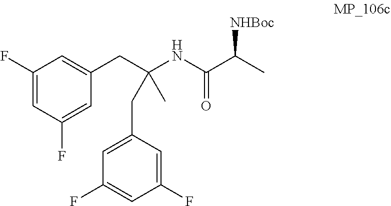 [SMILES: C[C@H](NC(=O)OC(C)(C)C)C(=O)NC(C)(Cc1cc(F)cc(F)c1)Cc1cc(F)cc(F)c1]

To a solution of N-(1,3-bis(3,5-difluorophenyl)-2-methylpropan-2-yl)-2-chloroacetamide MP_106b (50 mg, 0.13 mmol) in 5 mL of ethanol was added thiourea (12 mg, 0.16 mmol) along with 1 mL of acetic acid. The reaction mixture was allowed to reflux overnight. The reaction mixture was cooled and poured into 40 mL of cold water. The solution was made alkaline with 4 M NaOH (pH 11) and extracted with EtOAc (20 mL×2). The combined organic layers were washed with saturated NaHCO₃ and brine, dried over Na₂SO₄ and the solvent was evaporated under reduced pressure to afford the crude amine product. The crude amine product was placed under high vacuum overnight to remove any residual solvent. The crude amine product was used for the next step without purification.

To a solution of tertiary amine synthesized from previous step in 5 ml of DCM was added 4-dimethylaminopyridine (3 mg, 0.03 mmol) and N-(tert-butoxycarbonyl)-L-alanine (49 mg, 0.26 mmol). The mixture was allowed to stir for 10 min at 0° C. Then, N-(3-dimethylaminopropyl)-N′-ethylcarbodiimide hydrochloride (50 mg, 0.26 mmol) was added to the solution and the mixture was stirred for 1 h at the same temperature. After being stirred for 12 h at room temperature, reaction mixture was quenched with water. The organic layer was extracted by ethyl acetate (20 mL×2), washed with brine (10 mL), dried over Na₂SO₄, filtered and concentrated under reduced pressure. The residue was subjected to flash column chromatography using EtOAc/hexane to afford product MP_106c (24 mg, 0.05 mmol) in 39% yield.

TLC (SiO₂) R_{f}=0.7 (EtOAc:hexane=1:2, visualized by ninhydrin)

¹H NMR (500 MHz, CDCl₃) δ 6.66 (d, J=7.0 Hz, 6H), 5.96 (s, 1H), 4.85 (s, 1H), 4.08-3.94 (m, 1H), 3.69 (d, J=13.0 Hz, 1H), 3.62 (d, J=12.9 Hz, 1H), 2.74 (d, J=12.2 Hz, 1H), 2.63 (d, J=13.2 Hz, 1H), 1.36-1.21 (m, 12H), 1.01 (s, 3H).

¹³C NMR (126 MHz, CDCl₃) δ 172.78, 162.64 (dd, J=248.0, 12.9 Hz), 162.60 (dd, J=247.9, 12.8 Hz), 140.94 (t, J=7.4 Hz), 140.87 (t, J=8.9 Hz), 113.53 (d, J=5.9 Hz), 113.49 (d, J=5.8 Hz), 113.38 (d, J=5.8 Hz), 113.34 (d, J=5.9 Hz), 102.15 (t, J=25.2 Hz). 102.12 (t, J=25.2 Hz), 80.42, 56.44, 50.36, 43.53, 43.29, 28.05, 24.34, 17.10.

### Preparation of (S)-2-amino-N-(1,3-bis(3,5-difluorophenyl)-2-methylpropan-2-yl)propanamide hydrochloride (MP_106) (lab note volume 4 page 137, y-40 and page 139, y-404)

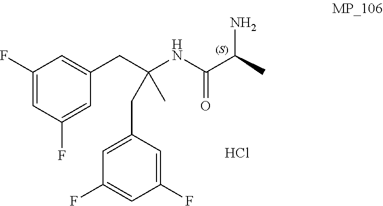

In a round bottom flask equipped with a stir bar, (S)-tert-butyl (1-((1,3-bis(3,5-difluorophenyl)-2-methylpropan-2-yl)amino)-1-oxopropan-2-yl)carbamate MP_106c (24 mg, 0.05 mmol) was cooled to 0° C. and was added 4M HCl in dioxane (2 mL). The reaction mixture was allowed to stir 2 h at room temperature. The solution was concentrated under reduced pressure, and was added Et₂O. The hydrochloride salt was allowed to precipitate, and the product MP_106 (12 mg, 0.03 mmol) was filtered and dried in high vacuum (58% yield). The hydrochloride salt product could also be triturated with Et₂O.

¹H NMR (500 MHz, DMSO) δ 8.29 (s, 3H), 7.72 (s, 1H), 7.12-7.04 (m, 2H), 6.94-6.87 (m, 2H), 6.85-6.77 (m, 2H), 3.73-3.66 (m, 1H), 3.59 (d, J=13.0 Hz, 1H), 3.42 (d, J=13.1 Hz, 1H), 2.78 (d, J=13.1 Hz, 1H), 2.71 (d, J=13.0 Hz, 1H), 1.23 (d, J=7.0 Hz, 3H), 0.91 (s, 3H).

¹³C NMR (126 MHz, DMSO) δ 170.0 (s), 162.5 (dd, J=245.5, 13.7 Hz), 162.39 (dd, J=245.4, 14.8 Hz), 142.3 (t, J=9.4 Hz), 142.0 (t, J=9.4 Hz), 114.19 (d, J=5.3 Hz), 114.04 (d, J=5.2 Hz), 113.89 (d, J=5.0 Hz), 102.45 (t, J=25.5 Hz), 102.44 (t, J=26.3 Hz), 56.93 (s), 48.90 (s), 43.2 (s), 43.0 (s), 28.58 (s), 23.52 (s), 17.73 (s).

HRMS (DART, m/z): Calculated for C₁₉H₂₀F₄N₂OH [M+H]⁺=369.1590; found 369.1584

### Preparation of (S)-4-amino-7-chloro-4,5-dihydro-1H-benzo[c]azepin-3(2H)-one (MP_109)

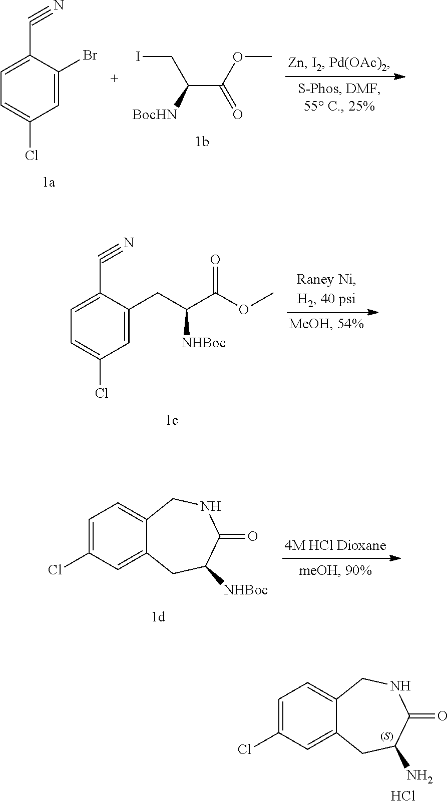

Methyl (S)-2-((tert-butoxycarbonyl)amino)-3-(5-chloro-2-cyanophenyl)propanoate. Step 1. Zinc-100 mesh (3.24 g, 49.99 mmol, 3.60 eq.) was added to a heat gun dried flask under argon. 5 mL of anhydrous DMF was added and the resulting mixture was stirred for five min. Iodine (245 mg, 0.97 mmol, 170 mEq.) was then dissolved in 2 mL of dry DMF and added to Zinc-DMF mixture. The resulting mixture was stirred for five minutes, and the yellow colored reaction became a colorless and clear. In a separate round bottom flask, 8 mL of dry DMF was added to 1b (4.79 g, 14.6 mmol, 1.05 eq.), and the resulting mixture was stirred at r.t for 10 min. Then, the 1b solution was added to Zinc-Iodine-DMF reaction mixture dropwise for 10 min. After completion of addition, the internal temperature of the reaction was slowly raised to 55° C. in 10 min. The resulting mixture was then slowly cooled to r.t. In a separate flask, S-Phos (569 mg, 1.39 mmol, 0.1 eq.), Pd(OAc)2 (155 mg, 0.693 mmol, 50.0 mEq.), 1a (3.0 g 13.9 mmol, 1.0 eq.) and 9 mL of anhydrous DMF were added and the resulting mixture was stirred at r.t for 5 min before being added to the first reaction mixture using a syringe under argon. The resulting mixture was stirred at 55° C. overnight. The reaction flask was cooled to r.t, and reaction mixture was quenched with 20 ml of aq. NH4Cl and diluted with 100 mL of EtOAc. The resulting mixture was stirred at r.t for 30 min. The organic layer was then extracted, separated, dried over Na₂SO₄, and concentrated. The resulting residue was purified by flash column chromatography to yield 1.50 g of impure product. The impure material was then recrystallized using DCM/Hex (1:10) to yield pure 1c as a light yellow solid (950 mg, yield=20%). 1H NMR (300 MHz, CHLOROFORM-d) δ ppm 7.57 (d, J=8.21 Hz, 1H) 7.32-7.39 (m, 2H) 5.15 (br d, J=7.03 Hz, 1H) 4.57-4.69 (m, 1H) 3.80 (s, 3H) 3.39 (br d, J=5.86 Hz, 1H) 3.09-3.21 (m, 1H) 1.40 (s, 10H). LC-MS: (M-Boc)+=238.10 was observed.

1.2 Tert-butyl (S)-(7-chloro-3-oxo-2,3,4,5-tetrahydro-1H-benzo[c]azepin-4-yl) carbamate. Step 2. To a 20 mL vial containing approximately 0.5 g of Raney Nickel was added methanol (8.40 mL, 12 vol.) and 1c (700 mg, 1.07 mmol, 1.0 eq.). The vial was kept in a high-pressure hydrogenation flask which was initially flushed with argon and filled with hydrogen. The flask was then sealed under H2 at 40 psi. The reaction mixture was then stirred at r.t. for 6 h, then the hydrogenation flask was evacuated and flushed with argon. The reaction mixture was quenched with 5 g of celite and the material was filtered through a thick pad of celite. The cake was rinsed with ethanol and the filtrate was concentrated and diluted with EtOAc and water. The organic layer was then separated, dried over Na₂SO₄, and concentrated. The resulting residue was purified by flash column chromatography to yield pure Id (282 mg, 44%). 210 mg of impure Id was isolated (with impurity being dechlorinated 1d). 1H NMR (300 MHz, CHLOROFORM-d) δ ppm 7.20-7.27 (m, 1H) 7.04-7.13 (m, 2H) 6.95 (d, J=8.21 Hz, 1H) 5.85 (br d, J=5.86 Hz, 1H) 4.90-5.06 (m, 1H) 4.70-4.84 (m, 1H) 3.96 (dd, J=16.70, 7.33 Hz, 1H) 3.34 (br d, J=4.10 Hz, 1H) 2.91 (dd, J=17.00, 12.89 Hz, 1H) 1.46 (s, 9H). LC-MS: (M-Boc)+=210.10.

1.3 (S)-4-amino-7-chloro-1,2,4,5-tetrahydro-3H-benzo[c]azepin-3-one. Step 3. 1d (0.29 mmol, 1.0 equiv.) was taken in heat gun dried flask, Methanol (0.765 mL, 8.50 vol.) was added, reaction flask was cooled to 0° C., HCl in Dioxane (0.311 mL, 1.25 mmol, 4.3 equiv.) was added to the reaction mixture dropwise at 0° C., reaction flask was warm to r.t, resulting reaction mixture was stirred at r.t for 3 hours. Reaction mixture was concentrated and triturated with diethyl ether, ether decanted in separate flask, white solid was dried under vacuum to yield target molecule as a white solid (55 mg, yield=90.2%). (1H NMR (300 MHz, DMSO-d6) δ ppm 8.70 (br t, J=5.86 Hz, 1H) 8.44 (br s, 3H) 7.34 (s, 1H) 7.25 (d, J=1.17 Hz, 2H) 4.73-4.89 (m, 2H) 3.99 (dd, J=16.41, 7.03 Hz, 1H) 3.37 (br s, 1H) 3.08 (dd, J=17.00, 12.89 Hz, 1H). LC-MS: (M+H)+=211.4 observed.

### Example 3: In Vivo Test of Exemplary Compounds

### Affinity Purification and Proteomics Analysis Suggest PRMT-A03 Interaction

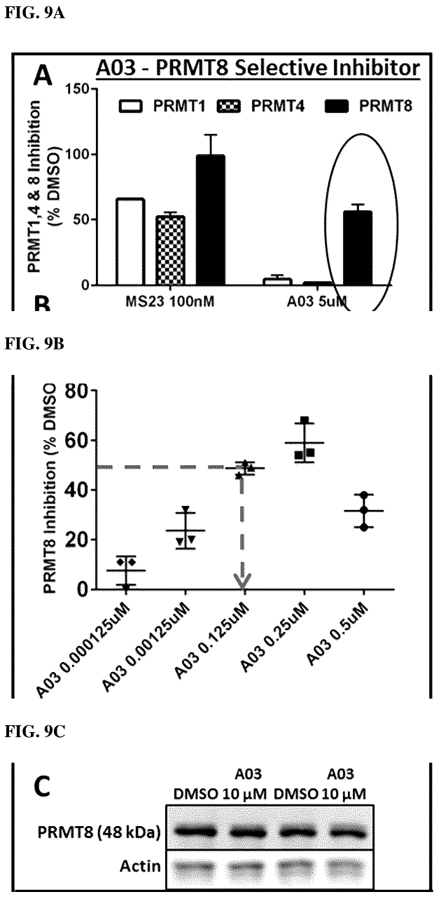

A variety of methods and approaches were used to identify the target of A03 that elicits SirT1 enhancement. One method used was affinity purification (FIG. 7) to discover which proteins interact with an analog of A03. To do this, A03 analog DDL215 was conjugated to azide-agarose beads using click chemistry; beads that underwent the same protocol but in the absence of DDL215 were used as a control. The chromatography columns were packed with DDL215 conjugated or control beads (resin) and equilibrated with M-PER buffer. After confirming that A03 treatment of human neuroblastoma SH-SY5Y cells transiently transfected with ApoE4 resulted in SirT1 increases similar to those seen in the E4-N2a cells used for screening, we ran SH-SY5Y lysates through the column because they are a human, rather than murine (E4-N2a), neuronal cell line. SH-SY5Y cell lysates from ten 10 cm plates of 70-80% confluence were used. Cells were washed with PBS, lysed in M-PER buffer (PIC w/o EDTA) for 40 minutes on ice, and then centrifuged at 10,000×g for 20 min at 4° C. The lysates were combined and 3 mL of each were used per column. All at 4° C., lysates were run through the columns 3 times at 0.5 mL/min and then incubated overnight with shaking. Columns were washed with 10 mL of PBS (pH 7.4) and bound protein eluted with a gradient of A03 analog DDL210: 1 mM, 2 mM, 3 mM, 4 mM, and 5 mM at 0.5 mL/min; 0.5 mL fractions (Fx) were collected. Protein concentrations in the collected fractions was determined using a BCA assay.

Fractions from 1 mM DDL210 (Fx 28) and 2 mM DDL210 (Fx 33) elution were collected, lyophilized and underwent electrophoresis in a polyacrylamide gel. Silver staining was then performed (Thermo Scientific 24600) and bands excised, de-stained, digested with trypsin (in-gel) and subjected to proteomic LC-MS analysis.

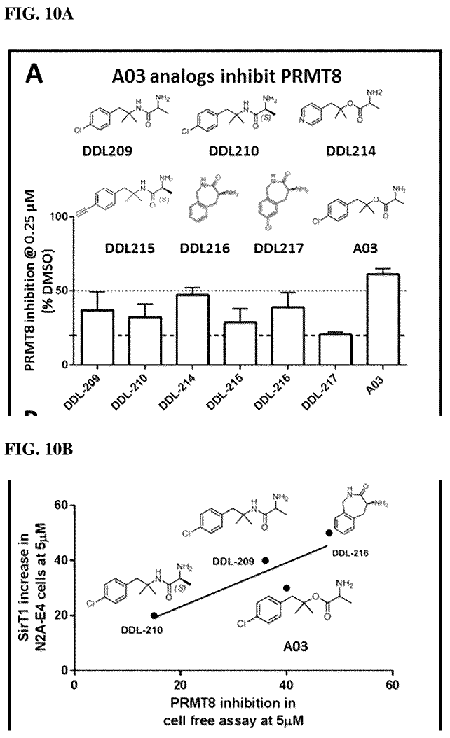

Proteomics performed on affinity-purified samples revealed a large number of proteins associated with the A03 analogs, so to narrow down the list for further investigation, proteins with known associations with SIRT1 were revealed by protein-protein association network analysis (STRING; FIG. 8). Of these, protein arginine methyltransferase 4 (PRMT4; CARM1) was particularly intriguing because the literature indicates that it may transcriptionally regulate SIRT1 levels.

### PRMT8 as a Target of A03 and Analogs

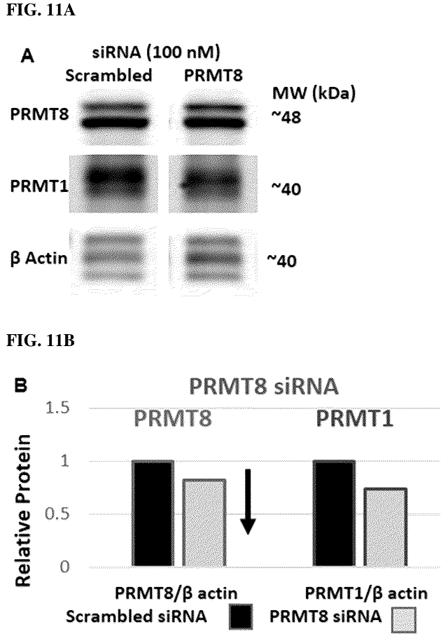

Due both the sequence homology and functional similarity of PRMT family members, in follow-up experiments PRMT 1, 4, and 8 inhibitors as well as assays for inhibition by those PRMTs were used. In a cell-free enzyme activity assay, A03 at 5 μM was found to inhibit PRMT8, but not PRMT1 or 4 (FIG. 9A) using MS23 as a control as it inhibits all 3 enzymes. The dose-response (FIG. 9B) indicates 50% inhibition of PRMT8 is achieved at submicromolar (IC50˜0.125 μM) dose of A03. The dose response shows a hormetic profile reaching a maximum at 0.25 μM and then plateauing when tested up to 10 μM. This profile may be due to its binding to an allosteric site on the PRMT8 enzyme and needs further kinetic analysis. Enantiomers of A03 were evaluated and data shows that one enantiomer is better than the other, similar to what was observed for SirT1 enhancement (FIG. 4). The mode & inhibition kinetics to be investigated in the renewal grant. Immunoblot analysis shows that A03 treatment in E4-N2a cells does not alter PRMT8 protein levels at either 10 μM (FIG. 9C).

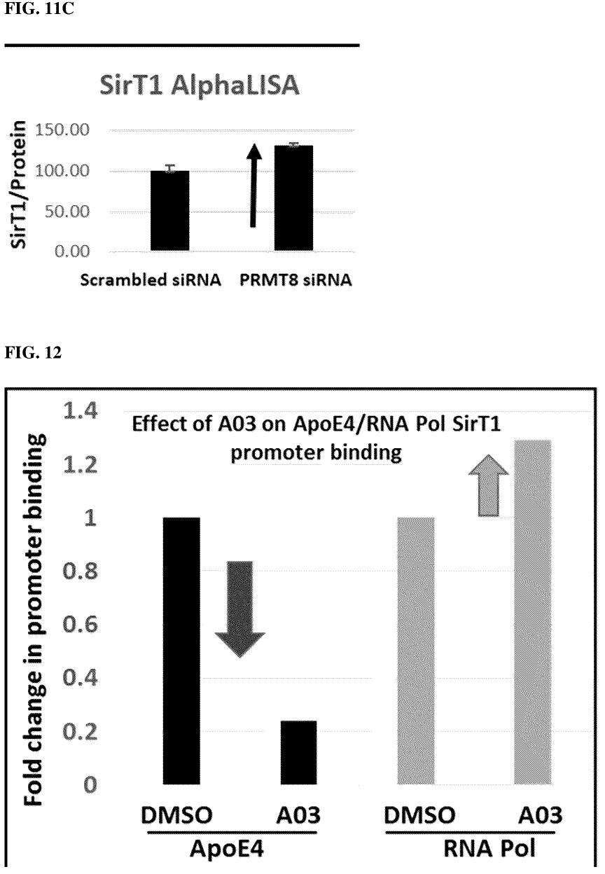

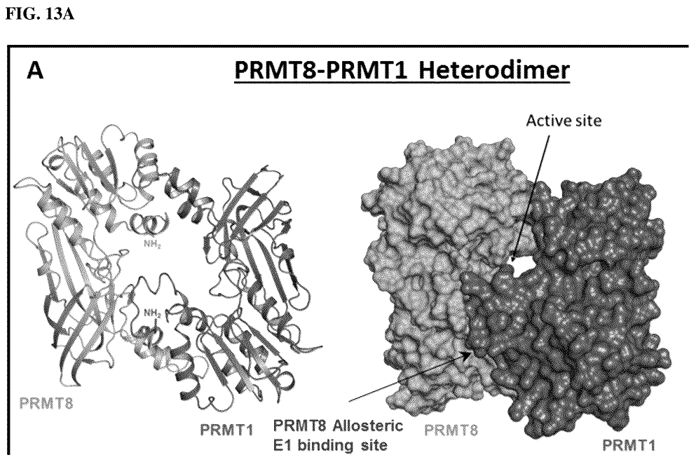

The effects of several A03 analogs were also tested in the cell-free PRMT8 assay, and as shown in FIG. 10A. DDL209, 214 and 216 showed 50% inhibition of PRMT8 at 0.25 μM. Testing of additional A03 analogs are ongoing. For affinity purification, DDL215 that was used through clicking to beads. PRMT8 inhibited the enzyme ˜30% at 0.25 μM and enhanced SirT1 levels in E4-N2a cells. An initial correlation between cell-free PRMT8 inhibition and neuronal SirT1 enhancement (in cells) at 5 μM was seen (FIG. 10B). Furthermore, recent in vivo testing with the A03 analog DDL214, oral treatment for 56 days, shows a trend to increase SirT1 in hippocampi of E4AD mice and improvement in cognition using the discrimination index in the NOR memory testing paradigm.

### PRMT8 siRNA Knockdown Increases SirT1

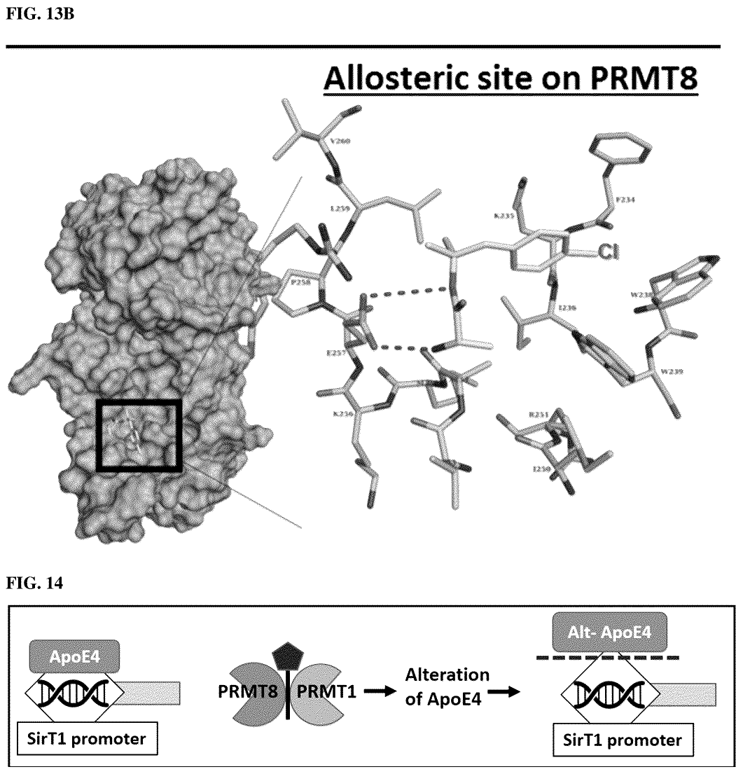

Because of PRMT8's brain-specificity and the finding that A03 inhibits PRMT8, studies were performed to determine if siRNA knockdown of PRMT8 would result in an increase in SirT1. E4-N2a cells were transfected with PRMT8 or a scrambled peptide siRNAs (Santa Cruz Biotechnology) and 8 hours later, cells were collected, lysed, electrophoresed, and immunoblotted using anti-PRMT1, PRMT8, and β-actin (control) antibodies. Lysates also underwent SirT1 AlphaLISA with adjustment to total protein. Interestingly, as shown in FIG. 11, PRMT8 siRNA (100 nM) knockdown decreased both PRMT1 and PRMT8 protein levels relative to β-actin (immunoblots shown in FIG. 11A, densitometry analysis in FIG. 11B). A key finding was that PRMT8 siRNA knockdown, increases SirT1 (FIG. 11C).

### A03 decreases ApoE4 Interaction with the SirT1 Promoter and Increases RNA Polymerase Interaction

As ApoE4 directly interacts with the SirT1 promoter at a CLEAR DNA sequence, it was important to determine if A03 had an effect on the promoter binding interaction and if A03 could affect SirT1 transcription. Therefore, N2a-E4 cells (3×10⁶) were treated with 50 μM A03 or vehicle (DMSO) for 6 hours. Cells were then fixed with formaldehyde, lysed with SDS buffer, and sonicated using a temperature-controlled Epishear sonication platform at −20° C. to obtain DNA fragments between 200-1000 bp. An EZ-ChIP kit (EMD Millipore) was then used for chromatin immunoprecipitation using anti-ApoE4 mAb (Novus Biologicals) and anti-RNA Polymerase II mAb (Millipore) antibodies. After ChIP crosslinks were reversed and purified ChIP DNA was then analyzed by real time quantitative PCR using SYBR green master mix (Thermo) in a CFX Connect Real-Time PCR Detection System (Bio-Rad) using primers to amplify the ApoE4 binding site in the mouse SirT1 promoter: 5′ACCTCGTCCGCCATCTTC3′ and 5′GGTCACGTGACGGGGTTT3′ (amplicon size of 125 bp). The Signals from each sample were normalized with signal of the input followed by the delta-delta-Ct method and fold change calculated with respect to the vehicle (DMSO).

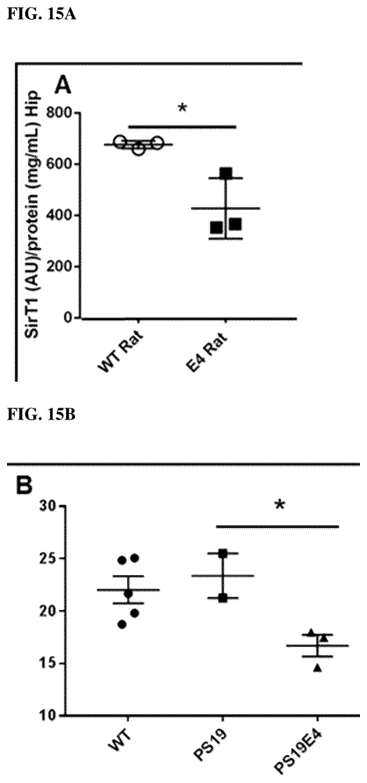

As shown in FIG. 12, under these conditions, there was a pronounced decrease in the interaction of ApoE4 with the SirT1 promoter in the presence of A03 at 6 hours after drug treatment and this was associated with increased binding of RNA polymerase II to the SirT1 promoter which suggests transcription was enhanced. Indeed, at a 10 hour time point, SirT1 mRNA level with A03 treatment was 12% higher than DMSO control. In continued studies, the timing and dynamics of A03/analog effects on PRMT8 and the methylation/citrullination state of ApoE4, ApoE4 interaction with the SirT1 promoter, RNA Pol II binding, and mRNA and protein levels will be investigated.

### A03 Enantiomer E1 Binds to an Allosteric Site on PRMT8.

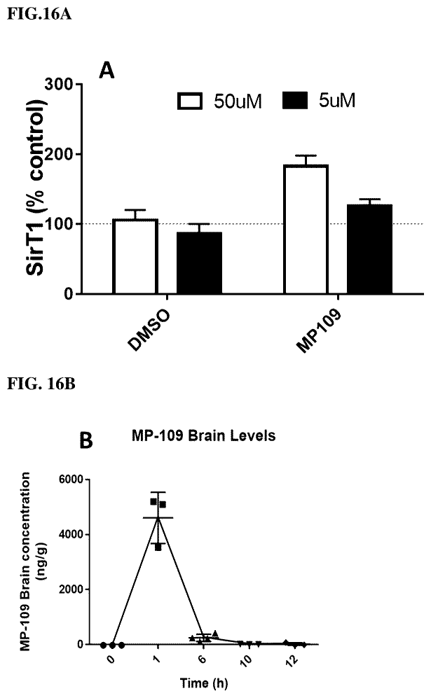

Through docking and simulation it was shown that PRMT8 and PRMT1 form a tight heterodimeric complex. For these studies PRMT8 with PRMT1 was modeled based on the molecular packing observed in the crystal structure of PRTM8 (pdbid 5DST) and PRMT 1 (pdbid 6NT2). The modeled complex was then subjected to energy minimization with AMBER to relieve steric clashes. Molecular dynamics (MD) simulations were performed to determine the binding free energy of PRMT1 binding to PRMT8. Here, PRMT8 was treated as a receptor and the PRMT1 as a ligand to calculate the binding free energy. After adding hydrogens, the modeled PRMT1-PRMT8 complex was solvated in a truncated octahedral TIP3P box of 12 Å, and the system was neutralized with sodium ions. Periodic boundary conditions, Particle Mesh Ewald summation and SHAKE-enabled 2-femto seconds time steps were used. Langevin dynamics temperature control was employed with a collision rate equal to 1.0 ps-1. A cutoff of 13 Å was used for nonbonding interactions. Initial configurations were subjected to a 1000-step minimization with the harmonic constraints of 10 kcal·mol-1·A°-2 on the protein heavy atoms. The systems were gradually heated from 0 to 300 K over a period of 50 ps with harmonic constraints. The simulations at 300° K were then continued for 50 ps during which the harmonic constraints were gradually lifted. The systems were then equilibrated for a period of 500 ps before the 50 ns production runs. All simulations were carried out in the NPT ensemble. Equilibration and production run simulations were carried out using the Sander and PMEMD modules (optimized for CUDA) of AMBER 18.0 (ff14SB), respectively. All analyses were performed using the cpptraj module of AmberTools 18. The binding free energy for the PRMT1 binding to PRMT8 were estimated using the MMPBSA module in AMBER by taking snapshots (10000) at every 5 ps from the 50 ns production run and was found to be 130.36 kcal/mol. In silico molecular docking analyses were performed to the predicted E1-PRMT8 interaction using the published crystal structure of PRMT8 protein (PDBID: 4X41). Modeling of A03 active enantiomer (E1) interaction revealed a potential allosteric binding site on PRMT8 (black boxes, FIG. 13B) that are located at the PRMT8-PRMT1 interface. This allosteric binding of the enantiomer can potentially inhibit PRMT8 activity and also modulate PRMT8-PRMT1 transcriptional rheostat. Further characterization of this allosteric binding sites by photoaffinity labeling and crystallization is planned.

Putative Model: A03 Alters ApoE4 Interaction with the SirT1 Promoter Via Modulation of the PRMT8:PRMT1 Complex

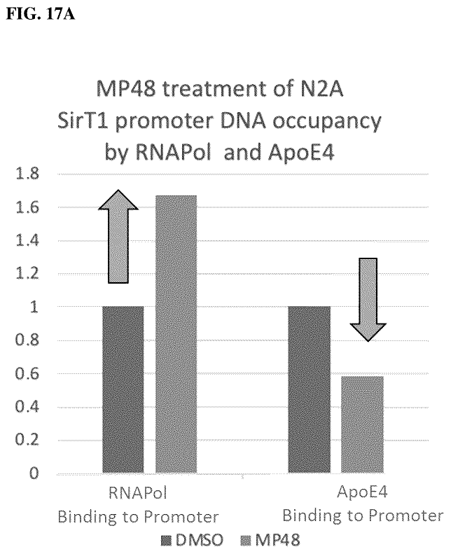

Data suggests that A03 and analogs interact with PRMT8 at an allosteric site so that its interaction with PRMT1 and heterodimer complex formation in neurons is affected. ApoE4 interaction with the SirT1 promoter (FIG. 14A) may be affected by A03/analog mediated modulation of the PRMT8:PRMT1 complex (FIG. 14B). Modulation may alter PRMT8 and/or PRMT1 enzymatic activity and transcriptional effects, increasing levels of PAD or activity of another PRMT such as CARM1 (PRMT4) that could result in a significant change in the methylation or citrullination state of key Arg amino acids in ApoE4 (FIG. 14C) that are involved in its binding to the SirT1 promoter. This has particular importance to ApoE4 effects, because domain-specific Arg residues such as Arg-61 interaction with glutamic acid-255 in ApoE4 distinguishes it from ApoE3 and is associated with some of ApoE4's deleterious effects, and Arg-112 is directly involved in ApoE4-DNA binding activity, as mentioned above. Arg methylation could disrupt ApoE4's binding interaction with the SirT1 promoter, that is, ‘release the ApoE4 brake’ (FIG. 14D) and result in increased SirT1 expression, for elucidation in this renewal proposal.

As for the relevance of these finding to AD, in addition to catalyzing Arg methylation, PRMT8 is involved in production of asymmetric dimethyl arginines (ADMAs), endogenous regulators of nitric oxide synthase (NOS). While knocking out PRMT8 protein levels in mice is deleterious and associated with decreased ADMAs, in AD, homocysteine and ADMAs are increased and concentrations of nitric oxide are decreased in plasma. Inhibition of endothelial NOS by ADMA impairs cerebral blood flow, which may contribute to the development of AD. Thus, in the case of AD, a PRMT8 inhibitor may have similar but more potent effects as those compounds found in red wine that decrease ADMAs in a SirT1-dependent manner. Taken together, this data indicate PRMT8 inhibitors that allosterically inhibit the enzyme but do not change its protein levels can be SirT1 enhancers have the potential to ameliorate deleterious ApoE4 effects, modulate tau pathology and may be truly disease-modifying.

### PRMT Overexpression in N2a-E4 Cells

N2a-E4 (75 k/well in 24 well plate) cells were transfected with 0.75 ug of Type-I PRMT vectors (Origene), These include PRMT1, PRMT4 and PRMT8. For Type-II PRMT similar overexpression with PRMT-5 vector (Origene) was done. Similarly for Type-III PRMT similar overexpression with PRMT-7 vector (Origene) were done. In all cases, pCMV6 entry vector transfection was used as a control for 48 h. Sirt1 levels were measured by alphaLISA followed by normalization with protein concentration. mRNA and CHIP analysis were done from these transfected cells.

### Summary of Data

A03 and analogs were found to be promising SirT1 enhancers both in vitro and in vivo in an ApoE4-expressing FAD mouse model, increasing SirT1 in brain tissue and, in the case of A03, improving cognition. Our mechanistic studies, starting with target identification by affinity chromatography and proteomics to identify proteins that interact with A03 analogs, first pointed to CARM1 (PRMT4), but follow-up studies using cell-free enzyme assays revealed that A03 and analogs modulate activity of PRMTs to induce the observed SirT1 increase. Our inhibition studies show that only inhibition of the brain-specific PRMT8 enzyme activity modulates SirT1 levels. While initial correlation studies show inhibition of PRMT8 by A03 and select analogs correlate with increases in SirT1, additional correlation studies are ongoing. It was also found that siRNA PRMT8 knockdown in cells results in a SirT1 increase and, by ChIP/real time PCR, A03 both decreases ApoE4 interaction with the SirT1 promoter and increases RNA polymerase II interaction, revealing a new mechanism by which these analogs target ApoE4 and SirT1. In silico docking revealed A03 is predicted to interact with an allosteric site(s) on PRMT8. These data taken together led to the development of the hypothesis that A03 interaction/inhibition of PRMT8 and/or its complex formation with PRMT1 results in enhancement of SirT1, perhaps by altering ApoE4 and abrogating its SirT1 promoter binding effects. Our overexpression studies show that the Type-II PRMT-5 overexpression results in increased SirT1 levels in N2a-E4 cells compared to pCMV entry vector control. Both the inhibition and overexpression studies suggest that A03 and analogs can modulate PRMTs result in modulation of ApoE4 SirT1 promoter binding resulting in the release of the ApoE4 binding to the promoter and increasing the levels of the RNAPol binding to the promoter and increase in SirT1 mRNA and SirT1 levels.

### D.3.7 SirT1 Promoter Interaction

N2a-E4 cells will be grown in 10 cm plates (3×10⁶ cells). Cells will be treated with 5-50 M (the actual concentration will be based on the findings in dose-response studies described above) hit or vehicle (typically DMSO) for 6 or 24 h after which cells will be fixed using formaldehyde, lysed using SDS lysis buffer, and sonicated. The Epishear sonication platform with a temperature-controlled −20° C. tube holder will be used and sonication optimized to obtain fragments between 200-1000 bp. This is done using a 1.7 mL Eppendorf tube holding 500 μL of sample containing 5×10⁵ cells which is then sonicated with a ⅛-inch probe set 5 mm from the bottom of the tube, at 25% power for 20 seconds on and 40 seconds off (to prevent heating) for a total of 18 cycles. Chromatin immunoprecipitation will then be performed using the EZ-ChIP kit from EMD Millipore (catalog no. 17-371) per the manufacturer's instructions. For IP, a mouse monoclonal anti-ApoE4 antibody (Novus biologicals, NBP1-49529), mouse monoclonal anti-RNA Polymerase II antibody (provided with EZ-ChIP kit, Millipore 05-623B) and normal anti-mouse IgG (provided with EZ-ChIP kit, Millipore 12-371B) will be used. Uncrosslinked and purified DNA will be then analyzed by real time quantitative PCR using SYBR green master mix (Thermo fisher, A25742) in a CFX Connect Real-Time PCR Detection System from Bio-Rad. The following primers have been designed to amplify the ApoE4 binding site in the mouse Sirt1 promoter with an amplicon size of 125 bp: 5′ ACCTCGTCCGCCATCTTC 3′ and 5′ GGTCACGTGACGGGGTTT 3′. Regarding the qPCR primer design for the SirT1 gene, with one primer overlapping the ApoE4 binding site in the SirT1 promoter and a PCR amplicon size of 125 bp, it is estimated that 75% of the ApoE4-bound chromatin fragments (sheared to 200-1000 bp in length) will be detected by PCR. It is possible up to 25% will go undetected because of genomic DNA substrate shearing fragmentation sites within the 125 bp amplicon region of the genomic DNA, but as long as the shearing fragmentation remains unbiased by experimental conditions, this should not affect the relative quantitative measurement of ApoE4 occupancy of the SirT1 promoter. The real time PCR data will be analyzed using CFX Manager Software. Briefly, the signals from each sample will be normalized with signal of the input followed by the delta delta Ct method and fold change calculated with respect to the vehicle (DMSO).

### Determination of Target Engagement and Efficacy of Optimized PRMT8 Inhibitors/SirT1 in an E4AD Mouse Model.

In these studies, E4AD mice homozygous for human ApoE4 and hemizygous for human APP with K670N/M671L, V717I, and I716V mutations and human presenilin 1 containing the M146L and L286V mutations under the control of Thy1 promoter will be used. Mice will be treated at 5 months of age for 8 weeks by the oral route. There would be 3 groups (n=12/group; male and female): non-transgenic (NTg) vehicle-only (Veh), E4AD (Tg) Veh, and Tg test compound. Safety analysis would include monitoring body weight and cage side activity along with gross organ analysis at end of study after chronic treatment studies for any side effects including tumorigenic effects.

### Cognitive Analysis of E4AD Mice.

In the last 2 weeks of treatment, cognition of the mice will be assessed using Open Field (OF) and the NOR testing paradigm in which a positive discrimination index will indicate novelty preference. Spatial memory of the mice will be assessed using the Barnes Maze and the time to reach goal box and the number of errors will be used as parameters.

### Biochemical Analysis of Brain Tissue.

At the end of treatment, mice will be euthanized by ketamine/xylazine over-anesthesia, transcardial blood collection and saline perfusion; and right brain region (hippocampus, entorhinal cortex, and frontal cortex) tissues will be collected and snap frozen on dry ice and kept at −80° C. until processing for AlphaLISA, ELISA and immunoblot analyses. The left hemisphere will be submerged in 4% paraformaldehyde and processed for immunohistochemistry (IHC). For biochemical analysis, brain tissues will be homogenized in buffer complemented with protease and phosphatase inhibitors then SirT1, SirT2, PRMT8 brain activity, sAPPα, sAPPβ total tau, p-tau, acetyl-tau will be determined by AlphaLISA and Aβ by ELISA; immunoblot will be used for other proteins. IHC will be used to label Aβ with 6E10 antibody on PFA-fixed sections. In addition, we will label microglia with anti-Iba-1 and astrocytes with anti-GFAP antibodies. Another biomarker for analysis in brain tissue will be the microRNA34a (miR-34a), a known regulator of SirT1 mRNA that is upregulated in AD and related to cognitive impairment.

### Efficacy Testing in PSJ9-E4 Mice.

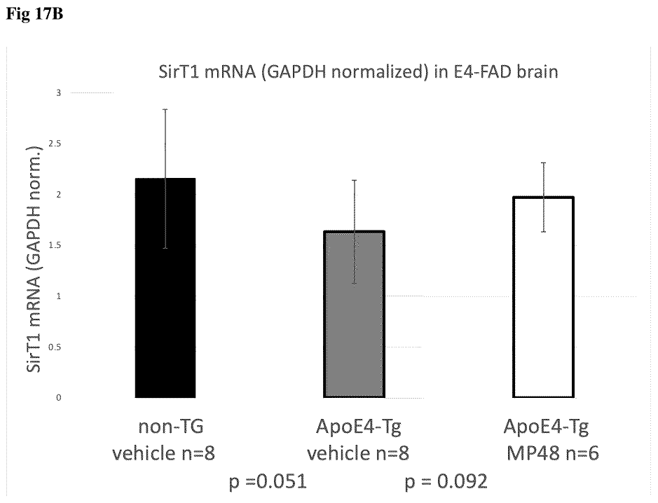

Compounds that are tested in the E4AD mice (ApoE4:5×FAD-TR mice) will be tested in the ApoE-expressing tauopathy mouse model generated by crossing ApoE4 targeted-replacement mice to mice expressing human tau with a P201S mutation (PS19) mice. These mice are homozygous for ApoE4 and hemizygous for tau P301S (E4PS19) and show lower levels of SirT1 in the hippocampus than both PS19 and wildtype mice of the same C57 background (FIG. 15B). This model is available in the PIs lab. The study design will be the same as that for the E4-AD model mice. Along with SirT1 levels we will also measure p-tau levels and p-tau/tau ratios in drug treated groups versus vehicle. Similarly, as in D.4.2 we will conduct cognitive analysis—NOR and Barnes Maze.

### Efficacy Testing in E4 Rats.

Candidates that show efficacy in E4AD model/E4PS19 mice will undergo efficacy testing in E4 KI rats (Envigo). The E4 rats express significantly less SirT1 in the hippocampus than WT rats (FIG. 15A) of the same age/gender on the same background (SD). The efficacy study will be performed as described for the mice testing, but without cognitive testing as these rats only express ApoE4 (not APP, PS1, or tau mutations) and are not cognitively impaired. ADMA levels in CSF and plasma of E4-rats treated with the drugs would also be determined.

Global Proteomics and Analysis for ADMA/Citrullination Effects on Brain Tissue after Lead Treatment.

Brain tissue remaining after removal of hip/entorhinal cortex and frontal cortex will undergo integrated global and phosphoproteomic analyses using liquid chromatography tandem mass spectrometry (LC-MS/MS). Isobaric tandem mass tags (TMT), high pH reversed phase chromatography fractionation, and complementary phosphopeptide enrichment strategies (TiO2 & IMAC) will be employed to provide relative quantitation, increased proteome coverage, and to investigate altered cellular signaling pathways, respectively. A series of bioinformatics methods (comprehensive gene set enrichment, pathway, functional protein association network, and kinase substrate enrichment analyses) will be utilized to investigate the mechanisms underlying A03-mediated SirT1 enhancement, if efficacy is seen. Tissue will be prepared by vortexing minced tissue in 100 μL 5 mM phosphate buffer (pH 7.0), shaking at RT for 1 hr, sonication for 5 min., followed by addition of 100 μL of trifluoroethanol (TFE) (Sigma). Samples will be incubated at 60° C. for 2 h, sonicated for 2 min, then disulfide bonds reduced by 5 mM tributylphosphine (TBP) (Sigma) for 30 min at 60° C. Trypsin (Promega) will be added enzyme (1):protein (50) to five-fold 50 mM NH4HCO3 (pH 7.8) diluted samples, and digested ON at 37° C. SPE C18 column (Supelco)-purified and 80% acetonitrile with 0.1% trifluoroacetic acid (TFA) eluted peptides will be concentrated by lyophilization before LC-MS analysis.

### Example 4: Exemplary Activity of Compounds of the Disclosure

| | | | | | | | SirT1 | |
|---|---|---|---|---|---|---|---|---|
| | | | | | | | SμM | |
| Code | Structure | CAS # | MW | clogp | TPSA | Status | SOμM | PAMPA |
| A03 | 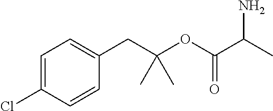 [SMILES: CC(N)C(=O)OC(C)(C)Cc1ccc(Cl)cc1] | 60719-82-6* | 255.74 | 3.08 | 52.32 | com- mer- cial- bought | A, C | 3.9 4.04 |
| MP- 01 | 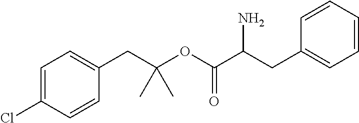 [SMILES: CC(C)(Cc1ccc(Cl)cc1)OC(=O)C(N)Cc1ccccc1] | 66171-65-1 773001-35-9 | 331.84 | 4.50 | 52.32 | done JP | A, A | 4.72 |
| MP- 02 | 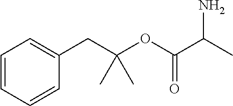 [SMILES: CC(N)C(=O)OC(C)(C)Cc1ccccc1] | 57469-93-9* (S) | 221.30 | 2.37 | 52.32 | done JP | A, A | 3.17 |
| MP- 03 | 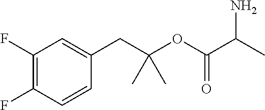 [SMILES: CC(N)C(=O)OC(C)(C)Cc1ccc(F)c(F)c1] | 1882934-33-9* | 257.28 293.74 | 2.58 | 52.32 | done JP | B, C | 2.37 |
| MP- 04 | 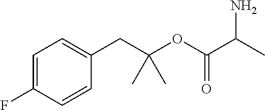 [SMILES: CC(N)C(=O)OC(C)(C)Cc1ccc(F)cc1] | 142393-68-6* (S) 66171-55-9* (S) | 239.29 | 2.51 | 52.32 | done JP | A, B | 2.75 |
| MP- 05 | 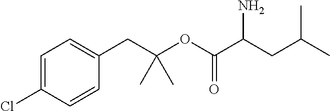 [SMILES: CC(C)CC(N)C(=O)OC(C)(C)Cc1ccc(Cl)cc1] | 120493-80-7* (S) | 297.82 | 4.54 | 52.32 | done JP | A, B | 5.63 |
| MP- 06 | 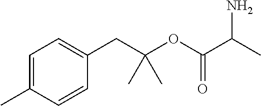 [SMILES: Cc1ccc(CC(C)(C)OC(=O)C(C)N)cc1] | 66171-56-0 (S) 142393-65-5* (S) | 235.33 | 2.87 | 52.32 | done JP | A, B | 4.22 |
| MP- 07 | 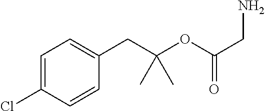 [SMILES: CC(C)(Cc1ccc(Cl)cc1)OC(=O)CN] | 120493-68-7* 66171-59-3* | 241.71 | 2.77 | 52.32 | done JP | A, B | 4.15 |
| MP- 08 | 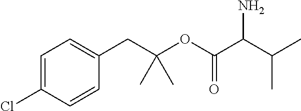 [SMILES: CC(C)C(N)C(=O)OC(C)(C)Cc1ccc(Cl)cc1] | 120493-42-7* (S) | 283.80 | 4.01 | 52.32 | done JP | A, C | 4.65 |
| MP- 09 | 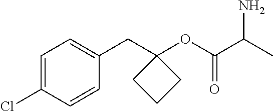 [SMILES: CC(N)C(=O)OC1(Cc2ccc(Cl)cc2)CCC1] | 1882934-34- 0*(S) | 267.75 | 3.27 | 52.32 | done JP | A, A | 4.16 |
| MP- 10 | 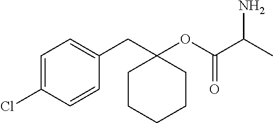 [SMILES: CC(N)C(=O)OC1(Cc2ccc(Cl)cc2)CCCCC1] | 1882934-35- 1*(S) | 295.81 | 4.39 | 52.32 | done JP | A, B | 6.58 |
| MP- 11 | 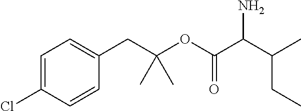 [SMILES: CCC(C)C(N)C(=O)OC(C)(C)Cc1ccc(Cl)cc1] | 120493-52- 9*(SS) | 297.83 | 3.95 | 52.33 | done JP | A, A | 5.28 |
| MP- 12 | 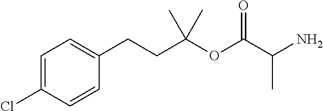 [SMILES: CC(N)C(=O)OC(C)(C)CCc1ccc(Cl)cc1] | 1882934-36- 2*(S) | 269.77 | 3.19 | 52.33 | done JP | A, A | 4.95 |
| MP- 13 | 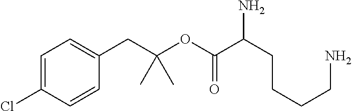 [SMILES: CC(C)(Cc1ccc(Cl)cc1)OC(=O)C(N)CCCCN] | 1882934-37- 3*(S) | 312.84 | 2.18 | 78.35 | done JP | A, C | 6.49 |
| MP- 14 | 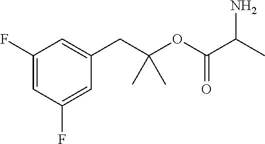 [SMILES: CC(N)C(=O)OC(C)(C)Cc1cc(F)cc(F)c1] | 1882934-38- 4*(S) | 257.28 | 2.25 | 52.33 | done JP | A, A | 2.17 |
| MP- 15 | 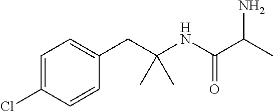 [SMILES: CC(N)C(=O)NC(C)(C)Cc1ccc(Cl)cc1] | 66316-95-8* | 254.76 | 1.92 | 55.12 | done JP | A, B | 2.74 |
| MP- 16 | 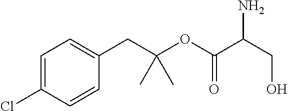 [SMILES: CC(C)(Cc1ccc(Cl)cc1)OC(=O)C(N)CO] | 1882934-39- 5*(S) | 271.74 | 1.70 | 72.56 | done JP | A, A | 2.53 |
| MP- 17 | 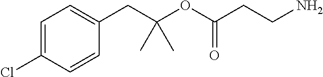 [SMILES: CC(C)(Cc1ccc(Cl)cc1)OC(=O)CCN] | 1882934-40-8* | 255.74 | 2.23 | 52.33 | done JP | A, C | 3.86 |
| MP- 18 | 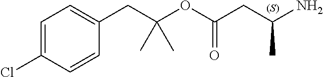 | 1882934-41- 9*(S) | 269.77 | 2.64 | 52.33 | done JP | a, A | 4.01(2) |
| MP- 19 | 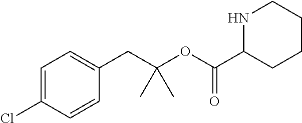 [SMILES: CC(C)(Cc1ccc(Cl)cc1)OC(=O)C1CCCCN1] | 1882934-42- 0*(S) | 295.80 | 4.15 | 38.33 | done JP | A, A | 2.61 |
| MP- 20 | 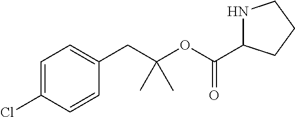 [SMILES: CC(C)(Cc1ccc(Cl)cc1)OC(=O)C1CCCN1] | 1882934-43- 1*(S) | 281.78 | 3.64 | 38.33 | done JP | A, B | 2.66 |
| MP- 21 | 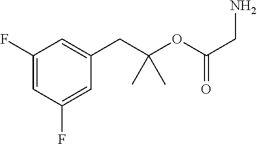 [SMILES: CC(C)(Cc1cc(F)cc(F)c1)OC(=O)CN] | NCE | 243.25 | 1.85 | 52.33 | done JP | A, A | 2.55m |
| MP- 22 | 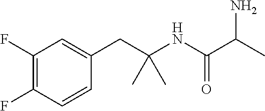 [SMILES: CC(N)C(=O)NC(C)(C)Cc1ccc(F)c(F)c1] | 1882934-45-3* | 256.80 | 1.49 | 55.12 | done JP | A, C | 1.7 |
| MP- 23 | 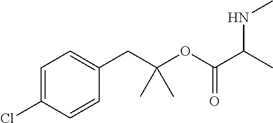 [SMILES: CNC(C)C(=O)OC(C)(C)Cc1ccc(Cl)cc1] | 1882934-45- 4*(S) | 269.77 | 3.58 | 38.33 | done JP | a, a | 3.03m |
| MP- 24 | 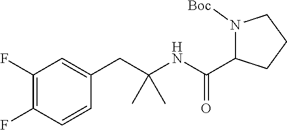 [SMILES: CC(C)(Cc1ccc(F)c(F)c1)NC(=O)C1CCCN1C(=O)OC(C)(C)C] | NCE | 332.45 | 4.42 | 58.64 | done JP | a, a | 1.27m |
| MP- 25 | 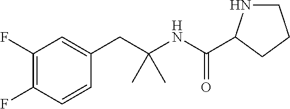 [SMILES: CC(C)(Cc1ccc(F)c(F)c1)NC(=O)C1CCCN1] | 1882934-47-5* | 282.33 | 2.46 | 41.12 | done JP | A, B | 1.23(2) |
| MP- 26 | 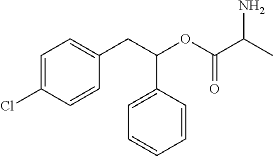 [SMILES: CC(N)C(=O)OC(Cc1ccc(Cl)cc1)c1ccccc1] | NCE | 303.79 | 3.44 | 52.33 | done JP | A, A | 3.45 |
| MP- 27 | 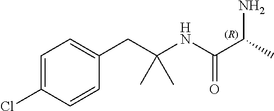 | 66316-95-8* | 254.76 | 1.92 | 55.12 | done JP | a, a | 2.72 |
| MP- 28 | 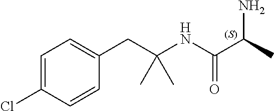 | NCE | 254.76 | 1.92 | 55.12 | done JP | A, B | 2.78 2.86 |
| MP- 29 | 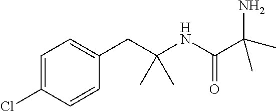 [SMILES: CC(C)(Cc1ccc(Cl)cc1)NC(=O)C(C)(C)N] | NCE | 268.78 | 2.93 | 55.12 | done JP | a, a | 2.1 |
| MP- 30 | 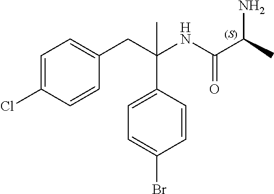 | NCE | 395.73 | 3.94 | 55.12 | done JP | A, A | 2.89 |
| MP- 31 | 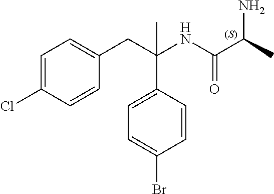 | NCE | 395.73 | 3.94 | 55.12 | done JP | A, B | 2.89 |
| MP- 32 | 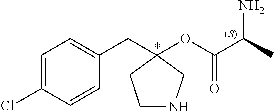 | NCE | 282.77 | 1.84 | 64.36 | done JP | A, C | 0.68 |
| MP- 33 | 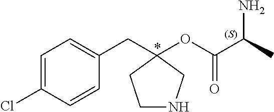 | NCE | 282.77 | 1.84 | 64.36 | done JP | A, C | 0.70m |
| MP- 34 | 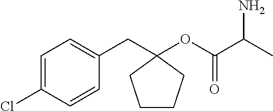 [SMILES: CC(N)C(=O)OC1(Cc2ccc(Cl)cc2)CCCC1] | NCE | 281.78 | 3.33 | 52.33 | done JP | A, A | 4.92 |
| MP- 35 | 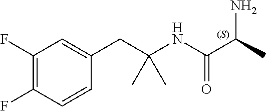 | 1882934-45-3* | 256.30 | 1.49 | 55.12 | done JP | A, A | 1.7 |
| MP- 36 | 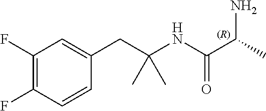 | 1882934-45-3* | 256.30 | 1.49 | 55.12 | done JP | a, a | 1.66 |
| MP- 37 | 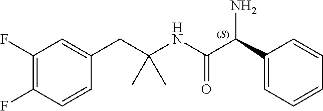 | NCE | 318.38 | 1.40 | 55.12 | done JP | A, a | 1.81 |
| MP- 38 | 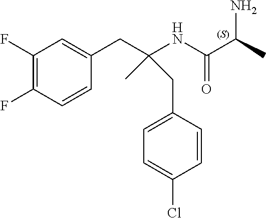 | NCE | 366.84 | 3.60 | 55.12 | done JP | A, A | 3.07 |
| MP- 39 | 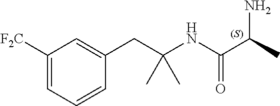 | NCE | 288.31 | 2.11 | 55.12 | done JP | A, A | 1.7 |
| MP- 40 | 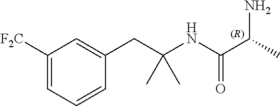 | NCE | 288.31 | 2.11 | 52.12 | done JP | A, A | 1.78 |
| MP- 41 | 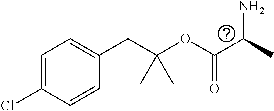 [SMILES: C[C@H](N)C(=O)OC(C)(C)Cc1ccc(Cl)cc1] | 66171-75-3* (S) 57469-92-8 HCl | 255.74 | 3.08 | 52.32 | com- mer- cial JP | A, A | 3.88 |
| MP- 42 | 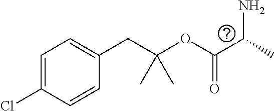 [SMILES: C[C@@H](N)C(=O)OC(C)(C)Cc1ccc(Cl)cc1] | 66171-73-1 | 255.74 | 3.08 | 55.32 | com- mer- cial JP | a, a | 3.9 |
| MP- 43 | 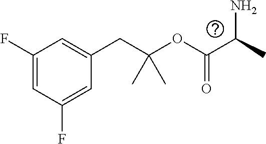 [SMILES: C[C@H](N)C(=O)OC(C)(C)Cc1cc(F)cc(F)c1] | NCE | 256.30 | 1.49 | 55.12 | done JP | A, A | 1.59 |
| MP- 44 | 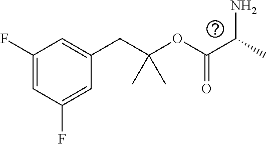 [SMILES: C[C@@H](N)C(=O)OC(C)(C)Cc1cc(F)cc(F)c1] | NCE | 256.30 | 1.49 | 55.12 | done JP | a, A | 1.57 |
| MP- 45 | 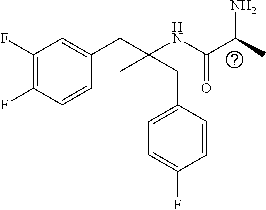 [SMILES: C[C@H](N)C(=O)NC(C)(Cc1ccc(F)cc1)Cc1ccc(F)c(F)c1] | NCE | 350.38 | 3.09 | 55.12 | done JP | A, a | 2.11 |
| MP- 46 | 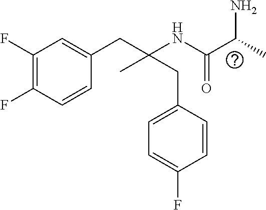 [SMILES: C[C@@H](N)C(=O)NC(C)(Cc1ccc(F)cc1)Cc1ccc(F)c(F)c1] | NCE | 350.39 | 3.09 | 55.12 | done JP | a, A | 2.19 |
| MP- 47 | 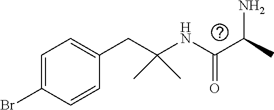 [SMILES: C[C@H](N)C(=O)NC(C)(C)Cc1ccc(Br)cc1] | 1825167-15-4 | 299.21 | 2.05 | 55.12 | done JP | B, A | 1.76 |
| MP- 48 | 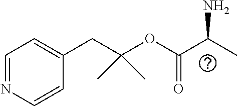 [SMILES: C[C@H](N)C(=O)OC(C)(C)Cc1ccncc1] | NCE | 222.29 258.74 | 0.70 | 65.22 | done JP | A, B | 0.4 |
| MP- 49 | 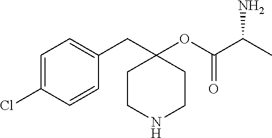 [SMILES: C[C@@H](N)C(=O)OC1(Cc2ccc(Cl)cc2)CCNCC1] | NCE | 296.80 | 1.76 | 64.36 | done JP | A, B | 1.02(2) |
| MP- 50 | 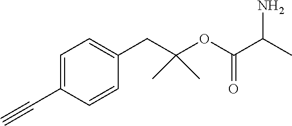 [SMILES: C#Cc1ccc(CC(C)(C)OC(=O)C(C)N)cc1] | NCE | 245.32 | 1.76 | 52.33 | done JP | A, B | 3.36 |
| MP- 51 | 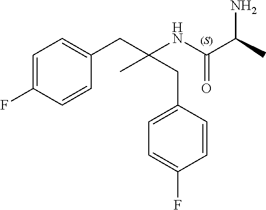 | NCE | 332.39 | 2.99 | 55.12 | done JP | A, C | 2.25 2.38 |
| MP- 52 | 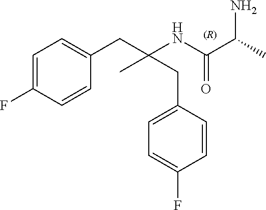 | NCE | 332.39 | 2.99 | 55.12 | done JP | A, A | 1 |
| MP- 53 | 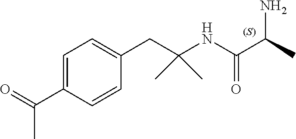 | NCE | 262.35 | 1.43 | 72.19 | done JP | A, B | 0.49 |
| MP- 54 | 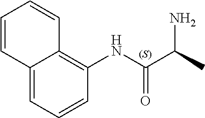 | NCE | 214.27 | 1.66 | 55.12 | done JP | A, C | |
| MP- 55 | 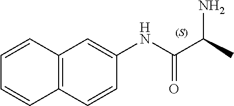 | 720-82-1 1211-51-4 HCl | 214.27 | 1.73 | 55.12 | done JP comm SIGMA +++ S108 1 g | A, B | 5.06 |
| MP- 56 | 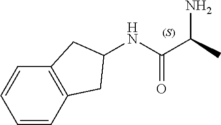 | 1689899-76-0 | 204.27 | 0.68 | 55.12 | done JP anal- getic, anti- perten- sive | A, C | 1.81 |
| MP- 57 | 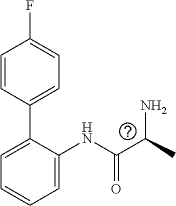 [SMILES: C[C@H](N)C(=O)Nc1ccccc1-c1ccc(F)cc1] | NCE | 258.30 | 1.61 | 55.12 | done JP | A, B | 1.6(2) |
| MP- 58 | 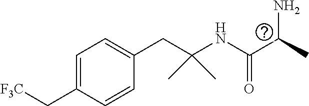 [SMILES: C[C@H](N)C(=O)NC(C)(C)Cc1ccc(CC(F)(F)F)cc1] | NCE | 302.34 | 2.25 | 55.12 | done JP | A, C | 1.28 |
| MP- 59 | 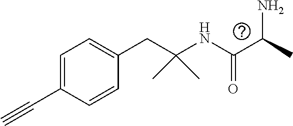 [SMILES: C#Cc1ccc(CC(C)(C)NC(=O)[C@H](C)N)cc1] | NCE | 244.34 | 1.78 | 55.12 | done JP | A, A | 2.49m |
| MP- 60 | 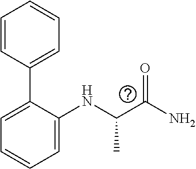 [SMILES: C[C@H](Nc1ccccc1-c1ccccc1)C(N)=O] | 1214795-79-6 | 240.31 | 2.47 | 55.12 | com- mer- cial- bought | A, B | 2 peaks |
| MP- 61 | 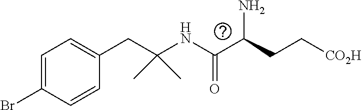 [SMILES: CC(C)(Cc1ccc(Br)cc1)NC(=O)[C@@H](N)CCC(=O)O] | NCE | 357.25 | −0.79 | 92.42 | done JP | A, B | 0.29 |
| MP- 62 | 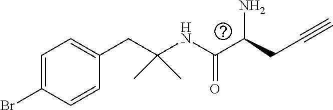 [SMILES: C#CC[C@H](N)C(=O)NC(C)(C)Cc1ccc(Br)cc1] | NCE | 323.23 | 2.42 | 55.12 | done JP | A, B | 1.67 |
| MP- 70 | 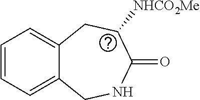 | 443925-80-2 | 234.26 | 0.57 | 67.43 | done YsK | A, A | 0.59 |
| MP- 71 | 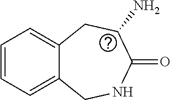 [SMILES: N[C@H]1Cc2ccccc2CNC1=O] | 133899-27-1 | 176.22 | −0.02 | 55.12 | done YsK | B, C | −10.37 |
| MP- 72 | 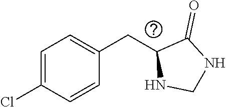 [SMILES: O=C1NCN[C@H]1Cc1ccc(Cl)cc1] | NCE | 210.66 | 1.34 | 41.13 | done YsK | A, A | 11.07(2) |
| MP- 73 | 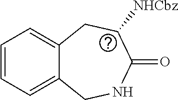 [SMILES: O=C(N[C@H]1Cc2ccccc2CNC1=O)OCc1ccccc1] | NCE | 310.35 | 2.27 | 67.43 | done YsK | a, a | 0.68 |
| MP- 74 | 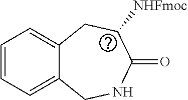 | NCE | 398.46 | 4.09 | 67.43 | done YsK | a, a | 1.55m |
| MP- 75 | 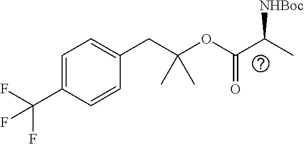 [SMILES: C[C@H](NC(=O)OC(C)(C)C)C(=O)OC(C)(C)Cc1ccc(C(F)(F)F)cc1] | NCE | 389.42 | 5.07 | 64.63 | done YsK | a, a | |
| MP- 76 | 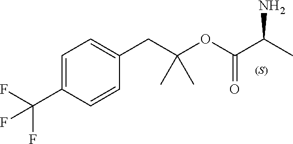 | 735214-27-4 rac | 289.30 | 3.25 | 52.32 | done YsK | A, A | 2.71m |
| MP- 77 | 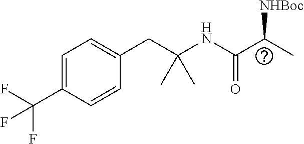 [SMILES: C[C@H](NC(=O)OC(C)(C)C)C(=O)NC(C)(C)Cc1ccc(C(F)(F)F)cc1] | NCE | 388.43 | 4.22 | 67.43 | done YsK | a, a | 1.61 |
| MP- 78 | 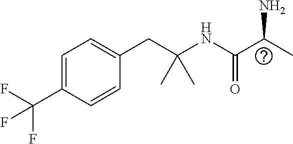 [SMILES: C[C@H](N)C(=O)NC(C)(C)Cc1ccc(C(F)(F)F)cc1] | NCE | 289.73 | 2.33 | 55.12 | done YsK | A, B | 1.93 |
| MP- 79 | 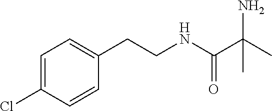 [SMILES: CC(C)(N)C(=O)NCCc1ccc(Cl)cc1] | 1062493-60-4 | 240.73 | 1.83 | 55.12 | done YsK Alclab Chem- icals | A, B | 1.74 |
| MP- 80 | 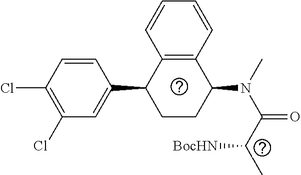 [SMILES: C[C@H](NC(=O)OC(C)(C)C)C(=O)N(C)[C@H]1CC[C@@H](c2ccc(Cl)c(Cl)c2)c2ccccc21] | NCE | 477.43 | 6.5 | 58.64 | done YsK | a, a | NA |
| MP- 81 | 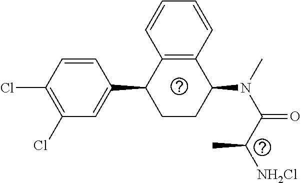 | NCE | 377.31 413.77 | 4.67 | 46.33 | done YsK | B, B | NA |
| MP- 82 | 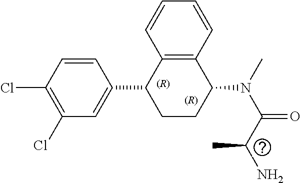 | NCE | 377.31 | 4.67 | 46.33 | done YsK | A, B | NA |
| MP- 83 | 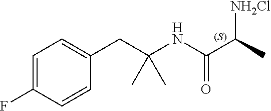 | NCE 1518565- 65-5rac | 222.28 274.76 | 2.68 | 47.7 | done YsK | a, A | NA |
| MP- 84 | 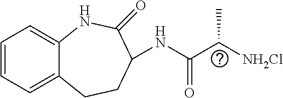 | NCE | 247.30 283.75 | 1.46 | 76.8 | done YsK | a, A | NA |
| MP- 85 | 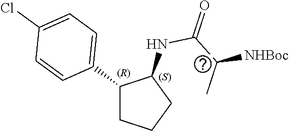 | NCE Germ.Pat.1995 fungicides (iPro) | 366.89 | 4.27 | 67.43 | done YsK | a, a | NA |
| | 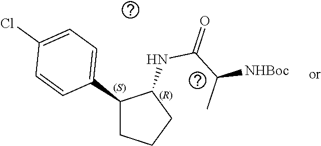 | | | | | | | |
| MP- 86 | 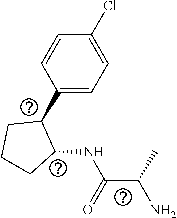 [SMILES: C[C@H](N)C(=O)N[C@@H]1CCC[C@H]1c1ccc(Cl)cc1] | NCE R isomer (NH2) CAS# 1570517-04-2 | 266.77 | 2.45 | 55.12 | done YsK | A, B | NA |
| MP- 87 | 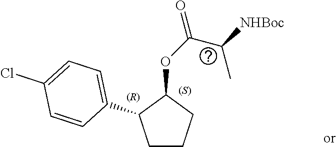 | NCE | 367.87 | 5.13 | 64.63 | done YsK | a, a | NA |
| | 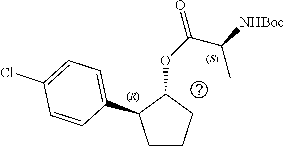 | | | | | | | |
| MP- 88 | 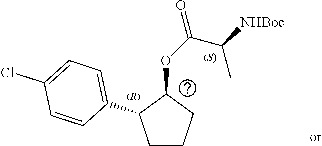 | NCE | 367.87 | 5.13 | 64.63 | done YsK | a, a | NA |
| | 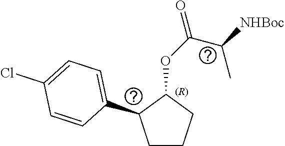 | | | | | | | |
| MP- 89 | 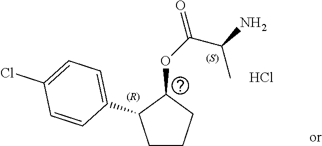 | NCE | 304.21 HCl salt | 3.3 | 52.32 | done YsK | a, A | NA |
| | 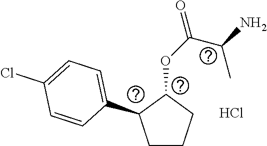 [SMILES: C[C@H](N)C(=O)O[C@@H]1CCC[C@H]1c1ccc(Cl)cc1 ; Cl] | | | | | | | |
| MP- 90 | 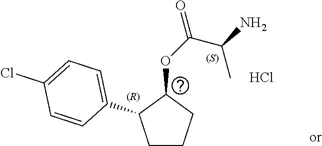 | NCE | 304.21 HCl salt | 3.3 | 52.32 | done YsK | a, a | NA |
| | 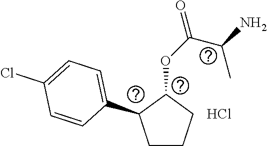 [SMILES: C[C@H](N)C(=O)O[C@@H]1CCC[C@H]1c1ccc(Cl)cc1 ; Cl] | | | | | | | |
| MP- 91 | 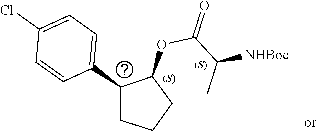 | NCE | 367.87 | 5.13 | 64.63 | done YsK | a, a | NA |
| | 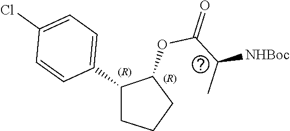 | | | | | | | |
| MP- 92 | 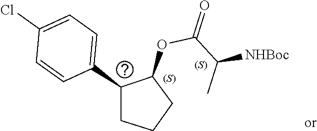 | NCE | 367.87 | 5.13 | 64.63 | done YsK | a, a | NA |
| | 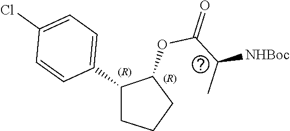 | | | | | | | |
| MP- 93 | 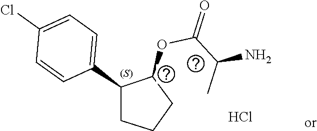 | NCE | 304.21 HCl salt | 3.3 | 52.32 | done YsK | A, C | NA |
| | 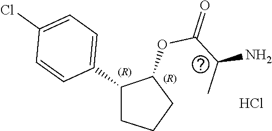 | | | | | | | |
| MP- 94 | 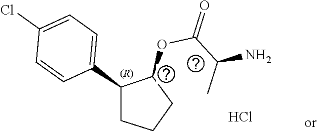 | NCE | 304.21 HCl salt | 3.3 | 52.32 | done YsK | a, A | NA |
| | 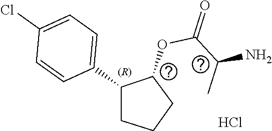 | | | | | | | |
| MP- 95 | 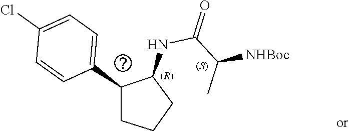 | NCE | 366.89 | 4.27 | 67.43 | done YsK | a, a | NA |
| | 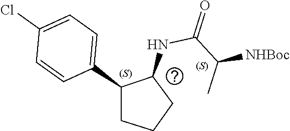 | | | | | | | |
| MP- 96 | 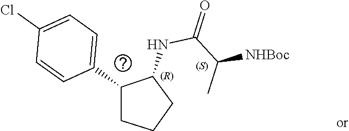 | NCE | 386.89 | 4.27 | 67.43 | done YsK | a, a | NA |
| | 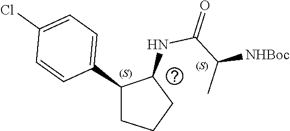 | | | | | | | |
| MP- 97 | 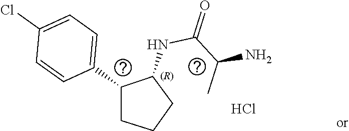 | NCE 1586153-54-9 rac | 303.23 HCl salt | 2.45 | 55.12 | done YsK | A, C | NA |
| | 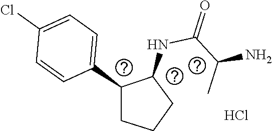 [SMILES: C[C@H](N)C(=O)N[C@H]1CCC[C@H]1c1ccc(Cl)cc1 ; Cl] | | | | | | | |
| MP- 98 | 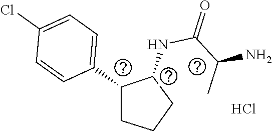 [SMILES: C[C@H](N)C(=O)N[C@@H]1CCC[C@@H]1c1ccc(Cl)cc1 ; Cl] | NCE 1586153-54-9 rac | 303.23 HCl salt | 2.45 | 55.12 | done YsK | a, A | NA |
| | 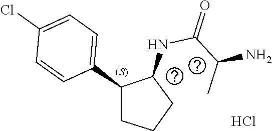 | | | | | | | |
| MP- 99 | 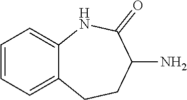 [SMILES: NC1CCc2ccccc2NC1=O] | CAS# 86499-35-6 Aldrich | 176.22 | 0.69 | 55.12 | done YsK | | NA |
| MP- 100 | 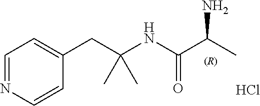 | NCE | 257.76 HCl salt | 0.012 | 67.48 | done YsK | A, A | NA |
| MP- 101 | 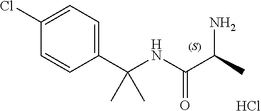 | 1822002-87-8 | 277.19 HCl salt | 2 | 55.12 | done YsK | a, B | NA |
| MP- 102 | 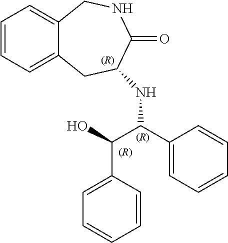 | 168059-25-4 (R, R, S) | 372.47 | 2.96 | 61.36 | done YsK | a, a | 1995 Patent |
| MP- 103 | 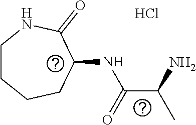 | 1526787-66-5 rac | 235.11 HCl salt | −0.79 | 84.22 | done YsK | a, a | NA |
| MP- 104 | 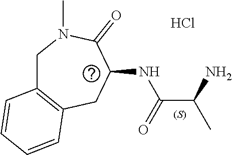 | NCE | 297.78 HCl salt | 0.39 | 75.43 | done YsK | a, A | NA |
| MP- 105 | 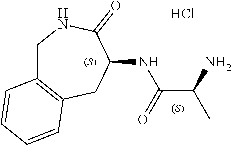 | NCE | 282.79 HCl salt | −0.28 | 84.22 | done YsK | a, a | NA |
| MP- 106 | 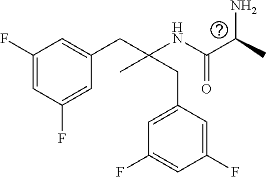 [SMILES: C[C@H](N)C(=O)NC(C)(Cc1cc(F)cc(F)c1)Cc1cc(F)cc(F)c1] | NCE | 404.83 HCl salt | 3.5 | 55.12 | done YsK | a, a | NA |
| MP- 107 | 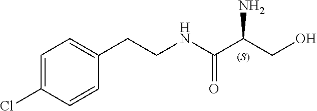 | 155162-38-2 151582-94-4 HCl | 242.7 | 0.5 | 75.35 | done YsK | A, A | NA |
| MP- 108 | 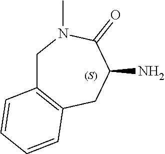 | 147239-68-7 | 190.25 | 0.65 | 46.33 | done YsK | A, A | NA |
| MP- 109 | 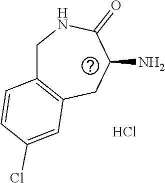 [SMILES: N[C@H]1Cc2cc(Cl)ccc2CNC1=O ; Cl] | NCE | 247.12 | 0.69 | 55.12 | Volo- chem | A, B | NA |

Activity of certain compounds of the diclsoure. SirT1 incrase: a <250%; A >20%; B >50%; C = 100% increase versus control.

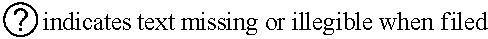

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound represented by formula Ia, Ib, Ic, Id, or Ie or a pharmaceutically acceptable salt thereof: 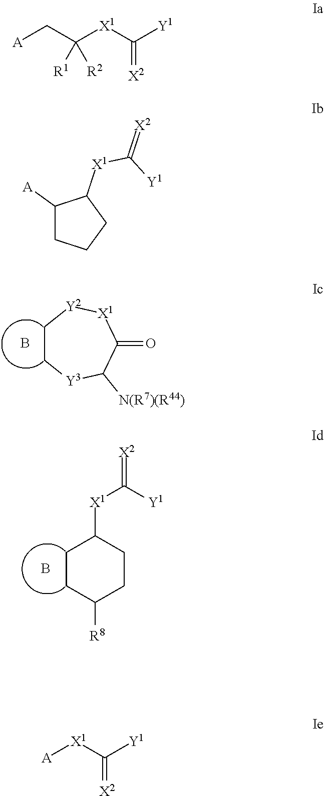
wherein
A and B are each independently, cycloalkyl, aryl, heteroaryl, or heterocyclyl;
X¹ is O, NR⁴¹, S, or C(R³)(R⁴);
X² is O, NR⁴², or S;
Y¹ is alkyl, aminoalkyl, amino, aminoaralkyl, or carbamate; or Y¹ combines with X¹ to form a heterocyclyl;
Y² is NR⁴³ or C(R⁵)(R⁶);
Y³ is a bond or C(R¹³)(R¹⁴);
R¹ and R² are each independently H, alkyl, or aralkyl; or R¹ and R² combine with the carbon that separates them to complete a cycloalkyl or heterocyclyl;
R³, R⁴, R⁵ R⁶, and R¹⁴ are each independently H, alkyl, or aryl;
R⁷ is H, alkyl, aralkyl, carbamate, or alkylacyl;
R⁸ is H, alkyl, cycloalkyl, aryl, heteroaryl, or heterocyclyl;
R¹³ is H, cycloalkyl, aryl, heteroaryl, or heterocyclyl;
R⁴¹, R⁴², R⁴³, and R⁴⁴ are each independently H, alkyl, or aralkyl; and
provided that the compound is not

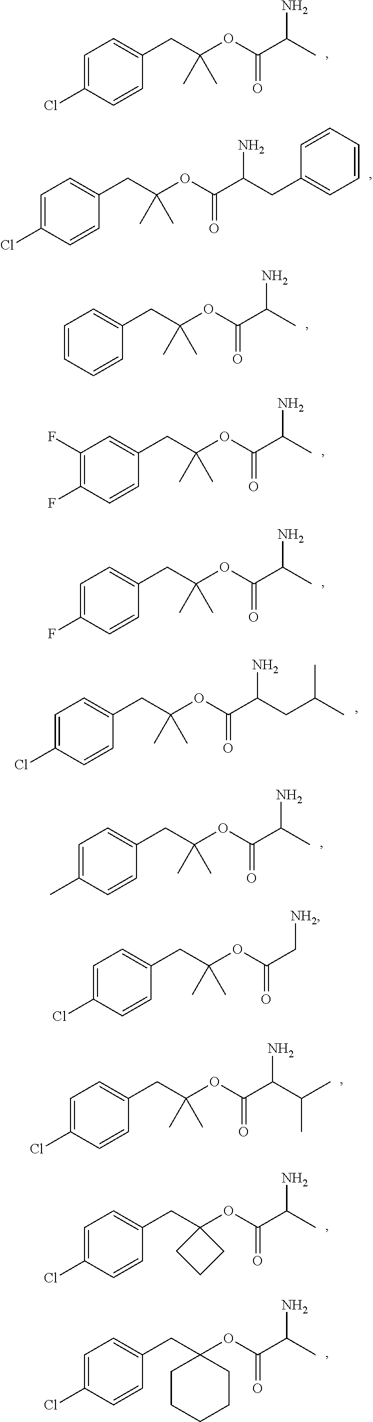

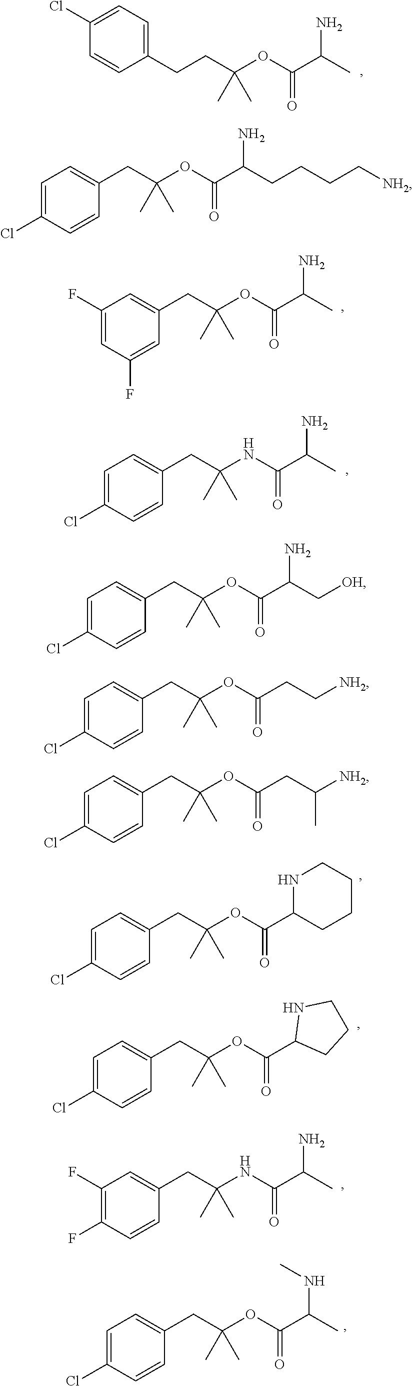

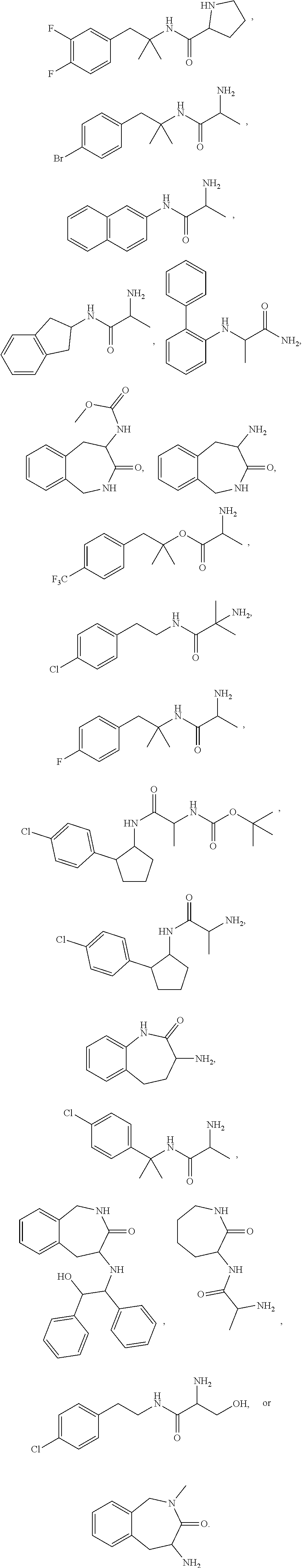

2. (canceled)

3. The compound of claim 1, wherein:
X¹ is O;
X¹ is NR^{1′} and R^{1′} is H or alkyl: or
X¹ is C(R³)(R⁴): R³ is alkyl; and R⁴ is H.

4. (canceled)

5. (canceled)

6. The compound of claim 1, wherein X² is O.

7. (canceled)

8. (canceled)

9. The compound of claim 1, wherein Y¹ is substituted with aryl, carboxyalkyl, alkynylalkyl, or aminoalkylacyl.

10. The compound of claim 1, wherein the compound is represented by formula Ia, Ib, or Ie or a pharmaceutically acceptable salt thereof:

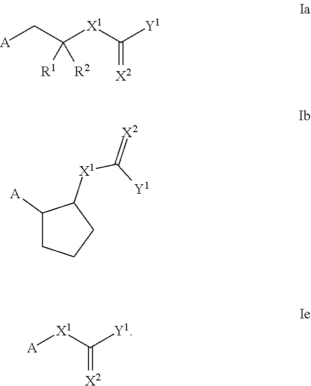

11-17. (canceled)

18. The compound of claim 1, wherein the compound is represented by formula IIa, IIb, IIc, or IIIa or a pharmaceutically acceptable salt thereof: 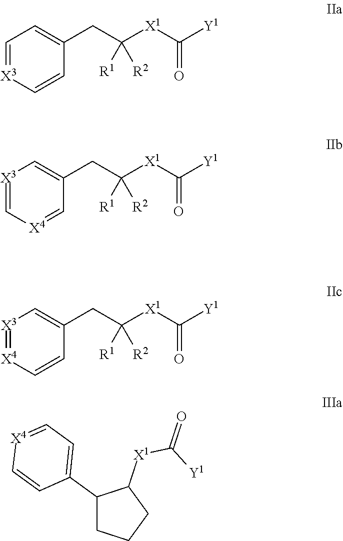
wherein
each X³ is independently N or CR⁹
each X⁴ is each independently N or CR¹⁰; and
each R⁹ and R¹⁰ is selected from H, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, heterocyclylalkyl, heteroaralkyl, sulfonamide, aryl, heteroaryl, heterocyclyl, and aralkyl.

19. The compound of claim 18, wherein the compound is represented by formula IIa, IIb, IIc, or IIIa, or a pharmaceutically acceptable salt thereof:

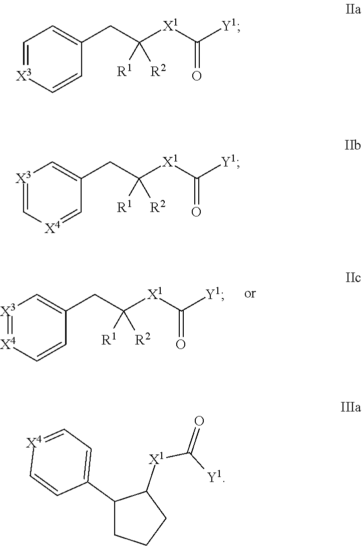

20-28. (canceled)

29. The compound of claim 1, wherein the compound is represented by formula Ia or Ib or a pharmaceutically acceptable salt thereof:

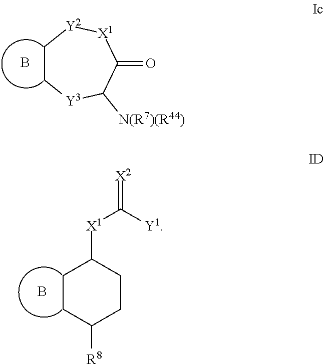

30. (canceled)

31. (canceled)

32. The compound of claim 29, wherein the compound is represented by formula IVa or a pharmaceutically acceptable salt thereof: 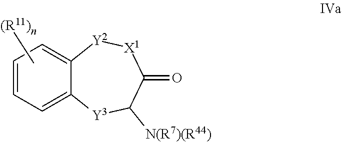
wherein,
each R¹¹ is independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, heterocyclylalkyl, heteroaralkyl, sulfonamide, aryl, heteroaryl, heterocyclyl, and aralkyl; and
n is 0, 1, 2, 3, or 4.

33. The compound of claim 32, wherein R¹¹ is halo.

34-38. (canceled)

39. The compound of claim 32, wherein Y² is C(R⁵)(R⁶).

40. The compound of 1, wherein R⁵ is aryl or H.

41-46. (canceled)

47. The compound of claim 1, wherein R¹³ is aryl.

48. (canceled)

49. The compound of claim 47, wherein R¹³ is substituted with halo.

50. (canceled)

51. (canceled)

52. The compound of claim 1, wherein the compound is represented by formula Va or a pharmaceutically acceptable salt thereof: 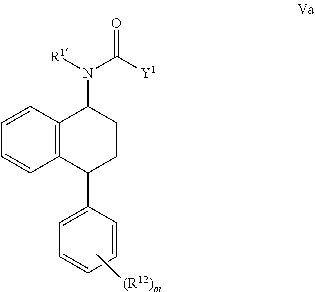
R^{1′} is H or alkyl;
each R¹² is independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, cycloalkyl, heterocyclylalkyl, heteroaralkyl, sulfonamide, aryl, heteroaryl, heterocyclyl, and aralkyl; and
m is 0, 1, 2, 3, or 4.

53. The compound of claim 52, wherein R¹² is halo.

54-57. (canceled)

58. The compound of claim 1, wherein the compound is selected from 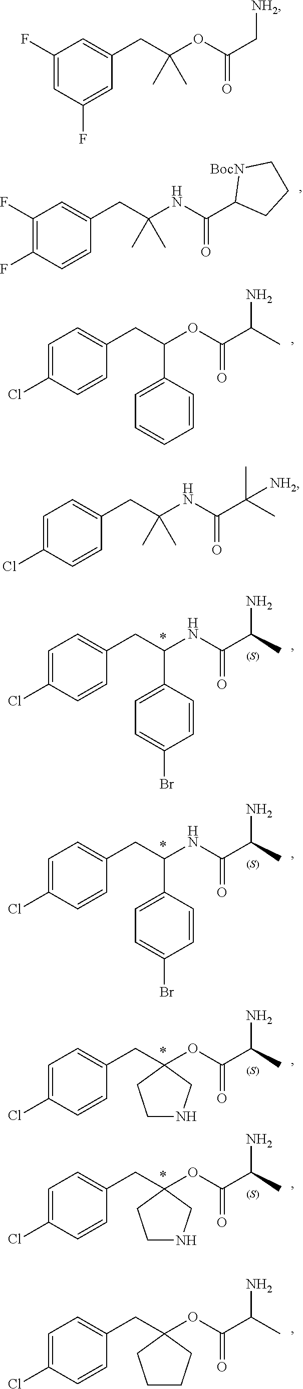 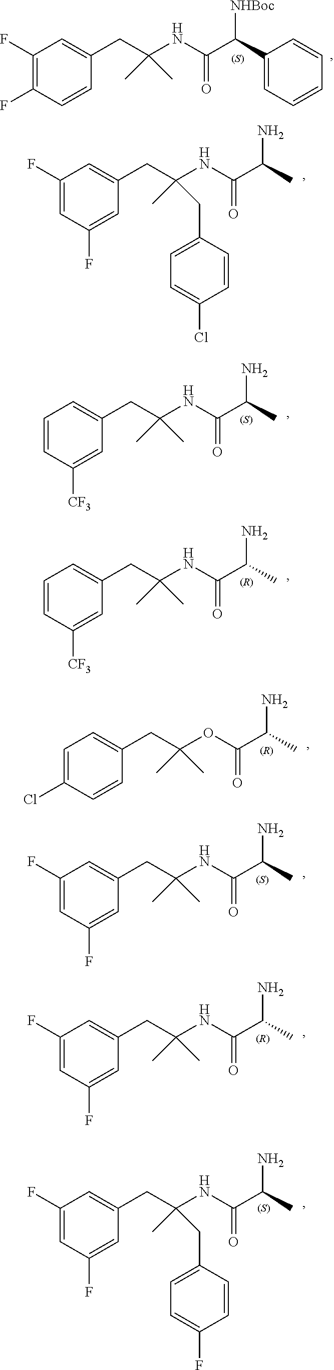 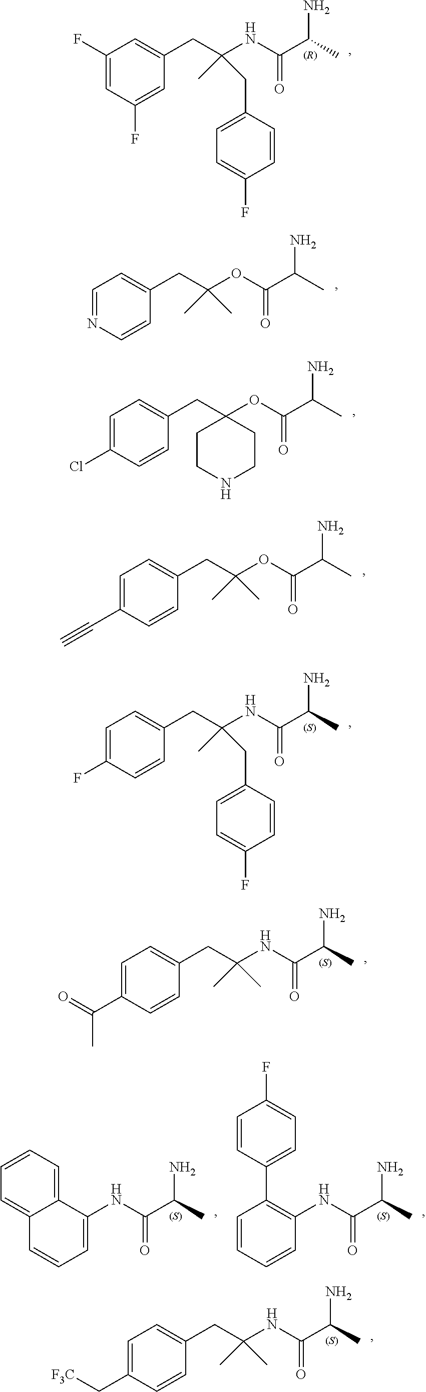 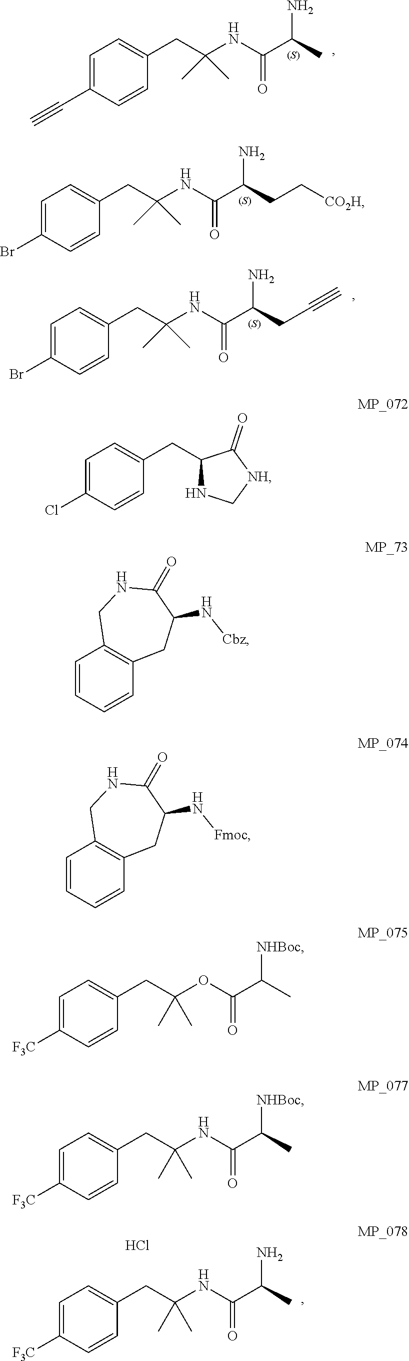 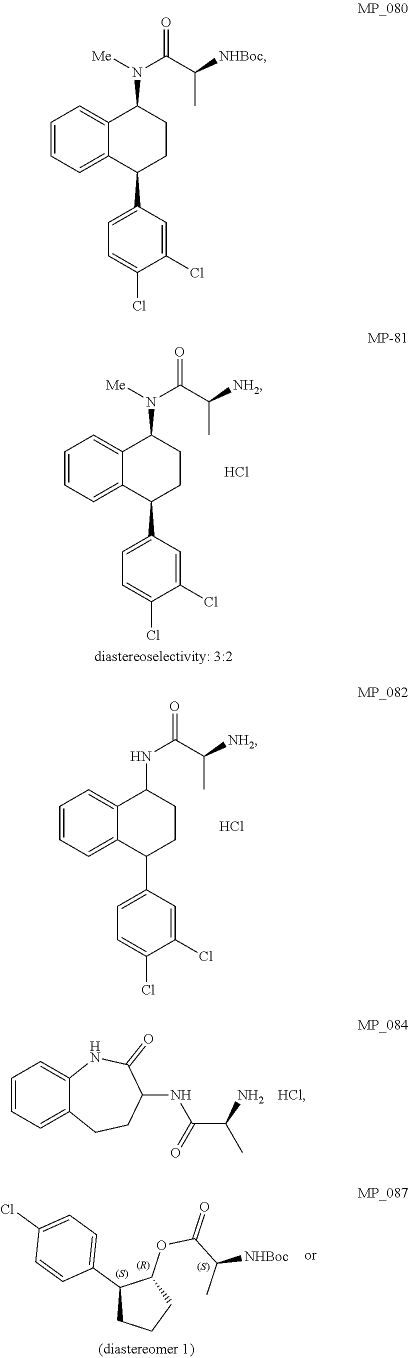 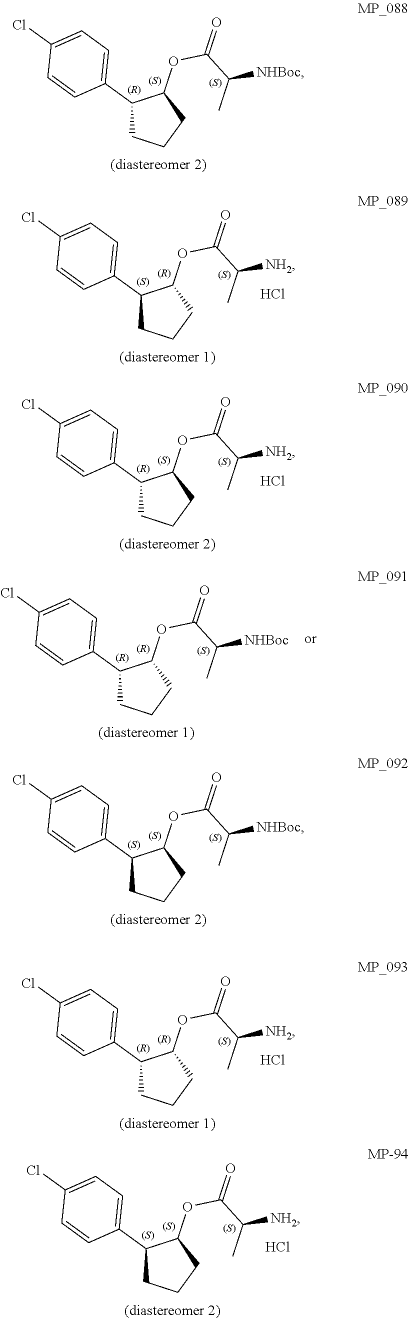 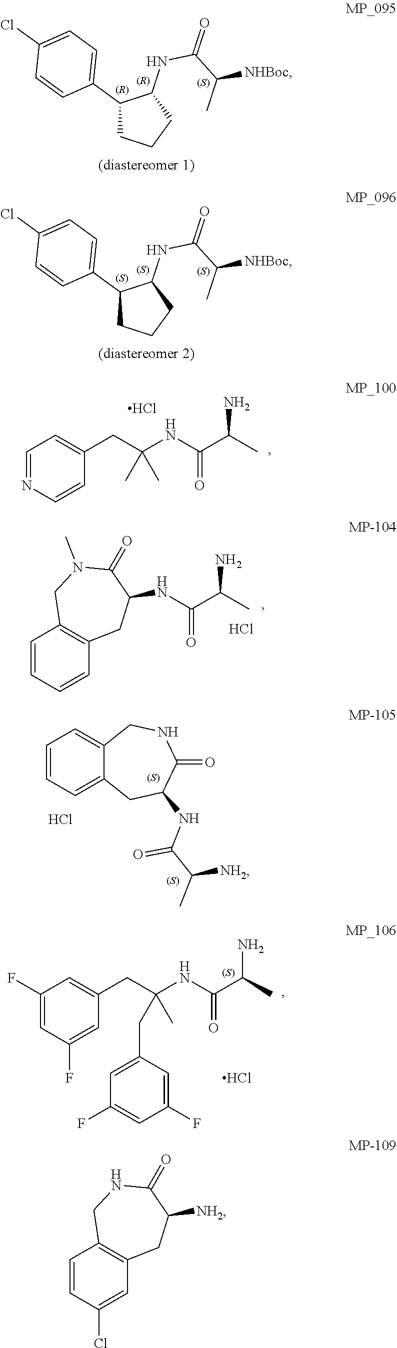 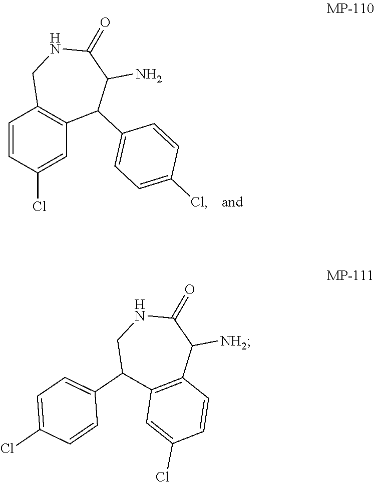 [SMILES: NC1C(=O)NCc2ccc(Cl)cc2C1c1ccc(Cl)cc1 ; "and" ; NC1C(=O)NCC(c2ccc(Cl)cc2)c2cc(Cl)ccc21]
or a pharmaceutically acceptable salt thereof.

59. A pharmaceutical composition comprising a compound of claim 1 and a pharmaceutically acceptable excipient.

60. A method of treating a neurodegenerative disease or disorder in a subject in need thereof, comprising administering a compound of claim 1 or a pharmaceutically acceptable salt thereof or a compound selected from 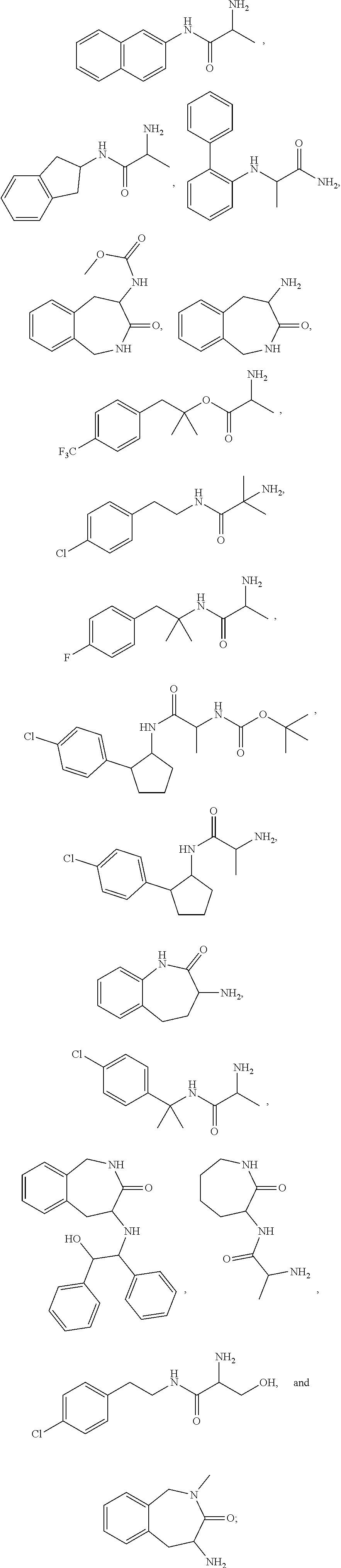
or a pharmaceutically acceptable salt thereof to the subject.

61. (canceled)

62. A method of treating a neurodegenerative disease or disorder in a subject in need thereof, comprising administering a PRMT8 inhibitor to the subject.

63-77. (canceled)
